(19) United States
(12) Patent Application Publication
(10) Pub. No.: US 2009/0203577 A1
(43) Pub. Date: Aug. 13, 2009

(54) Azepinoindole Derivatives As Pharmaceutical Agents

(75) Inventors: Tae-Gon Baik, Foster City, CA (US); Chris A. Buhr, Redwood City, CA (US); Brett B. Busch, San Diego, CA (US); Diva Sze-Ming Chan, Emeryville, CA (US); Brenton T. Flatt, Poway, CA (US); Xiao-Hui Gu, Shanghai (CN); Vasu Jammalamadaka, Pleasanton, CA (US); Richard George Khoury, Redwood City, CA (US); Katherine Lara, San Mateo, CA (US); Sunghoon Ma, Foster City, CA (US); Richard Martin, San Diego, CA (US); Raju Mohan, Encinitas, CA (US); John M. Nuss, Danville, CA (US); Jason Jevious Parks, Sacramento, CA (US); Jason H. Wu, San Diego, CA (US); Bryan K. S. Yeung, Chromos (SG); Wei Xu, Danville, CA (US); Tie-Lin Wang, San Diego, CA (US); Longcheng Wang, Palo Alto, CA (US)

Correspondence Address: MCDONNELL BOEHNEN HULBERT @ BERGHOFF LLP, 300 SOUTH WACKER DRIVE, SUITE 3100, CHICAGO, IL, 60606, US

(73) Assignee: EXELIXIS, INC., South San Francisco, CA (US)

(21) Appl. No.: 12/096,961

(22) PCT Filed: Dec. 12, 2006
(86) PCT No.: PCT/US2006/061928

§ 371 Date: Feb. 13, 2009

Related U.S. Application Data
(60) Provisional application No. 60/750,679, filed on Dec. 15, 2005.
(60) Provisional application No. 60/750,634, filed on Dec. 15, 2005.

(51) Int. Cl. A61K 31/55; C07D 487/04; A61P 3/00; A61K 38/22; A61K 38/28

(52) U.S. Cl. 514/4; 540/580; 514/215; 514/12

(57) ABSTRACT

The present invention relates to compounds of formula I, which exhibit affinity for the farnesoid X receptor.

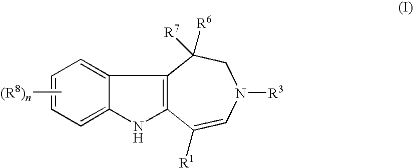

### CROSS-REFERENCE TO RELATED APPLICATIONS

This application claims priority to U.S. Provisional Application No. 60/750,634, filed Dec. 15, 2005, and U.S. Provisional Application No. 60/750,679, filed Dec. 15, 2005, both of which are hereby incorporated by reference in their entirety.

### FIELD OF THE INVENTION

Compounds, compositions and methods are provided for modulating the activity of receptors and for the treatment, prevention, or amelioration of one or more symptoms of disease or disorder related to the activity of the receptors.

### BACKGROUND OF THE INVENTION

### Nuclear Receptors

Nuclear receptors are a superfamily of regulatory proteins that are structurally and functionally related and are receptors for, e.g., steroids, retinoids, vitamin D and thyroid hormones (see, e.g., Evans (1988) Science 240:889-895). These proteins bind to cis-acting elements in the promoters of their target genes and modulate gene expression in response to ligands for the receptors.

Nuclear receptors can be classified based on their DNA binding properties (see, e.g., Evans, supra and Glass (1994) Endocr. Rev. 15:391-407). For example, one class of nuclear receptors includes the glucocorticoid, estrogen, androgen, progestin and mineralocorticoid receptors which bind as homodimers to hormone response elements (HREs) organized as inverted repeats (see, e.g., Glass, supra). A second class of receptors, including those activated by retinoic acid, thyroid hormone, vitamin D₃, fatty acids/peroxisome proliferators (i.e., peroxisome proliferator activated receptor (PPAR)) and ecdysone, bind to HREs as heterodimers with a common partner, the retinoid X receptors (i.e., RXRs, also known as the 9-cis retinoic acid receptors; see, e.g., Levin et al (1992) Nature 355:359-361 and Heyman et al. (1992) Cell 68:397-406).

RXRs are unique among the nuclear receptors in that they bind DNA as a homodimer and are required as a heterodimeric partner for a number of additional nuclear receptors to bind DNA (see, e.g., Mangelsdorf et al. (1995) Cell 83:841-850). The latter receptors, termed the class II nuclear receptor subfamily, include many which are established or implicated as important regulators of gene expression. There are three RXR genes (see, e.g., Mangelsdorf et al. (1992) Genes Dev. 6:329-344), coding for RXRα, -β, and -γ, all of which are able to heterodimerize with any of the class II receptors, although there appear to be preferences for distinct RXR subtypes by partner receptors in vivo (see, e.g., Chiba et al. (1997) Mol. Cell. Biol. 17:3013-3020). In the adult liver, RXRα is the most abundant of the three RXRs (see, e.g., Mangelsdorf et al. (1992) Genes Dev. 6:329-344), suggesting that it might have a prominent role in hepatic functions that involve regulation by class II nuclear receptors. See also, Wan et al. (2000) Mol. Cell. Biol 20:4436-4444.

Orphan Nuclear Receptors

Included in the nuclear receptor superfamily of regulatory proteins are nuclear receptors for which the ligand is known and those which lack known ligands. Nuclear receptors falling in the latter category are referred to as orphan nuclear receptors. The search for activators for orphan receptors has led to the discovery of previously unknown signaling pathways (see, e.g., Levin et al., (1992), supra and Heyman et al., (1992), supra). For example, it has been reported that bile acids, which are involved in physiological processes such as cholesterol catabolism, are ligands for the farnesoid X receptor (infra).

Since it is known that products of intermediary metabolism act as transcriptional regulators in bacteria and yeast, such molecules may serve similar functions in higher organisms (see, e.g., Tomkins (1975) Science 189:760-763 and O'Malley (1989) Endocrinology 125:1119-1120). For example, one biosynthetic pathway in higher eukaryotes is the mevalonate pathway, which leads to the synthesis of cholesterol, bile acids, porphyrin, dolichol, ubiquinone, carotenoids, retinoids, vitamin D, steroid hormones and farnesylated proteins.

Farnesoid X Receptor

The farnesoid X receptor (originally isolated as RIP14 (retinoid X receptor-interacting protein-14), see, e.g., Seol et al. (1995) Mol. Endocrinol. 9:72-85) is a member of the nuclear hormone receptor superfamily and is primarily expressed in the liver, kidney and intestine (see, e.g., Seol et al., supra and Forman et al. (1995) Cell 81:687-693). It functions as a heterodimer with the retinoid X receptor (RXR) and binds to response elements in the promoters of target genes to regulate gene transcription. The farnesoid X receptor-RXR heterodimer binds with highest affinity to an inverted repeat-1 (IR-1) response element, in which consensus receptor-binding hexamers are separated by one nucleotide. The farnesoid X receptor is part of an interrelated process, in that the receptor is activated by bile acids (the end product of cholesterol metabolism) (see, e.g., Makishima et al. (1999) Science 284:1362-1365, Parks et al. (1999) Science 284:1365-1368, Wang et al. (1999) Mol. Cell. 3:543-553), which serve to inhibit cholesterol catabolism. See also, Urizar et al. (2000) J. Biol. Chem. 275:39313-39317.

Nuclear Receptors and Disease

Nuclear receptor activity, including the farnesoid X receptor and/or orphan nuclear receptor activity, has been implicated in a variety of diseases and disorders, including, but not limited to, hyperlipidemia and hypercholesterolemia, and complications thereof, including without limitation coronary artery disease, angina pectoris, carotid artery disease, strokes, cerebral arteriosclerosis and xanthoma, (see, e.g., International Patent Application Publication No. WO 00/57915), osteoporosis and vitamin deficiency (sec, e.g., U.S. Pat. No. 6,316,5103), hyperlipoproteinemia (see, e.g., International Patent Application Publication No. WO 01/60818), hypertriglyceridemia, lipodystrophy, peripheral occlusive disease, ischemic stroke, hyperglycemia and diabetes mellitus (see, e.g., International Patent Application Publication No. WO 01/82917), disorders related to insulin resistance including the cluster of disease states, conditions or disorders that make up “Syndrome X” such as glucose intolerance, an increase in plasma triglyceride and a decrease in high-density lipoprotein cholesterol concentrations, hypertension, hyperuricemia, smaller denser low-density lipoprotein particles, and higher circulating levels of plasminogen activator inhibitor-1, atherosclerosis and gallstones (see, e.g., International Patent Application Publication No. WO 00/37077), disorders of the skin and mucous membranes (see, e.g., U.S. Pat. Nos. 6,184,215 and 6,187,814, and International Patent Application Publication No. WO 98/32444), obesity, acne (see, e.g., International Patent Application Publication No. WO 00/49992), and cancer, cholestasis, Parkinson's disease and Alzheimer's disease (see, e.g., International Patent Application Publication No. WO 00/17334).

The activity of nuclear receptors, including the farnesoid X receptor and/or orphan nuclear receptors, has been implicated in physiological processes including, but not limited to, triglyceride metabolism, catabolism, transport or absorption, bile acid metabolism, catabolism, transport, absorption, re-absorption or bile pool composition, cholesterol metabolism, catabolism, transport, absorption, or re-absorption. The modulation of cholesterol 7α-hydroxylase gene (CYP7A1) transcription (see, e.g., Chiang et al. (2000) J. Biol. Chem. 275:10918-10924), HDL metabolism (see, e.g., Urizar et al. (2000) J. Biol. Chem. 275:39313-39317), hyperlipidemia, cholestasis, and increased cholesterol efflux and increased expression of ATP binding cassette transporter protein (ABC1) (see, e.g., International Patent Application Publication No. WO 00/78972) are also modulated or otherwise affected by the farnesoid X receptor.

Thus, there is a need for compounds, compositions and methods of modulating the activity of nuclear receptors, including the farnesoid X receptor and/or orphan nuclear receptors. Such compounds are useful in the treatment, prevention, or amelioration of one or more symptoms of diseases or disorders in which nuclear receptor activity is implicated.

Commonly owned U.S. Patent Application No. 60/383,574, entitled “Azepinoindole and pyridoindole modulators of nuclear receptors,” filed May 24, 2002, to Martin et al., and U.S. patent application Ser. No. 10/447,302, filed May 27, 2003, to Martin et al., entitled “Azepinoindole and pyridoindole modulators of nuclear receptors,” which are hereby incorporated by reference in their entirety, disclose novel compounds that bind to the farnesoid X receptor.

The present inventors have identified a novel class of such compounds that exhibit extremely high affinity for the farnesoid X receptor, and high potency in vivo. Unexpectedly such compounds show the ability to reduce both plasma triglyceride and cholesterol levels in normal and hyperlipidemic animal models.

### SUMMARY OF THE INVENTION

Compounds for use in pharmaceutical compositions and methods for modulating the activity of nuclear receptors are provided. In particular, compounds for use in compositions and methods for modulating the farnesoid X receptor, and/or orphan nuclear receptors, are provided. In one embodiment, the compounds provided herein are agonists of the farnesoid X receptor. In another embodiment, the compounds provided herein are antagonists of the farnesoid X receptor. In another embodiment, the compounds provided herein are inverse agonists, partial agonists or partial antagonists of the farnesoid X receptor. Agonists that exhibit low efficacy are, in certain embodiments, antagonists.

In one embodiment, the compounds for use in the compositions and methods provided herein have formula (I):

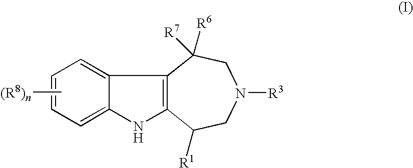

or a pharmaceutically acceptable derivative thereof; wherein:
R¹ is —C(J)R¹¹, —C(J)OR¹¹, or —C(J)NR¹⁰NR¹¹;
J is a direct bond, O or —NR¹⁰;
n is 0 to 4;
R³ is hydrogen, —C(O)R⁹, or CON(R¹¹)(R¹²);

R⁶ or R⁷ is independently optionally substituted alkyl, optionally substituted cycloalkyl or optionally substituted cycloalkylalkyl;

R⁸ is selected from the group consisting of hydroxy, optionally substituted alkyl, optionally substituted alkenyl, optionally substituted alkynyl, halo, haloalkyl, haloalkoxy, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, optionally substituted heteroaralkyl, —OC(O)N(R¹⁵)(R¹⁶), —OC(O)R¹¹, or —OR²⁰;

R⁹ is selected from the group consisting of optionally substituted alkyl, optionally substituted alkenyl, optionally substituted alkynyl, optionally substituted aryl, optionally substituted aralkyl, optionally substituted cycloalkyl, optionally substituted cycloalkylalkyl, optionally substituted heteroaryl, optionally substituted heteroaralkyl, optionally substituted heterocyclylalkyl, optionally substituted heterocyclyl, OR¹⁰ and N(R¹²)(R¹³);

R¹⁰ is independently hydrogen, optionally substituted alkyl, optionally substituted alkenyl or optionally substituted alkynyl; optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, optionally substituted heteroaralkyl;

each R¹¹ is independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, optionally substituted heteroaralkyl, —OR¹⁴ and —N(R¹⁵)(R¹⁶);

R¹² and R¹³ are each independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, and optionally substituted heteroaralkyl; or R¹² and R¹³, together with the nitrogen atom to which they are attached, form an optionally substituted heterocyclyl or an optionally substituted heteroaryl;

R¹⁰, R¹¹ R¹² and R¹³ are selected as in (a) or (b) as follows: (a)R¹⁰, R¹¹ R¹² and R¹³ each independently hydrogen, optionally substituted alkyl, optionally substituted alkenyl, optionally substituted alkynyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl, or optionally substituted heteroaralkyl; or (b) R¹⁰, R¹¹, R¹² and R¹³ together with the atoms to which they are attached, form an optionally substituted heterocyclic ring or an optionally substituted heteroaryl ring; and the others of R¹⁰, R¹¹, R¹², and R¹³, are selected as in (a), above.

each R¹⁴ is independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, optionally substituted heteroaralkyl, —OR¹⁸, —SR¹⁸ and —N(R²⁰)(R²¹);

R¹⁵ and R¹⁶ are each independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, optionally substituted heteroaralkyl, —OR¹⁸, —SR¹⁸ and —N(R²⁰)(R²¹);

or R¹⁵ and R¹⁶, together with the nitrogen atom to which they are attached, form an optionally substituted heterocyclyl ring or an optionally substituted heteroaryl ring;

R¹⁷ is hydrogen, optionally substituted alkyl, optionally substituted alkenyl or optionally substituted alkynyl;

each R¹⁸ is independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, or optionally substituted heteroaralkyl;

R¹⁹ is alkylene or direct bond;

R²⁰ and R²¹ are each independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, or optionally substituted heteroaralkyl; or

R²⁰ and R²¹, together with the nitrogen atom to which they are attached, form an optionally substituted heterocyclyl or an optionally substituted heteroaryl;

each R²² independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, optionally substituted heteroaralkyl, —R¹⁹—OR²³, —R¹⁹—N(R²³)(R²⁴), R¹⁹—C(J)R²³, —R¹⁹—C(J)OR²³, and R¹⁹—C(J)N(R²³)(R²⁴);

each R²³ and R²⁴ is independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, optionally substituted heteroaralkyl, —R¹⁹—OR²⁵, —R¹⁹—N(R²⁵)(R²⁶), —R¹⁹—C(J)R²⁵, —R¹⁹—C(J)OR²⁵, and —R¹⁹—C(j)N(R²⁵)(R²⁶);

or R²³ and R²⁴, together with the nitrogen atom to which they are attached, form an optionally substituted heterocyclyl or an optionally substituted heteroaryl;

each R²⁵ and R²⁶ is independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl and optionally substituted heteroaralkyl;

each R¹-R²⁶, when substituted, are substituted with one or more substituents, each independently selected from Q¹;

where Q¹ is halo, pseudohalo, hydroxy, oxo, thia, nitrile, nitro, formyl, mercapto, amino, hydroxyalkyl, hydroxyalkylaryloxy, hydroxyaryl, hydroxyalkylaryl, hydroxycarbonyl, hydroxycarbonylalkyl, alkyl, haloalkyl, polyhaloalkyl, aminoalkyl, diaminoalkyl, alkonyl containing 1 to 2 double bonds, alkynyl containing 1 to 2 triple bonds, cycloalkyl, cycloalkylalkyl, heterocyclyl, heterocyclylalkyl, aryl, diaryl, hydroxyaryl, alkylaryl, heteroaryl, aralkyl, aralkenyl, aralkynyl, alkylaralkyl, heteroarylalkyl, trialkylsilyl, dialkylarylsilyl, alkyldiarylsilyl, triarylsilyl, alkylidene, arylalkylidene, alkylcarbonyl, alkylarylcarbonyl, arylcarbonyl, heterocyclylcarbonyl, heteroarylcarbonyl, heteroarylalkoxycarbonyl, alkoxycarbonyl, alkoxycarbonylalkyl, alkoxycarbonylaryloxy, aryloxycarbonyl, aryloxycarbonylalkyl, heterocyclylcarbonylalkylaryl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, haloalkoxy, alkoxyaryloxy, alkylaryloxy, diaryloxy, alkylaryloxyalkyl, alkyldiaryloxy, perfluoroalkoxy, alkenyloxy, alkynyloxy, aryloxyalkaoxy, aralkoxyaryloxy, alkylarylcycloalkyloxy, heterocycloxy, alkoxyalkyl, alkoxyalkoxyalkyl, alkylheteroaryloxy, alkylcycloalkoxy, cycloalkyloxy, heterocyclyloxy, aralkoxy, haloaryloxy, heteroaryloxy, alkylheteroaryloxy, alkoxycarbonylheterocycloxy, alkylcarbonylaryloxy, alkylcarbonyloxy, arylcarbonyloxy, aralkylcarbonyloxy, alkoxycarbonyloxy, aryloxycarbonyloxy, alkoxyaryloxy, aralkoxycarbonyloxy, ureido, alkylureido, arylureido, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, haloalkylarylamino, arylamino, diarylamino, alkylarylamino, aralkylamino, alkylcarbonylamino, aralkylcarbonylamino, haloalkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylenedioxyalkyl, dialkylalkylenedioxyalkyl, alkylsulfonylamino, arylsulfonylamino, azido, dialkylphosphonyl, alkylarylphosphonyl, diarylphosphonyl, alkylthio, arylthio, perfluoroalkylthio, hydroxycarbonylalkylthio, thiocyano, isothiocyano, alkylsulfinyl, alkylsulfonyl, arylsulfinyl, arylsulfonyl, aminosulfonyl, alkylaminosulfonyl, dialkylaminosulfonyl, aryl aminosulfonyl, diarylaminosulfonyl or alkylarylaminosulfonyl; or two Q¹ groups, which substitute atoms in a 1,2 or 1,3 arrangement, together form alkylenedioxy (i.e., —O—(CH₂)_{z}—O—), thioalkylenoxy (i.e., —S—(CH₂)_{z}—O—) or alkylenedithioxy (i.e., —S—(CH₂)_{z}—S—) where z is 1 or 2; and

each Q¹ is independently unsubstituted or substituted with one or more substituents, in one embodiment one to three or four substituents, each independently selected from Q², where Q² is halo, pseudohalo, hydroxy, oxo, thia, nitrile, nitro, formyl, mercapto, amino, hydroxyalkyl, hydroxyaryl, hydroxycarbonyl, alkyl, haloalkyl, polyhaloalkyl, aminoalkyl, diaminoalkyl, alkenyl containing 1 to 2 double bonds, alkynyl containing 1 to 2 triple bonds, cycloalkyl, heterocyclyl, aryl, heteroaryl, aralkyl, aralkenyl, aralkynyl, alkoxycarbonyl, aryloxycarbonyl, aralkoxycarbonyl, arylcarbonylalkyl, aminocarbonyl, alkoxy, aryloxy, aralkoxy, alkylenedioxy, amino, aminoalkyl, dialkylamino, arylamino, diarylamino, alkylamino, dialkylamino, haloalkylamino, arylamino, diarylamino, alkylarylamino, aralkylamino, alkoxycarbonylamino, arylcarbonylamino, alkylthio or arylthio.

Such compounds can bind to the farnesoid X receptor with high affinity and modulate its activity. Typically such compounds exhibit an EC₅₀ or IC₅₀ of less than 0.5 μM, and in certain embodiments, less than about 250 nM, 100 nM or 50 nM.

Also of interest are any pharmaceutically-acceptable derivatives, including salts, esters, enol ethers, enol esters, solvates, hydrates and prodrugs of the compounds described herein. Pharmaceutically-acceptable salts, include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc, aluminum, and other metal salts, such as but not limited to sodium hydrogen phosphate and disodium phosphate; and also including, but not limited to, salts of mineral acids, such as but not limited to hydrochloride and sulfates; and salts of organic acids, such as but not limited to acetates, lactates, malates, tartrates, citrates, ascorbates, succinates, butyrates, valerates and fumarates.

Pharmaceutical compositions formulated for administration by an appropriate route and means containing effective concentrations of one or more of the compounds provided herein, or pharmaceutically acceptable derivatives thereof, that deliver amounts effective for the treatment, prevention, or amelioration of one or more symptoms of diseases or disorders that are modulated or otherwise affected by nuclear receptor activity, including the farnesoid X receptor and/or orphan nuclear receptor activity, or in which nuclear receptor activity, including the farnesoid X receptor and/or orphan nuclear receptor activity, is implicated, are also provided. The effective amounts and concentrations are effective for ameliorating any of the symptoms of any of the diseases or disorders.

Methods for treatment, prevention, inhibition or amelioration of one or more symptoms of diseases or disorders mediated by or in which nuclear receptor activity, including the farnesoid X receptor and/or orphan nuclear receptor activity, is implicated, are provided. Such methods include methods of treatment, prevention and amelioration of one or more symptoms of hypercholesterolemia, hyperlipoproteinemia, hypertriglyceridemia, lipodystrophy, hyperglycemia, diabetes mellitus, dyslipidemia, atherosclerosis, gallstone disease, acne vulgaris, acneiform skin conditions, diabetes, Parkinson's disease, cancer, Alzheimer's disease, inflammation, immunological disorders, lipid disorders, obesity, conditions characterized by a perturbed epidermal barrier function, hyperlipidemia, cholestasis, peripheral occlusive disease, ischemic stroke, conditions of disturbed differentiation or excess proliferation of the epidermis or mucous membrane, or cardiovascular disorders, using one or more of the compounds provided herein, or pharmaceutically acceptable derivatives thereof.

Methods of modulating the activity of nuclear receptors, including the farnesoid X receptor and/or orphan nuclear receptors, using the compounds and compositions provided herein are also provided. The compounds and compositions provided herein are active in assays that measure the activity of nuclear receptors, including the farnesoid X receptor and/or orphan nuclear receptors, including the assays provided herein. These methods include inhibiting and up-regulating the activity of nuclear receptors, including the farnesoid X receptor and/or orphan nuclear receptors.

Methods of reducing cholesterol levels in a subject in need thereof by administration of one or more compounds or compositions provided herein are also provided.

Methods of modulating cholesterol metabolism using the compounds and compositions provided herein are provided.

Methods of treating, preventing, inhibiting or ameliorating one or more symptoms of diseases or disorders which are affected by cholesterol, triglyceride, or bile acid levels by administration of one or more of the compounds and compositions provided herein are also provided.

Methods of reducing plasma cholesterol levels and of directly or indirectly modulating cholesterol metabolism, catabolism, synthesis, absorption, re-absorption, secretion or excretion are provided through administering the claimed compounds and compositions provided herein.

Methods of reducing plasma triglyceride levels and of directly or indirectly modulating triglyceride metabolism, catabolism, synthesis, absorption, re-absorption, secretion or excretion are provided through administering the claimed compounds and compositions provided herein.

Methods of reducing bile acid levels and of directly or indirectly modulating bile acid metabolism, catabolism, synthesis, absorption, re-absorption, secretion, excretion, or bile acid pool composition are provided through administering the claimed compounds and compositions provided herein.

Methods of treatment, prevention, inhibition or amelioration of one or more symptoms of a disease or disorder affecting cholesterol, triglyceride, or bile acid levels, or any combination thereof, are provided using the compounds and compositions provided herein.

Methods are provided for the treatment, prevention, inhibition or amelioration of one or more symptoms of, as well as treating the complications of, hyperlipidemia, hypercholesterolemia, hypertriglyceridemia, dyslipidemia and lipodystrophy.

Methods are also provided for the treatment, prevention, or amelioration of one or more symptoms of atherosclerosis, atherosclerotic disease, atherosclerotic disease events and atherosclerotic cardiovascular diseases.

Additionally, the instant invention also provides a method for preventing, inhibiting or reducing the risk of a first or subsequent occurrence of an atherosclerotic disease event comprising the administration of a prophylactically effective amount of a compound or composition of the present invention to a patient at risk for such an event. The patient may already have atherosclerotic disease at the time of administration, or may be at risk for developing it.

In another aspect, the method of this invention also serves to remove cholesterol from tissue deposits such as atherosclerotic plaques or xanthomas in a patient with atherosclerotic disease manifest by clinical signs such as angina, claudication, bruits, one that has suffered a myocardial infarction or transient ischemic attack, or one diagnosed by angiography, sonography or MRI.

Methods of treatment, prevention, inhibition or amelioration of one or more of the symptoms of diabetes mellitus, as well as treating the complications of diabetes mellitus, are also provided using the compounds and compositions provided herein.

Methods of treatment, prevention, inhibition or amelioration of one or more of the symptoms of insulin insensitivity or resistance as well as treating the complications of insulin insensitivity or resistance are also provided using the compounds and compositions provided herein.

Methods of treatment, prevention, inhibition or amelioration of one or more of the symptoms of hyperglycemia as well as treating the complications of hyperglycemia are also provided using the compounds and compositions provided herein.

Methods of treatment, prevention, inhibition or amelioration of any disorders related to diabetes, hyperglycemia or insulin resistance including the cluster of disease states, conditions or disorders that make up “Syndrome X” are provided.

Additionally the instant invention also provides a method for preventing, inhibiting or reducing the risk of developing hyperglycemia, insulin resistance or diabetes in a patient, comprising the administration of a prophylactically effective amount of a compound or composition of the present invention to a patient at risk for such an event.

Further provided herein are methods for the treatment, prevention, inhibition or amelioration of one or more symptoms of cholestasis, as well as for the treatment of the complications of cholestasis by administering a compound or composition provided herein.

Accordingly, compounds or compositions provided herein may be used for the treatment, prevention, inhibition or amelioration of one or more symptoms of intrahepatic or extrahepatic cholestasis, including without limitation, biliary atresia, obstetric cholestasis, neonatal cholestasis, drug induced cholestasis, cholestasis arising from Hepatitis C infection, chronic cholestatic liver disease such as primary biliary cirrhosis (PBC) and primary sclerosing cholangitis (PSC).

Further provided by this invention are methods for treating obesity, as well as treating the complications of obesity, by administering a compound or composition of the present invention.

Also contemplated herein is combination therapy using more or more compounds or compositions provided herein, or a pharmaceutically acceptable derivative thereof, in combination with one or more of the following: antihyperlipidemic agents, plasma HDL-raising agents, antihypercholesterolermic agents, cholesterol biosynthesis inhibitors (such as HMG CoA reductase inhibitors, such as lovastatin, simvastatin, pravastatin, fluvastatin, atorvastatin and rivastatin), acyl-coenzyme A: cholesterol acytransferase (ACAT) inhibitors, probucol, raloxifene, nicotinic acid, niacinamide, cholesterol absorption inhibitors, bile acid sequestrants (such as anion exchange resins, or quaternary amines (e.g., cholestyramine or colestipol)), low density lipoprotein receptor inducers, clofibrate, fenofibrate, benzofibrate, cipofibrate, gemfibrizol, vitamin B₆, vitamin B₁₂, anti-oxidant vitamins, β-blockers, anti-diabetes agents, angiotensin II antagonists, angiotensin converting enzyme inhibitors, platelet aggregation inhibitors, fibrinogen receptor antagonists, LXR α or β agonists, antagonists or partial agonists, aspirin or fibric acid derivatives. The compound, or composition provided herein, or pharmaceutically acceptable derivative thereof, is administered simultaneously with, prior to, or after administration of one or more of the above agents. Pharmaceutical compositions containing a compound provided herein and one or more of the above agents are also provided.

In practicing the methods, effective amounts of the compounds or compositions containing therapeutically effective concentrations of the compounds, which are formulated for systemic delivery, including parenteral, oral, or intravenous delivery, or for local or topical application for the treatment of nuclear receptor, including the farnesoid X receptor and/or orphan nuclear receptor, mediated diseases or disorders, or diseases or disorders in which nuclear receptor activity, including the farnesoid X receptor and/or orphan nuclear receptor activity, is implicated, including, but not limited to, hypercholesterolemia, hyperlipoproteinemia, hypertriglyceridemia, lipodystrophy, hyperglycemia, diabetes mellitus, dyslipidemia, atherosclerosis, gallstone disease, acne vulgaris, acneiform skin conditions, diabetes, Parkinson's disease, cancer, Alzheimer's disease, inflammation, immunological disorders, lipid disorders, obesity, conditions characterized by a perturbed epidermal barrier function, hyperlipidemia, cholestasis, peripheral occlusive disease, ischemic stroke, conditions of disturbed differentiation or excess proliferation of the epidermis or mucous membrane, or cardiovascular disorders, are administered to an individual exhibiting the symptoms of these diseases or disorders. The amounts are effective to ameliorate or eliminate one or more symptoms of the diseases or disorders.

Articles of manufacture containing packaging material, a compound or composition, or pharmaceutically acceptable derivative thereof, provided herein, which is effective for modulating the activity of nuclear receptors, including the farnesoid X receptor and/or orphan nuclear receptors, or for treatment, prevention or amelioration of one or more symptoms of nuclear receptor, including the farnesoid X receptor and/or orphan nuclear receptor mediated diseases or disorders, or diseases or disorders in which nuclear receptor activity, including the farnesoid X receptor and/or orphan nuclear receptor activity, is implicated, within the packaging material, and a label that indicates that the compound or composition, or pharmaceutically acceptable derivative thereof, is used for modulating the activity of nuclear receptors, including the farnesoid X receptor and/or orphan nuclear receptors, or for treatment, prevention or amelioration of one or more symptoms of nuclear receptor, including the farnesoid X receptor and/or orphan nuclear receptor, mediated diseases or disorders, or diseases or disorders in which nuclear receptor activity, including the farnesoid X receptor and/or orphan nuclear receptor activity, is implicated, are provided.

### BRIEF DESCRIPTION OF THE DRAWINGS

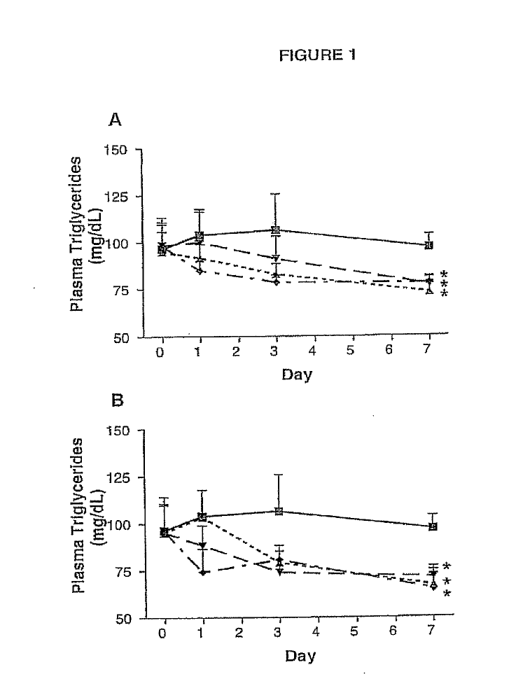

### FIG. 1

### Compound Effects in Normolipidemic Mice

FIG. 1 shows plasma triglyceride levels in male C57BL/6 mice either treated with Compound A (FIG. 1A) or Compound B (FIG. 1B) daily by oral gavage at doses of 0.1 mg/kg/day (filled triangles), 1.0 mg/kg/day (Upside down filled triangles) or 10 mg/kg/day (Diamonds) for seven days (n=6/group) compared to vehicle alone (filled squares).

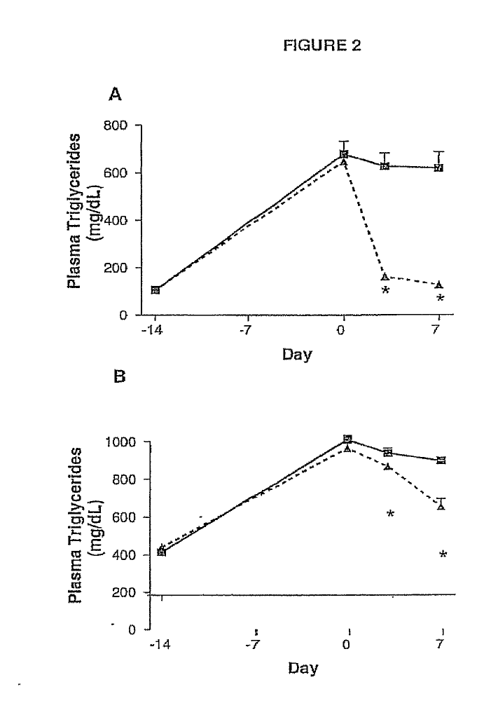

### FIG. 2

### Compound Effects in Diet-Induced Hyperlipidemic LDLR^{-/-} Mice

FIG. 2A shows plasma triglyceride levels in male LDLR−/− mice fed a “Western” diet (˜21% fat, 0.02% cholesterol w/w) ad libitum, for two weeks prior to and during treatment with Compound C daily by oral gavage at a dose of 10 mg/kg/day for 7 days (n=9-10/group) (filled triangles) compared to vehicle-treated controls (filled squares). FIG. 2B shows plasma cholesterol levels in the same mice treated with Compound C (filled triangles) compared to vehicle-treated controls (filled squares).

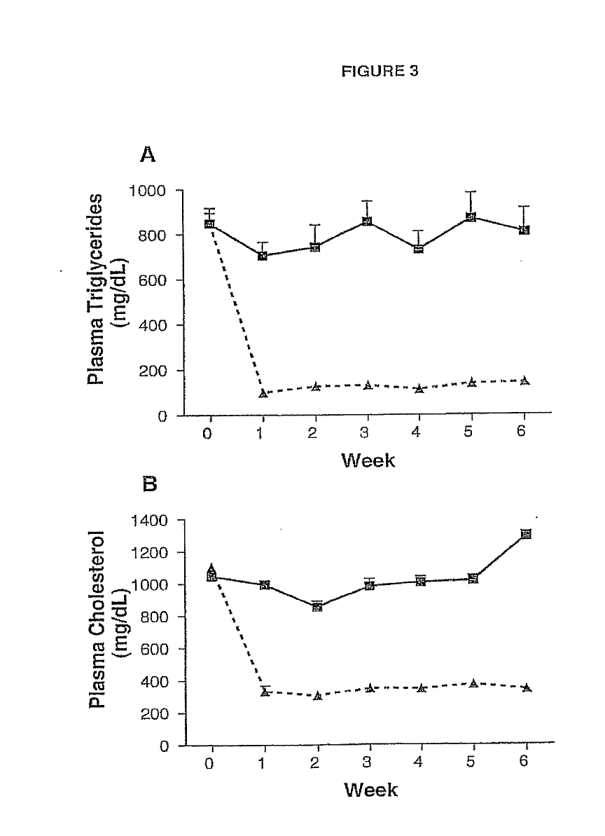

### FIG. 3

### Longer Term Effects of Compound C in Diet-Induced Hyperlipidemic LDLR^{-/-} Mice

FIG. 3A shows plasma triglyceride levels in male LDLR^{-/-} mice fed a “Western” diet (˜21% fat, 0.02% cholesterol w/w) ad libitum, for eight weeks prior to and during treatment with Compound B by oral gavage at a dose of 10 mg/kg/day for 6 weeks (n=12-16/group) compared to vehicle-treated controls (filled squares). FIG. 3B shows plasma cholesterol levels in the same mice treated with Compound B (filled triangles) compared to the vehicle-treated controls (filled squares).

### DETAILED DESCRIPTION OF THE INVENTION

### A. Definitions

Unless defined otherwise, all technical and scientific terms used herein have the same meaning as is commonly understood by one of ordinary skill in the art to which this invention belongs. All patents, applications, published applications and other publications are incorporated by reference in their entirety. In the event that there are a plurality of definitions for a term herein, those in this section prevail unless stated otherwise.

As used herein, a nuclear receptor is a member of a superfamily of regulatory proteins that are receptors for, e.g., steroids, retinoids, vitamin D and thyroid hormones. These proteins bind to cis-acting elements in the promoters of their target genes and modulate gene expression in response to a ligand therefor. Nuclear receptors may be classified based on their DNA binding properties. For example, the glucocorticoid, estrogen, androgen, progestin and mineralocorticoid receptors bind as homodimers to hormone response elements (HREs) organized as inverted repeats. Another example are receptors, including those activated by retinoic acid, thyroid hormone, vitamin D₃, fatty acids/peroxisome proliferators and ecdysone, that bind to HREs as heterodimers with a common partner, the retinoid X receptor (RXR). Among the latter receptors is the farnesoid X receptor.

As used herein, an orphan nuclear receptor is a gene product that embodies the structural features of a nuclear receptor that was identified without any prior knowledge of their association with a putative ligand and/or for which the natural ligand is unknown. Under this definition, orphan nuclear receptors include, without limitation, farnesoid X receptors, liver X receptors (LXR α & β), retinoid X receptors (RXR α, β & γ), and peroxisome proliferator activator receptors (PPAR α, β & γ) (see, Giguere, Endocrine Reviews (1999), Vol. 20, No. 5: pp. 689-725).

As used herein, farnesoid X receptor refers to all mammalian forms of such receptor including, for example, alternative splice isoforms and naturally occurring isoforms (see, e.g. Huber et al, Gene (2002), Vol. 290, pp. 35-43). Representative farnesoid X receptor species include, without limitation the rat (GenBank Accession No. NM_{—}021745), mouse (Genbank Accession No. NM_{—}009108), and human (GenBank Accession No. NM_{—}005123) forms of the receptor.

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization. The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates. Pharmaceutically acceptable esters include, but are not limited to, alkyl, alkenyl, alkynyl, aryl, heteroaryl, aralkyl, heteroaralkyl, cycloalkyl and heterocyclyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C═C(OR) where R is hydrogen, alkyl, alkenyl, alkynyl, aryl, heteroaryl, aralkyl, heteroaralkyl, cycloalkyl or heterocyclyl. Pharmaceutically acceptable enol esters include, but are not limited to, derivatives of formula C═C(OC(O)R) where R is hydrogen, alkyl, alkenyl, alkynyl, aryl, heteroaryl, aralkyl, heteroaralkyl, cycloalkyl or heterocyclyl. Pharmaceutically acceptable solvates and hydrates are complexes of a compound with one or more solvent or water molecules, or 1 to about 100, or 1 to about 10, or one to about 2, 3 or 4, solvent or water molecules.

Derivatization of drugs containing a carbonyl, carboxylic, amines, amidines is well known to one of ordinary skill in the art. Camille Georges Wennuth's “Practice of Medicinal Chemistry”, Second Ed. (2003); Shan, D., Nicolau, M., Buchardt, R., Wang, B., J. Pharm. Sci, 86(7):765-767 (1997); Prodrug strategies based on Intramolecular Cyclization Reaction; Prodrugs are converted to active drugs by metabolic transformation. Various mechanisms of drug activation such as carrier linked prodrugs for several functional groups, carrier linked bipartite prodrugs, or tripartite drugs are disclosed. DeClerq, E. et al., Anitviral Drugs-development of successful prodrug strategies for antiviral chemotherapy, Brit. J. Pharm., 147:1-11 (2006); Silverman, R., Organic Chemistry of Drug Design and Drug Action, 2^{nd} ed.

Compounds of this invention having an —OH, —NH—, —SH, or —COOH moiety can have attached therethrough a prodrug-forming moiety which is removed by metabolic processes to release the compounds of this invention having the freed —OH, —NH—, —SH, or —COOH moiety in vivo. Prodrugs are useful for adjusting such pharmacokinetic properties of the compounds of this invention or the salts thereof, as solubility, hydrophobicity, absorption in the gastrointestinal tract, bioavailability, tissue penetration, and rate of clearance. Those of ordinary skill in the art have the knowledge and means to accomplish this without undue experimentation. Various forms of prodrugs are well known in the art. For examples of such prodrug derivatives, see, e.g., a) Design of Prodrugs, Bundgaard, A. Ed., Elsevier, 1985 and Method in Enzymology, Widder, K. et al., Ed.; Academic, 1985, vol. 42, p. 309 396.

As used herein, treatment means any manner in which one or more of the symptoms of a disease or disorder are ameliorated or otherwise beneficially altered. Treatment also encompasses any pharmaceutical use of the compositions herein, such as use for treating a nuclear receptor mediated diseases or disorders, or diseases or disorders in which nuclear receptor activity, including the farnesoid X receptor or orphan nuclear receptor activity, is implicated.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, IC₅₀ refers to an amount, concentration or dosage of a particular test compound that achieves a 50% inhibition of a maximal response, such as modulation of nuclear receptor, including the farnesoid X receptor, activity, in an assay that measures such response.

As used herein, EC₅₀ refers to a dosage, concentration or amount of a particular test compound that elicits a dose-dependent response at 50% of maximal expression of a particular response that is induced, provoked or potentiated by the particular test compound.

As used herein, a prodrug is a compound that, upon in vivo administration, is metabolized by one or more steps or processes or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

The term “prodrugs”, as the term is used herein, are intended to include any covalently bonded carriers which release an active parent drug of the present invention in vivo when such prodrug is administered to a patient. Since prodrugs are known to enhance numerous desirable qualities of pharmaceuticals (i.e., solubility, bioavailability, manufacturing, etc.) the compounds of the present invention may be delivered inprodrug form. Thus, the skilled artisan will appreciate that the present invention encompasses prodrugs of the presently claimed compounds, methods of delivering the same, and compositions containing the same. Prodrugs of the present invention are prepared by modifying functional groups present in the compound in such a way that the modifications are cleaved, either in routine manipulation or in vivo, to form the parent compound. The transformation in vivo may be, for example, as the result of some metabolic process, such as chemical or enzymatic hydrolysis of a carboxylic, phosphoric or sulphate ester, or reduction or oxidation of a susceptible functionality. Prodrugs include compounds of the present invention wherein a hydroxy, amino, or sulfhydryl group is bonded to any group that, when the prodrug of the present invention is administered to a patient, it cleaves to form a free hydroxyl, free amino, or free sulfydryl group, respectively. Functional groups which may be rapidly transformed, by metabolic cleavage, in vivo form a class of groups reactive with the carboxyl group of the compounds of this invention. They include, but are not limited to such groups as alkanoyl (such as acetyl, propionyl, butyryl, and the like), unsubstituted and substituted aroyl (such as benzoyl and substituted benzoyl), alkoxycarbonyl (such as ethoxycarbonyl), trialkylsilyl (such as trimethyl- and triethysilyl), monoesters formed with dicarboxylic acids (such as succinyl), and the like. Because of the ease with which the metabolically cleavable groups of the compounds useful according to this invention are cleaved in vivo, the compounds bearing such groups can act as prodrugs. The compounds bearing the metabolically cleavable groups have the advantage that they may exhibit improved bioavailability as a result of enhanced solubility and/or rate of absorption conferred upon the parent compound by virtue of the presence of the metabolically cleavable group. A thorough discussion of prodrugs is provided in the following: Design of Prodrugs, H. Bundgaard, ed., Elsevier, 1985; Methods in Enzymology, K. Widder et al., Ed., Academic Press, 42, p. 309 396, 1985; A Textbook of Drug Design and Development, Krogsgaard-Larsen and H. Bundgaard, ed., Chapter 5; “Design and Applications of Prodrugs” p. 113 191, 1991; Advanced Drug Delivery Reviews, H. Bundgard, 8, p. 138, 1992; Journal of Pharmaceutical Sciences, 77, p. 285, 1988; Chem. Pharm. Bull., N. Nakeya et al, 32, p. 692, 1984; Pro-drugs as Novel Delivery Systems, T. Higuchi and V. Stella, Vol. 14 of the A.C.S. Symposium Series, and Bioreversible Carriers in Drug Design, Edward B. Roche, ed., American Pharmaceutical Association and Pergamon Press, 1987; Bundgaard, H., Advanced Drug Delivery Review, 1992, 8, 1 38. and are incorporated herein by reference.

When the compounds described herein contain olefinic double bonds or other centers of geometric asymmetry, and unless specified otherwise, it is intended that the compounds include both E and Z geometric isomers. Likewise, all tautomeric forms are also intended to be included.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. In the case of amino acid residues, such residues may be of either the L- or D-form. The configuration for naturally occurring amino acid residues is generally L. When not specified the residue is the L form. As used herein, the term “amino acid” refers to α-amino acids which are racemic, or of either the D- or L-configuration. The designation “d” preceding an amino acid designation (e.g., dAla, dSer, dVal, etc.) refers to the D-isomer of the amino acid. The designation “dl” preceding an amino acid designation (e.g., dlpip) refers to a mixture of the L- and D-isomers of the amino acid. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

Optically active (+) and (−), (R)- and (S)-, or (D)- and (L)-isomers may be prepared using chiral synthons or chiral reagents, or resolved using conventional techniques, such as reverse phase HPLC.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, “alkyl”, “alkenyl” and “alkynyl” are straight or branched hydrocarbon chains, and if not specified, contain from 1 to 20 carbons or 2 to 20 carbons, preferably from 1 to 16 carbons or 2 to 16 carbons. Alkenyl carbon chains having 2 to 20 carbons, in certain embodiments, contain 1 to 8 double bonds and alkenyl carbon chains having 2 to 16 carbons, in certain embodiments, contain 1 to 5 double bonds. Alkynyl carbon chains having 2 to 20 carbons, in certain embodiments, contain 1 to 8 triple bonds, and the alkynyl carbon chains having 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, allyl (propenyl) and propargyl (propynyl). As used herein, lower alkyl, lower alkenyl, and lower alkynyl refer to carbon chains having from about 1 or about 2 carbons up to about 6 carbons. As used herein, “alk(en)(yn)yl” refers to an alkyl group containing at least one double bond and at least one triple bond.

As used herein, “alkylene” refers to a straight, branched or cyclic divalent aliphatic hydrocarbon group wherein the alkylene is attached to the rest of the molecule through two different bonds in the alkylene. In one embodiment the alkylene has from 1 to about 20 carbon atoms, in another embodiment the alkylene has from 1 to 12 carbons. The term “lower alkylene” refers to alkylene groups having 1 to 6 carbons. In certain embodiments, alkylene groups are lower alkylene, including alkylene of 1 to 3 carbon atoms.

As used herein, “alkylidene” refers to a divalent group, such as ═CR^{p}R^{q}, wherein the alkylidene is attached to an atom of another group through the same carbon in the alkylidene, forming a double bond. Alkylidene groups include, but are not limited to, methylidene (═CH₂) and ethylidene (═CHCH₃). Alkylidenes may be optionally substituted with halo, cyano, nitro, haloalkyl or pseudohalo substituents. As used herein, “arylalkylidene” refers to an alkylidene group in which either R^{p} or R^{q} is an aryl group; “heteroaralkylidene” refers to an alkylidene group in which either R^{p} or R^{q} is a heteroaryl group; “cycloalkylidene” refer to an alkylidene group wherein R^{p} and R^{q}, together with the carbon to which they are attached, form a cycloalkyl group, or wherein at least one of R^{p} and R^{q} is a cycloalkyl ring; and “heterocyclylidene” refer to an alkylidene group wherein R^{p} and R^{q}, together with the carbon to which they are attached, form a heterocyclyl group, or wherein at least one of R^{p} and R^{q} is a heterocyclyl ring.

As used herein, “amidino” refers to a radical having the formula —C(═NR^{m})N(Rⁿ)R^{o} where R^{m}, Rⁿ and R^{o} are each independently hydrogen or alkyl.

As used herein, “aralkyl” refers to a radical of the formula —R^{a}R^{d} where R^{a} is an alkyl radical as defined above, substituted by R^{d}, an aryl radical, as defined herein, e.g., benzyl. Both the alkyl and aryl radicals may be optionally substituted as described herein.

As used herein, “aryl” refers to aromatic monocyclic or multicyclic ring system containing from 6 to 19 carbon atoms, where the ring system may be partially or fully saturated. Aryl groups include, but are not limited to groups such as unsubstituted or substituted fluorenyl, unsubstituted or substituted phenyl, and unsubstituted or substituted naphthyl.

As used herein, “cycloalkyl” refers to a saturated mono- or multi-cyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion. “Cycloalk(en)(yn)yl” refers to a cycloalkyl group containing at least one double bond and at least one triple bond.

As used herein, “cycloalkylalkyl” refers to a radical of the formula —R^{a}R^{b} where R^{a} is an alkyl radical as defined above and R^{b} is a cycloalkyl radical as defined above. The alkyl radical and the cycloalkyl radical may be optionally substituted as defined above.

As used herein, “guanidino” refers to a radical having the formula —N(R^{p})C(═NR^{q})NR^{r}R^{s} wherein R^{p}, R^{q}, R^{r} and R^{s} are each independently hydrogen or alkyl.

As used herein, “heteroaralkyl” refers to a radical of the formula —R^{a}R^{e} where R^{a} is an alkyl radical as defined above and R^{w} is a heteroaryl radical as defined herein. The alkyl radical and the heterocyclyl radical may be optionally substituted as defined herein.

As used herein, “heteroaryl” refers to a monocyclic or multicyclic aromatic heterocyclyl, as defined herein, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrimidinyl, tetrazolyl, thienyl, pyridyl, pyrrolyl, thiazolyl, isothiazolyl, oxazolyl, isoxazolyl, triazolyl, quinolinyl and isoquinolinyl.

As used herein, a “heteroarylium” group is a heteroaryl group that is positively charged on one or more of the heteroatoms.

As used herein, “heterocyclyl” refers to a stable 3- to 18-membered ring radical which consists of carbon atoms and from one to five heteroatoms selected from the group consisting of nitrogen, oxygen and sulfur. For purposes of this invention, the heterocyclyl radical may be a monocyclic, bicyclic, tricyclic or tetracyclic ring system, which may include fused or bridged ring systems; and the nitrogen, carbon or sulfur atoms in the heterocyclyl radical may be optionally oxidized; the nitrogen atom may be optionally quaternized; and the ring radical may be aromatic or partially or fully saturated. Examples of such heterocyclyl radicals include, but are not limited to, azepinyl, acridinyl, benzimidazolyl, benzothiazolyl, benzindolyl, benzothiadiazolyl, benzonaphthofuranyl, benzoxazolyl, benzodioxolyl, benzodioxinyl, benzopyranyl, benzopyranonyl, benzofuranyl, benzofuranonyl, benzothiophenyl, benzotriazolyl, benzo[4,6]imidazo[1,2-a]pyridinyl; carbazolyl, cinnolinyl, dioxolanyl, dibenzofuranyl, decahydroisoquinolyl, furanyl, furanonyl, isothiazolyl, imidazolyl, imidazolinyl, imidazolidinyl, isothiazolidinyl, indolyl, indazolyl, isoindolyl, indolinyl, isoindolinyl, indolizinyl, isoxazolyl, isoxazolidinyl, morpholinyl, naphthyridinyl, oxadiazolyl, octahydroindolyl, octahydroisoindolyl, 2-oxopiperazinyl, 2-oxopiperidinyl, 2-oxopyrrolidinyl, 2-oxoazepinyl, oxazolyl, oxazolidinyl, oxiranyl, piperidinyl, piperazinyl, 4-piperidonyl, phenazinyl, phenothiazinyl, phenoxazinyl, phthalazinyl, pteridinyl, purinyl, pyrrolyl, pyrrolidinyl, pyrazolyl, pyrazolidinyl, pyridinyl, pyrazinyl, pyrimidinyl, pyridazinyl, quinazolinyl, quinoxalinyl, quinolinyl, quinuclidinyl, isoquinolinyl, thiazolyl, thiazolidinyl, thiadiazolyl, triazolyl, tetrazolyl, tetrahydrofuryl, triazinyl, tetrahydropyranyl, thiophenyl, thiamorpholinyl, thiamorpholinyl sulfoxide, and thiamorpholinyl sulfone.

As used herein, “heterocyclylalkyl” refers to a radical of the formula —R^{a}R^{c} where R^{a} is an alkyl radical as defined above and R^{c} is a heterocyclyl radical as defined herein. The alkyl radical and the heterocyclyl radical may be optionally substituted as defined herein.

As used herein, “aralkyl” refers to a radical of the formula —R^{a}R^{d} where R^{a} is an alkyl group radical as defined herein and R^{d} is an aryl radical as defined herein. The alkyl radical and the aryl radical may be optionally substituted as defined herein.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyanide, cyanate, thiocyanate, selenocyanate, trifluoromethoxy, and azide.

As used herein, “haloalkyl” refers to an allyl group in which one or more of the hydrogen atoms are replaced by halogen. Such groups include, but are not limited to, chloromethyl, trifluoromethyl and 1-chloro-2-fluoroethyl.

As used herein, “hydrazone” refers to a divalent group such as ═NNR^{t} which is attached to a carbon atom of another group, forming a double bond, wherein R^{t} is hydrogen or alkyl.

As used herein, “imino” refers to a divalent group such as ═NR, which is attached to a carbon atom of another group, forming a double bond, wherein R is hydrogen or alkyl.

“Optionally substituted alkyl”, “optionally substituted alkenyl” and “optionally substituted alkynyl” refer to alkyl radicals, alkenyl radicals and alkynyl radicals, as defined herein, respectively, that may be optionally substituted by one or more substituents independently selected from the group consisting of nitro, halo, azido, cyano, cycloalkyl, heteroaryl, heterocyclyl, —ORx, —N(Ry)(Rz), —SRx, —C(J)Rx, —C(J)ORx, —C(J)N(Ry)(Rz), —C(J)SRx, —S(O)tRx (where t is 1 or 2), —OC(J)Rx, —OC(J)ORx, —OC(J)N(Ry)(Rz), —OC(J)SRx, —N(Rx)C(J)Rx, —N(Rx)C(J)ORx, —N(Rx)C(J)N(Ry)(Rz), —N(Rx)C(J)SRx, —Si(Rw)₃, —N(Rx)S(O)₂Rw, —N(Rx)S(O)₂N(Ry)(Rz), —S(O)₂N(Ry)(Rz), —N(Rx)C(J)Rx, —P(O)(Rv)₂, —OP(O)(Rv)₂, —C(J)N(Rx)S(O)₂Rx, —C(J)N(Rx)N(Rx)S(O)₂Rx, —C(Rx)=N(ORx), and —C(Rx)=NN(Ry)(Rz), wherein each Rx is independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; Ry and Rz are each independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; or Ry and Rz, together with the nitrogen atom to which they are attached, form a heterocyclyl or heteroaryl; each Rw is independently alkyl, alkenyl, alkynyl, cycloalkyl, cycloalkylalkyl, heterocyclyl, heterocyclylalkyl, aryl, aralkyl, heteroaryl, or heteroaralkyl; each Rv is independently alkyl, alkenyl, alkynyl, cycloalkyl, cycloalkylalkyl, heterocyclyl, heterocyclylalkyl, aryl, aralkyl, heteroaryl, heteroaralkyl, hydroxy, —ORx or —N(Ry)(Rz); and each J is independently O, NRx or S.

“Optionally substituted aryl”, “Optionally substituted aralkyl”, “optionally substituted cycloalkyl”, “optionally substituted cycloalkylalkyl”, “optionally substituted heteroaryl”, “optionally substituted heteroaralkyl”, “optionally substituted heterocyclyl” and “optionally substituted heterocyclylalkyl” refer to aryl, aralkyl, cycloalkyl, cycloalkylalkyl, heterocyclyl, heterocyclylalkyl, heteroaryl and heteroaralkyl radicals, respectively, as defined herein, that are optionally substituted by one or more substituents selected from the group consisting of nitro, halo, azido, cyano, alkyl, haloalkyl, alkenyl, alkynyl, cycloalkyl, cycloalkylalkyl, heterocyclyl, heterocyclylalkyl, aryl, aralkyl, heteroaryl, heteroaralkyl, —Ru—ORx, —Ru—N(Ry)(Rz), —Ru—SRx, —Ru—C(J)Rx, —Ru—C(J)ORx, —Ru—C(J)N(Ry)(Rz), —Ru—C(J)SRx, —Ru—S(O)tRx (where t is 1 or 2), —Ru—OC(J)Rx, —Ru—OC(J)ORx, —Ru—OC(J)N(Ry)(Rz), —Ru—OC(J)SRx, —Ru—N(Rx)C(J)Rx, —Ru—N(Rx)C(J)ORx, —Ru—N(Rx)C(J)N(Ry)(Rz), —Ru—N(Rx)C(J)SRx, —Ru—Si(Rw)₃, —Ru—N(Rx)S(O)₂Rw, —Ru—N(Rx)S(O)₂N(Ry)(Rz), —Ru—S(O)₂N(Ry)(Rz), —Ru—N(Rx)C(J)Rx, —Ru—P(O)(Rv)₂, —Ru—OP(O)(Rv)₂, —Ru—C(J)N(Rx)S(O)₂Rx, —Ru—C(J)N(Rx)N(Rx)S(O)₂Rx, —Ru—C(Rx)=N(ORx), and —Ru—C(Rx)=NN(Ry)(Rz), wherein each Ru is independently alkylene or a direct bond; each Rv is independently alkyl, alkenyl, alkynyl, cycloalkyl, cycloalkylalkyl, heterocyclyl, heterocyclylalkyl, aryl, aralkyl, heteroaryl, heteroaralkyl, hydroxy, —ORx or —N(Ry)(Rz); each Rw is independently alkyl, alkenyl, alkynyl, cycloalkyl, cycloalkylalkyl, heterocyclyl, heterocyclylalkyl, aryl, aralkyl, heteroaryl, or heteroaralkyl; each Rx is independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; Ry and Rz are each independently hydrogen, alkyl, alkenyl, alkynyl, cycloalkyl, cycloalkylalkyl, heterocyclyl, heterocyclylalkyl, aryl, aralkyl, heteroaryl, or heteroaralkyl; or Ry and Rz, together with the nitrogen atom to which they are attached, form a heterocyclyl or heteroaryl; and each J is O, NRx or S.

Unless stated otherwise specifically in the specification, it is understood that the substitution can occur on any atom of the aryl, aralkyl, cycloalkyl, cycloalkylalkyl, heterocyclyl, heterocyclylalkyl, heteroaryl and heteroaralkyl groups.

Optionally substituted cycloalkyl, optionally substituted heterocyclyl and optionally substituted aryl may additionally be substituted with oxo, thioxo, imino, oxime or hydrazone, on a saturated carbon of their respective ring system.

As used herein, “oxime” refers to a divalent group such as ═N—OH, which is attached to a carbon atom of another group, forming a double bond.

As used herein, “oxo” refers to an oxygen atom doubly bonded to a carbon.

As used herein, pseudohalides or pseudohalo groups are groups that behave substantially similar to halides. Such compounds can be used in the same manner and treated in the same manner as halides. Pseudohalides include, but are not limited to, cyanide, cyanate, thiocyanate, selenocyanate, trifluoromethoxy, and azide.

As used herein, “thioxo” refers to a sulfur atom doubly bonded to a carbon.

Where the number of any given substituent is not specified (e.g., haloalkyl), there may be one or more substituents present. For example, “haloalkyl” may include one or more of the same or different halogens. As another example, “C1-3alkoxyphenyl” may include one or more of the same or different alkoxy groups containing one, two or three carbons.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

If employed herein, the following terms have their accepted meaning in the chemical literature.
AcOH acetic acid
CDI carbodiimide
CHCl₃ chloroform
cone concentrated
DBU 1,8-diazabicyclo[5.4.0]undec-7-ene
DCM dichloromethane
DDQ 2,3-dichloro-5,6-dicyano-1,4-benzoquninone
DIEA diisopropyl ethylamine
DMAP 4-(dimethylamino) pyridine
DME 1,2-dimethoxyethane
DMF N,N-dimethylformamide
DMSO dimethylsulfoxide
ELSD Evaporative light scattering detector
EtOAc ethyl acetate
EtOH ethanol (100%)
Et₂O diethyl ether
HBTU 1-H-Benzotriazolium, 1-[bis(dimethylamino)methylene]-hexafluorophosphate(1-),3-oxide O-(Benzotriazol-1-yl)-N,N,N′,N′ tetramethyluronium hexafluorophosphate
Hex hexanes
H₂SO₄ sulfuric acid
LDA Lithium di(iso-propyl)amide
McCN acetonitrile
MeOH methanol
NaBH₃CN sodium cyanoborohydride
Pd/C palladium on activated carbon
TEA triethylamine
THF tetrahydrofuran
TFA trifluoroacetic acid

### B. Formulation of Pharmaceutical Compositions

The pharmaceutical compositions provided herein contain therapeutically effective amounts of one or more of the nuclear receptor activity modulators provided herein that are useful in the prevention, treatment, or amelioration of one or more of the symptoms of diseases or disorders associated with nuclear receptor activity, including the farnesoid X receptor and/or orphan nuclear receptor activity. Such diseases or disorders include, but are not limited to, hypercholesterolemia, hyperlipoproteinemia, hypertriglyceridemia, lipodystrophy, hyperglycemia, diabetes mellitus, dyslipidemia, atherosclerotic disease events, gallstone disease, acne vulgaris, acneiform skin conditions, type II diabetes, Parkinson's disease, cancer, Alzheimer's disease, inflammation, immunological disorders, lipid disorders, obesity, conditions characterized by a perturbed epidermal barrier function, hyperlipidemia, cholestasis, peripheral occlusive disease, ischemic stroke, conditions of disturbed differentiation or excess proliferation of the epidermis or mucous membrane, and cardiovascular disorders.

Further the pharmaceutical compositions provided herein contain therapeutically effective amounts of one or more of the nuclear receptor activity modulators provided herein that are useful in the prevention, treatment, or amelioration of one or more of the symptoms of diseases or disorders that are not directly associated with a nuclear receptor, but for which a complication of the disease or disorder is treatable with claimed compounds and compositions. By way of example, without limitation, Cystic Fibrosis is not typically associated with a nuclear receptor activity, but can result in cholestasis, which may be treated with the subject compounds and compositions.

The compositions contain one or more compounds provided herein. The compounds are preferably formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives is (are) mixed with a suitable pharmaceutical carrier or vehicle. The compounds may be derivatized as the corresponding salts, esters, enol ethers or esters, acids, bases, solvates, hydrates or prodrugs prior to formulation, as described above. The concentrations of the compounds in the compositions are effective for delivery of an amount, upon administration, that treats, prevents, or ameliorates one or more of the symptoms of diseases or disorders associated with nuclear receptor activity or in which nuclear receptor activity is implicated. Such diseases or disorders include, but are not limited to, hypercholesterolemia, hyperlipoproteinemia, hypertriglyceridemia, lipodystrophy, hyperglycemia, diabetes mellitus, dyslipidemia, atherosclerotic disease events, gallstone disease, acne vulgaris, acneiform skin conditions, type II diabetes, Parkinson's disease, cancer, Alzheimer's disease, inflammation, immunological disorders, lipid disorders, obesity, conditions characterized by a perturbed epidermal barrier function, hyperlipidemia, cholestasis, peripheral occlusive disease, ischemic stroke, conditions of disturbed differentiation or excess proliferation of the epidermis or mucous membrane, and cardiovascular disorders.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and in International Patent Application Publication Nos. 99/27365 and 00/25134 and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of diseases or disorders associated with nuclear receptor activity or in which nuclear receptor activity is implicated, as described herein.

Typically a therapeutically effective dosage should produce a serum concentration of active ingredient of from about 0.1 ng/ml to about 50-100 μg/ml. The pharmaceutical compositions typically should provide a dosage of from about 0.001 mg to about 2000 mg of compound per kilogram of body weight per day. Pharmaceutical dosage unit forms are prepared to provide from about 1 mg to about 1000 mg and preferably from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Pharmaceutically acceptable derivatives include acids, bases, enol ethers and esters, salts, esters, hydrates, solvates and prodrug forms. The derivative is selected such that its pharmacokinetic properties are superior to the corresponding neutral compound.

Thus, effective concentrations or amounts of one or more of the compounds described herein or pharmaceutically acceptable derivatives thereof are mixed with a suitable pharmaceutical carrier or vehicle for systemic, topical or local administration to form pharmaceutical compositions. Compounds are included in an amount effective for ameliorating one or more symptoms of, or for treating or preventing diseases or disorders associated with nuclear receptor activity or in which nuclear receptor activity is implicated, as described herein. The concentration of active compound in the composition will depend on absorption, inactivation, excretion rates of the active compound, the dosage schedule, amount administered, particular formulation as well as other factors known to those of skill in the art.

The compositions are intended to be administered by a suitable route, including orally, parenterally, rectally, topically and locally. For oral administration, capsules and tablets are presently preferred. The compositions are in liquid, semi-liquid or solid form and are formulated in a manner suitable for each route of administration. Preferred modes of administration include parenteral and oral modes of administration. Oral administration is presently most preferred.

Solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampoules, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using co-solvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the active ingredient: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidone, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, acetate, sodium citrate, cyclodextrin derivatives, sorbitan monolaurate, triethanolamine sodium acetate, triethanolamine olcate, and other such agents Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount sufficient to alleviate the symptoms of the treated subject.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain 0.001%-100% active ingredient, preferably 0.1-85%, typically 75-95%.

The active compounds or pharmaceutically acceptable derivatives may be prepared with carriers that protect the compound against rapid elimination from the body, such as time release formulations or coatings. The compositions may include other active compounds to obtain desired combinations of properties. The compounds provided herein, or pharmaceutically acceptable derivatives thereof as described herein, may also be advantageously administered for therapeutic or prophylactic purposes together with another pharmacological agent known in the general art to be of value in treating one or more of the diseases or medical conditions referred to hereinabove, such as diseases or disorders associated with nuclear receptor activity or in which nuclear receptor activity is implicated. It is to be understood that such combination therapy constitutes a further aspect of the compositions and methods of treatment provided herein.

### Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, preferably capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose, and starch paste. Lubricants include talc, starch, magnesium or calcium stearate, lycopodium and stearic acid. Diluents include, for example, lactose, sucrose, starch, kaolin, salt, mannitol, and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate, and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate, and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603. Briefly, such formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl other wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

### Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

Implantation of a slow-release or sustained-release system, such that a constant level of dosage is maintained (see, e.g., U.S. Pat. No. 3,710,795) is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, preferably more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

### Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg, preferably 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, preferably 5-35 mg, more preferably about 9-30 mg of lyophilized powder, is added per mL of sterile water or other suitable carrier. The precise amount depends upon the selected compound. Such amount can be empirically determined.

### Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

### Compositions for Other Routes of Administration

Other routes of administration, such as topical application, transdermal patches, and rectal administration are also contemplated herein.

Transdermal patches, including iotophoretic and electrophoretic devices, are well known to those of skill in the art. For example, such patches are disclosed in U.S. Pat. Nos. 6,267,983, 6,261,595, 6,256,533, 6,167,301, 6,024,975, 6,010715, 5,985,317, 5,983,134, 5,948,433, and 5,860,957.

Pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (olyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

### Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759,542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

### Articles of Manufacture

The compounds or pharmaceutically acceptable derivatives may be packaged as articles of manufacture containing packaging material, a compound or pharmaceutically acceptable derivative thereof provided herein, which is effective for modulating the activity of nuclear receptors, including the farnesoid X receptor and/or orphan nuclear receptors, or for treatment, prevention or amelioration of one or more symptoms of nuclear receptor, including the farnesoid X receptor and/or orphan nuclear receptor, mediated diseases or disorders, or diseases or disorders in which nuclear receptor activity, including the farnesoid X receptor and/or orphan nuclear receptor activity, is implicated, within the packaging material, and a label that indicates that the compound or composition, or pharmaceutically acceptable derivative thereof, is used for modulating the activity of nuclear receptors, including the farnesoid X receptor and/or orphan nuclear receptors, or for treatment, prevention or amelioration of one or more symptoms of nuclear receptor, including the farnesoid X receptor and/or orphan nuclear receptor, mediated diseases or disorders, or diseases or disorders in which nuclear receptor activity, including the farnesoid X receptor and/or orphan nuclear receptor activity, is implicated.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any disease or disorder in which nuclear receptor activity, including the farnesoid X receptor and/or orphan nuclear receptor activity, is implicated as a mediator or contributor to the symptoms or cause.

### C. Evaluation of the Activity of the Compounds

Standard physiological, pharmacological and biochemical procedures are available for testing the compounds to identify those that possess biological activities that modulate the activity or nuclear receptors, including the farnesoid X receptor and/or orphan nuclear receptors. Such assays include, for example, biochemical assays such as binding assays, fluorescence polarization assays, FRET based coactivator recruitment assays (see generally Glickman et al., J. Biomolecular Screening, 7 No. 13-10 (2002)), as well as cell based assays including the co-transfection assay, the use of LBD-Gal 4 chimeras and protein-protein interaction assays (see, Lehmann et al., J Biol. Chem., 272(6) 3137-3140 (1997).

High throughput screening systems are commercially available (see, e.g., Zymark Corp., Hopkinton, Mass.; Air Technical Industries, Mentor, Ohio; Beckman Instruments Inc., Fullerton, Calif.; Precision Systems, Inc., Natick, Mass.) that enable these assays to be run in a high throughput mode. These systems typically automate entire procedures, including all sample and reagent pipetting, liquid dispensing timed incubations, and final readings of the microplate in detector(s) appropriate for the assay. These configurable systems provide high throughput and rapid start up as well as a high degree of flexibility and customization. The manufacturers of such systems provide detailed protocols for various high throughput systems. Thus, for example, Zymark Corp. provides technical bulletins describing screening systems for detecting the modulation of gene transcription, ligand binding, and the like.

Assays that do not require washing or liquid separation steps are preferred for such high throughput screening systems and include biochemical assays such as fluorescence polarization assays (see for example, Owicki, J., Biomol Screen 2000 October; 5(5):297) scintillation proximity assays (SPA) (see for example, Carpenter et al., Methods Mol Biol 2002; 190:31-49) and fluorescence resonance energy transfer energy transfer (FRET) or time resolved FRET based coactivator recruitment assays (Mukherjee et al., J Steroid Biochem Mol Biol 2002 July; 81(3):217-25; (Zhou et al., Mol Endocrinol. 1998 October; 12(10): 1594-604). Generally such assays can be preformed using either the full length receptor, or isolated ligand binding domain (LBD). In the case of the farnesoid X receptor, the LBD comprises amino acids 244 to 472 of the full length sequence.

If a fluorescently labeled ligand is available, fluorescence polarization assays provide a way of detecting binding of compounds to the nuclear receptor of interest by measuring changes in fluorescence polarization that occur as a result of the displacement of a trace amount of the label ligand by the compound. Additionally this approach can also be used to monitor the ligand dependent association of a fluorescently labeled coactivator peptide to the nuclear receptor of interest to detect ligand binding to the nuclear receptor of interest.

The ability of a compound to bind to a receptor, or heterodimer complex with RXR, can also be measured in a homogeneous assay format by assessing the degree to which the compound can compete off a radiolabelled ligand with known affinity for the receptor using a scintillation proximity assay (SPA). In this approach, the radioactivity emitted by a radiolabelled compound generates an optical signal when it is brought into close proximity to a scintillant such as a Ysi-copper containing bead, to which the nuclear receptor is bound. If the radiolabelled compound is displaced from the nuclear receptor the amount of light emitted from the nuclear receptor bound scintillant decreases, and this can be readily detected using standard microplate liquid scintillation plate readers such as, for example, a Wallac MicroBeta reader.

The heterodimerization of the farnesoid X receptor with RXRα can also be measured by fluorescence resonance energy transfer (FRET), or time resolved FRET, to monitor the ability of the compounds provided herein to bind to the farnesoid X receptor or other nuclear receptors. Both approaches rely upon the fact that energy transfer from a donor molecule to an acceptor molecule only occurs when donor and acceptor are in close proximity. Typically the purified LBD of the nuclear receptor of interest is labeled with biotin then mixed with stoichiometric amounts of europium labeled streptavidin (Wallac Inc.), and the purified LBD of RXRα is labeled with a suitable fluorophore such as CY5™. Equimolar amounts of each modified LBD are mixed together and allowed to equilibrate for at least 1 hour prior to addition to either variable or constant concentrations of the sample for which the affinity is to be determined. After equilibration, the time-resolved fluorescent signal is quantitated using a fluorescent plate reader. The affinity of the compound can then be estimated from a plot of fluorescence versus concentration of compound added.

This approach can also be exploited to measure the ligand dependent interaction of a co-activator peptide with a nuclear receptor in order to characterize the agonist or antagonist activity of the compounds disclosed herein. Typically the assay in this case involves the use a recombinant Glutathione-5-transferase (GST)-nuclear receptor ligand binding domain (LBD) fusion protein and a synthetic biotinylated peptide sequenced derived from the receptor interacting domain of a co-activator peptide such as the steroid receptor coactivator 1 (SRC-1). Typically GST-LBD is labeled with a europium chelate (donor) via a europium-tagged anti-GST antibody, and the coactivator peptide is labeled with allophycocyanin via a streptavidin-biotin linkage.

In the presence of an agonist for the nuclear receptor, the peptide is recruited to the GST-LBD bringing europium and allophycocyanin into close proximity to enable energy transfer from the europium chelate to the allophycocyanin. Upon excitation of the complex with light at 340 nm excitation energy absorbed by the europium chelate is transmitted to the allophycocyanin moiety resulting in emission at 665 nm. If the europium chelate is not brought in to close proximity to the allophycocyanin moiety there is little or no energy transfer and excitation of the europium chelate results in emission at 615 nm. Thus the intensity of light emitted at 665 nm gives an indication of the strength of the protein-protein interaction. The activity of a nuclear receptor antagonist can be measured by determining the ability of a compound to competitively inhibit (i.e., IC₅₀) the activity of an agonist for the nuclear receptor.

In addition a variety of cell based assay methodologies may be successfully used in screening assays to identify and profile the specificity of compounds of the present invention. These approaches include the co-transfection assay, translocation assays, complementation assays and the use of gene activation technologies to over express endogenous nuclear receptors.

Three basic variants of the co-transfection assay strategy exist, co-transfection assays using full-length nuclear receptor, co transfection assays using chimeric nuclear receptors comprising the ligand binding domain of the nuclear receptor of interest fused to a heterologous DNA binding domain, and assays based around the use of the mammalian two hybrid assay system.

The basic co-transfection assay is based on the co-transfection into the cell of an expression plasmid to express the nuclear receptor of interest in the cell with a reporter plasmid comprising a reporter gene whose expression is under the control of DNA sequence that is capable of interacting with that nuclear receptor. (See for example U.S. Pat. Nos. 5,071,773; 5,298,429, 6,416,957, WO 00/76523). Treatment of the transfected cells with an agonist for the nuclear receptor increases the transcriptional activity of that receptor which is reflected by an increase in expression of the reporter gene, which may be measured by a variety of standard procedures.

For those receptors that function as heterodimers with RXR, such as the farnesoid X receptor, the co-transfection assay typically includes the use of expression plasmids for both the nuclear receptor of interest and RXR. Typical co-transfection assays require access to the full-length nuclear receptor and suitable response elements that provide sufficient screening sensitivity and specificity to the nuclear receptor of interest.

Genes encoding the following full-length previously described proteins, which are suitable for use in the co-transfection studies and profiling the compounds described herein, include rat farnesoid X receptor (GenBank Accession No. NM_{—}021745), human farnesoid X receptor (GenBank Accession No. NM_{—}005123), human RXR α (GenBank Accession No. NM_{—}002957), human RXR β (GenBank Accession No. XM_{—}042579), human RXR γ (GenBank Accession No. XM_{—}053680), human LXR α (GenBank Accession No. NM_{—}005693), human LXR β (GenBank Accession No. NM_{—}007121), human PPAR α (GenBank Accession No. NM_{—}005036) and human PPAR δ (GenBank Accession No. NM_{—}006238).

Reporter plasmids may be constructed using standard molecular biological techniques by placing cDNA encoding for the reporter gene downstream from a suitable minimal promoter. For example luciferase reporter plasmids may be constructed by placing cDNA encoding firefly luciferase immediately down stream from the herpes virus thymidine kinase promoter (located at nucleotides residues −105 to +51 of the thymidine kinase nucleotide sequence) which is linked in turn to the various response elements.

Numerous methods of co-transfecting the expression and reporter plasmids are known to those of skill in the art and may be used for the co-transfection assay to introduce the plasmids into a suitable cell type. Typically such a cell will not endogenously express nuclear receptors that interact with the response elements used in the reporter plasmid.

Numerous reporter gene systems are known in the art and include, for example, alkaline phosphatase Berger, J., et al., (1988) Gene 66 1-10; Kain, S. R. (1997) Methods. Mol. Biol. 63 49-60), □-galactosidase (See, U.S. Pat. No. 5,070,012, issued Dec., 3, 1991 to Nolan et al., and Bronstein, I., et al., (1989) J. Chemilum. Biolum. 4 99-111), chloramphenicol acetyltransferase (See Gorman et al., Mol Cell Biol. (1982) 2 1044-51), □-glucuronidase, peroxidase, □-lactamase (U.S. Pat. Nos. 5,741,657 and 5,955,604), catalytic antibodies, luciferases (U.S. Pat. Nos. 5,221,623; 5,683,888; 5,674,713; 5,650,289; 5,843,746) and naturally fluorescent proteins (Tsien, R. Y. (1998) Annu. Rev. Biochem. 67 509-44).

The use of chimeras comprising the ligand binding domain (LBD) of the nuclear receptor of interest to a heterologous DNA binding domain (DBD) expands the versatility of cell based assays by directing activation of the nuclear receptor in question to defined DNA binding elements recognized by defined DNA binding domain (see WO95/18380). This assay expands the utility of cell based co-transfection assays in cases where the biological response or screening window using the native DNA binding domain is not satisfactory.

In general the methodology is similar to that used with the basic co-transfection assay, except that a chimeric construct is used in place of the full-length nuclear receptor. As with the full-length nuclear receptor, treatment of the transfected cells with an agonist for the nuclear receptor LBD increases the transcriptional activity of the heterologous DNA binding domain which is reflected by an increase in expression of the reporter gene as described above. Typically for such chimeric constructs, the DNA binding domains from defined nuclear receptors, or from yeast or bacterially derived transcriptional regulators such as members of the GAL 4 and Lex A/Umud super families are used.

A third cell based assay of utility for screening compounds of the present invention is a mammalian two-hybrid assay that measures the ability of the nuclear hormone receptor to interact with a cofactor in the presence of a ligand. (See for example, U.S. Pat. Nos. 5,667,973, 5,283,173 and 5,468,614). The basic approach is to create three plasmid constructs that enable the interaction of the nuclear receptor with the interacting protein to be coupled to a transcriptional readout within a living cell. The first construct is an expression plasmid for expressing a fusion protein comprising the interacting protein, or a portion of that protein containing the interacting domain, fused to a GAL4 DNA binding domain. The second expression plasmid comprises DNA encoding the nuclear receptor of interest fused to a strong transcription activation domain such as VP16, and the third construct comprises the reporter plasmid comprising a reporter gene with a minimal promoter and GAL4 upstream activating sequences.

Once all three plasmids are introduced into a cell, the GAL4 DNA binding domain encoded in the first construct allows for specific binding of the fusion protein to GAL4 sites upstream of a minimal promoter. However because the GAL4 DNA binding domain typically has no strong transcriptional activation properties in isolation, expression of the reporter gene occurs only at a low level. In the presence of a ligand, the nuclear receptor-VP16 fusion protein can bind to the GAL4-interacting protein fusion protein bringing the strong transcriptional activator VP16 in close proximity to the GAL4 binding sites and minimal promoter region of the reporter gene. This interaction significantly enhances the transcription of the reporter gene, which can be measured for various reporter genes as described above. Transcription of the reporter gene is thus driven by the interaction of the interacting protein and nuclear receptor of interest in a ligand dependent fashion.

Any compound which is a candidate for activation of the farnesoid X receptor may be tested by these methods. Generally, compounds are tested at several different concentrations to optimize the chances that activation of the receptor will be detected and recognized if present. Typically assays are performed in triplicate and vary within experimental error by less than 15%. Each experiment is typically repeated three or more times with similar results.

Activity of the reporter gene can be conveniently normalized to the internal control and the data plotted as fold activation relative to untreated cells. A positive control compound (agonist) may be included along with DMSO as high and low controls for normalization of the assay data. Similarly, antagonist activity can be measured by determining the ability of a compound to competitively inhibit the activity of an agonist.

Additionally the compounds and compositions can be evaluated for their ability to increase or decrease the expression of genes known to be modulated by the farnesoid X receptor and other nuclear receptors in vivo, using Northern-blot, RT PCR or oligonucleotide microarray analysis to analyze RNA levels. Western-blot analysis can be used to measure expression of proteins encoded by farnesoid X receptor target genes. Genes that are known to be regulated by the farnesoid X receptor include cholesterol 7 α-hydroxylase (CYP7A1), the rate limiting enzyme in the conversion of cholesterol to bile acids, the small heterodimer partner-1 (SHP-1), the bile salt export pump (BSEP, ABCB11), canalicular bile acid export protein, sodium taurocholate cotransporting polypeptide (NTCP, SLC10A1) and intestinal bile acid binding protein (I-BABP).

Established animal models exist for a number of diseases of direct relevance to the claimed compounds and these can be used to further profile and characterize the claimed compounds. These model systems include diabetic dislipidemia using Zucker (fa/fa) rats or (db/db) mice, spontaneous hyperlipidemia using apolipoprotein E deficient mice (ApoE^{-/-}), diet-induced hyperlipidemia, using low density lipoprotein receptor deficient mice (LDR^{-/-}) and atherosclerosis using both the Apo E(^{-/-}) and LDL(^{-/-}) mice fed a western diet, (21% fat, 0.05% cholesterol). Additionally farnesoid X receptor or LXR animal models (e.g., knockout mice) can be used to further evaluate the present compounds and compositions in vivo (see, for example, Sinai, et al., Cell, 102: 731-744 (2000), Peet, et al., Cell, 93:693-704 (1998)).

### D. Methods of Use of the Compounds and Compositions

Methods of use of the compounds and compositions provided herein are also provided. The methods involve both in vitro and in vivo uses of the compounds and compositions for altering nuclear receptor activity, including the farnesoid X receptor and/or orphan nuclear receptor activity, and for treatment, prevention, or amelioration of one or more symptoms of diseases or disorder that are modulated by nuclear receptor activity, including the farnesoid X receptor and/or orphan nuclear receptor activity, or in which nuclear receptor activity, including the farnesoid X receptor and/or orphan nuclear receptor activity, is implicated. Such compounds or compositions will typically exhibit farnesoid X receptor agonist, partial agonist, partial antagonist or antagonist activity in one of the in vitro assays described herein.

Methods of altering nuclear receptor activity, including the farnesoid X receptor, and/or orphan nuclear receptor activity, by contacting the receptor with one or more compounds or compositions provided herein, are provided.

Methods of reducing plasma cholesterol levels and of directly or indirectly modulating cholesterol metabolism, catabolism, synthesis, absorption, re-absorption, secretion or excretion are provided through administering the claimed compounds and compositions provided herein. Methods of reducing dietary cholesterol absorption (see, e.g., International Patent Application Publication No. 00/40965) using the compounds and compositions are provided herein. Also provided, are methods of increasing the expression of ATP-Binding Cassette (ABCA1), thereby increasing reverse cholesterol transport in mammalian cells using the claimed compounds and compositions (see, e.g., International Patent Application Publication No. WO 00/78972).

Methods of reducing plasma triglyceride levels and of directly or indirectly modulating triglyceride metabolism, catabolism, synthesis, absorption, re-absorption, secretion or excretion are provided through administering the claimed compounds and compositions provided herein.

Methods of reducing bile acid levels and of directly or indirectly modulating bile acid metabolism, catabolism, synthesis, absorption, re-absorption, secretion, excretion, or bile acid pool size or composition are provided through administering the claimed compounds and compositions provided herein.

Bile acid chenodeoxycholic acid (CDCA) is a potent ligand for FXR, with EC50s in the range of biologic levels of the bile acid. Parks, D. J. et al., Bile acids: Natural ligands for an orphan nuclear receptor. Science. 284:1365-1368 (1999); Makishima, M. et al., Identification of a nuclear receptor for bile acids. Science. 284:1362-1365 (1999). Wang, H. Et al., Endogenous bile acids are ligands for the nuclear receptor FXR/BAR. Mol. Cell. 3:543-553 (1999).

Studies in humans showed that administration of CDCA in humans significantly lowers plasma triglyceride levels in patients with hypertriglyceridemia. The identification of FXR's role as a bile acid sensor demonstrates that the effect is mediated through the FXR receptor. Camarri E. et al., Hypotriglyceridemic effect of chenodeoxycholic acid after a short time of administration, Int J Clin Pharmacol Biopharm. November:16(11):527-8 (1978); Camarri E. et al., Influence of chenodeoxycholic acid on serum triglycerides in patients with primary hypertriglyceridemia, Int J Clin Pharmacol Biopharm., November:16(11):523-6 (1978); Camarri E. et al., The hypotriglyceridemic effect of chenodeoxycholic acid in type IV hyperlipemia, The hypotriglyceridemic effect of chenodeoxycholic acid in type IV hyperlipemia. Biomedicine, Oct:29(6):193-8 (1978). The lack of effect on plasma lipids from treatment with the bile acid UDCA is consistent with FXR's role in the CDCA mediated plasma lipid modulation, as UDCA is not a ligand for FXR. Carulli N. et al., Chenodeoxycholic acid and ursodeoxycholic acid effects in endogenous hypertriglyceridemias. A controlled double-blind trial. J Clin Pharmacol., Oct:21(10):436-42 (1981); Tint et al., Effect of ursodeoxycholic acid chenodeoxycholic acid on cholesterol and bile acid metabolism. Gastroenterology, 91:1007-1018 (1986); Iser and Sali, Chenodeoxycholic acid: a review of its pharmacological properties and therapeutic use. Drugs, 21:90-119 (1981); Angelin et al., Effects of cholestyramine and chenodeoxycholic acid on the metabolism of endogenous triglyceride in hyperlipoproteinemia. J. of Lipid Research, 19:1017-1023 (1978).

The role of FXR in regulating lipid metabolism. For example, FXR can upregulate PPARα. Activation of PPARα is known to lower plasma lipids, as well as, improve insulin resistance and glucose metabolism. Pineda Torra I. et al., Bile acids induce the expression of the human peroxisome proliferator-activated receptor alpha gene via activation of the farnesoid X receptor., Mol. Endocrinol. Feb; 17(2):259-72 (2003).

The FXR knock out mice show increased plasma non-HDL cholesterol and triglycerides, apoβ containing lipoprotein synthesis and intestinal cholesterol absorption indicating the loss of FXR function is potentially atherogenic. Thus, the activation of FXR with agonists has antiatherogenic effects. Lambert G. et al., The Farnesoid X-receptor is an essential regulator of cholesterol homeostasis, J Biol. Chem. Jan 24; 278 (4):2563-70 (2003); Sinal C J, Tohkin M. et al., Targeted disruption of the nuclear receptor FXR/BAR impairs bile acid and lipid homeostasis, Cell, Sep 15; 102(6):731-44 (2000); Edwards P. A. et al., BAREing it all: the adoption of LXR and FXR and their roles in lipid homeostasis, J Lipid Res. Jan: 43(1):2-12 (2002); Kast H. R. et al., Farnesoid X-activated receptor induces apolipoprotein C-II transcription: a molecular mechanism linking plasma triglyceride levels to bile acids, Mol. Endocrinol. Oct; 15(10):1720-8 (2001).

Methods of treatment, prevention, or amelioration of one or more symptoms of a disease or disorder affecting cholesterol, triglyceride, or bile acid levels, or any combination thereof, are provided using the compounds and compositions provided herein.

Methods are provided for the treatment, prevention, or amelioration of one or more symptoms of, as well as treating the complications of, hyperlipidemia, hypercholesterolemia, dyslipidemia and lipodystrophy.

The term “hyperlipidemia” refers to the presence of an abnormally elevated level of lipids in the blood. Hyperlipidemia can appear in at least three forms: (1) hypercholesterolemia, i.e., an elevated LDL cholesterol level (120 mg/dL and above); (2) hypertriglyceridemia, i.e., an elevated triglyceride level; (150 mg/dL and above) and (3) combined hyperlipidemia, i.e., a combination of hypercholesterolemia and hypertriglyceridemia.

The term “dyslipidemia” refers to abnormal levels of lipoproteins in blood plasma including both depressed and/or elevated levels of lipoproteins (e.g., elevated levels of Low Density Lipoprotein, (LDL), Very Low Density Lipoprotein (VLDL) and depressed levels of High Density Lipoprotein (HDL) (less than 40 mg/dL)).

Methods are also provided for the treatment, prevention, or amelioration of one or more symptoms of atherosclerosis, atherosclerotic disease, atherosclerotic disease events and atherosclerotic cardiovascular diseases.

Atherosclerosis is the process in which deposits of fatty substances, cholesterol, cellular waste products, calcium and other substances build up in the inner lining of an artery. This buildup is called plaque. It initially affects large and medium-sized arteries. Some hardening of arteries often occurs when people grow older.

Plaques can grow large enough to significantly reduce the blood's flow through an artery. However significant damage to the body can also occur when the artery walls become fragile and rupture. Atherosclerotic plaques that rupture can cause blood clots to form that can block blood flow or break off and travel to another part of the body. If either happens and the blood clot blocks a blood vessel that feeds the heart, it can cause a heart attack. If the blood clot blocks a blood vessel that feeds the brain, it can cause a stroke. And if blood supply to the arms or legs is reduced, it can cause difficulty walking and eventually gangrene.

Accordingly atherosclerosis encompasses a range of vascular diseases and conditions that arise as a result of the primary disease modality. Atherosclerotic cardiovascular diseases can be recognized and understood by physicians practicing in the relevant fields of medicine and include the following: Restenosis following revascularization procedures, coronary heart disease (also known as coronary artery heart disease or ischemic heart disease), cerebrovascular disease including ischemic stroke, multi-infarct dementia, and peripheral vessel disease, including erectile dysfunction.

A compound or composition of the present invention may be administered to prevent or reduce the risk of occurrence, or recurrence where the potential exists, of coronary heart disease event, a cerebrovascular event, and/or intermittent claudication.

Coronary heart disease (CHD) events are intended to include CHD death, myocardial infarction (i.e., a heart attack), and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease. It is intended that persons who have previously experienced one or more non-fatal atherosclerotic disease event are those for whom the potential for recurrence of such an event exists.

Persons to be treated with the instant therapy include those at risk of developing atherosclerotic disease and of having an atherosclerotic disease event. Standard atherosclerotic disease risk factors are known to the average physician practicing in the relevant fields of medicine. Such known risk factors include, but are not limited to, hypertension, smoking, diabetes, low levels of high density lipoprotein cholesterol, high levels of low density lipoprotein cholesterol, and a family history of atherosclerotic cardiovascular disease. Published guidelines for determining those who are at risk of developing atherosclerotic disease and of having an atherosclerotic disease event can be found in: Third Report of the National Cholesterol Education Program, Expert Panel on Detection, Evaluation, and Treatment of high Blood Cholesterol in Adults (Adult Treatment Panel III), National Institutes of Health, National Heart Lung and Blood Institute, NIH Publication No. 01-3670, May 2001; National Cholesterol Education Program, Second report of the Expert Panel on Detection, Evaluation, and Treatment of High Blood Cholesterol in Adults (Adult Treatment Panel II), National Institute of Health, National Heart Lung and Blood Institute, NIH Publication No. 93-3095, September 1993; abbreviated version: Expert Panel on Detection, Evaluation, and Treatment of High Blood Cholesterol in Adults, Summary of the second report of the national cholesterol education program (NCEP) Expert Panel on Detection, Evaluation, and Treatment of High Blood Cholesterol in Adults (Adult Treatment Panel II), JAMA, 1993, 269, pp. 3015-23. People identified as having one or more of the above-noted risk factors, as well as people who already have atherosclerosis, are intended to be included within the group of people considered to be at risk for having an atherosclerotic disease event.

Coronary heart disease events are intended to include coronary heart disease death, myocardial infarction and coronary revascularization procedures. Cerebrovascular events are intended to include ischemic or hemorrhagic stroke (also known as cerebrovascular accidents) and transient ischemic attacks. Intermittent claudication is a clinical manifestation of peripheral vessel disease.

The term “atherosclerotic disease event” as used herein is intended to encompass coronary heart disease events, cerebrovascular events, and intermittent claudication. It is intended that person who have previously experienced one or more non-fatal atherosclerotic disease events are those for whom the potential for recurrence of such an event exists.

Additionally, the instant invention also provides a method for preventing or reducing the risk of a first or subsequent occurrence of an atherosclerotic disease event comprising the administration of a prophylactically effective amount of a compound or composition of the present invention to a patient at risk for such an event. The patient may already have atherosclerotic disease at the time of administration, or may be at risk for developing it.

Risk factors for developing atherosclerotic disease events include increasing age (65 and over), male gender, a family history of atherosclerotic disease events, high blood cholesterol (especially LDL or “bad” cholesterol over 100 mg/dL), cigarette smoking and exposure to tobacco smoke, high blood pressure, Diabetes mellitus, obesity and physical inactivity.

In another aspect, the method of this invention also serves to remove cholesterol from tissue deposits such as atherosclerotic plaques or xanthomas in a patient with atherosclerotic disease manifest by clinical signs such as angina, claudication, bruits, one that has suffered a myocardial infarction or transient ischemic attack, or one diagnosed by angiography, sonography or MRI.

Methods of treatment, prevention, or amelioration of one or more of the symptoms of diabetes mellitus, as well as treating the complications of diabetes mellitus, (see, e.g., International Patent Application Publication No. WO 01/82917) are also provided using the compounds and compositions provided herein.

Diabetes mellitus, commonly called diabetes, refers to a disease or condition that is generally characterized by metabolic defects in production and utilization of glucose which result in the failure to maintain appropriate blood sugar levels in the body (see, e.g., LeRoith, D. et al., (eds.), DIABETES MELLITUS (Lippincott-Raven Publishers, Philadelphia, Pa. U.S.A. 1996)).

In the case of diabetes of the type 2 form, the disease is characterized by insulin resistance, in which insulin loses its ability to exert its biological effects across a broad range of concentrations. This resistance to insulin responsiveness results in insufficient insulin activation of glucose uptake, oxidation and storage in muscle and inadequate insulin repression of lipolysis in adipose tissue and of glucose production and secretion in liver (see, e.g., Reaven, G. M., J. Basic & Clin. Phys. & Pharm. (1998) 9: 387-406 and Flier, J. Ann Rev. Med. (1983) 34:145-60). The resulting condition is elevated blood glucose, which is called “hyperglycemia.” Uncontrolled hyperglycemia is associated with increased and premature mortality due to an increased risk for microvascular and macrovascular diseases, including retinopathy (the impairment or loss of vision due to blood vessel damage in the eyes); neuropathy (nerve damage and foot problems due to blood vessel damage to the nervous system); and nephropathy (kidney disease due to blood vessel damage in the kidneys), hypertension, cerebrovascular disease and coronary heart disease. Therefore, control of glucose homeostasis is an important approach for the treatment of diabetes.

Methods of treatment, prevention, or amelioration of one or more of the symptoms of insulin insensitivity or resistance as well as treating the complications of insulin insensitivity or resistance (see, e.g., International Patent Application Publication No. WO 01/82917) are also provided using the compounds and compositions provided herein.

Methods of treatment, prevention, or amelioration of one or more of the symptoms of hyperglycemia as well as treating the complications of hyperglycemia (see, e.g., International Patent Application Publication No. WO 01/82917) are also provided using the compounds and compositions provided herein.

Insulin resistance has been hypothesized to unity the clustering of hypertension, glucose intolerance, hyperinsulinemia, increased levels of triglyceride and decreased HDL cholesterol, and central and overall obesity. The association of insulin resistance with glucose intolerance, an increase in plasma triglyceride and a decrease in high-density lipoprotein cholesterol concentrations, hypertension, hyperuricemia, smaller denser low-density lipoprotein particles, and higher circulating levels of plasminogen activator inhibitor-1, has been referred to as “Syndrome X” (see, e.g., Reaven, G. M., Physiol. Rev. (1995) 75: 473-486). Accordingly, methods of treatment, prevention, or amelioration of any disorders related to diabetes, hyperglycemia or insulin resistance including the cluster of disease states, conditions or disorders that make up “Syndrome X” are provided.

Additionally the instant invention also provides a method for preventing or reducing the risk of hyperglycemia, insulin resistance or diabetes development in a patient, comprising the administration of a prophylactically effective amount of a compound or composition of the present invention to a patient at risk for such an event. The patient may already be obese, (BMI of 30.0 or greater), overweight (BMI of 25.0 to 30.0) or possess other risk factors for developing diabetes including age, family history and physical inactivity.

Further provided herein are methods for the treatment, prevention, or amelioration of one or more symptoms of cholestasis, as well as for the treatment of the complications of cholestasis by administering a compound or composition provided herein.

Cholestasis is typically caused by factors within the liver (intrahepatic) or outside the liver (extrahepatic) and leads to the accumulation of bile salts, bile pigment bilirubin, and lipids in the blood stream instead of being eliminated normally.

Intrahepatic cholestasis is characterized by widespread blockage of small ducts or by disorders, such as hepatitis, that impair the body's ability to eliminate bile. Intrahepatic cholestasis may also be caused by alcoholic liver disease, primary biliary cirrhosis, cancer that has spread (metastasized) from another part of the body, primary sclerosing cholangitis, gallstones, biliary colic and acute cholecystitis. It can also occur as a complication of surgery, serious injury, cystic fibrosis, infection, or intravenous feeding or be drug induced. Cholestasis may also occur as a complication of pregnancy and often develops during the second and third trimesters.

Extrahepatic cholestasis is most often caused by choledocholithiasis (Bile Duct Stones), benign biliary strictures (non-cancerous narrowing of the common duct), cholangiocarcinoma (ductal carcinoma), and pancreatic carcinoma. Extrahepatic cholestasis can occur as a side effect of many medications.

Accordingly, compounds or compositions provided herein may be used for the treatment, prevention, or amelioration of one or more symptoms of intrahepatic or extrahepatic cholestasis, including without limitation, biliary atresia, obstetric cholestasis, neonatal cholestasis, drug induced cholestasis, cholestasis arising from Hepatitis C infection, chronic cholestatic liver disease such as primary biliary cirrhosis (PBC) and primary sclerosing cholangitis (PSC).

Further provided by this invention are methods for treating obesity, as well as treating the complications of obesity, by administering a compound or composition of the present invention. The terms “obese” and “obesity” refers to, according to the World Health Organization, a Body Mass Index (BMI) greater than 27.8 kg/m² for men and 27.3 kg/m² for women (BMI equals weight (kg)/height (m²). Obesity is linked to a variety of medical conditions including diabetes and an atherosclerotic disease event. (See, e.g., Barrett-Conner, E., Epidemol. Rev. (1989) 11: 172-181; and Knowler, et al., Am. J. Clin. Nutr. (1991) 53:1543-1551). Accordingly the claimed compounds or compositions that may be used for treating obesity or its complications, and can be identified, formulated, and administered as previously described above.

### E. Combination Therapy

Also contemplated herein is combination therapy using one or more compounds or compositions provided herein, or a pharmaceutically acceptable derivative thereof, in combination with one or more of the following: antihyperlipidemic agents, plasma HDL-raising agents, antihypercholesterolemic agents, cholesterol biosynthesis inhibitors (such as HMG CoA reductase inhibitors, such as lovastatin, simvastatin, pravastatin, fluvastatin, atorvastatin and rivastatin), acyl-coenzyme A: cholesterol acytransferase (ACAT) inhibitors, probucol, raloxifene, nicotinic acid, niacinamide, cholesterol absorption inhibitors, bile acid sequestrants (such as anion exchange resins, or quaternary amines (e.g., cholestyramine or colestipol)), low density lipoprotein receptor inducers, clofibrate, fenofibrate, benzofibrate, cipofibrate, gemfibrizol, vitamin B₆, vitamin B₁₂, anti-oxidant vitamins, β-blockers, anti-diabetes agents, angiotensin II antagonists, angiotensin converting enzyme inhibitors, platelet aggregation inhibitors, fibrinogen receptor antagonists, LXR α or β agonists, antagonists or partial agonists, aspirin or fibric acid derivatives. The compound or composition provided herein, or pharmaceutically acceptable derivative thereof, is administered simultaneously with, prior to, or after administration of one or more of the above agents. Pharmaceutical compositions containing a compound provided herein and one or more of the above agents are also provided.

Combination therapy includes administration of a single pharmaceutical dosage formulation which contains a compound of the present invention and one or more additional active agents, as well as administration of a compound of the present invention and each active agent in its own separate pharmaceutical dosage formulation. For example, a farnesoid X receptor agonist, partial agonist, partial antagonist, or antagonist of the present invention and an HMG-CoA reductase inhibitor can be administered to the patient together in a single oral dosage composition such as a tablet or capsule, or each agent administered in separate oral dosage formulations. Where separate dosage formulations are used, the compounds described herein and one or more additional active agents can be administered at essentially the same time, i.e., concurrently, or at separately staggered times, i.e., sequentially; combination therapy is understood to include all these regimens.

An example of combination therapy that modulates, or prevents the onset of the symptoms, or associated complications of atherosclerosis, is administered with one or more of the following active agents: an antihyperlipidemic agent; a plasma HDL-raising agent; an antihypercholesterolemic agent, such as a cholesterol biosynthesis inhibitor, e.g., an hydroxymethylglutaryl (HMG) CoA reductase inhibitor (also referred to as statins, such as lovastatin, simvastatin, pravastatin, fluvastatin, and atorvastatin), an HMG-CoA synthase inhibitor, a squalene epoxidase inhibitor, or a squalene synthetase inhibitor (also known as squalene synthase inhibitor); an acyl-coenzyme A cholesterol acyltransferase (ACAT) inhibitor, such as melinamide; probucol; nicotinic acid and the salts thereof and niacinamide; a cholesterol absorption inhibitor, such as β-sitosterol; a bile acid sequestrant anion exchange resin, such as cholestyramine, colestipol or dialkylaminoalkyl derivatives of a cross-linked dextran; an LDL (low density lipoprotein) receptor inducer; fibrates, such as clofibrate, bezafibrate, fenofibrate, and gemfibrizol; vitamin B₆ (also known as pyridoxine) and the pharmaceutically acceptable salts thereof, such as the HCl salt; vitamin B₁₂ (also known as cyanocobalamin); vitamin B₃ (also known as nicotinic acid and niacinamide, supra); anti-oxidant vitamins, such as vitamin C and E and beta carotene; a beta-blocker; LXR α or β agonists, antagonists, or partial agonists, an angiotensin II antagonist; an angiotensin converting enzyme inhibitor; and a platelet aggregation inhibitor, such as fibrinogen receptor antagonists (i.e., glycoprotein IIb/IIIa fibrinogen receptor antagonists) and aspirin.

A compound or composition of the present invention is preferably administered with a cholesterol biosynthesis inhibitor, particularly an HMG-CoA reductase inhibitor. The term HMG-CoA reductase inhibitor is intended to include all pharmaceutically acceptable salt, ester, free acid and lactone forms of compounds which have HMG-CoA reductase inhibitory activity and, therefore, the use of such salts, esters, free acids and lactone forms is included within the scope of this invention. Other HMG-CoA reductase inhibitors can be readily identified using assays well-known in the art. For instance, suitable assays are described or disclosed in U.S. Pat. No. 4,231,938 and WO 84/02131. Examples of suitable HMG-CoA reductase inhibitors include, but are not limited to, lovastatin (MEVACOR®; see, U.S. Pat. No. 4,231,938); simvastatin (ZOCOR®; see, U.S. Pat. No. 4,444,784); pravastatin sodium (PRAVACHOL®; see, U.S. Pat. No. 4,346,227); fluvastatin sodium (LESCOL®; see, U.S. Pat. No. 5,354,772); atorvastatin calcium (LIPITOR®; see, U.S. Pat. No. 5,273,995) and rivastatin (also known as cerivastatin; see, U.S. Pat. No. 5,177,080). The structural formulas of these and additional HMG-CoA reductase inhibitors that can be used in the methods of the present invention are described at page 87 of M. Yalpani, “Cholesterol Lowering Drugs,” Chemistry & Industry, pp. 85-89 (5 Feb. 1996). In one embodiment, the HMG-CoA reductase inhibitor is selected from lovastatin and simvastatin.

Dosage information for HMG-CoA reductase inhibitors is well known in the art, since several HMG-CoA reductase inhibitors are marketed in the U.S. In particular, the daily dosage amounts of the HMG-CoA reductase inhibitor may be the same or similar to those amounts which are employed for anti-hypercholesterolemic treatment and which are described in the Physicians' Desk Reference (PDR). For example, see the 50th Ed. of the PDR, 1996 (Medical Economics Co); in particular, see at page 216 the heading “Hypolipidemics,” sub-heading “HMG-CoA Reductase Inhibitors,” and the reference pages cited therein. Preferably, the oral dosage amount of HMG-CoA reductase inhibitor is from about 1 to 200 mg/day and, more preferably, from about 5 to 160 mg/day. However, dosage amounts will vary depending on the potency of the specific HMG-CoA reductase inhibitor used as well as other factors as noted above. An HMG-CoA reductase inhibitor which has sufficiently greater potency may be given in sub-milligram daily dosages.

As examples, the daily dosage amount for simvastatin may be selected from 5 mg, 10 mg, 20 mg, 40 mg, 80 mg and 160 mg for lovastatin, 10 mg, 20 mg, 40 mg and 80 mg; for fluvastatin sodium, 20 mg, 40 mg and 80 mg; and for pravastatin sodium, 10 mg, 20 mg, and 40 mg. The daily dosage amount for atorvastatin calcium may be in the range of from 1 mg to 160 mg and, more particularly, from 5 mg to 80 mg. Oral administration may be in a single or divided doses of two, three, or four times daily, although a single daily dose of the HMG-CoA reductase inhibitor is preferred.

Diabetic patients are likely to suffer from premature development of atherosclerotic disease events and increased rate of cardiovascular and peripheral vascular diseases. Hyperlipidemia and dyslipidemia are important precipitating factors for these diseases. See, e.g., Wilson, J. et al., (ed.), Disorders of Lipid Metabolism, Chapter 23, Textbook of Endocrinology, 9th Edition, (W. B. Sanders Company, Philadelphia, Pa. U.S.A. 1998). Dyslipidemia is characterized by abnormal levels of lipoproteins in blood plasma (e.g. elevated levels of LDL, VLDL and depressed levels of HDL), and has been shown to be one of the main contributors to the increased incidence of coronary events and deaths among diabetic subjects (see, e.g., Joslin, E. Ann. Chim. Med. (1927) 5: 1061-1079). Epidemiological studies since then have confirmed the association and have shown a several-fold increase in coronary deaths among diabetic subjects when compared with non-diabetic subjects (see, e.g., Garcia, M. J. et al., Diabetes (1974) 23: 105-11 (1974); and Laakso, M. and Lehto, S., Diabetes Reviews (1997) 5(4): 294-315).

The methods of the present invention can be used effectively in combination with one or more additional active anti-diabetes agents depending on the desired target therapy (see, e.g., Turner, N. et al. Prog. Drug Res. (1998) 51: 33-94; Haffner, S. Diabetes Care (1998) 21: 160-178; and DeFronzo, R. et al. (eds.), Diabetes Reviews (1997) Vol. 5 No. 4).

A number of studies have investigated the benefits of combination therapies with oral agents (see, e.g., Mahler, R., J. Clin. Endocrinol. Metab. (1999) 84: 1165-71; United Kingdom Prospective Diabetes Study Group: UKPDS 28, Diabetes Care (1998) 21: 87-92; Bardin, C. W.,(ed.), CURRENT THERAPY IN ENDOCRINOLOGY AND METABOLISM, 6th Edition (Mosby-Year Book, Inc., St. Louis, Mo. 1997); Chiasson, J. et al., Ann. Intern. Med. (1994) 121: 928-935; Coniff, R. et al., Clin. Ther. (1997) 19: 16-26; Coniff, R. et al., Am. J. Med. (1995) 98: 443-451; and Iwamoto, Y. et al, Diabet. Med. (1996) 13 365-370; Kwiterovich, P. Am. J. Cardiol (1998) 82(12A): 3U-17U). These studies indicate that the modulation of hyperlipidemia associated with diabetes can further improve the treatment outcome of diabetics.

Accordingly, another combination therapy claimed herein is suitable for treating diabetes and its related symptoms, complications, and disorders, and includes the co-administration of the compounds or compositions provided herein with for example, sulfonylureas (such as chlorpropamide, tolbutamide, acetohexamide, tolazamide, glyburide, gliclazide, glynase, glimepiride, and glipizide), biguanides (such as metformin), thiazolidinediones (such as ciglitazone, pioglitazone, troglitazone, and rosiglitazone); and related insulin sensitizers, such as selective and non-selective activators of PPARα PPARβ and PPARγ; LXR α or β agonists, antagonists and partial agonists, dehydroepiandrosterone (also referred to as DHEA or its conjugated sulphate ester, DHEA-SO₄); antiglucocorticoids; TNFα-inhibitors; α-glucosidase inhibitors (such as acarbose, miglitol, and voglibose), pramlintide (a synthetic analog of the human hormone amylin), other insulin secretogogues (such as repaglinide, gliquidone, and nateglinide), insulin, as well as the active agents discussed above for treating atherosclerosis.

Another example of combination therapy claimed herein is the co-administration of the claimed compounds or compositions provided herein with compounds or compositions for treating obesity or obesity-related disorders, wherein the claimed compounds can be effectively used in combination with, for example, phenylpropanolamine, phentermine, diethylpropion, mazindol; fenfluramine, dexfenfluramine, phentiramine, β₃ adrenoceptor agonist agents; sibutramine, gastrointestinal lipase inhibitors (such as orlistat), LXR α or β agonists, antagonists and partial agonists, and leptins. Other agents used in treating obesity or obesity-related disorders include neuropeptide Y, enterostatin, cholecytokinin, bombesin, amylin, histamine H₃ receptors, dopamine D₂ receptors, melanocyte stimulating hormone, corticotrophin releasing factor, galanin and gamma amino butyric acid (GABA).

Another example of a claimed combination therapy is the co-administration of the claimed compound or composition provided herein with compounds or compositions for treating cholestasis and its related symptoms, complications, and disorders. Such co-administered compounds include for example, Actigall (Ursodeoxycholic acid—UDCA), corticosteroids, anti-infective agents (Rifampin, Rifadin, Rimactane), anti-viral agents, Vitamin D, Vitamin A, phenobarbital, cholestyramine, UV light, antihistamines, oral opiate receptor antagonists and biphosphates, for the treatment, prevention, or amelioration of one or more symptoms of intrahepatic or extrahepatic cholestasis. Dosage information for these agents is well known in the art.

### F. Preferred Embodiments

In one preferred embodiment, the compound is a compound of formula (I) in the Summary of the Invention wherein R¹ is —C(J)OR¹¹; J is O; R³ is COR⁹; R⁹ is optionally substituted alkyl, optionally substituted aryl, optionally substituted aralkyl, optionally substituted heteroaryl, optionally substituted heteroaralkyl, optionally substituted heterocyclyl or optionally substituted heterocyclylalkyl; R⁶ or R⁷ is optionally substituted alkyl; n is 0-3; R⁸ is optionally substituted alkyl or halo, preferably fluoro, chloro or bromo.

In one preferred embodiment, the compound is a compound of formula (I) in the Summary of the Invention wherein R⁹ is optionally substituted alkyl, preferably methyl, ethyl, propyl, isopropyl, butyl, isobutyl or pentyl.

In one preferred embodiment, the compound is a compound of formula (I) in the Summary of the Invention wherein R⁹ is optionally substituted heteroaryl, optionally substituted heteroaralkyl, optionally substituted heterocyclyl or optionally substituted heterocyclylalkyl.

In one preferred embodiment, the compound is a compound of formula (I) in the Summary of the Invention wherein R⁹ is optionally substituted heteroaryl or optionally substituted heteroaralkyl.

In one preferred embodiment, the compound is a compound of formula (I) in the Summary of the Invention wherein R⁹ is optionally substituted heterocyclyl or optionally substituted heterocyclylalkyl.

In one preferred embodiment, the compound is a compound of formula (I) in the Summary of the Invention wherein R¹¹ is optionally substituted alkyl, preferably methyl, ethyl, propyl, isopropyl, butyl, isobutyl or pentyl.

In one preferred embodiment, the compound is a compound of formula (I) in the Summary of the Invention wherein R¹ is —C(J)OR¹¹; J is O; R³ is CON(R¹¹)(R¹²); R¹¹ is hydrogen or optionally substituted alkyl; R¹² is optionally substituted alkyl, optionally substituted aryl, optionally substituted aralkyl, optionally substituted cycloalkyl, optionally substituted cycloalkylalkyl, optionally substituted heterocyclyl or optionally substituted heterocyclylalkyl; R⁶ or R⁷ is optionally substituted alkyl; and n is 0-3.

In one preferred embodiment, the compound is a compound of formula (I) in the Summary of the Invention, wherein R¹¹ and R¹² together to which they are attached form optionally substituted heterocyclyl or optionally substituted heterocyclylalkyl.

In one preferred embodiment, the compound is a compound of formula (I) in the Summary of the Invention, wherein R¹¹ and R¹² together to which they are attached form optionally substituted heterocyclyl or optionally substituted heterocyclylalkyl, optionally substituted with one or more Q¹.

In one preferred embodiment, the compound is a compound of formula (I) in the Summary of the Invention, when R³ is CON(R¹¹)(R¹²); R¹¹ is hydrogen and R¹² is selected from the group consisting of methyl, ethyl, propyl, isopropyl, butyl, isobutyl, dimethylaminoethyl, dimethylaminopropyl, diethylaminoethyl, diethylamino, dimethylamino, cyclopntyl, cyclohexyl, cycloheptyl, phenyl, 2-morpholin-4-ylethyl, 3-morpholin-4-ylpropyl, 3-morpholin-4-ylpropyl)amino, and piperidinyl.

In one preferred embodiment, the compound is a compound of formula (I) in the Summary of the Invention, wherein R¹¹ and R¹² together to which they are attached form optionally substituted heterocyclyl or optionally substituted heterocyclylalkyl selected from the group consisting of pyrrolidin-1-yl, 4-pyrrolidin-1-yl, piperidin-1-yl, 4-methylpiperazin-1-yl, 4-ethylpiperazin-1-yl, 4-piperazin-1-yl, 4-propylpiperazin-1-yl, piperidin-3-yl, piperidinyl, (1S,4S)-5-methyl-2,5-diazabicyclo[2.2.1]hept-2-yl and azepanyl.

In one preferred embodiment, the compound is a compound of formula (I) in the Summary of the Invention, wherein R⁹ is optionally substituted aryl or aralkyl, optionally substituted with one or more Q¹.

In one preferred embodiment, the compound is a compound of formula (I) in the Summary of the Invention, wherein Q¹ is selected from the group consisting of methyl, ethyl, propyl, diethylamino, dimethylamino, diethylaminomethyl, diethylaminoethyl, dimethylaminopropyloxymethyl, phenyl, phenylmethyl, pyrrolidinyl, piperazinyl, piperidinyl, methylpiperidinyl, methylpiperazinyl, 2-oxo-2-pyrrolidin-1ylethyl, and morpholino-4-methyl.

In one preferred embodiment, the compound is a compound of formula (I) in the Summary of the Invention, wherein Q¹ is selected from the group consisting of hydroxy, cyano, 2-methyl; 3-methyl; methylpiperazinyl, 3-chloromethyl, 3,4-difluoro; 3-methyl, 4-methyl; 2-methyloxy; 3-methyloxy; 4-methyloxy; 3-fluoro-4-methyl; 4-fluoro-3-methyl; 2-trifluoromethyloxy; 2-chloro; 3-chloro; 4-chloro; 2,4-dichloro; 2-chloro-3,6-difluoro, 3-chloro-2,6-difluoro, 2-fluoro; 3-fluoro; 2-bromo; 3-trifluoromethyl; 2,3-difluoro; 2,4-difluoro; 2,5-difluoro; 2,6-difluoro; 3,4-difluoro; 3,6-difluoro; 3,4-difluoro; 2,3-difluoro-4-trifluoromethyl; 2-fluoro-4-trifluoromethyl; 2-fluoro-3-trifluoromethyl; 3-fluoro-5-trifluoromethyl; 2,5-bistrifluoromethyl; 3,5-bistrifluoromethyl; 3-chloro-2-fluoro-4-trifluoromethy; 3-fluoro-4-trifluoromethyl; 4-fluoro-3-trifluoromethyl; 4-fluoro-2-trifluoromethyl; 2-chloro-4-fluoro; 3-chloro-4-fluoro; 2-trifluoromethyl; 4-trifluoromethyl; 2,3,4-trifluoro; 2,4,6-trifluoro; 2,4,5-trifluoro; 3,4-bis(methyloxy); 3-phenylmethyloxy; methyloxyphenylmethyloxy, 4-piperidin-4-yl, 3-piperidin-4-yl, 3-piperidin-4-ylmethyl, piperidin-4-ylmethyl, dimethylaminomethyl, diethylaminomethyl, dimethylaminoethyloxy, dimethylaminopropyloxy, diethylaminopropyloxy, 4-methylsulfonylpiperazin-1-yl, 3-azepan-1-ylmethyl, 4-methyl-1,4-diazepan-1-yl, 3-pyrrolidin-1-ylethyl, 4-methylpiperazin-1-ylmethyl; 4-ethylpiperazin-1-ylmethyl; 3-piperazin-1-ylmethyl; morpholin-4-ylmethyl; 3-morpholin-4-ylmethyl; 2-morpholin-4-yl ethyl oxy; 2-piperidin-1-yl ethyloxy; 3-morpholin-4-ylpropyloxy 1H-pyrazol-1-yl, 4-trifluoromethyl-1H-pyrazol-1-yl, 4-acetylpiperazin-1-ylmethyl; methylbenzotriazolyl, dimethylethyloxycarbonylpiperazin-1-ylmethyl, 4-phenylsulfonylpiperazin-1-ylmethyl, 4-fluorophenylsulfonylpiperazin-1-yl, 4-ethylsulfonylpiperazin-1-ylmethyl, 4-cyclopropyl carbonylpiperazin-1-ylmethyl, 2-methylpropanoylpiperazin-1-ylmethyl, 4-phenylcarbonyl piperazin-1-ylmethyl, 3-azocan-1-ylmethyl, 4-acetyl-1,4-diazepan-1-yl, 4-phenylamino carbonylpiperazin-1-ylmethyl; 4-ethylaminocarbonylpiperazin-1-ylmethyl; 3-piperidin-1-ylpropyloxy, 2-pyrrolidin-1-ylethyloxy; 3-piperidin-1-ylpropyloxy; and 3-morpholin-4-ylpropyloxy.

In one preferred embodiment, the compound is a compound of formula (I) in the Summary of the Invention, wherein R⁹ is optionally substituted heteroaryl or optionally substituted heteroaralkyl, optionally substituted with one or more Q¹.

In one preferred embodiment, the compound is a compound of formula (I) in the Summary of the Invention, wherein Q¹ is selected from the group consisting of optionally substituted alkyl, halo and haloalkyl.

In one preferred embodiment, the compound is a compound of formula (I) in the Summary of the Invention, wherein R⁹ is optionally substituted heterocyclyl or optionally substituted heterocyclylalkyl, optionally substituted with one or more Q¹.

In one preferred embodiment, the compound is a compound of formula (I) in the Summary of the Invention, wherein Q¹ is selected from the group consisting of optionally substituted alkyl, optionally substituted heterocyclyl, optionally substituted heterocyclylalkyl, optionally substituted aryl, optionally substituted aralkyl.

In one preferred embodiment, the compound is a compound of formula (I) in the Summary of the Invention, wherein R⁹ is selected from the group consisting of methyl, ethyl, propyl, isopropyl, butyl, isobutyl, pentyl, cyclopentyl, cyclohexyl, cycloheptyl; dimethylaminopropyl, 4-methylpentyl; (3s,5s,7s)-tricyclo{3.3.1.1˜3,7˜]dec-1-yl; 1S,4S)-5-methyl-2,5-diazabicyclo[2.2.1]hept-2-yl]; phenyl, isoxazolyl, piperidinyl, piperazinyl, pyrrolidinyl, morpholinyl, benzodioxolyl, and benzotriazolyl.

In one preferred embodiment, the compound is a compound of formula (I) in the Summary of the Invention, wherein Q¹ is selected from the group consisting of optionally substituted alkyl, optionally substituted aryl, optionally substituted aralkyl, optionally substituted cycloalkyl, optionally substituted cycloalkylalkyl, optionally substituted heteroaryl, optionally substituted heteroaralkyl, optionally substituted heterocyclyl and optionally substituted heterocyclylalkyl.

In one preferred embodiment, the compound is a compound of formula (I) in the Summary of the Invention, wherein R⁸ is hydroxy, halogen, optionally substituted alkyl, optionally substituted aryl, optionally substituted aralkyl, optionally substituted cycloalkyl, optionally substituted cycloalkylalkyl, optionally substituted heteroaryl, optionally substituted heteroaralkyl, optionally substituted heterocyclyl and optionally substituted heterocyclylalkyl.

In one preferred embodiment, the compound is a compound of formula (I) in the Summary of the Invention, wherein R⁸ is selected from the group consisting of methyl, ethyl, propyl, isopropyl, butyl, and isobutyl.

In one preferred embodiment, the compound is a compound of formula (I) in the Summary of the Invention, wherein n is 0, 1, 2, 3 or 4.

In one preferred embodiment, the compound is a compound of formula (I) in the Summary of the Invention, wherein R⁶ or R⁷ is optionally substituted alkyl, preferably methyl, ethyl, propyl, isopropyl, butyl, isobutyl or pentyl.

In one preferred embodiment, the compound is a compound of formula (I) in the Summary of the Invention, wherein R¹ is —C(J)OR¹¹ and R¹¹ is selected from the group consisting of 2,2-dimethyl-1,3-dioxolan-4-yl; 2-piperidin-1-ylethylaminocarbonyl; 2,3-dihydroxypropyl or 2-fluoro-1-(fluoromethyl)ethyl, hydroxyethyl, phenylmethyloxyethyl, 3,4-difluorophenylcarbonyloxy-1-methylethyl, and 2-hydroxy-1-methylethyl.

In one preferred embodiment, the compound is a compound of formula (I) in the Summary of the Invention, wherein R¹ is C(J)N(R¹⁰)(R¹¹) and R¹¹ is optionally substituted alkyl, selected from the group consisting of isopropyl; beta-alanine, 2,3-dihydroxypropyl; and 2-hydroxy-1-(hydroxymethyl)ethyl.

In one preferred embodiment, the compound is a compound of formula (I) in the Summary of the Invention, wherein Q¹ is selected from the group consisting of optionally substituted alkyl, and halogen, preferably methyl, chloro, bromo, fluoro, or 3,4-difluoro.

In one preferred embodiment, the compound is a compound of formula (I) in the Summary of the Invention where R¹ is —C(J)OR¹¹ and R¹¹ is optionally substituted alkyl; preferably methyl, ethyl, propyl, isopropyl, butyl or isobutyl; more preferably, isopropyl; J is O; R⁶ or R⁷ is optionally substituted alkyl; preferably methyl, ethyl, or propyl, more preferably, methyl; n is 0; R³ is COR⁹ wherein R⁹ is optionally substituted heterocyclyl or optionally substituted heterocyclylalkyl, preferably piperidin-3-yl or pipen-din-4-yl; wherein R⁹ is optionally substituted with one or more Q¹; where Q¹ is selected from the group consisting of optionally substituted alkyl, halo and haloalkyl; preferably methyl, ethyl, propyl, isopropyl, butyl, isobutyl or methyethyldiethylamino; more preferably, isopropyl or methyethyldiethylamino.

In another preferred embodiment, the compound is a compound of formula (I) in the Summary of the Invention, where R¹ is —C(J)OR¹¹; R¹¹ is optionally substituted alkyl, preferably methyl, ethyl, propyl, isopropyl, butyl, isobutyl, or pentyl; more preferably, methyl; J is O; R⁶ or R⁷ is optionally substituted alkyl; preferably methyl, ethyl, or propyl; more preferably, methyl; and n is 0. R³ is COR⁹; R⁹ is optionally substituted alkyl, optionally substituted aryl, optionally substituted aralkyl, optionally substituted cycloalkyl, optionally substituted cycloalkylalkyl, optionally substituted heteroaryl, optionally substituted heteroaralkyl, optionally substituted heterocyclyl or optionally substituted heterocyclylalkyl; more preferably, R⁹ is methyl, ethyl, propyl, isopropyl, butyl, isobutyl, pentyl, cyclopentyl, cyclohexyl, cycloheptyl; dimethylaminopropyl, 4-methylpentyl; or (3s,5s,7s)-tricyclo[3.3.1.1˜3,7˜]dec-1-yl; more preferably, butyl, cyclohexyl or cycloheptyl. R⁹ is optionally substituted with one or more Q¹ selected from the group consisting of optionally substituted alkyl, optionally substituted cycloalkyl or optionally substituted cycloalkylalkyl; preferably methyl, ethyl, propyl, isopropyl, butyl, isobutyl, or pentyl; more preferably butyl, cyclohexyl or cycloheptyl. In another preferred embodiment, the compound is a compound of formula (I) in the Summary of the Invention, where R¹ is —C(J)OR¹¹; R¹¹ is optionally substituted alkyl, preferably methyl, ethyl, propyl, isopropyl, butyl, isobutyl, or pentyl; more preferably, methyl; J is O; R⁶ or k⁷ is independently optionally substituted alkyl, preferably methyl; n is 0-3; R⁸ is optionally substituted alkyl or halo, preferably, fluoro, chloro or bromo; R³ is CO(NR¹¹) (NR¹²); wherein R¹¹ is hydrogen or optionally substituted alkyl; preferably hydrogen, methyl, or ethyl; more preferably hydrogen; R¹² is optionally substituted alkyl, optionally substituted aryl, optionally substituted aralkyl, optionally substituted cycloalkyl, optionally substituted cycloalkylalkyl, optionally substituted heterocyclyl, or optionally substituted heterocyclylalkyl; preferably methyl, ethyl, propyl, isopropyl, butyl, isobutyl, dimethylaminoethyl, dimethylaminopropyl, diethylaminoethyl, diethylamino, dimethylamino, 2-morpholin-4-ylethyl, 3-morpholin-4-ylpropyl, 3-morpholin-4-ylpropyl)amino, or piperidinyl. R¹¹ and R¹² together to which they are attached form optionally substituted heterocylcyl, optionally substituted heterocyclylalkyl; preferably pyrrolidin-1-yl, 4-pyrrolidin-1-yl, piperidin-1-yl, 4-methylpiperazin-1-yl, 4-ethylpiperazin-1-yl, 4-piperazin-1-yl, 4-propylpiperazin-1-yl, piperidin-3-yl, piperidinyl, (1S,4S)-5-methyl-2,5-diazabicyclo[2.2.1]hept-2-yl or azepanyl;

R¹¹ and R¹² together is optionally substituted with one or more Q¹ selected from the group consisting of optionally substituted alkyl, optionally substituted aryl, optionally substituted heterocyclyl and optionally substituted heterocyclylalkyl; preferably methyl, ethyl, propyl, diethylamino, dimethylamino, diethylaminomethyl, diethylaminoethyl, dimethylaminopropyloxymethyl, phenyl, phenylmethyl, pyrrolidinyl, piperazinyl, piperidinyl, methylpiperidinyl, methylpiperazinyl, 2-oxo-2-pyrrolidin-1ylethyl, or morpholino-4-methyl.

In another preferred embodiment, the compound is a compound of formula (I) in the Summary of the Invention, where R¹ is —C(J)OR¹¹; R¹¹ is optionally substituted alkyl, preferably methyl, ethyl, propyl, isopropyl, butyl, isobutyl, or pentyl; more preferably, methyl; J is O; R⁶ or R⁷ is independently optionally substituted alkyl, preferably methyl; n is 0-3; R⁸ is halo, preferably fluoro, chloro or bromo; Each R¹⁰ is independently optionally substituted alkyl, preferably methyl, ethyl, propyl, isopropyl, butyl, isobutyl, or pentyl; more preferably, isopropyl. R³ is CO(NR¹¹)(NR¹²); Each R¹¹ is preferably hydrogen or optionally substituted alkyl; more preferably methyl, or ethyl; most preferably, hydrogen; R¹² is optionally substituted alkyl, preferably methyl, ethyl, propyl; optionally substituted cycloalkyl or optionally substituted cycloalkylalkyl, preferably, cyclopentyl, cyclohexyl, cyclopheptyl; or optionally substituted aryl or optionally substituted aralkyl, preferably phenylmethyl or phenyl.

In another preferred embodiment, the compound is a compound of formula (I) in the Summary of the Invention, where R¹ is —C(J)OR¹¹; R¹¹ is optionally substituted alkyl, preferably methyl, ethyl, propyl, isopropyl, butyl, isobutyl, or pentyl; more preferably, methyl; J is O; R⁶ or R⁷ is optionally substituted alkyl; preferably methyl, ethyl, or propyl; and n is 0. R³ is COR⁹ wherein R⁹ is optionally substituted aryl or optionally substituted aralkyl; preferably phenyl; wherein R⁹ is substituted with one or more Q¹ selected from the group consisting of hydroxy, halogen, haloalkyl, haloalkoxy, optionally substituted alkyl, alkoxy, cyano, optionally substituted aryl, optionally substituted aralkyl, optionally substituted heterocyclyl, or optionally substituted heterocyclylalkyl; Q¹ is preferably hydroxy, cyano, 2-methyl; 3-methyl; methylpiperazinyl, 3-chloromethyl, 3,4-difluoro; 3-methyl, 4-methyl; 2-methyloxy; 3-methyloxy; 4-methyloxy; 3-fluoro-4-methyl; 4-fluoro-3-methyl; 2-trifluoromethyloxy; 2-chloro; 3-chloro; 4-chloro; 2,4-dichloro; 2-chloro-3,6-difluoro, 3-chloro-2,6-difluoro, 2-fluoro; 3-fluoro; 2-bromo; 3-trifluoromethyl; 2,3-difluoro; 2,4-difluoro; 2,5-difluoro; 2,6-difluoro; 3,4-difluoro; 3,6-difluoro; 3,4-difluoro; 2,3-difluoro-4-trifluoromethyl; 2-fluoro-4-trifluoromethyl; 2-fluoro-3-trifluoromethyl; 3-fluoro-5-trifluoromethyl; 2,5-bistrifluoromethyl; 3,5-bistrifluoromethyl; 3-chloro-2-fluoro-4-trifluoromethy; 3-fluoro-4-trifluoromethyl; 4-fluoro-3-trifluoromethyl; 4-fluoro-2-trifluoromethyl; 2-chloro-4-fluoro; 3-chloro-4-fluoro; 2-trifluoromethyl; 4-trifluoromethyl; 2,3,4-trifluoro; 2,4,6-trifluoro; 2,4,5-trifluoro; 3,4-bis(methyloxy); 3-phenylmethyloxy; or methyloxyphenylmethyloxy.

In another preferred embodiment, the compound is a compound of formula (I) in the Summary of the Invention, where R¹ is —C(J)OR¹¹; J is O; R¹¹ is optionally substituted alkyl, preferably, methyl, ethyl, propyl, isopropyl, butyl, isobutyl, or pentyl; more preferably, methyl; R⁶ or R⁷ is optionally substituted alkyl; preferably methyl, ethyl, or propyl, more preferably, methyl; and n is 0. R³ is COR⁹ wherein R⁹ is optionally substituted aryl, optionally substituted aralkyl, optionally substituted heterocyclyl, optionally substituted heterocyclylalkyl, optionally substituted heteroaryl or optionally substituted heteroarylalkyl; more preferably 1,3 benzodioxol-5-yl or methylisoxazol-3-yl; wherein R⁹ is optionally substituted with one or more Q¹ selected from the group consisting of optionally substituted alkyl, halogen or haloalkyl; preferably methyl, ethyl, propyl, isopropyl, butyl, isobutyl, or pentyl; more preferably, methyl, fluoro, chloro or bromo.

In another preferred embodiment, the compound is a compound of Formula I in the Summary of the Invention, where R¹ is —C(J)OR¹¹; J is O; R¹¹ is optionally substituted alkyl, preferably methyl, ethyl, propyl, isopropyl, butyl, isobutyl, or pentyl; more preferably, methyl; R⁶ or R⁷ is optionally substituted alkyl; preferably methyl, ethyl, or propyl, more preferably, methyl; and n is 0. R³ is COR⁹ wherein R⁹ is optionally substituted aryl, or optionally substituted aralkyl, preferably, phenyl. R⁹ is optionally substituted with one or more Q¹ selected from the group consisting of independently optionally substituted alkyl, halogen, optionally substituted aryl, optionally substituted aralkyl, optionally substituted heterocyclyl, or optionally substituted heterocyclylalkyl; Q¹ is preferably 3,4-difluoro; 4-piperidin-4-yl, 3-piperidin-4-yl, 3-piperidin-4-ylmethyl, piperidin-4-ylmethyl, dimethylaminomethyl, diethylaminomethyl, dimethylaminoethyloxy, dimethylaminopropyloxy, diethylaminopropyloxy, 4-methylsulfonylpiperazin-1-yl, 3-azepan-1-ylmethyl, 4-methyl-1,4-diazepan-1-yl, 3-pyrrolidin-1-ylethyl, 4-methylpiperazin-1-ylmethyl; 4-ethylpiperazin-1-ylmethyl; 3-piperazin-1-ylmethyl; morpholin-4-ylmethyl; 3-morpholin-4-ylmethyl; 2-morpholin-4-ylethyloxy; 2-piperidin-1-ylethyloxy; 3-morpholin-4-ylpropyloxy 1H-pyrazol-1-yl, 4-trifluoromethyl-1H-pyrazol-1-yl, 4-acetylpiperazin-1-ylmethyl; methylbenzotriazolyl, dimethylethyloxycarbonylpiperazin-1-ylmethyl, 4-phenylsulfonylpiperazin-1-ylmethyl, 4-fluorophenylsulfonylpiperazin-1-yl, 4-ethylsulfonylpiperazin-1-ylmethyl, 4-cyclopropyl carbonylpiperazin-1-ylmethyl, 2-methylpropanoylpiperazin-1-ylmethyl, 4-phenylcarbonyl piperazin-1-ylmethyl, 3-azocan-1-ylmethyl, 4-acetyl-1,4-diazepan-1-yl, 4-phenylamino carbonylpiperazin-1-ylmethyl; 4-ethylaminocarbonylpiperazin-1-ylmethyl; 3-piperidin-1-ylpropyloxy, 2-pyrrolidin-1-ylethyloxy; 3-piperidin-1-ylpropyloxy; or 3-morpholin-4-ylpropyloxy.

In another preferred embodiment, the compound is a compound of Formula I in the Summary of the Invention, where R¹ is —C(J)OR¹¹; J is O; R¹¹ is optionally substituted alkyl, preferably methyl, ethyl, propyl, isopropyl, butyl, isobutyl, or pentyl; more preferably, methyl; R⁶ or R⁷ is optionally substituted alkyl; preferably methyl, ethyl, or propyl, more preferably, methyl; and n is 0. R³ is COR⁹ wherein R⁹ is optionally substituted aryl, or optionally substituted aralkyl, preferably, phenyl. R⁹ is optionally substituted with —O—(CH₂)ₚ—R²⁸. p is 1-3; R²⁸ is optionally substituted alkyl, optionally substituted aryl, optionally substituted aralkyl, optionally substituted heterocyclyl, or optionally substituted heterocyclylalkyl, preferably, R²⁸ is phenyl, dimethylamino, diethylamino, N-ethyl, N-methyl amino, morpholinyl, piperidinyl, piperazinyl, pyrrolidinyl, morpholinyl, or 4-methyloxyphenyl.

In another preferred embodiment, the compound is a compound of Formula I in the Summary of the Invention, where R¹ is —C(J)OR¹¹; J is O; R¹¹ is optionally substituted alkyl, preferably methyl, ethyl, propyl, isopropyl, butyl, isobutyl, or pentyl; more preferably, methyl; R⁶ or R⁷ is optionally substituted alkyl; preferably methyl, ethyl, or propyl, more preferably, methyl; and n is 0. R³ is COR⁹ wherein R⁹ is optionally substituted aryl, optionally substituted aralkyl, preferably, phenyl. R⁹ is optionally substituted with one or more Q¹. R⁹ is optionally substituted with —(CH₂)ₚ—R²⁹; p is 1-3; R²⁹ is halogen, optionally substituted alkyl, optionally substituted aryl, optionally substituted aralkyl, optionally substituted heterocyclyl, or optionally substituted heterocyclylalkyl; preferably, R²⁹ is dimethylamino, diethylamino, N-ethyl, N-methyl amino, chloro, morpholinyl, piperidinyl, piperazinyl, piperazin-1-ylmethyl, piperazin-1-ylethyl, pyrrolidinyl, morpholinyl, methyloxyphenyl; 4-acetylpiperazin-1-yl; 4-methylsulfonylpiperazin-1-yl; azepanyl; azocan-1-yl; 4-methyl-1,4-diazepan-1-yl; 4-acetyl-1,4-diazepan-1-yl; dimethylethyloxy carbonylpiperazin-1-yl; 4-phenylsulfonyl piperazin-1-yl; 4-fluorophenylsulfonylpiperazin-1-yl; ethylsulfonyl piperazin-1-yl; cyclopropylcarbonyl piperazin-1-yl; 2-methylpropanoyl piperazin-1-yl; phenylcarbonyl piperazin-1-yl; 4-phenylaminocarbonylpiperazin-1-yl; or 4-ethylaminocarbonylpiperazin-1-yl; Q¹ is halogen or optionally substituted alkyl, preferably, methyl, chloro, fluoro or bromo; and m is 0-3.

In another preferred embodiment, the compound is a compound of Formula I in the Summary of the Invention, where R¹ is —C(J)OR¹¹; J is O; R⁶ or R⁷ is optionally substituted alkyl; preferably methyl, ethyl, or propyl, more preferably, methyl; and n is 0. R³ is COR⁹ wherein R⁹ is optionally substituted aryl, or optionally substituted aralkyl, preferably, phenyl. R⁹ is optionally substituted with one or more Q¹. Each R¹¹ is independently optionally substituted alkyl, optionally substituted aryl, optionally substituted aralkyl, optionally substituted heterocyclyl, or optionally substituted heterocyclylalkyl. Preferably, R¹¹ is 2,2-dimethyl-1,3-dioxolan-4-yl; 2-piperidin-1-ylethylaminocarbonyl; 2,3-dihydroxypropyl or 2-fluoro-1-(fluoromethyl)ethyl, hydroxyethyl, phenylmethyloxyethyl, 3,4-difluorophenylcarbonyloxy-1-methylethyl, 2-hydroxy-1-methylethyl; p is 1-3; Q¹ is halogen or optionally substituted alkyl, preferably methyl, chloro, fluoro, bromo or 3,4-difluoro.

In another embodiment, the compound is a compound of Formula I in the Summary of the Invention, where R¹ is —C(J)N(R¹⁰)(R¹¹); J is O; R¹⁰ is independently hydrogen or optionally substituted alkyl, more preferably, hydrogen; R¹¹ is independently optionally substituted alkyl, preferably isopropyl; beta-alanine, 2,3-dihydroxypropyl; or 2-hydroxy-1-(hydroxymethyl)ethyl; R⁶ or R⁷ is optionally substituted alkyl; preferably methyl, ethyl, or propyl, more preferably, methyl; and n is 0-3; R⁸ is optionally substituted alkyl or halo, preferably chloro, bromo or fluoro. R³ is COR⁹ wherein R⁹ is optionally substituted aryl, or optionally substituted aralkyl, preferably, phenyl. R¹ is optionally substituted with one or more Q¹ selected from the group consisting of halogen and optionally substituted alkyl, preferably methyl, chloro, fluoro, bromo or 3,4-difluoro.

In another embodiment, R¹ is —C(J)OR¹¹, J is O; R¹ is optionally substituted alkyl, preferably methyl, ethyl, propyl, isopropyl, butyl, isobutyl, or pentyl; more preferably, methyl; R⁶ or R⁷ is optionally substituted alkyl; preferably methyl, ethyl, or propyl, more preferably, methyl; R⁸ is OR wherein R is independently hydrogen, optionally substituted alkyl, optionally substituted aryl, optionally substituted aralkyl, optionally substituted heterocyclyl or optionally substituted heterocyclylalkyl. Preferably, R is 2-(dimethylamino)ethylaminocarbonyl; 1,1-dimethylethyloxycarbonyl; 2-diethylaminoethylaminocarbonyl; dimethylaminopropyl; dimethylaminoethyl; methylamino carbonyl; diethylaminoethyl; methyloxyethyl; dimethylaminopropylaminocarbonyl; phenylmethyl; hydroxy; 2-pyrrolidinyl-1-ylaminocarbonyl; and n is 1-3. R³ is COR⁹ wherein R⁹ is optionally substituted aryl or optionally substituted aralkyl, preferably, phenyl. R⁹ is optionally substituted with one or more Q¹ selected from the group consisting of halogen and optionally substituted alkyl, preferably methyl, chloro, fluoro, bromo and 3,4-difluoro.

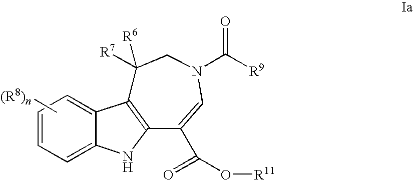

In Embodiment 1, the invention provides a compound of Formula Ia, wherein each R⁶ and R⁷ is independently optionally substituted alkyl; preferably methyl; n is 0; R¹¹ is optionally substituted alkyl, preferably methyl, ethyl, propyl, isopropyl, butyl or isobutyl; more preferably, methyl. R⁹ is optionally substituted alkyl, optionally substituted heterocyclyl or optionally substituted heterocyclylalkyl. Preferably, R⁹ is piperidin-3-yl or piperidin-4-yl. R⁹ is optionally substituted with one or more Q¹ selected from the group consisting of optionally substituted alkyl, halo and haloalkyl; preferably methyl, ethyl, propyl, isopropyl, butyl, or methylethyldiethylamino; more preferably, methyl or methylethyldiethylamino.

Preferred compounds of Embodiment 1 are selected from the group consisting of:
1-methylethyl 1,1-dimethyl-3-[(1-methylpiperidin-3-yl)carbonyl]-1,2,3,6-tetrahydroazepino[4,5-b]indole-5-carboxylate;
1-methylethyl 1,1-dimethyl-3-[(1-methylpiperidin-4-yl)carbonyl]-1,2,3,6-tetrahydroazepino[4,5-b]indole-5-carboxylate; and
1-methylethyl 3-[4-(dimethylamino)butanoyl]-1,1-dimethyl-1,2,3,6-tetrahydroazepino[4,5-b]indole-5-carboxylate.

In Embodiment 2, the invention provides a compound of Formula Ia wherein each R⁶ and R⁷ is independently optionally substituted alkyl; preferably methyl; n is 0; R⁹ is optionally substituted alkyl, optionally substituted aryl, optionally substituted aralkyl, optionally substituted cycloalkyl, optionally substituted heterocyclyl, optionally substituted heteroaryl, or optionally substituted heteroarylalkyl; Preferably, R⁹ is methyl, ethyl, propyl, isopropyl, butyl, isobutyl, pentyl, cyclopentyl, cyclohexyl, cycloheptyl; dimethylaminopropyl, 4-methylpentyl; (3s,5s,7s)-tricyclo[3.3.1.1˜3,7˜]dec-1-yl; more preferably, butyl, cyclohexyl or cycloheptyl. R⁹ is optionally substituted with one or more Q¹, selected from the group consisting of optionally substituted alkyl, preferably methyl, ethyl, propyl, isopropyl, butyl, isobutyl, and pentyl. Each R¹⁰ is independently optionally substituted alkyl, preferably methyl, ethyl, propyl, isopropyl, butyl, isobutyl, or pentyl.

Preferred compounds of Embodiment 2 are selected from the group consisting of:
1-methylethyl 3-(cyclohexylcarbonyl)-1,1-dimethyl-1,2,3,6-tetrahydroazepino[4,5-b]indole-5-carboxylate;
1-methylethyl 3-acetyl-1,1-dimethyl-1,2,3,6-tetrahydroazepino[4,5-b]indole-5-carboxylate;
1-methylethyl 3-butanoyl-1,1-dimethyl-1,2,3,6-tetrahydroazepino[4,5-b]indole-5-carboxylate;
1-methylethyl 1,1-dimethyl-3-pentanoyl-1,2,3,6-tetrahydroazepino[4,5-b]indole-5-carboxylate;
1-methylethyl 3-(cyclopentylcarbonyl)-1,1-dimethyl-1,2,3,6-tetrahydroazepino[4,5-b]indole-5-carboxylate;
1-methylethyl 3-(2,2-dimethyl propanoyl)-1,1-dimethyl-1,2,3,6-tetrahydroazepino[4,5-b]indole-5-carboxylate;
1-methylethyl 3-(2-ethylbutanoyl)-1,1-dimethyl-1,2,3,6-tetrahydroazepino[4,5-b]indole-5-carboxylate;
1-methylethyl 1,1-dimethyl-3-(3-methylbutanoyl)-1,2,3,6-tetrahydroazepino[4,5-b]indole-5-carboxylate;
1-methylethyl 3-(cycloheptylcarbonyl)-1,1-dimethyl-1,2,3,6-tetrahydroazepino[4,5-b]indole-5-carboxylate;
1-methylethyl 1,1-dimethyl-3-propanoyl-1,2,3,6-tetrahydroazepino[4,5-b]indole-5-carboxylate;
1-methylethyl 1,1-dimethyl-3-[(3s,5s,7s)-tricyclo[3.3.1.1˜3,7˜]dec-1-ylcarbonyl]-1,2,3,6-tetrahydroazepino[4,5-b]indole-5-carboxylate; and
1-methylethyl 1,1-dimethyl-3-(4-methylpentanoyl)-1,2,3,6-tetrahydroazepino[4,5-b]indole-5-carboxylate;

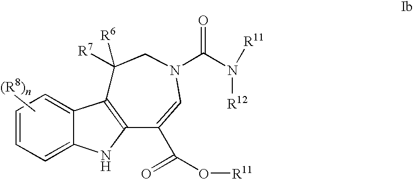

In Embodiment 3, the invention provides compound of Formula Ib wherein each R⁶ and R⁷ is independently optionally substituted alkyl, preferably methyl; n is 0; Each R¹¹ is hydrogen or optionally substituted alkyl; preferably hydrogen, methyl, or ethyl; more preferably hydrogen; R¹² is optionally substituted alkyl, optionally substituted aryl, optionally substituted aralkyl, optionally substituted cycloalkyl, optionally substituted cycloalkylalkyl, optionally substituted heterocyclyl, optionally substituted heterocyclylalkyl; preferably methyl, ethyl, propyl, isopropyl, butyl, isobutyl, dimethylaminoethyl, dimethylaminopropyl, diethylaminoethyl, diethylamino, dimethylamino, 2-morpholin-4-ylethyl, 3-morpholin-4-ylpropyl, 3-morpholin-4-ylpropyl)amino, or piperidinyt. R¹ and R¹² together to which they are attached form optionally substituted heterocyclyl, optionally substituted heterocyclylalkyl; preferably pyrrolidin-1-yl, 4-pyrrolidin-1-yl, piperidin-1-yl, 4-methylpiperazin-1-yl, 4-ethylpiperazin-1-yl, 4-piperazin-1-yl, 4-propylpiperazin-1-yl, piperidin-3-yl, piperidinyl, (1S,4S)-5-methyl-2,5-diazabicyclo[2.2.1]hept-2-yl or azepanyl.

R¹¹ and R¹² together is optionally substituted with one or more Q¹ selected from the group consisting of optionally substituted alkyl, optionally substituted aryl, optionally substituted heterocyclyl and optionally substituted heterocyclylalkyl; preferably methyl, ethyl, propyl, diethylamino, dimethylamino, diethylaminomethyl, diethylaminoethyl, dimethylaminopropyloxymethyl, phenyl, phenylmethyl, pyrrolidinyl, piperazinyl, piperidinyl, methylpiperidinyl, methylpiperazinyl, 2-oxo-2-pyrrolidin-1 ylethyl, or morpholino-4-methyl.

Preferred compounds of Embodiment 3 are selected from the group consisting of:
1-methylethyl 1,1-dimethyl-3-{[(1S,4S)-5-methyl-2,5-diazabicyclo[2.2.1]hept-2-yl]carbonyl}-1,2,3,6-tetrahydroazepino[4,5-b]indole-5-carboxylate;
1-methylethyl 1,1-dimethyl-3-[(4-pyrrolidin-1-ylpiperidin-1-yl)carbonyl]-1,2,3,6-tetrahydroazepino[4,5-b]indole-5-carboxylate;
1-methylethyl 1,1-dimethyl-3-(piperidin-1-ylcarbonyl)-1,2,3,6-tetrahydroazepino[4,5-b]indole-5-carboxylate;
1-methylethyl 3-({[3-(dimethylamino)propyl]amino}carbonyl)-1,1-dimethyl-1,2,3,6-tetrahydroazepino[4,5-b]indole-5-carboxylate;
1-methylethyl 1,1-dimethyl-3-({[4-(4-methylpiperazin-1-yl)phenyl]amino}carbonyl)-1,2,3,6-tetrahydroazepino[4,5-b]indole-5-carboxylate;
1-methylethyl 1,1-dimethyl-3-(pyrrolidin-1-ylcarbonyl)-1,2,3,6-tetrahydroazepino[4,5-b]indole-5-carboxylate;
1-methylethyl 1,1-dimethyl-3-[(4-methylpiperazin-1-yl)carbonyl]-1,2,3,6-tetrahydroazepino[4,5-b]indole-5-carboxylate;
1-methylethyl 3-({[2-(dimethylamino)ethyl]amino}carbonyl)-1,1-dimethyl-1,2,3,6-tetrahydroazepino[4,5-b]indole-5-carboxylate;
1-methylethyl 1,1-dimethyl-3-{[(3-morpholin-4-ylpropyl)amino]carbonyl}-1,2,3,6-tetrahydroazepino[4,5-b]indole-5-carboxylate;
1-methylethyl 1,1-dimethyl-3-{[(2-morpholin-4-ylethyl)amino]carbonyl}-1,2,3,6-tetrahydroazepino[4,5-b]indole-5-carboxylate;
1-methylethyl 3-[(4-ethylpiperazin-1-yl)carbonyl]-1,1-dimethyl-1,2,3,6-tetrahydroazepino[4,5-b]indole-5-carboxylate;
1-methylethyl 1,1-dimethyl-3-(piperazin-1-ylcarbonyl)-1,2,3,6-tetrahydroazepino[4,5-b]indole-5-carboxylate;
1-methylethyl 3-({[2-(diethylamino)ethyl](ethyl)amino}carbonyl)-1,1-dimethyl-1,2,3,6-tetrahydroazepino[4,5-b]indole-5-carboxylate;
1-methylethyl 1,1-dimethyl-3-({-4-[(1-methylpiperidin-4-yl)methyl]piperazin-1-yl}carbonyl)-1,2,3,6-tetrahydroazepino[4,5-b]indole-5-carboxylate;
1-methylethyl 1,1-dimethyl-3-{[4-(1-methylethyl)piperazin-1-yl]carbonyl}-1,2,3,6-tetrahydroazepino[4,5-b]indole-5-carboxylate;
1-methylethyl 1,1-dimethyl-3-[(4-propylpiperazin-1-yl)carbonyl]-1,2,3,6-tetrahydroazepino[4,5-b]indole-5-carboxylate;
1-methylethyl 9-fluoro-1,1-dimethyl-3-[(4-methylpiperazin-1-yl)carbonyl]-1,2,3,6-tetrahydroazepino[4,5-b]indole-5-carboxylate;
1-methylethyl 3-{[4-(diethylamino) piperidin-1-yl]carbonyl}-1,1-dimethyl-1,2,3,6-tetrahydroazepino[4,5-b]indole-5-carboxylate;
1-methylethyl 8-fluoro-1,1-dimethyl-3-[(4-methylpiperazin-1-yl)carbonyl]-1,2,3,6-tetrahydroazepino[4,5-b]indole-5-carboxylate;
1-methylethyl 3-[(4-ethylpiperazin-1-yl)carbonyl]-8-fluoro-1,1-dimethyl-1,2,3,6-tetrahydroazepino[4,5-b]indole-5-carboxylate;
1-methylethyl 1,1-dimethyl-3-{[4-(2-oxo-2-pyrrolidin-1-ylethyl)piperazin-1-yl]carbonyl}-1,2,3,6-tetrahydroazepino[4,5-b]indole-5-carboxylate;
1-methylethyl 3-({4-[2-(diethylamino)ethyl]piperazin-1-yl}carbonyl)-1,1-dimethyl-1,2,3,6-tetrahydroazepino[4,5-b]indole-5-carboxylate;
1-methylethyl 3-{[3-(dimethylamino)piperidin-1-yl]carbonyl}-1,1-dimethyl-1,2,3,6-tetrahydroazepino[4,5-b]indole-5-carboxylate;
1-methylethyl 3-(azepan-1-ylcarbonyl)-1,1-dimethyl-1,2,3,6-tetrahydroazepino[4,5-b]indole-5-carboxylate;
1-methylethyl 1,1-dimethyl-3-{[4-(4-methylpiperazin-1-yl)piperidin-1-yl]carbonyl}-1,2,3,6-tetrahydroazepino[4,5-b]indole-5-carboxylate;
1-methylethyl 1,1-dimethyl-3-[(4-methyl-1,4-diazepan-1-yl)carbonyl]-1,2,3,6-tetrahydroazepino[4,5-b]indole-5-carboxylate;
1-methylethyl 1,1-dimethyl-3-(morpholin-4-ylcarbonyl)-1,2,3,6-tetrahydroazepino[4,5-b]indole-5-carboxylate;
1-methylethyl 3-({3-[(dimethylamino)methyl]piperidin-1-yl}carbonyl)-1,1-dimethyl-1,2,3,6-tetrahydroazepino[4,5-b]indole-5-carboxylate;
1-methylethyl 3-({(3S)-3-[(dimethylamino)methyl]piperidin-1-yl}carbonyl)-1,1-dimethyl-1,2,3,6-tetrahydroazepino[4,5-b]indole-5-carboxylate;
1-methylethyl 3-({(3R)-3-[(dimethylamino)methyl]piperidin-1-yl}carbonyl)-1,1-dimethyl-1,2,3,6-tetrahydroazepino[4,5-b]indole-5-carboxylate;
1-methylethyl 3-[(diethylamino)carbonyl]-1,1-dimethyl-1,2,3,6-tetrahydroazepino[4,5-b]indole-5-carboxylate;
1-methylethyl 1,1-dimethyl-3-{[3-(morpholin-4-ylmethyl)piperidin-1-yl]carbonyl}-1,2,3,6-tetrahydroazepino[4,5-b]indole-5-carboxylate;
1-methylethyl 1,1-dimethyl-3-{[(3S)-piperidin-3-ylamino]carbonyl}-1,2,3,6-tetrahydroazepino[4,5-b]indole-5-carboxylate;
1-methylethyl 3-{[3-({[3-(dimethylamino)propyl]oxy}methyl)piperidin-1-yl]carbonyl}-1,1-dimethyl-1,2,3,6-tetrahydroazepino[4,5-b]indole-5-carboxylate;
1-methylethyl 1,1-dimethyl-3-[(piperidin-3-ylamino)carbonyl]-1,2,3,6-tetrahydroazepino[4,5-b]indole-5-carboxylate;
1-methylethyl 1,1-dimethyl-3-{[(3R)-3-(morpholin-4-ylmethyl)piperidin-1-yl]carbonyl}-1,2,3,6-tetrahydroazepino[4,5-b]indole-5-carboxylate;
1-methylethyl 1,1-dimethyl-3-{[(3R)-3-(piperidin-1-ylmethyl)piperidin-1-yl]carbonyl}-1,2,3,6-tetrahydroazepino[4,5-b]indole-5-carboxylate;
1-methylethyl 1,1-dimethyl-3-{[4-(phenylmethyl)-1,4-diazepan-1-yl]carbonyl}-1,2,3,6-tetrahydroazepino[4,5-b]indole-5-carboxylate; and
1-methylethyl 3-[(3′R)-1,3′-bipiperidin-1′-ylcarbonyl]-1,1-dimethyl-1,2,3,6-tetrahydroazepino[4,5-b]indole-5-carboxylate.

In Embodiment 4, the invention provides compound of Formula Ib wherein each R⁶ and R⁷ is independently optionally substituted alkyl, preferably methyl; n is 0; Each R¹⁰ is independently optionally substituted alkyl, preferably methyl, ethyl, propyl, isopropyl, butyl, isobutyl, or pentyl; more preferably, isopropyl. Each R¹¹ is preferably hydrogen or optionally substituted alkyl; preferably methyl, or ethyl; more preferably, hydrogen; R¹² is optionally substituted alkyl, preferably methyl, ethyl, propyl; optionally substituted cycloalkyl or optionally substituted cycloalkylalkyl, preferably, cyclopentyl, cyclohexyl, cyclopheptyl; optionally substituted aryl or optionally substituted aralkyl, preferably phenylmethyl or phenyl.

Preferred compounds of Embodiment 4 are selected from the group consisting of:
1-methylethyl 1,1-dimethyl-3-[(propylamino)carbonyl]-1,2,3,6-tetrahydroazepino[4,5-b]indole-5-carboxylate;
1-methylethyl 3-[(cyclopentylamino)carbonyl]-1,1-dimethyl-1,2,3,6-tetrahydroazepino[4,5-b]indole-5-carboxylate;
1-methylethyl 3-[(cyclohexylamino)carbonyl]-1,1-dimethyl-1,2,3,6-tetrahydroazepino[4,5-b]indole-5-carboxylate;
1-methylethyl 3-[(cycloheptylamino)carbonyl]-1,1-dimethyl-1,2,3,6-tetrahydroazepino[4,5-b]indole-5-carboxylate; and
1-methylethyl 1,1-dimethyl-3-{[(phenylmethyl)amino]carbonyl}-1,2,3,6-tetrahydroazepino[4,5-b]indole-5-carboxylate.

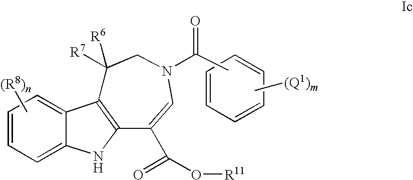

In Embodiment 5, the invention provides a compound of Formula Ic wherein each R⁶ and R⁷ is independently optionally substituted alkyl, preferably methyl; n is 0-3; R⁸ is optionally substituted alkyl or halo, preferably fluoro, chloro or bromo; Each R¹¹ is independently optionally substituted alkyl, preferably methyl, ethyl, propyl, isopropyl, butyl, isobutyl, or pentyl; more preferably, isopropyl. Q¹ is independently hydroxy, halogen, haloalkyl, haloalkoxy, optionally substituted alkyl, alkoxy, cyano, optionally substituted aryl, optionally substituted aralkyl, optionally substituted heterocyclyl, or optionally substituted heterocyclylalkyl; Q¹ is preferably hydroxy, cyano, 2-methyl; 3-methyl; methylpiperazinyl, 3-chloromethyl, 3,4-difluoro; 3-methyl, 4-methyl; 2-methyloxy; 3-methyloxy; 4-methyloxy; 3-fluoro-4-methyl; 4-fluoro-3-methyl; 2-trifluoromethyloxy; 2-chloro; 3-chloro; 4-chloro; 2,4-dichloro; 2-chloro-3,6-difluoro, 3-chloro-2,6-difluoro, 2-fluoro; 3-fluoro; 2-bromo; 3-trifluoromethyl; 2,3-difluoro, 2,4-difluoro, 2,5-difluoro; 2,6-difluoro; 3,4-difluoro; 3,6-difluoro; 3,4-difluoro; 2,3-difluoro-4-trifluoromethyl; 2-fluoro-4-trifluoromethyl; 2-fluoro-3-trifluoromethyl; 3-fluoro-5-trifluoromethyl; 2,5-bistrifluoromethyl; 3,5-bistrifluoromethyl; 3-chloro-2-fluoro-4-trifluoromethy; 3-fluoro-4-trifluoromethyl; 4-fluoro-3-trifluoromethyl; 4-fluoro-2-trifluoromethyl; 2-chloro-4-fluoro; 3-chloro-4-fluoro; 2-trifluoromethyl; 4-trifluoromethyl; 2,3,4-trifluoro; 2,4,6-trifluoro; 2,4,5-trifluoro; 3,4-bis(methyloxy); 3-phenylmethyloxy; or methyloxyphenylmethyloxy; m is 0-3.

Preferred compounds of Embodiment 5 are selected from the group consisting of:
1-methylethyl 3-[(2-chloro-3,6-difluorophenyl)carbonyl]-1,1-dimethyl-1,2,3,6-tetrahydroazepino[4,5-b]indole-5-carboxylate;
1-methylethyl 1,1-dimethyl-3-(phenylcarbonyl)-1,2,3,6-tetrahydroazepino[4,5-b]indole-5-carboxylate;
1-methylethyl 3-[(2-fluorophenyl)carbonyl]-1,1-dimethyl-1,2,3,6-tetrahydroazepino[4,5-b]indole-5-carboxylate;
1-methylethyl 1,1-dimethyl-3-{[2-(trifluoromethyl)phenyl]carbonyl}-1,2,3,6-tetrahydroazepino[4,5-b]indole-5-carboxylate;
1-methylethyl 1,1-dimethyl-3-{[4-(trifluoromethyl)phenyl]carbonyl}-1,2,3,6-tetrahydroazepino[4,5-b]indole-5-carboxylate;
1-methylethyl 3-[(2-chlorophenyl)carbonyl]-1,1-dimethyl-1,2,3,6-tetrahydro azepino[4,5-b]indole-5-carboxylate;
1-methylethyl 3-[(2-bromophenyl)carbonyl]-1,1-dimethyl-1,2,3,6-tetrahydro azepino[4,5-b]indole-5-carboxylate;
1-methylethyl 1,1-dimethyl-3-[(2-methylphenyl)carbonyl]-1,2,3,6-tetrahydro azepino[4,5-b]indole-5-carboxylate;
1-methylethyl 1,1-dimethyl-3-{[2-(methyloxy)phenyl]carbonyl}-1,2,3,6-tetrahydro azepino[4,5-b]indole-5-carboxylate;
1-methylethyl 1,1-dimethyl-3-({2-[(trifluoromethyl)oxy]phenyl}carbonyl)-1,2,3,6-tetrahydro azepino[4,5-b]indole-5-carboxylate;
1-methylethyl 3-[(2-fluorophenyl)carbonyl]-1,1-dimethyl-1,2,3,6-tetrahydroazepino[4,5-b]indole-5-carboxylate;
1-methylethyl 3-[(3-fluorophenyl)carbonyl]-1,1-dimethyl-1,2,3,6-tetrahydroazepino[4,5-b]indole-5-carboxylate;
1-methylethyl 3-[(2,4-difluorophenyl)carbonyl]-1,1-dimethyl-1,2,3,6-tetrahydro azepino[4,5-b]indole-5-carboxylate;
1-methylethyl 3-[(2,3-difluorophenyl)carbonyl]-1,1-dimethyl-1,2,3,6-tetrahydro azepino[4,5-b]indole-5-carboxylate;
1-methylethyl 3-[(2,6-difluorophenyl)carbonyl]-1,1-dimethyl-1,2,3,6-tetrahydro azepino[4,5-b]indole-5-carboxylate;
1-methylethyl 3-[(2,5-difluorophenyl)carbonyl]-1,1-dimethyl-1,2,3,6-tetrahydro azepino[4,5-b]indole-5-carboxylate;
1-methylethyl 1,1-dimethyl-3-[(2,3,4-trifluorophenyl)carbonyl]-1,2,3,6-tetrahydro azepino[4,5-b]indole-5-carboxylate;
1-methylethyl 1,1-dimethyl-3-[(2,4,6-trifluorophenyl)carbonyl]-1,2,3,6-tetrahydro azepino[4,5-b]indole-5-carboxylate;
1-methylethyl 1,1-dimethyl-3-[(2,4,5-trifluorophenyl)carbonyl]-1,2,3,6-tetrahydro azepino[4,5-b]indole-5-carboxylate;
1-methylethyl 3-[(3-chlorophenyl)carbonyl]-1,1-dimethyl-1,2,3,6-tetrahydro azepino[4,5-b]indole-5-carboxylate;
1-methylethyl 3-[(4-chlorophenyl)carbonyl]-1,1-dimethyl-1,2,3,6-tetrahydro azepino[4,5-b]indole-5-carboxylate;
1-methylethyl 3-{[4-fluoro-3-(trifluoromethyl)phenyl]carbonyl}-1,1-dimethyl-1,2,3,6-tetrahydroazepino[4,5-b]indole-5-carboxylate;
1-methylethyl 3-{[3-fluoro-4-(trifluoromethyl)phenyl]carbonyl}-1,1-dimethyl-1,2,3,6-tetrahydroazepino[4,5-b]indole-5-carboxylate;
1-methylethyl 1,1-dimethyl-3-[(3-methylphenyl)carbonyl]-1,2,3,6-tetrahydro azepino[4,5-b]indole-5-carboxylate;
1-methylethyl 1,1-dimethyl-3-[(4-methylphenyl)carbonyl]-1,2,3,6-tetrahydro azepino[4,5-b]indole-5-carboxylate;
1-methylethyl 1,1-dimethyl-3-{[3-(methyloxy)phenyl]carbonyl}-1,2,3,6-tetrahydro azepino[4,5-b]indole-5-carboxylate;
1-methylethyl 1,1-dimethyl-3-{[4-(methyloxy)phenyl]carbonyl}-1,2,3,6-tetrahydro azepino[4,5-b]indole-5-carboxylate;
3-[(3,4-difluorophenyl)carbonyl]-1,1-dimethyl-N-(1-methylethyl)-1,2,3,4,5,6-hexahydro azepino[4,5-b]indole-5-carboxamide;
1-methylethyl 3-{[3,4-bis(methyloxy)phenyl]carbonyl}-1,1-dimethyl-1,2,3,6-tetrahydroazepino[4,5-b]indole-5-carboxylate;
1-{3-[(3,4-difluorophenyl)carbonyl]-1,1-dimethyl-1,2,3,6-tetrahydroazepino[4,5-b]indol-5-yl}ethanone;
1-methylethyl 1,1-dimethyl-3-[(5-methylisoxazol-3-yl)carbonyl]-1,2,3,6-tetrahydro azepino[4,5-b]indole-5-carboxylate;
1-methylethyl 3-{[4-fluoro-2-(trifluoromethyl)phenyl]carbonyl}-1,1-dimethyl-1,2,3,6-tetrahydroazepino[4,5-b]indole-5-carboxylate;
1-methylethyl 3-[(2-chloro-4-fluorophenyl)carbonyl]-1,1-dimethyl-1,2,3,6-tetrahydroazepino[4,5-b]indole-5-carboxylate;
1-methylethyl 3-{[3-(chloromethyl)phenyl]carbonyl}-1,1-dimethyl-1,2,3,6-tetrahydroazepino[4,5-b]indole-5-carboxylate;
2-chloro-1-{3-[(3,4-difluorophenyl)carbonyl]-1,1-dimethyl-1,2,3,6-tetrahydro azepino[4,5-b]indol-5-yl}ethanone;
methyl 3-[(3,4-difluorophenyl)carbonyl]-1,1-dimethyl-1,2,3,6-tetrahydro azepino[4,5-b]indole-5-carboxylate;
1-methylethyl 1,1-dimethyl-3-({3-[phenylmethyl)oxy]phenyl}carbonyl)-1,2,3,6-tetrahydroazepino[4,5-b]indole-5-carboxylate;
1-methylethyl 1,1-dimethyl-3-{[3-(trifluoromethyl)phenyl]carbonyl}-1,2,3,6-tetrahydroazepino[4,5-b]indole-5-carboxylate;
1-methylethyl 3-[(3-fluoro-4-methylphenyl)carbonyl]-1,1-dimethyl-1,2,3,6-tetrahydroazepino[4,5-b]indole-5-carboxylate;
1-methylethyl 3-{[2-fluoro-4-(trifluoromethyl)phenyl]carbonyl}-1,1-dimethyl-1,2,3,6-tetrahydroazepino[4,5-b]indole-5-carboxylate;
1-methylethyl 3-{[3-chloro-2-fluoro-4-(trifluoromethyl)phenyl]carbonyl}-1,1-dimethyl-1,2,3,6-tetrahydroazepino[4,5-b]indole-5-carboxylate;
1-methylethyl 3-{[2-fluoro-3-(trifluoromethyl)phenyl]carbonyl}-1,1-dimethyl-1,2,3,6-tetrahydroazepino[4,5-b]indole-5-carboxylate;
1-methylethyl 3-{[3-fluoro-5-(trifluoromethyl)phenyl]carbonyl}-1,1-dimethyl-1,2,3,6-tetrahydroazepino[4,5-b]indole-5-carboxylate;
1-methylethyl 3-{[3,5-bis(trifluoromethyl)phenyl]carbonyl}-1,1-dimethyl-1,2,3,6-tetrahydroazepino[4,5-b]indole-5-carboxylate;
1-methylethyl 3-{[2,5-bis(trifluoromethyl)phenyl]carbonyl}-1,1-dimethyl-1,2,3,6-tetrahydroazepino[4,5-b]indole-5-carboxylate;
1-methylethyl 3-{[2,3-difluoro-4-(trifluoromethyl)phenyl]carbonyl}-1,1-dimethyl-1,2,3,6-tetrahydroazepino[4,5-b]indole-5-carboxylate;
1-methylethyl 3-[(3-hydroxyphenyl)carbonyl]-1,1-dimethyl-1,2,3,6-tetrahydro azepino[4,5-b]indole-5-carboxylate;
1-methylethyl 3-[(3-cyanophenyl)carbonyl]-1,1-dimethyl-1,2,3,6-tetrahydro azepino[4,5-b]indole-5-carboxylate;
1-methylethyl 3-[(2,4-dichlorophenyl)carbonyl]-1,1-dimethyl-1,2,3,6-tetrahydro azepino[4,5-b]indole-5-carboxylate;
1-methylethyl 3-[(4-fluoro-3-methylphenyl)carbonyl]-1,1-dimethyl-1,2,3,6-tetrahydroazepino[4,5-b]indole-5-carboxylate;
1-methylethyl 3-[(3-chloro-2,6-difluorophenyl)carbonyl]-1,1-dimethyl-1,2,3,6-tetrahydroazepino[4,5-b]indole-5-carboxylate;
1-methylethyl 3-[(3-chloro-4-fluorophenyl)carbonyl]-1,1-dimethyl-1,2,3,6-tetrahydroazepino[4,5-b]indole-5-carboxylate;
1-methylethyl 3-[(3,4-dichlorophenyl)carbonyl]-1,1-dimethyl-1,2,3,6-tetrahydro azepino[4,5-b]indole-5-carboxylate;
1-methylethyl 3-[(4-chloro-2,5-difluorophenyl)carbonyl]-1,1-dimethyl-1,2,3,6-tetrahydro azepino[4,5-b]indole-5-carboxylate;
1-methylethyl 3-[(3-bromo-4-fluorophenyl)carbonyl]-1,1-dimethyl-1,2,3,6-tetrahydro azepino[4,5-b]indole-5-carboxylate; and
1-methylethyl 3-{[3,4-difluoro-5-({[4-(methyloxy)phenyl]methyl}oxy)phenyl]carbonyl}-1,1-dimethyl-1,2,3,6-tetrahydroazepino[4,5-b]indole-5-carboxylate.

In Embodiment 6, the invention provides compound of Formula Ia wherein each R⁶ and R⁷ is independently optionally substituted alkyl; preferably methyl; n is 0; R⁹ is optionally substituted aryl, optionally substituted aralkyl, optionally substituted heterocyclyl, optionally substituted heterocyclylalkyl, optionally substituted heteroaryl or optionally substituted heteroarylalkyl. Preferably, R⁹ is 1,3-benzodioxol-5-yl or methylisoxazol-3-yl. R⁹ is optionally substituted with one or more Q¹ selected from the group consisting of optionally substituted alkyl, halogen, and haloalkyl; preferably methyl or halogen, more preferably, methyl, F, Cl, or Br.

Preferred compounds of Embodiment 6 are selected from the group consisting of:
1-methylethyl 3-(1,3-benzodioxol-5-ylcarbonyl)-1,1-dimethyl-1,2,3,6-tetrahydro azepino[4,5-b]indole-5-carboxylate;
1-methylethyl 3-[(2,2-difluoro-1,3-benzodioxol-4-yl)carbonyl]-1,1-dimethyl-1,2,3,6-tetrahydroazepino[4,5-b]indole-5-carboxylate;
1-methylethyl 3-[(2,2-difluoro-1,3-benzodioxol-5-yl)carbonyl]-1,1-dimethyl-1,2,3,6-tetrahydroazepino[4,5-b]indole-5-carboxylate; and
1-methylethyl 1,1-dimethyl-3-[(5-methylisoxazol-3-yl)carbonyl]-1,2,3,6-tetrahydro azepino[4,5-b]indole-5-carboxylate.

In Embodiment 7, the invention provides compound of Formula Ic, wherein each R⁶ and R⁷ is independently optionally substituted alkyl, preferably methyl; n is 0; Each R¹¹ is independently optionally substituted alkyl, preferably methyl, ethyl, propyl, isopropyl, butyl, isobutyl, or pentyl; more preferably, isopropyl. Q¹ is independently optionally substituted alkyl, halogen, optionally substituted aryl, optionally substituted aralkyl, optionally substituted heterocyclyl, or optionally substituted heterocyclylalkyl; m is 0-3; Q¹ is preferably 3,4-difluoro; 4-piperidin-4-yl, 3-piperidin-4-yl, 3-piperidin-4-ylmethyl, piperidin-4-ylmethyl, dimethylaminomethyl, diethylaminomethyl, dimethylaminoethyloxy, dimethylaminopropyloxy, diethylaminopropyloxy, 4-methylsulfonylpiperazin-1-yl, 3-azepan-1-ylmethyl, 4-methyl-1,4-diazepan-1-yl, 3-pyrrolidin-1-ylethyl, 4-methylpiperazin-1-ylmethyl; 4-ethylpiperazin-1-ylmethyl; 3-piperazin-1-ylmethyl; morpholin-4-ylmethyl; 3-morpholin-4-ylmethyl; 2-morpholin-4-yl ethyloxy; 2-piperidin-1-yl ethyloxy; 3-morpholin-4-ylpropyloxy 1H-pyrazol-1-yl, 4-trifluoromethyl-1H-pyrazol-1-yl, 4-acetylpiperazin-1-ylmethyl; methylbenzotriazolyl, dimethylethyloxycarbonylpiperazin-1-ylmethyl, 4-phenylsulfonylpiperazin-1-ylmethyl, 4-fluorophenylsulfonylpiperazin-1-yl, 4-ethylsulfonylpiperazin-1-ylmethyl, 4-cyclopropyl carbonylpiperazin-1-ylmethyl, 2-methylpropanoylpiperazin-1-ylmethyl, 4-phenylcarbonyl piperazin-1-ylmethyl, 3-azocan-1-ylmethyl, 4-acetyl-1,4-diazepan-1-yl, 4-phenylamino carbonylpiperazin-1-ylmethyl; 4-ethylaminocarbonylpiperazin-1-ylmethyl; 3-piperidin-1-ylpropyloxy, 2-pyrrolidin-1-ylethyloxy; 3-piperidin-1-ylpropyloxy; or 3-morpholin-4-ylpropyloxy.

Preferred compounds of Embodiment 7 are selected from the group consisting of:
1-methylethyl 1,1-dimethyl-3-[(4-piperidin-4-ylphenyl)carbonyl]-1,2,3,6-tetrahydroazepino[4,5-b]indole-5-carboxylate;
1-methylethyl 1,1-dimethyl-3-[(3-piperidin-4-ylphenyl)carbonyl]-1,2,3,6-tetrahydroazepino[4,5-b]indole-5-carboxylate;
1-methylethyl 3-({4-[(dimethylamino)methyl]phenyl}carbonyl)-1,1-dimethyl-1,2,3,6-tetrahydroazepino[4,5-b]indole-5-carboxylate;
1-methylethyl 3-({3-[(dimethylamino)methyl]phenyl}carbonyl)-1,1-dimethyl-1,2,3,6-tetrahydroazepino[4,5-b]indole-5-carboxylate;
1-methylethyl 3-({3-[(diethylamino)methyl]phenyl}carbonyl)-1,1-dimethyl-1,2,3,6-tetrahydroazepino[4,5-b]indole-5-carboxylate;
1-methylethyl 1,1-dimethyl-3-{[3-(pyrrolidin-1-ylmethyl)phenyl]carbonyl}-1,2,3,6-tetrahydroazepino[4,5-b]indole-5-carboxylate;
1-methylethyl 1,1-dimethyl-3-{[3-(piperidin-1-ylmethyl)phenyl]carbonyl}-1,2,3,6-tetrahydroazepino[4,5-b]indole-5-carboxylate;
1-methylethyl 1,1-dimethyl-3-({3-[(4-methylpiperazin-1-yl)methyl]phenyl}carbonyl)-1,2,3,6-tetrahydroazepino[4,5-b]indole-5-carboxylate;
1-methylethyl 3-({3-[(4-ethylpiperazin-1-yl)methyl]phenyl}carbonyl)-1,1-dimethyl-1,2,3,6-tetrahydroazepino[4,5-b]indole-5-carboxylate;
1-methylethyl 1,1-dimethyl-3-{[3-(morpholin-4-ylmethyl)phenyl]carbonyl}-1,2,3,6-tetrahydroazepino[4,5-b]indole-5-carboxylate;
1-methylethyl 3-[(3-{[2-(dimethylamino)ethyl]oxy}phenyl)carbonyl]-1,1-dimethyl-1,2,3,6-tetrahydroazepino[4,5-b]indole-5-carboxylate;
1-methylethyl 1,1-dimethyl-3-{[4-(1H-pyrazol-1-yl)phenyl]carbonyl}-1,2,3,6-tetrahydroazepino[4,5-b]indole-5-carboxylate;
1-methylethyl 3-({3-[(4-acetylpiperazin-1-yl)methyl]phenyl}carbonyl)-1,1-dimethyl-1,2,3,6-tetrahydroazepino[4,5-b]indole-5-carboxylate;
1-methylethyl 3-[(3-{[3-(dimethylamino)propyl]oxy}phenyl)carbonyl]-1,1-dimethyl-1,2,3,6-tetrahydroazepino[4,5-b]indole-5-carboxylate;
1-methylethyl 1,1-dimethyl-3-[(3-{[4-(methylsulfonyl)piperazin-1-yl]methyl}phenyl)carbonyl]-1,2,3,6-tetrahydroazepino[4,5-b]indole-5-carboxylate;
1-methylethyl 3-{[3-(azepan-1-ylmethyl)phenyl]carbonyl}-1,1-dimethyl-1,2,3,6-tetrahydroazepino[4,5-b]indole-5-carboxylate;
1-methylethyl 1,1-dimethyl-3-({3-[(4-methyl-1,4-diazcpan-1-yl)methyl]phenyl}carbonyl)-1,2,3,6-tetrahydroazepino[4,5-b]indole-5-carboxylate;
1-methylethyl 3-{[2-fluoro-5-(morpholin-4-ylmethyl)phenyl]carbonyl}-1,1-dimethyl-1,2,3,6-tetrahydroazepino[4,5-b]indole-5-carboxylate;
1-methylethyl 3-{[4-fluoro-3-(morpholin-4-ylmethyl)phenyl]carbonyl}-1,1-dimethyl-1,2,3,6-tetrahydroazepino[4,5-b]indole-5-carboxylate;
1-methylethyl 1,1-dimethyl-3-[(1-methyl-1H-1,2,3-benzotriazol-5-yl)carbonyl]-1,2,3,6-tetrahydroazepino[4,5-b]indole-5-carboxylate;
1-methylethyl 1,1-dimethyl-3-({4-[4-(trifluoromethyl)-1H-pyrazol-1-yl]phenyl}carbonyl)-1,2,3,6-tetrahydroazepino[4,5-b]indole-5-carboxylate;
1-methylethyl 1,1-dimethyl-3-({3-[(2-piperidin-1-ylethyl)oxy]phenyl}carbonyl)-1,2,3,6-tetrahydroazepino[4,5-b]indole-5-carboxylate;
1-methylethyl 1,1-dimethyl-3-({3-[(2-morpholin-4-ylethyl)oxy]phenyl}carbonyl)-1,2,3,6-tetrahydroazepino[4,5-b]indole-5-carboxylate;
1-methylethyl 3-{[2-fluoro-5-(piperidin-1-ylmethyl)phenyl]carbonyl}-1,1-dimethyl-1,2,3,6-tetrahydroazepino[4,5-b]indole-5-carboxylate;
1-methylethyl 3-{[4-fluoro-3-(piperidin-1-ylmethyl)phenyl]carbonyl}-1,1-dimethyl-1,2,3,6-tetrahydroazepino[4,5-b]indole-5-carboxylate;
1-methylethyl 3-({3-[(4-{[(1,1-dimethylethyl)oxy]carbonyl}piperazin-1-yl)methyl]phenyl}carbonyl)-1,1-dimethyl-1,2,3,6-tetrahydroazepino[4,5-b]indole-5-carboxylate;
1-methylethyl 1,1-dimethyl-3-[(3-{[4-(phenylsulfonyl)piperazin-1-yl]methyl}phenyl)carbonyl]-1,2,3,6-tetrahydroazepino[4,5-b]indole-5-carboxylate;
1-methylethyl 3-{[3-({4-[(4-fluorophenyl)sulfonyl]piperazin-1-yl}methyl)phenyl]carbonyl}-1,1-dimethyl-1,2,3,6-tetrahydroazepino[4,5-b]indole-5-carboxylate;
1-methylethyl 3-[(3-{[4-(ethylsulfonyl)piperazin-1-yl]-methyl}phenyl)carbonyl]-1,1-dimethyl-1,2,3,6-tetrahydroazepino[4,5-b]indole-5-carboxylate;
1-methylethyl 3-[(3-{[4-(cyclopropylcarbonyl)piperazin-1-yl]methyl}phenyl)carbonyl]-1,1-dimethyl-1,2,3,6-tetrahydroazepino[4,5-b]indole-5-carboxylate;
1-methylethyl 1,1-dimethyl-3-[(3-{[4-(2-methylpropanoyl)piperazin-1-yl]methyl}phenyl)carbonyl]-1,2,3,6-tetrahydroazepino[4,5-b]indole-5-carboxylate;
1-methylethyl 1,1-dimethyl-3-[(3-{[4-(phenylcarbonyl)piperazin-1-yl]methyl}phenyl)carbonyl]-1,2,3,6-tetrahydroazepino[4,5-b]indole-5-carboxylate;
1-methylethyl 3-{[3-(azocan-1-ylmethyl)phenyl]carbonyl}-1,1-dimethyl-1,2,3,6-tetrahydroazepino[4,5-b]indole-5-carboxylate;
1-methylethyl 3-({3-[(4-acetyl-1,4-diazepan-1-yl)methyl]phenyl}carbonyl)-1,1-dimethyl-1,2,3,6-tetrahydroazepino[4,5-b]indole-5-carboxylate;
1-methylethyl 1,1-dimethyl-3-{[3-(piperazin-1-ylmethyl)phenyl]carbonyl}-1,2,3,6-tetrahydroazepino[4,5-b]indole-5-carboxylate;
1-methylethyl 3-({3,4-difluoro-5-[(2-morpholin-4-ylethyl)oxy]phenyl}carbonyl)-1,1-dimethyl-1,2,3,6-tetrahydroazepino[4,5-b]indole-5-carboxylate;
1-methylethyl 3-({3,4-difluoro-5-[(2-piperidin-1-ylethyl)oxy]phenyl}carbonyl)-1,1-dimethyl-1,2,3,6-tetrahydroazepino[4,5-b]indole-5-carboxylate;
1-methylethyl 1,1-dimethyl-3-({4-[(2-morpholin-4-ylethyl)oxy]phenyl}carbonyl)-1,2,3,6-tetrahydroazepino[4,5-b]indole-5-carboxylate;
1-methylethyl 1,1-dimethyl-3-({4-[(2-piperidin-1-ylethyl)oxy]phenyl}carbonyl)-1,2,3,6-tetrahydroazepino[4,5-b]indole-5-carboxylate;
1-methylethyl 1,1-dimethyl-3-({3-[(3-morpholin-4-ylpropyl)oxy]phenyl}carbonyl)-1,2,3,6-tetrahydroazepino[4,5-b]indole-5-carboxylate;
1-methylethyl 1,1-dimethyl-3-{[3-({4-[(phenylamino)carbonyl]piperazin-1-yl}methyl)phenyl]carbonyl}-1,2,3,6-tetrahydroazepino[4,5-b]indole-5-carboxylate;
1-methylethyl 3-{[3-({4-[(ethylamino)carbonyl]piperazin-1-yl}methyl)phenyl]carbonyl}-1,1-dimethyl-1,2,3,6-tetrahydroazepino[4,5-b]indole-5-carboxylate;
1-methylethyl 1,1-dimethyl-3-({3-[(3-piperidin-1-ylpropyl)oxy]phenyl}carbonyl)-1,2,3,6-tetrahydroazepino[4,5-b]indole-5-carboxylate;
1-methylethyl 3-[(4-{[2-(dimethylamino)ethyl]oxy}phenyl)carbonyl]-1,1-dimethyl-1,2,3,6-tetrahydroazepino[4,5-b]indole-5-carboxylate;
1-methylethyl 3-[(3-{[3-(diethylamino)propyl]oxy}phenyl)carbonyl]-1,1-dimethyl-1,2,3,6-tetrahydroazepino[4,5-b]indole-5-carboxylate;
1-methylethyl 3-[(4-{[3-(dimethylamino)propyl]oxy}phenyl)carbonyl]-1,1-dimethyl-1,2,3,6-tetrahydroazepino[4,5-b]indole-5-carboxylate;
1-methylethyl 1,1-dimethyl-3-({4-[(2-pyrrolidin-1-ylethyl)oxy]phenyl}carbonyl)-1,2,3,6-tetrahydroazepino[4,5-b]indole-5-carboxylate;
1-methylethyl 1,1-dimethyl-3-({4-[(3-piperidin-1-ylpropyl)oxy]phenyl}carbonyl)-1,2,3,6-tetrahydroazepino[4,5-b]indole-5-carboxylate;
1-methylethyl 1,1-dimethyl-3-({4-[(3-morpholin-4-ylpropyl)oxy]phenyl}carbonyl)-1,2,3,6-tetrahydroazepino[4,5-b]indole-5-carboxylate.

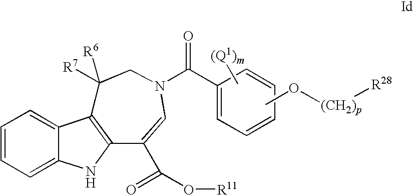

In Embodiment 8, the invention provides compound of Formula Id wherein each R⁶ and R⁷ is independently optionally substituted alkyl, preferably methyl. Each R¹¹ is independently optionally substituted alkyl, preferably methyl, ethyl, propyl, isopropyl, butyl, isobutyl, or pentyl; more preferably, isopropyl; p is 1-3; m is 0-3; Q¹ is optionally substituted alkyl or halo; R²⁸ is optionally substituted alkyl, optionally substituted aryl, optionally substituted aralkyl, optionally substituted heterocyclyl, or optionally substituted heterocyclylalkyl, preferably, R²⁸ is phenyl, dimethylamino, diethylamino, N-ethyl, N-methyl amino, morpholinyl, piperidinyl, piperazinyl, pyrrolidinyl, morpholinyl, or 4-methyloxyphenyl.

Preferred compounds of Embodiment 8 are selected from the group consisting of:
1-methylethyl 1,1-dimethyl-3-({3-[(phenylmethyl)oxy]phenyl}carbonyl)-1,2,3,6-tetrahydroazepino[4,5-b]indole-5-carboxylate;
1-methylethyl 3-[(3-{[2-(dimethylamino)ethyl]oxy}phenyl)carbonyl]-1,1-dimethyl-1,2,3,6-tetrahydroazepino[4,5-b]indole-5-carboxylate;
1-methylethyl 3-[(3-{[3-(dimethylamino)propyl]oxy}phenyl)carbonyl]-1,1-dimethyl-1,2,3,6-tetrahydroazepino[4,5-b]indole-5-carboxylate;
1-methylethyl 1,1-dimethyl-3-({3-[(2-piperidin-1-ylethyl)oxy]phenyl}carbonyl)-1,2,3,6-tetrahydroazepino[4,5-b]indole-5-carboxylate;
1-methylethyl 1,1-dimethyl-3-({3-[(2-morpholin-4-ylethyl)oxy]phenyl}carbonyl)-1,2,3,6-tetrahydroazepino[4,5-b]indole-5-carboxylate;
1-methylethyl 3-{[3,4-difluoro-5-({[4-(methyloxy)phenyl]methyl}oxy)phenyl]carbonyl}-1,1-dimethyl-1,2,3,6-tetrahydroazepino[4,5-b]indole-5-carboxylate;
1-methylethyl 3-({3,4-difluoro-5-[(2-morpholin-4-ylethyl)oxy]phenyl}carbonyl)-1,1-dimethyl-1,2,3,6-tetrahydroazepino[4,5-b]indole-5-carboxylate;
1-methylethyl 3-({3,4-difluoro-5-[(2-piperidin-1-ylethyl)oxy]phenyl}carbonyl)-1,1-dimethyl-1,2,3,6-tetrahydroazepino[4,5-b]indole-5-carboxylate,
1-methylethyl 1,1-dimethyl-3-({4-[(2-morpholin-4-ylethyl)oxy]phenyl}carbonyl)-1,2,3,6-tetrahydroazepino[4,5-b]indole-5-carboxylate;
1-methylethyl 1,1-dimethyl-3-({4-[(2-piperidin-1-ylethyl)oxy]phenyl}carbonyl)-1,2,3,6-tetrahydroazepino[4,5-b]indole-5-carboxylate;
1-methylethyl 1,1-dimethyl-3-({3-[(3-morpholin-4-ylpropyl)oxy]phenyl}carbonyl)-1,2,3,6-tetrahydroazepino[4,5-b]indole-5-carboxylate;
1-methylethyl 1,1-dimethyl-3-({3-[(3-piperidin-1-ylpropyl)oxy]phenyl}carbonyl)-1,2,3,6-tetrahydroazepino[4,5-b]indole-5-carboxylate;
1-methylethyl 3-[(4-{[2-(dimethylamino)ethyl]oxy}phenyl)carbonyl]-1,1-dimethyl-1,2,3,6-tetrahydroazepino[4,5-b]indole-5-carboxylate;
1-methylethyl 3-[(3-{[3-(diethylamino)propyl]oxy}phenyl)carbonyl]-1,1-dimethyl-1,2,3,6-tetrahydroazepino[4,5-b]indole-5-carboxylate;
1-methylethyl 3-[(4-{[3-(dimethylamino)propyl]oxy}phenyl)carbonyl]-1,1-dimethyl-1,2,3,6-tetrahydroazepino[4,5-b]indole-5-carboxylate;
1-methylethyl 1,1-dimethyl-3-({4-[(2-pyrrolidin-1-ylethyl)oxy]phenyl}carbonyl)-1,2,3,6-tetrahydroazepino[4,5-b]indole-5-carboxylate;
1-methylethyl 1,1-dimethyl-3-({4-[(3-piperidin-1-ylpropyl)oxy]phenyl}carbonyl)-1,2,3,6-tetrahydroazepino[4,5-b]indole-5-carboxylate; and
1-methylethyl 1,1-dimethyl-3-({4-[(3-morpholin-4-ylpropyl)oxy]phenyl}carbonyl)-1,2,3,6-tetrahydroazepino[4,5-b]indole-5-carboxylate;

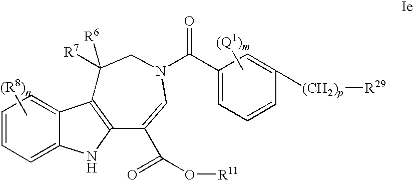

In Embodiment 9, the invention provides a compound of Formula Ie wherein each R⁶ and R⁷ is independently optionally substituted alkyl, preferably methyl; n is 0; Each R¹¹ is independently optionally substituted alkyl, preferably methyl, ethyl, propyl, isopropyl, butyl, isobutyl, or pentyl; more preferably, isopropyl; p is 1-3; R²⁹ is halogen, optionally substituted alkyl, optionally substituted aryl, optionally substituted aralkyl, optionally substituted heterocyclyl, or optionally substituted heterocyclylalkyl; preferably, R²⁹ is dimethylamino, diethylamino, N-ethyl, N-methyl amino, chloro, morpholinyl, piperidinyl, piperazinyl, piperazin-1-ylmethyl, piperazin-1-ylethyl, pyrrolidinyl, morpholinyl, methyloxyphenyl; 4-acetylpiperazin-1-yl; 4-methylsulfonylpiperazin-1-yl; azepanyl; azocan-1-yl; 4-methyl-1,4-diazepan-1-yl; 4-acetyl-1,4-diazepan-1-yl; dimethylethyloxy carbonylpiperazin-1-yl; 4-phenylsulfonyl piperazin-1-yl; 4-fluorophenylsulfonylpiperazin-1-yl; ethylsulfonyl piperazin-1-yl; cyclopropylcarbonyl piperazin-1-yl; 2-methylpropanoyl piperazin-1-yl; phenylcarbonyl piperazin-1-yl; 4-phenylaminocarbonylpiperazin-1-yl; or 4-ethylaminocarbonylpiperazin-1-yl; Q¹ is halogen or optionally substituted alkyl, preferably, methyl, chloro, fluoro or bromo; m is 0-3.

Preferred compounds of Embodiment 9 are selected from the group consisting of:
1-methylethyl 3-({3-[(dimethylamino)methyl]phenyl}carbonyl)-1,1-dimethyl-1,2,3,6-tetrahydroazepino[4,5-b]indole-5-carboxylate;
1-methylethyl 3-{[3-(chloromethyl)phenyl]carbonyl}-1,1-dimethyl-1,2,3,6-tetrahydroazepino[4,5-b]indole-5-carboxylate;
1-methylethyl 3-({3-[(diethylamino)methyl]phenyl}carbonyl)-1,1-dimethyl-1,2,3,6-tetrahydroazepino[4,5-b]indole-5-carboxylate;
1-methylethyl 1,1-dimethyl-3-{[3-(pyrrolidin-1-ylmethyl)phenyl]carbonyl}-1,2,3,6-tetrahydroazepino[4,5-b]indole-5-carboxylate;
1-methylethyl 1,1-dimethyl-3-{[3-(piperidin-1-ylmethyl)phenyl]carbonyl}-1,2,3,6-tetrahydroazepino[4,5-b]indole-5-carboxylate;
1-methylethyl 1,1-dimethyl-3-({3-[(4-methylpiperazin-1-yl)methyl]phenyl}carbonyl)-1,2,3,6-tetrahydroazepino[4,5-b]indole-5-carboxylate;
1-methylethyl 3-({3-[(4-ethylpiperazin-1-yl)methyl]phenyl}carbonyl)-1,1-dimethyl-1,2,3,6-tetrahydroazepino[4,5-b]indole-5-carboxylate;
1-methylethyl 1,1-dimethyl-3-{[3-(morpholin-4-ylmethyl)phenyl]carbonyl}-1,2,3,6-tetrahydroazepino[4,5-b]indole-5-carboxylate;
1-methylethyl 3-({3-[(4-acetylpiperazin-1-yl)methyl]phenyl}carbonyl)-1,1-dimethyl-1,2,3,6-tetrahydroazepino[4,5-b]indole-5-carboxylate;
1-methylethyl 1,1-dimethyl-3-[(3-{[4-(methylsulfonyl)piperazin-1-yl]methyl}phenyl)carbonyl]-1,2,3,6-tetrahydroazepino[4,5-b]indole-5-carboxylate;
1-methylethyl 3-{[3-(azepan-1-ylmethyl)phenyl]carbonyl}-1,1-dimethyl-1,2,3,6-tetrahydroazepino[4,5-b]indole-5-carboxylate;
1-methylethyl 1,1-dimethyl-3-({3-[(4-methyl-1,4-diazepan-1-yl)methyl]phenyl}carbonyl)-1,2,3,6-tetrahydroazepino[4,5-b]indole-5-carboxylate;
1-methylethyl 3-{[2-fluoro-5-(morpholin-4-ylmethyl)phenyl]carbonyl}-1,1-dimethyl-1,2,3,6-tetrahydroazepino[4,5-b]indole-5-carboxylate;
1-methylethyl 3-{[4-fluoro-3-(morpholin-4-ylmethyl)phenyl]carbonyl}-1,1-dimethyl-1,2,3,6-tetrahydroazepino[4,5-b]indole-5-carboxylate;
1-methylethyl 3-{[2-fluoro-5-piperidin-1-ylmethyl)phenyl]carbonyl}-1,1-dimethyl-1,2,3,6-tetrahydroazepino[4,5-b]indole-5-carboxylate;
1-methylethyl 3-([4-fluoro-3-(piperidin-1-ylmethyl)phenyl]carbonyl)-1,1-dimethyl-1,2,3,6-tetrahydroazepino[4,5-b]indole-5-carboxylate;
1-methylethyl 3-({3-[(4-{[(1,1-dimethylethyl)oxy]carbonyl}piperazin-1-yl)methyl]phenyl}carbonyl)-1,1-dimethyl-1,2,3,6-tetrahydroazepino[4,5-b]indole-5-carboxylate;
1-methylethyl 1,1-dimethyl-3-[(3-{[4-(phenylsulfonyl)piperazin-1-yl]methyl}phenyl)carbonyl]-1,2,3,6-tetrahydroazepino[4,5-b]indole-5-carboxylate;
1-methylethyl 3-{[3-({4-[(4-fluorophenyl)sulfonyl]piperazin-1-yl}methyl)phenyl]carbonyl}-1,1-dimethyl-1,2,3,6-tetrahydroazepino[4,5-b]indole-5-carboxylate;
1-methylethyl 3-[(3-{[4-(ethylsulfonyl)piperazin-1-yl]methyl}phenyl)carbonyl]-1,1-dimethyl-1,2,3,6-tetrahydroazepino[4,5-b]indole-5-carboxylate;
1-methylethyl 3-[(3-{[4-(cyclopropylcarbonyl)piperazin-1-yl]methyl}phenyl)carbonyl]-1,1-dimethyl-1,2,3,6-tetrahydroazepino[4,5-b]indole-5-carboxylate;
1-methylethyl 1,1-dimethyl-3-[(3-{[4-(2-methylpropanoyl)piperazin-1-yl]methyl}phenyl)carbonyl]-1,2,3,6-tetrahydroazepino[4,5-b]indole-5-carboxylate;
1-methylethyl 1,1-dimethyl-3-[(3-{[4-(phenylcarbonyl)piperazin-1-yl]methyl}phenyl)carbonyl]-1,2,3,6-tetrahydroazepino[4,5-b]indole-5-carboxylate;
1-methylethyl 3-{[3-(azocan-1-ylmethyl)phenyl]carbonyl}-1,1-dimethyl-1,2,3,6-tetrahydroazepino[4,5-b]indole-5-carboxylate;
1-methylethyl 3-({3-[(4-acetyl-1,4-diazepan-1-yl)methyl]phenyl}carbonyl)-1,1-dimethyl-1,2,3,6-tetrahydroazepino[4,5-b]indole-5-carboxylate;
1-methylethyl 1,1-dimethyl-3-{[3-(piperazin-1-ylmethyl)phenyl]carbonyl}-1,2,3,6-tetrahydroazepino[4,5-b]indole-5-carboxylate;
1-methylethyl 1,1-dimethyl-3-{[3-({4-[(phenylamino)carbonyl]piperazin-1-yl}methyl)phenyl]carbonyl}-1,2,3,6-tetrahydroazepino[4,5-b]indole-5-carboxylate; and
1-methylethyl 3-{[3-({4-[(ethylamino)carbonyl]piperazin-1-yl}methyl)phenyl]carbonyl}-1,1-dimethyl-1,2,3,6-tetrahydroazepino[4,5-b]indole-5-carboxylate;

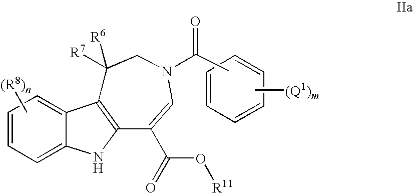

In Embodiment 10, the invention provides a compound of Formula IIa wherein each R⁶ and R⁷ is independently optionally substituted alkyl, preferably methyl; n is 0. Each R¹¹ is independently optionally substituted alkyl, optionally substituted aryl, optionally substituted aralkyl, optionally substituted heterocyclyl, or optionally substituted heterocyclylalkyl. Preferably, R¹¹ is 2,2-dimethyl-1,3-dioxolan-4-yl, 2-piperidin-1-ylethylaminocarbonyl; 2,3-dihydroxypropyl or 2-fluoro-1-(fluoromethyl)ethyl, hydroxyethyl, phenylmethyloxyethyl, 3,4-difluorophenylcarbonyloxy-1-methylethyl, 2-hydroxy-1-methylethyl; p is 1-3; Q¹ is halogen or optionally substituted alkyl, preferably methyl, chloro, fluoro, bromo or 3,4-difluoro; m is 0-3.

Preferred compounds of Embodiment 10 are selected from the group consisting of:
(2,2-dimethyl-1,3-dioxolan-4-yl)methyl 3-[(3,4-difluorophenyl)carbonyl]-1,1-dimethyl-1,2,3,6-tetrahydroazepino[4,5-b]indole-5-carboxylate;
2,3-dihydroxypropyl 3-[(3,4-difluorophenyl)carbonyl]-1,1-dimethyl-1,2,3,6-tetrahydroazepino[4,5-b]indole-5-carboxylate;
(2R)-2,3-dihydroxypropyl 3-[(3,4-difluorophenyl)carbonyl]-1,1-dimethyl-1,2,3,6-tetrahydroazepino[4,5-b]indole-5-carboxylate;
2-fluoro-1-(fluoromethyl)ethyl 3-[(3,4-difluorophenyl)carbonyl]-1,1-dimethyl-1,2,3,6-tetrahydroazepino[4,5-b]indole-5-carboxylate;
1-methylethyl 3-[(3,4-difluorophenyl)carbonyl]-1,1-dimethyl-8-({[(2-piperidin-1-ylethyl)amino]carbonyl}oxy)-1,2,3,6-tetrahydroazepino[4,5-b]indole-5-carboxylate;
(2S)-2,3-dihydroxypropyl 3-[(3,4-difluorophenyl)carbonyl]-1,1-dimethyl-1,2,3,6-tetrahydroazepino[4,5-b]indole-5-carboxylate;
2-hydroxy-1-methylethyl 3-[(3,4-difluorophenyl)carbonyl]-1,1-dimethyl-1,2,3,6-tetrahydroazepino[4,5-b]indole-5-carboxylate;
2-{[(3,4-difluorophenyl)carbonyl]oxy}-1-methylethyl 3-[(3,4-difluorophenyl)carbonyl]-1,1-dimethyl-1,2,3,6-tetrahydroazepino[4,5-b]indole-5-carboxylate;
2-[(phenylmethyl)oxy]ethyl 3-[(3,4-difluorophenyl)carbonyl]-1,1-dimethyl-1,2,3,6-tetrahydroazepino[4,5-b]indole-5-carboxylate; and
2-hydroxyethyl 3-[(3,4-difluorophenyl)carbonyl]-1,1-dimethyl-1,2,3,6-tetrahydroazepino[4,5-b]indole-5-carboxylate.

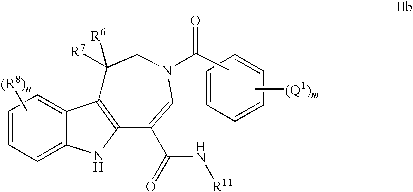

In Embodiment 11, the invention provides a compound of Formula IIb, wherein each R⁶ and R⁷ is independently optionally substituted alkyl, preferably methyl; n is 0-3; R⁸ is optionally substituted alkyl or halo, preferably chloro, bromo or fluoro. Each R¹¹ is independently optionally substituted alkyl, preferably isopropyl; beta-alanine, 2,3-dihydroxypropyl; or 2-hydroxy-1-(hydroxymethyl)ethyl; Q¹ is halogen or optionally substituted alkyl, preferably methyl, chloro, fluoro, bromo or 3,4-difluoro; m is 0-3.

Preferred compounds of Embodiment 11 are selected from the group consisting of:
N-({3-[(3,4-difluorophenyl)carbonyl]-1,1-dimethyl-1,2,3,4,5,6-hexahydroazepino[4,5-b]indol-5-yl}carbonyl)-beta-alanine;
N-({3-[(3,4-difluorophenyl)carbonyl]-1,1-dimethyl-1,2,3,6-tetrahydroazepino[4,5-b]indol-5-yl}carbonyl)-beta-alanine;
3-[(3,4-difluorophenyl)carbonyl]-N-[(2,3-dihydroxypropyl)oxy]-1,1-dimethyl-1,2,3,6-tetrahydroazepino[4,5-b]indole-5-carboxamide;
3-[(3,4-difluorophenyl)carbonyl]-N-(2,3-dihydroxypropyl)-1,1-dimethyl-1,2,3,6-tetrahydroazepino[4,5-b]indole-5-carboxamide;
3-[(3,4-difluorophenyl)carbonyl]-N-[2-hydroxy-1-(hydroxymethyl)ethyl]-1,1-dimethyl-1,2,3,6-tetrahydroazepino[4,5-b]indole-5-carboxamide; and
3-[(3,4-difluorophenyl)carbonyl]-1,1-dimethyl-N-(1-methylethyl)-1,2,3,4,5,6-hexahydroazepino[4,5-b]indole-5-carboxamide.

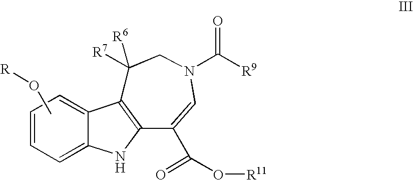

In Embodiment 12, the invention provides a compound of Formula III, wherein each R⁶ and R⁷ is independently optionally substituted alkyl, preferably methyl; R⁹ is optionally substituted aryl, preferably 3,4-difluorophenyl; Each R is independently hydrogen, optionally substituted alkyl, optionally substituted aryl, optionally substituted aralkyl, optionally substituted heterocyclyl or optionally substituted heterocyclylalkyl. Preferably, R is 2-(dimethylamino)ethylaminocarbonyl; 1,1-dimethylethyloxycarbonyl; 2-diethyl aminoethylaminocarbonyl; dimethylaminopropyl; dimethylaminoethyl; methylamino carbonyl; diethylaminoethyl; methyloxyethyl; dimethylaminopropylaminocarbonyl; phenylmethyl; hydroxy; 2-pyrrolidinyl-1-ylaminocarbonyl. Each R¹¹ is independently optionally substituted alkyl, preferably methyl, ethyl, propyl, isopropyl; more preferably, isopropyl.

Preferred compounds of Embodiment 12 are selected from the group consisting of:
1-methylethyl 3-[(3,4-difluorophenyl)carbonyl]-8-[({[2-(dimethylamino)ethyl]amino}carbonyl)oxy]-1,1-dimethyl-1,2,3,6-tetrahydroazepino[4,5-b]indole-5-carboxylate;
1-methylethyl 3-[(3,4-difluorophenyl)carbonyl]-8-({[(1,1-dimethylethyl)oxy]carbonyl}oxy)-1,1-dimethyl-1,2,3,6-tetrahydroazepino[4,5-b]indole-5-carboxylate;
1-methylethyl 8-[({[2-(diethylamino)ethyl]amino}carbonyl)oxy]-3-[(3,4-difluorophenyl)carbonyl]-1,1-dimethyl-1,2,3,6-tetrahydroazepino[4,5-b]indole-5-carboxylate;
1-methylethyl 3-[(3,4-difluorophenyl)carbonyl]-8-{[2-(dimethylamino)ethyl]oxy}-1,1-dimethyl-1,2,3,6-tetrahydroazepino[4,5-b]indole-5-carboxylate;
1-methylethyl 3-[(3,4-difluorophenyl)carbonyl]-8-{[3-(dimethylamino)propyl]oxy}-1,1-dimethyl-1,2,3,6-tetrahydroazepino[4,5-b]indole-5-carboxylate;
1-methylethyl 3-[(3,4-difluorophenyl)carbonyl]-1,1-dimethyl-8-{[(methylamino)carbonyl]oxy}-1,2,3,6-tetrahydroazepino[4,5-b]indole-5-carboxylate;
1-methylethyl 8-{[2-(diethylamino)ethyl]oxy}-3-[(3,4-difluorophenyl)carbonyl]-1,1-dimethyl-1,2,3,6-tetrahydroazepino[4,5-b]indole-5-carboxylate;
1-methylethyl 8-{[3-(diethylamino)propyl]oxy}-3-[(3,4-difluorophenyl)carbonyl]-1,1-dimethyl-1,2,3,6-tetrahydroazepino[4,5-b]indole-5-carboxylate;
1-methylethyl 3-[(3,4-difluorophenyl)carbonyl]-1,1-dimethyl-8-{[2-(methyloxy)ethyl]oxy}-1,2,3,6-tetrahydroazepino[4,5-b]indole-5-carboxylate;
1-methylethyl 8-[({[3-(diethylamino)propyl]amino}carbonyl)oxy]-3-[(3,4-difluorophenyl)carbonyl]-1,1-dimethyl-1,2,3,6-tetrahydroazepino[4,5-b]indole-5-carboxylate;
1-methylethyl 3-[(3,4-difluorophenyl)carbonyl]-1,1-dimethyl-9-[(phenylmethyl)oxy]-1,2,3,6-tetrahydroazepino[4,5-b]indole-5-carboxylate;
1-methylethyl 3-[(3,4-difluorophenyl)carbonyl]-9-hydroxy-1,1-dimethyl-1,2,3,6-tetrahydroazepino[4,5-b]indole-5-carboxylate; and
1-methylethyl 3-[(3,4-difluorophenyl)carbonyl]-1,1-dimethyl-8-({[(2-pyrrolidin-1-ylethyl)amino]carbonyl}oxy)-1,2,3,6-tetrahydroazepino[4,5-b]indole-5-carboxylate.

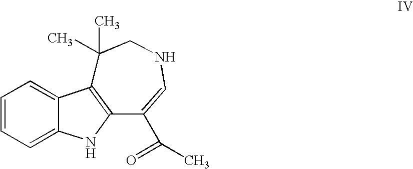

In Embodiment 12, the invention provides a compound of Formula I, wherein R⁶ or R⁷ is optionally substituted alkyl, preferably methyl; R¹ is C(J)R¹¹; wherein J is O and R¹¹ is optionally substituted alkyl, preferably methyl; n is 0 and R³ is hydrogen.

Specifically excluded from the scope of this invention are the compounds of Table 2:

TABLE 2
ethyl 3-[(4-fluorophenyl)carbonyl]-8-furan-3-yl-1,1-dimethyl-1,2,3,6-tetrahydro-
azepino[4,5-b]indole-5-carboxylate;
ethyl 8-furan-3-yl-1,1-dimethyl-1,2,3,6-tetrahydroazepino[4,5-b]indole-5-carboxylate;
ethyl 3-[(3,4-difluorophenyl)carbonyl]-8-furan-3-yl-1,1-dimethyl-1,2,3,6-tetrahydro-
azepino [4,5-b]indole-5-carboxylate;
ethyl 3-[(4-fluorophenyl)carbonyl]-1,1-dimethyl-8-[methyl(phenylmethyl)amino]-1,2,3,6-
tetrahydroazepino[4,5-b]indole-5-carboxylate;
ethyl 3-[(4-fluorophenyl)carbonyl]-1,1-dimethyl-8-[3-(methyloxy)phenyl]-1,2,3,6-
tetrahydroazepino[4,5-b]indole-5-carboxylate;
ethyl 3-[(3,4-difluorophenyl)carbonyl]-1,1-dimethyl-8-[3-(methyloxy)phenyl]-1,2,3,6-
tetrahydroazepino[4,5-b]indole-5-carboxylate;
ethyl 3-[(3,4-difluorophenyl)carbonyl]-8-{[(dimethylamino)carbonyl](methyl) amino}-
1,1-dimethyl-1,2,3,6-tetrahydroazepino[4,5-b]indole-5-carboxylate;
ethyl 3-[(4-fluorophenyl)carbonyl]-9-{[(4-fluorophenyl)carbonyl]amino}-1,1-dimethyl-
1,2,3,6-tetrahydroazepino[4,5-b]indole-5-carboxylate;
ethyl 9-(acetylamino)-3-[(4-fluorophenyl)carbonyl]-1,1-dimethyl-1,2,3,6-tetrahydro-
azepino[4,5-b]indole-5-carboxylate;
ethyl 9-[bis(phenylmethyl)amino]-3-[(4-fluorophenyl)carbonyl]-1,1-dimethyl-1,2,3,6-
tetrahydroazepino[4,5-b]indole-5-carboxylate;
ethyl 9-{[(dimethylamino)carbonyl]amino}-3-[(4-fluorophenyl)carbonyl]-1,1-dimethyl-
1,2,3,6-tetrahydroazepino[4,5-b]indole-5-carboxylate;
ethyl 3-[(4-fluorophenyl)carbonyl]-1,1-dimethyl-9-{[(methyloxy)acetyl]amino}-1,2,3,6-
tetrahydroazepino[4,5-b]indole-5-carboxylate;
ethyl 3-[(4-fluorophenyl)carbonyl]-1,1-dimethyl-9-[(morpholin-4-ylcarbonyl)amino]-
1,2,3,6-tetrahydroazepino[4,5-b]indole-5-carboxylate;
ethyl 3-[(4-fluorophenyl)carbonyl]-1,1-dimethyl-9-[(2-thienylacetyl)amino]-1,2,3,6-
tetrahydroazepino[4,5-b]indole-5-carboxylate;
ethyl 9-(dimethylamino)-3-[(4-fluorophenyl)carbonyl]-1,1-dimethyl-1,2,3,6-tetrahydro-
azepino[4,5-b]indole-5-carboxylate;
ethyl 3-[(4-fluorophenyl)carbonyl]-1,1-dimethyl-9-[(phenylmethyl)amino]-1,2,3,6-
tetrahydroazepino[4,5-b]indole-5-carboxylate;
ethyl 9-amino-3-[(4-fluorophenyl)carbonyl]-1,1-dimethyl-1,2,3,6-tetrahydroazepino[4,5-
b]indole-5-carboxylate;
ethyl 3-[(4-fluorophenyl)carbonyl]-1,1-dimethyl-9-[(phenylmethyl)(2-thienylacetyl)amino]-
1,2,3,6-tetrahydroazepino[4,5-b]indole-5-carboxylate;
ethyl 3-[(4-fluorophenyl)carbonyl]-1,1-dimethyl-9-({[(1-methylethyl)amino]carbonyl}amino)-
1,2,3,6-tetrahydroazepino[4,5-b]indole-5-carboxylate;
ethyl 8-{[(dimethylamino)carbonyl](methyl)amino}-1,1-dimethyl-1,2,3,6-tetrahydro-
azepino[4,5-b]indole-5-carboxylate;
ethyl 3-[(4-fluorophenyl)carbonyl]-1,1-dimethyl-9-[(methylsulfonyl)amino]-1,2,3,6-
tetrahydroazepino[4,5-b]indole-5-carboxylate;
ethyl 3-[(4-fluorophenyl)carbonyl]-1,1-dimethyl-9-{[(2,2,2-
trifluoroethyl)sulfonyl]amino}-1,2,3,6-tetrahydroazepino[4,5-b]indole-5-carboxylate;
ethyl 3-[(4-fluorophenyl)carbonyl]-1,1-dimethyl-9-(methyl{[(1-methylethyl)
amino]carbonyl}amino)-1,2,3,6-tetrahydroazepino[4,5-b]indole-5-carboxylate;
ethyl 3-[(4-fluorophenyl)carbonyl]-1,1-dimethyl-9-[methyl(2-thienylacetyl)amino]-
1,2,3,6-tetrahydroazepino[4,5-b]indole-5-carboxylate;
ethyl 3-[(4-fluorophenyl)carbonyl]-1,1-dimethyl-9-{[(phenylmethyl)sulfonyl]amino}-
1,2,3,6-tetrahydroazepino[4,5-b]indole-5-carboxylate;
ethyl 3-[(4-fluorophenyl)carbonyl]-1,1-dimethyl-9-[(3-methylbutanoyl)amino]-1,2,3,6-
tetrahydroazepino[4,5-b]indole-5-carboxylate;
ethyl 3-[(4-fluorophenyl)carbonyl]-1,1-dimethyl-9-[(phenylacetyl)amino]-1,2,3,6-
tetrahydroazepino[4,5-b]indole-5-carboxylate;
ethyl 3-[(4-fluorophenyl)carbonyl]-1,1-dimethyl-9-({[(phenylmethyl)amino]carbonyl}amino)-
1,2,3,6-tetrahydroazepino[4,5-b]indole-5-carboxylate;
ethyl 3-[(4-fluorophenyl)carbonyl]-1,1-dimethyl-9-({[(1-
methylethyl)oxy]carbonyl}amino)-1,2,3,6-tetrahydroazepino[4,5-b]indole-5-carboxylate;
ethyl 3-[(4-fluorophenyl)carbonyl]-1,1-dimethyl-9-
({[(phenylmethyl)oxy]carbonyl}amino)-1,2,3,6-tetrahydroazepino[4,5-b]indole-5-
carboxylate;
ethyl 3-[(4-fluorophenyl)carbonyl]-1,1-dimethyl-9-({[methyl(phenyl)amino]carbonyl}amino)-
1,2,3,6-tetrahydroazepino[4,5-b]indole-5-carboxylate;
ethyl 9-[(2,2-dimethylpropanoyl)amino]-3-[(4-fluorophenyl)carbonyl]-1,1-dimethyl-
1,2,3,6-tetrahydroazepino[4,5-b]indole-5-carboxylate;
ethyl 3-[(4-fluorophenyl)carbonyl]-1,1-dimethyl-9-[({[(1S)-1-phenylethyl]amino}carbonyl)amino]-
1,2,3,6-tetrahydroazepino[4,5-b]indole-5-carboxylate;
ethyl 9-{[(cyclopentylamino)carbonyl]amino}-3-[(4-fluorophenyl)carbonyl]-1,1-
dimethyl-1,2,3,6-tetrahydroazepino[4,5-b]indole-5-carboxylate;
ethyl 9-[acetyl(methyl)amino]-3-[(4-fluorophenyl)carbonyl]-1,1-dimethyl-1,2,3,6-
tetrahydroazepino[4,5-b]indole-5-carboxylate;
ethyl 3-[(4-fluorophenyl)carbonyl]-1,1-dimethyl-8-{methyl[(methylamino)carbonyl]amino}-
1,2,3,6-tetrahydroazepino[4,5-b]indole-5-carboxylate;
ethyl 3-[(4-fluorophenyl)carbonyl]-1,1-dimethy1-8-[methyl({[(1S)-1-
phenylethyl]amino}carbonyl)amino]-1,2,3,6-tetrahydroazepino[4,5-b]indole-5-
carboxylate;
ethyl 3-[(4-fluorophenyl)carbonyl]-1,1-dimethyl-8-(methyl{[(phenylmethyl)amino]carbonothioyl}amino)-
1,2,3,6-tetrahydroazepino[4,5-b]indole-5-carboxylate;
ethyl 3-[(4-fluorophenyl)carbonyl]-8-[{[(furan-2-ylmethyl)amino]carbonothioyl}(methyl)amino]-
1,1-dimethyl-1,2,3,6-tetrahydroazepino[4,5-b]indole-5-carboxylate;
ethyl 8-[bis(phenylmethyl)amino]-3-[(4-fluorophenyl)carbonyl]-1,1-dimethyl-1,2,3,6-
tetrahydroazepino[4,5-b]indole-5-carboxylate;
ethyl 1,1-dimethyl-1,2,3,6-tetrahydroazepino[4,5-b]indole-5-carboxylate;
N-cyclobutyl-3-[(3,4-difluorophenyl)carbonyl]-1-methyl-1,2,3,6-tetrahydroazepino[4,5-
b]indole-5-carboxamide;
ethyl 8-{[(dimethylamino)carbonyl]amino}-3-[(4-fluorophenyl)carbonyl]-1,1-dimethyl-
1,2,3,6-tetrahydroazepino[4,5-b]indole-5-carboxylate;
ethyl 8-{[(dimethylamino)carbonyl](methyl)amino}-3-[(4-fluorophenyl)carbonyl]-1,1-
dimethyl-1,2,3,6-tetrahydroazepino[4,5-b]indole-5-carboxylate;
N-cyclobutyl-3-[(3,4-difluorophenyl)carbonyl]-1,1-dimethyl-1,2,3,6-tetrahydro-
azepino[4,5-b]indole-5-carboxamide;
ethyl 8-[(2-chloroethyl)(methyl)amino]-3-[(4-fluorophenyl)carbonyl]-1,1-dimethyl-
1,2,3,6-tetrahydroazepino[4,5-b]indole-5-carboxylate;
ethyl 3-[(3,4-difluorophenyl)carbonyl]-1,1-dimethyl-8-[methyl(phenylmethyl)amino]-
1,2,3,6-tetrahydroazepino[4,5-b]indole-5-carboxylate;
ethyl 3-[(4-fluorophenyl)carbonyl]-1,1-dimethyl-8-[methyl(pyrrolidin-1-ylcarbonyl)amino]-
1,2,3,6-tetrahydroazepino[4,5-b]indole-5-carboxylate;
ethyl 3-[(4-fluorophenyl)carbonyl]-1,1-dimethyl-8-[methyl(morpholin-4-ylcarbonyl)amino]-
1,2,3,6-tetrahydroazepino[4,5-b]indole-5-carboxylate;
ethyl 9-{[(dimethylamino)carbonyl](phenylmethyl)amino}-3-[(4-fluorophenyl)carbonyl]-
1,1-dimethyl-1,2,3,6-tetrahydroazepino[4,5-b]indole-5-carboxylate;
ethyl 1,1,3,6-tetramethyl-1,2,3,6-tetrahydroazepino[4,5-b]indole-5-carboxylate;
ethyl 3-[(4-fluorophenyl)carbonyl]-1,1-dimethyl-8-(methyl{[(2-pyridin-2-ylethyl)amino]carbonyl}amino)-
1,2,3,6-tetrahydroazepino[4,5-b]indole-5-carboxylate;
ethyl 3-[(4-fluorophenyl)carbonyl]-8-{[(4-fluorophenyl)carbonyl](methyl)amino}-1,1-
dimethyl-1,2,3,6-tetrahydroazepino[4,5-b]indole-5-carboxylate;
ethyl 8-{[(cyclopropylamino)carbonyl](methyl)amino}-3-[(4-fluorophenyl)carbonyl]-1,1-
dimethyl-1,2,3,6-tetrahydroazepino[4,5-b]indole-5-carboxylate;
ethyl 3-[(4-fluorophenyl)carbonyl]-1,1-dimethyl-8-(methyl{[(pyridin-2-ylmethyl)amino]carbonyl}amino)-
1,2,3,6-tetrahydroazepino[4,5-b]indole-5-carboxylate;
ethyl 3-[(3,4-difluorophenyl)carbonyl]-3,6-dihydro-2H-spiro[azepino[4,5-b]indole-1,1′-
cyclopentane]-5-carboxylate;
ethyl 1,1-dimethyl-3-{[4-(methyloxy)phenyl]carbonyl}-1,2,3,6-tetrahydroazepino[4,5-
b]indole-5-carboxylate;
ethyl 3-[(4-fluorophenyl)carbonyl]-1,1-dimethyl-1,2,3,6-tetrahydroazepino[4,5-b]indole-
5-carboxylate; ethyl 3-[(4-fluorophenyl)carbonyl]-1-(phenylmethyl)-1,2,3,6-
tetrahydroazepino[4,5-b]indole-5-carboxylate;
ethyl 8-bromo-3-[(4-fluorophenyl)carbonyl]-1,1-dimethyl-1,2,3,6-tetrahydroazepino[4,5-
b]indole-5-carboxylate;
ethyl 9-fluoro-3-[(4-fluorophenyl)carbonyl]-1,1-dimethyl-1,2,3,6-tetrahydroazepino[4,5-
b]indole-5-carboxylate;
ethyl 3-[(3,4-difluorophenyl)carbonyl]-9-fluoro-1,1-dimethyl-1,2,3,6-
tetrahydroazepino[4,5-b]indole-5-carboxylate;
1-methylethyl 3-[(3,4-difluorophenyl)carbonyl]-9-fluoro-1,1-dimethyl-1,2,3,6-
tetrahydroazepino[4,5-b]indole-5-carboxylate;
1-methylethyl 9-fluoro-3-[(4-fluorophenyl)carbonyl]-1,1-dimethyl-1,2,3,6-tetrahydro-
azepino[4,5-b]indole-5-carboxylate;
ethyl 3-[(4-fluorophenyl)carbonyl]-1,2,3,6-tetrahydroazepino[4,5-b]indole-5-carboxylate;
ethyl 3-[(4-chlorophenyl)carbonyl]-1,1-dimethyl-1,2,3,6-tetrahydroazepino[4,5-b]indole-
5-carboxylate;
ethyl 3-[(3,4-difluorophenyl)carbonyl]-1,1-dimethyl-1,2,3,6-tetrahydroazepino[4,5-
b]indole-5-carboxylate;
ethyl 2-[(4-fluorophenyl)carbonyl]-4,4-dimethyl-2,3,4,9-tetrahydro-1H-beta-carboline-1-
carboxylate;
ethyl 2-[(3,4-difluorophenyl)carbonyl]-4,4-dimethyl-2,3,4,9-tetrahydro-1H-beta-
carboline-1-carboxylate;
1-methylethyl 3-[(3,4-difluorophenyl)carbonyl]-1,1-dimethyl-1,2,3,6-
tetrahydroazepino[4,5-b]indole-5-carboxylate;
ethyl 3-[(3,4-difluorophenyl)carbonyl]-1,1,6-trimethyl-1,2,3,6-tetrahydroazepino[4,5-
b]indole-5-carboxylate;
ethyl 3-[(4-fluorophenyl)carbonyl]-1,1-dimethyl-8-[(phenylmethyl)oxy]-1,2,3,6-
tetrahydroazepino[4,5-b]indole-5-carboxylate;
ethyl 3-[(4-fluorophenyl)carbonyl]-8-hydroxy-1,1-dimethyl-1,2,3,6-
tetrahydroazepino[4,5-b]indole-5-carboxylate;
ethyl 3-[(4-fluorophenyl)carbonyl]-1,1-dimethyl-8-[(2-morpholin-4-ylethyl)oxy]-1,2,3,6-
tetrahydroazepino[4,5-b]indole-5-carboxylate;
ethyl 3-[(4-fluorophenyl)carbonyl]-1,1-dimethyl-8-[(2-piperidin-1-ylethyl)oxy]-1,2,3,6-
tetrahydroazepino[4,5-b]indole-5-carboxylate;
ethyl 8-{[2-(ethyloxy)-2-oxoethyl]oxy}-3-[(4-fluorophenyl)carbonyl]-1,1-dimethyl-
1,2,3,6-tetrahydroazepino[4,5-b]indole-5-carboxylate;
ethyl 8-[(2-amino-2-oxoethyl)oxy]-3-[(4-fluorophenyl)carbonyl]-1,1-dimethyl-1,2,3,6-
tetrahydroazepino[4,5-b]indole-5-carboxylate;
ethyl 3-[(4-fluorophenyl)carbonyl]-1,1-dimethyl-8-
({[methyl(phenyl)amino]carbonyl}oxy)-1,2,3,6-tetrahydroazepino[4,5-b]indole-5-
carboxylate;
ethyl 8-[({[2-(dimethylamino)ethyl]amino}carbonyl)oxy]-3-[(4-fluorophenyl)carbonyl]-
1,1-dimethyl-1,2,3,6-tetrahydroazepino[4,5-b]indole-5-carboxylate;
ethyl 9-{[(dimethylamino)carbonyl]oxy}-3-[(4-fluorophenyl)carbonyl]-1,1-dimethyl-
1,2,3,6-tetrahydroazepino[4,5-b]indole-5-carboxylate;
ethyl 3-[(4-fluorophenyl)carbonyl]-1,1-dimethyl-9-[(morpholin-4-ylcarbonyl)oxy]-
1,2,3,6-tetrahydroazepino[4,5-b]indole-5-carboxylate;
ethyl 3-[(4-fluorophenyl)carbonyl]-1,1-dimethyl-9-[(pyrrolidin-1-ylcarbonyl)oxy]-
1,2,3,6-tetrahydroazepino[4,5-b]indole-5-carboxylate;
ethyl 3-[(3,4-difluorophenyl)carbonyl]-1,1-dimethyl-8-[(phenylmethyl)oxy]-1,2,3,6-
tetrahydroazepino[4,5-b]indole-5-carboxylate;
ethyl 3-[(4-fluorophenyl)carbonyl]-9-hydroxy-1,1-dimethyl-1,2,3,6-
tetrahydroazepino[4,5-b]indole-5-carboxylate;
ethyl 3-[(4-fluorophenyl)carbonyl]-8-[(3-hydroxypropyl)oxy]-1,1-dimethyl-1,2,3,6-
tetrahydroazepino[4,5-b]indole-5-carboxylate;
ethyl 8-{[(cyclopropylamino)carbonyl]oxy}-3-[(3,4-difluorophenyl)carbonyl]-1,1-
dimethyl-1,2,3,6-tetrahydroazepino[4,5-b]indole-5-carboxylate;
ethyl 3-[(3,4-difluorophenyl)carbonyl]-1,1-dimethyl-8-{[(methylamino)carbonyl]oxy}-
1,2,3,6-tetrahydroazepino[4,5-b]indole-5-carboxylate;
ethyl 3-[(3,4-difluorophenyl)carbonyl]-1,1-dimethyl-8-({[(pyridin-2-ylmethyl)amino]carbonyl}oxy)-
1,2,3,6-tetrahydroazepino[4,5-b]indole-5-carboxylate;
ethyl 3-[(4-fluorophenyl)carbonyl]-1,1-dimethyl-9-({[(2-thienylmethyl)amino]carbonyl}oxy)-
1,2,3,6-tetrahydroazepino[4,5-b]indole-5-carboxylate;
ethyl 3-[(4-fluorophenyl)carbonyl]-1,1-dimethyl-9-({[(pyridin-2-ylmethyl)amino]carbonyl}oxy)-
1,2,3,6-tetrahydroazepino[4,5-b]indole-5-carboxylate;
ethyl 3-[(3,4-difluorophenyl)carbonyl]-1,1-dimethyl-8-{[(propylamino)carbonyl]oxy}-
1,2,3,6-tetrahydroazepino[4,5-b]indole-5-carboxylate;
ethyl 3-[(3,4-difluorophenyl)carbonyl]-1,1-dimethyl-8-({[(phenylmethyl)amino]carbonyl}oxy)-
1,2,3,6-tetrahydroazepino[4,5-b]indole-5-carboxylate;
ethyl 3-[(3,4-difluorophenyl)carbonyl]-1,1-dimethyl-8-{[(phenylamino)carbonyl]oxy}-
1,2,3,6-tetrahydroazepino[4,5-b]indole-5-carboxylate;
ethyl 3-[(3,4-difluorophenyl)carbonyl]-8-[({[(4-
fluorophenyl)methyl]amino}carbonyl)oxy]-1,1-dimethyl-1,2,3,6-tetrahydroazepino[4,5-
b]indole-5-carboxylate;
ethyl 3-[(3,4-difluorophenyl)carbonyl]-1,1-dimethyl-8-[({[(1R)-1-phenylethyl]amino}carbonyl)oxy]-
1,2,3,6-tetrahydroazepino[4,5-b]indole-5-carboxylate;
ethyl 3-[(3,4-difluorophenyl)carbonyl]-1,1-dimethyl-8-[({[(1S)-1-phenylethyl]amino}carbonyl)oxy]-
1,2,3,6-tetrahydroazepino[4,5-b]indole-5-carboxylate;
ethyl 3-[(4-fluorophenyl)carbonyl]-1,1-dimethyl-8-({[(phenylmethyl)amino]carbonyl}oxy)-
1,2,3,6-tetrahydroazepino[4,5-b]indole-5-carboxylate;
′ethyl 3-[(3,4-difluorophenyl)carbonyl]-8-({[(1,1-dimethylethyl)amino]carbonyl}oxy)-
1,1-dimethyl-1,2,3,6-tetrahydroazepino[4,5-b]indole-5-carboxylate;
ethyl 8-({[(1,1-dimethylethyl)amino]carbonyl}oxy)-3-[(4-fluorophenyl)carbonyl]-1,1-
dimethyl-1,2,3,6-tetrahydroazepino[4,5-b]indole-5-carboxylate;
′ethyl 3-[(4-fluorophenyl)carbonyl]-1,1-dimethyl-8-[({[(1S)-1-phenylethyl]amino}carbonyl)oxy]-
1,2,3,6-tetrahydroazepino[4,5-b]indole-5-carboxylate;
ethyl 8-{[(2,3-dihydro-1-benzofuran-5-ylamino)carbonyl]oxy}-3-[(4-fluorophenyl)carbonyl]-
1,1-dimethyl-1,2,3,6-tetrahydroazepino[4,5-b]indole-5-carboxylate;
ethyl 8-{[(dimethylamino)carbonyl]oxy}-3-[(4-fluorophenyl)carbonyl]-1,1-dimethyl-
1,2,3,6-tetrahydroazepino[4,5-b]indole-5-carboxylate;
ethyl 8-{[(diethylamino)carbonyl]oxy}-3-[(4-fluorophenyl)carbonyl]-1,1-dimethyl-
1,2,3,6-tetrahydroazepino[4,5-b]indole-5-carboxylate;
ethyl 3-[(4-fluorophenyl)carbonyl]-1,1-dimethyl-8-[(morpholin-4-ylcarbonyl)oxy]-
1,2,3,6-tetrahydroazepino[4,5-b]indole-5-carboxylate;
ethyl 3-[(4-fluorophenyl)carbonyl]-1,1-dimethyl-8-[(piperidin-1-ylcarbonyl)oxy]-1,2,3,6-
tetrahydroazepino[4,5-b]indole-5-carboxylate;
ethyl 3-[(4-fluorophenyl)carbonyl]-1,1-dimethyl-8-[(pyrrolidin-1-ylcarbonyl)oxy]-
1,2,3,6-tetrahydroazepino[4,5-b]indole-5-carboxylate;
ethyl 8-{[(ethyloxy)carbonyl]oxy)-3-[(4-fluorophenyl)carbonyl]-1,1-dimethyl-1,2,3,6-
tetrahydroazepino[4,5-b]indole-5-carboxylate;
ethyl 3-[(4-fluorophenyl)carbonyl]-1,1-dimethyl-8-{[(phenyloxy)carbonyl]oxy}-1,2,3,6-
tetrahydroazepino[4,5-b]indole-5-carboxylate;
ethyl 3-[(4-fluorophenyl)carbonyl]-1,1-dimethyl-8-({[(phenylmethyl)oxy]carbonyl}oxy)-
1,2,3,6-tetrahydroazepino[4,5-b]indole-5-carboxylate;
ethyl 3-[(4-fluorophenyl)carbonyl]-1,1-dimethyl-8-({[methyl(phenyl)amino]carbonyl}oxy)-
1,2,3,6-tetrahydroazepino[4,5-b]indole-5-carboxylate;
ethyl 8-[({[2-(dimethylamino)ethyl]amino}carbonyl)oxy]-3-[(4-fluorophenyl)carbonyl]-
1,1-dimethyl-1,2,3,6-tetrahydroazepino[4,5-b]indole-5-carboxylate;
ethyl 8-[({[2-(dimethylamino)ethyl]oxy}carbonyl)oxy]-3-[(4-fluorophenyl)carbonyl]-1,1-
dimethyl-1,2,3,6-tetrahydroazepino[4,5-b]indole-5-carboxylate;
ethyl 3-[(4-fluorophenyl)carbonyl]-1,1-dimethyl-8-({[(2-pyridin-2-
ylethyl)amino]carbonyl}oxy)-1,2,3,6-tetrahydroazepino[4,5-b]indole-5-carboxylate;
ethyl 8-({[bis(1-methylethyl)amino]carbonyl}oxy)-3-[(4-fluorophenyl)carbonyl]-1,1-
dimethyl-1,2,3,6-tetrahydroazepino[4,5-b]indole-5-carboxylate;
′ethyl 3-[(4-fluorophenyl)carbonyl]-1,1-dimethyl-8- {[(2-oxoimidazolidin-1-yl)carbonyl]oxy}-
1,2,3,6-tetrahydroazepino[4,5-b]indole-5-carboxylate;
ethyl 3-[(4-fluorophenyl)carbonyl]-1,1-dimethyl-8-{[(4-methylpiperazin-1-yl)carbonyl]oxy}-
1,2,3,6-tetrahydroazepino[4,5-b]indole-5-carboxylate;
ethyl 3-[(4-fluorophenyl)carbonyl]-1,1-dimethyl-8-({[(pyridin-4-ylmethyl)amino]carbonyl}oxy)-
1,2,3,6-tetrahydroazepino[4,5-b]indole-5-carboxylate;
ethyl 8-[(azetidin-1-ylcarbonyl)oxy]-3-[(4-fluorophenyl)carbonyl]-1,1-dimethyl-1,2,3,6-
tetrahydroazepino[4,5-b]indole-5-carboxylate;
ethyl 3-[(4-fluorophenyl)carbonyl]-1,1-dimethyl-8-{[(4-pyridin-2-ylpiperazin-1-yl)carbonyl]oxy}-
1,2,3,6-tetrahydroazepino[4,5-b]indole-5-carboxylate;
ethyl 8-{[(cyclopropylamino)carbonyl]oxy}-3-[(4-fluorophenyl)carbonyl]-1,1-dimethyl-
1,2,3,6-tetrahydroazepino[4,5-b]indole-5-carboxylate;
ethyl 8-({[ethyl(1-methylethyl)amino]carbonyl}oxy)-3-[(4-fluorophenyl)carbonyl]-1,1-
dimethyl-1,2,3,6-tetrahydroazepino[4,5-b]indole-5-carboxylate;
ethyl 3-[(4-fluorophenyl)carbonyl]-1,1-dimethyl-8-({[(pyridin-2-ylmethyl)amino]carbonyl}oxy)-
1,2,3,6-tetrahydroazepino[4,5-b]indole-5-carboxylate;
ethyl 3-[(4-fluorophenyl)carbonyl]-1,1-dimethyl-8-({[(pyridin-3-ylmethyl)amino]carbonyl}oxy)-
1,2,3,6-tetrahydroazepino[4,5-b]indole-5-carboxylate;
ethyl 3-[(4-fluorophenyl)carbonyl]-1,1-dimethyl-8-({[(4-methylpiperazin-1-yl)amino]carbonyl}oxy)-
1,2,3,6-tetrahydroazepino[4,5-b]indole-5-carboxylate;
ethyl 3-[(4-fluorophenyl)carbonyl]-1,1-dimethyl-8-({[(2-phenylethyl)amino]carbonyl}oxy)-
1,2,3,6-tetrahydroazepino[4,5-b]indole-5-carboxylate;
ethyl 3-[(4-fluorophenyl)carbonyl]-1,1-dimethyl-8-({[(2-thienylmethyl)amino]carbonyl}oxy)-
1,2,3,6-tetrahydroazepino[4,5-b]indole-5-carboxylate;
ethyl 3-[(4-fluorophenyl)carbonyl]-8-({[(furan-2-ylmethyl)amino]carbonyl}oxy)-1,1-
dimethyl-1,2,3,6-tetrahydroazepino[4,5-b]indole-5-carboxylate;
ethyl 8-{[(cyclobutylamino)carbonyl]oxy}-3-[(4-fluorophenyl)carbonyl]-1,1-dimethyl-
1,2,3,6-tetrahydroazepino[4,5-b]indole-5-carboxylate;
ethyl 8-{[(cyclopentylamino)carbonyl]oxy}-3-[(4-fluorophenyl)carbonyl]-1,1-dimethyl-
1,2,3,6-tetrahydroazepino[4,5-b]indole-5-carboxylate;
ethyl 8-{[(cyclohexylamino)carbonyl]oxy}-3-[(4-fluorophenyl)carbonyl]-1,1-dimethyl-
1,2,3,6-tetrahydroazepino[4,5-b]indole-5-carboxylate;
ethyl 3-[(4-fluorophenyl)carbonyl]-1,1-dimethyl-8-[({[(5-methylpyrazin-2-yl)methyl]amino}carbonyl)oxy]-
1,2,3,6-tetrahydroazepino[4,5-b]indole-5-carboxylate;
ethyl 3-[(4-fluorophenyl)carbonyl]-1,1-dimethyl-8-{[(methylamino)carbonyl]oxy}-
1,2,3,6-tetrahydroazepino[4,5-b]indole-5-carboxylate;
ethyl 3-[(4-fluorophenyl)carbonyl]-1,1-dimethyl-8-({[(1-
methylethyl)amino]carbonyl}oxy)-1,2,3,6-tetrahydroazepino[4,5-b]indole-5-carboxylate;
3-[(3,4-difluorophenyl)carbonyl]-9-fluoro-1,1-dimethyl-N-(1-methylethyl)-1,2,3,6-
tetrahydroazepino[4,5-b]indole-5-carboxamide;
3-[(3,4-difluorophenyl)carbonyl]-1,1-dimethyl-N-(1-methylethyl)-1,2,3,6-tetrahydro-
azepino[4,5-b]indole-5-carboxamide;
ethyl 3-(1,3-benzodioxol-5-ylcarbonyl)-1-methyl-1,2,3,6-tetrahydroazepino[4,5-b]indole-
5-carboxylate;
ethyl 3-(1,3-benzodioxol-5-ylcarbonyl)-2-ethyl-2,2,3,6-tetrahydroazepino[4,5-b]indole-5-
carboxylate;
ethyl 3-(1,3-benzodioxol-5-ylcarbonyl)-1,1-dimethyl-1,2,3,6-tetrahydroazepino[4,5-
b]indole-5-carboxylate;
ethyl 3-[(3,4-difluorophenyl)carbonyl]-3,6-dihydro-2H-spiro[azepino[4,5-b]indole-1,1′-
cyclobutane]-5-carboxylate;
ethyl 3-[(3,4-difluorophenyl)carbonyl]-3,6-dihydro-2H-spiro[azepino[4,5-b]indole-1,1′-
cyclopropane]-5-carboxylate;
1-methylethyl 3-[(3,4-difluorophenyl)carbonyl]-3,6-dihydro-2H-spiro[azepino[4,5-
b]indole-1,1′-cyclopropane]-5-carboxylate; or
ethyl 3-[(3,4-difluorophenyl)carbonyl]-3,6-dihydro-2H-spiro[azepino[4,5-b]indole-1,1′-
cyclopentane]-5-carboxylate.

### Preparation of the Compounds of the Invention

Starting materials in the synthesis examples provided herein are either available from commercial sources or via literature procedures (e.g., March Advanced Organic Chemistry: Reactions, Mechanisms, and Structure, (1992) 4th Ed.; Wiley Interscience, New York). All commercially available compounds were used without further purification unless otherwise indicated. CDCl₃ (99.8% D, Cambridge Isotope Laboratories) was used in all experiments as indicated. Proton (¹H) nuclear magnetic resonance (NMR) spectra were recorded on a Bruker Avance 400 MHz NMR spectrometer. Significant peaks are tabulated and typically include: number of protons, and multiplicity (s, singlet; d, double; t, triplet; q, quartet; m, multiplet; br s, broad singlet). Chemical shifts are reported as parts per million (6) relative to tetramethylsilane. Low resolution mass spectra (MS) were obtained as electrospray ionization (ESI) mass spectra, which were recorded on a Perkin-Elmer SCIEX HPLC/MS instrument using reverse-phase conditions (acetonitrile/water, 0.05% trifluoroacetic acid). Flash chromatography was performed using Merck Silica Gel 60 (230-400 mesh) following standard protocol (Still et al. (1978) J. Org. Chem. 43:2923).

It is understood that in the following description, combinations of substituents and/or variables of the depicted formulae are permissible only if such contributions result in stable compounds under standard conditions.

It will also be appreciated by those skilled in the art that in the process described below the functional groups of intermediate compounds may need to be protected by suitable protecting groups. Such functional groups include hydroxy, amino, mercapto and carboxylic acid. Suitable protecting groups for hydroxy include trialkylsilyl or diarylalkylsilyl (e.g., t-butyldimethylsilyl, t-butyldiphenylsilyl or trimethylsilyl), tetrahydropyranyl, benzyl, and the like. Suitable protecting groups for amino, amidino and guanidino include t-butoxycarbonyl, benzyloxycarbonyl, and the like. Suitable protecting groups for mercapto include —C(O)—R (where R is alkyl, aryl or aralkyl), p-methoxybenzyl, trityl and the like. Suitable protecting groups for carboxylic acid include alkyl, aryl or aralkyl esters.

Protecting groups may be added or removed in accordance with standard techniques, which are well-known to those skilled in the art and as described herein. The use of protecting groups is described in detail in Green, T. W. and P. G. M. Wutz, Protective Groups in Organic Synthesis (1991), 2nd Ed., Wiley-Interscience.

In the following Schemes, unless otherwise noted, the various substituents R¹⁻²⁶, and R³²⁻³⁴ are as defined above in the Summary of the Invention, X is halo and Y is O, N or S, and A is S, O or NH. R⁸ groups in the following schemes also correspond to the R⁸ groups in the Summary of the Invention which are more specifically designated as R^{8a}, R^{8b}, R^{8c} and R^{8d}. One of ordinary skill in the art could easily ascertain which choices for each substituent are possible for the reaction conditions of each Scheme. Moreover, the substituents are selected from components as indicated in the specification heretofore, and may be attached to starting materials, intermediates, and/or final products according to schemes known to those of ordinary skill in the art.

Also it will be apparent that many of the products could exist as one or more isomers, that is E/Z isomers, enantiomers and/or diastereomers.

Scheme 1 below depicts the synthesis of compounds of formula (I). In general, heteroar-3-yl-2-ethylamines (1) are condensed with haloketones (2) (or haloaldehydes) and undergo subsequent rearrangement to give azepines (3), which then can react with electrophiles to afford products (4) of formula I. In particular, heteroar-3-yl-2-ethylamines 1 (R⁴-R⁸ as above) consist of optionally substituted tryptamines (A=NH), benzofuran-3-yl (A=O) and benzo[b]thiophene-3-yl-2-ethylamines (A=S). By example, a haloketone 2 can be chloro- or bromopyruvate (R¹=CO₂R and R²=H) and the electrophiles can be acyl or sulfonyl chlorides, chloroformates, isocyanates or isothiocyanates (R³=COR, SO₂R, CO₂R, CONRR′ and CSNRR′, respectively).

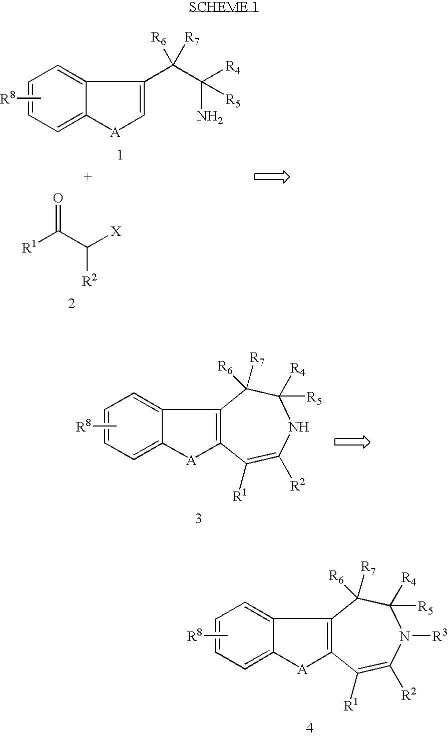

Many haloketones 2 (e.g. R¹ and R²=alkyl or aryl) are commercially available and can be prepared readily via common literature procedures. In addition, as depicted in Scheme 2 below, various 3-halopyruvates (6, R²=H) can be prepared by esterification of the corresponding alcohols (R¹OH) with 3-halopyruvic chloride (5) (Teague, et al, Bioorg. & Med. Chem. Lett. 1995, 5, 2341-2346).

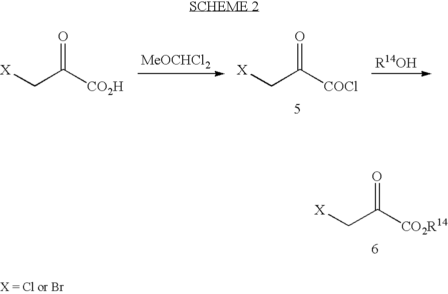

As depicted in Scheme 3 below, higher 3-halopyruvates 6b (e.g. R²=alkyl) can be synthesized via oxidative bromination of α-hydroxyesters (7) (Heterocycles 1991, 32, 693). While the non-hydrogen R² substituent can be incorporated into the final azepine products of formula I (e.g. 4), the following Schemes will feature examples that have been simplified by omission of R².

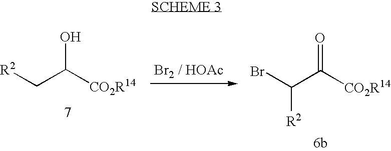

Some substituted tryptamines (11, A=NH) are commercially available, though many can be prepared from indoles (8, A=NH) as depicted in Scheme 4 below. For example, indoles 8 can be formylated to give aldehydes (9, A=NH) (Mor et al. J. Med. Chem. 1998, 41, 3831-3844). These 3-formylindoles 9 can undergo a Henry reaction Rosini Comp. Org. Syn. 1991, 2, 321-340) with 1-nitroalkanes to afford nitroalkenes (10, A=NH), which can be reduced (i.e., catalytic hydrogenation or lithium aluminum hydride) and then treated with HCl to yield tryptamine hydrochlorides 11. Likewise, other substituted heteroar-3-yl-2-ethylamines 11 (A=O or S) can be synthesized from their corresponding heterocycle 8, i.e. benzofurans and benzothiophenes. A variety of indoles also can be prepared via Fischer indole synthesis (Smith & March, March's Advanced Organic Chemistry, 5^{th} Ed., John Wiley and Sons:NY, 2001, pp 1453-24).

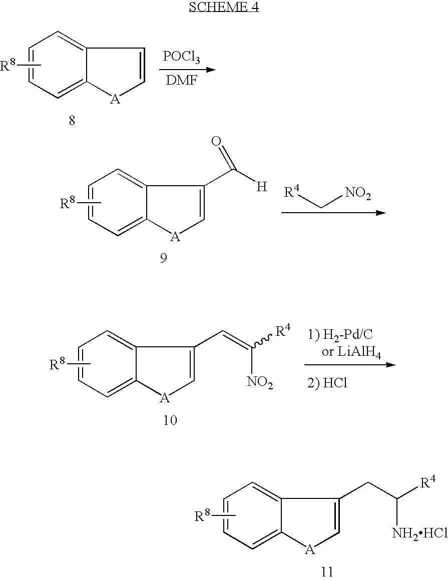

As depicted in Scheme 5 below, other substituted tryptamines (16) also can be prepared. Protection of 3-indolylacetonitriles (14), for example, with Boc (tert-butoxycarbonyl) followed by mono- or dialkylation, and then deprotection can yield substituted 3-indolylacetonitriles (15). Reduction of 15, e.g. with lithium aluminum hydride, followed by treatment with HCl affords tryptamine hydrochlorides 16. Thus, for example, monoalkyl species 15 (e.g. R₂=H, R⁶) can be prepared by addition of 1 equiv of alkyl halide. Gem-dialkyl species 15 (R=R⁶=R⁷) can be prepared from 2 equiv of alkyl halide and hetero-dialkyl species 15 (R₂=R⁶, R⁷) can be prepared upon sequential addition of 1 equiv each of two alkyl halides. Intermediates 14 can be prepared readily from gramines (13), which are either commercially available or synthesized from indoles (12) (Brown and Carrison, J. Chem. Chem. Soc. 1955, 77, 3839-3842). In general, gramines (13) can be treated with methyl iodide to form a quaternary ammonium salt, which can be displaced with cyanide to give 3-indolylacetonitriles 14. Benzofuran-3-yl and benzo[b]thiophene-3-yl ethylamines 7 (A=O and S) can be prepared using similar methods, in which protection and deprotection steps are not required.

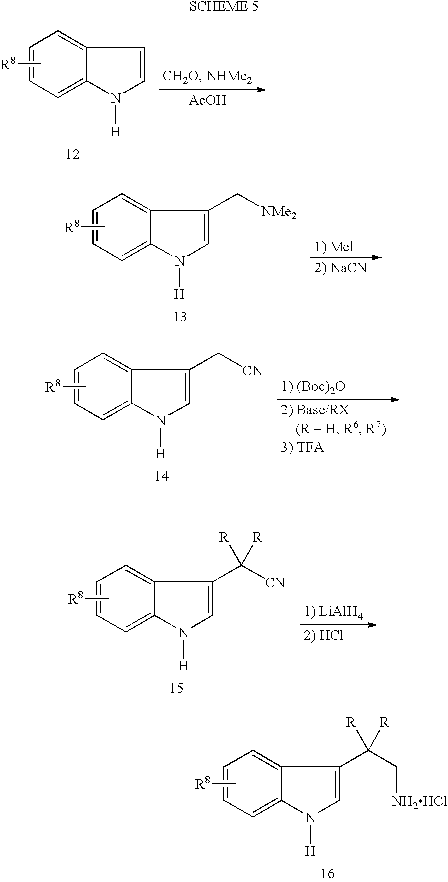

Preparation of spirocyclic analogs (18) of tryptamine also can be achieved as depicted in Scheme 6 below. For example, intermediate 14 can be protected with benzyl bromide followed by alkylation with an alkyl dihalide, e.g. 1,4-dibromobutane, to afford the corresponding intermediate (17, n=2). Subsequently 17 can be reduced, deprotected (e.g. with sodium metal in liquid ammonia) and treated with HCl to yield the spiro-substituted tryptamine hydrochloride 18.

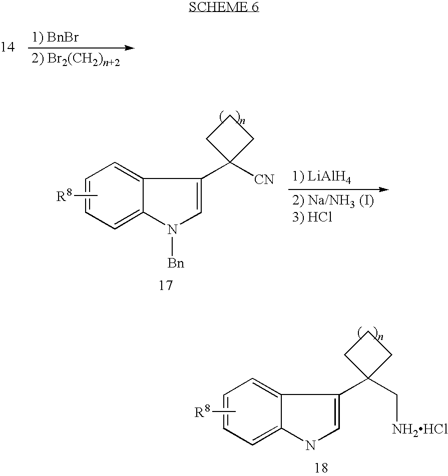

As depicted in Scheme 7 below, substituted tryptamines (21, A=NH) can also be prepared by Knoevenagel condensation of 3-indolylacetonitrile (19, A=NH) with an aldehyde to afford acrylonitriles (20, A=NH). Subsequent reduction, e.g. Rancy nickel, and treatment with HCl can yield tryptamine hydrochlorides 21. Analogous benzofuran-3-yl and benzo[b]thiophene-3-yl ethylamines 21 (A=O and S) also can be prepared using similar methods.

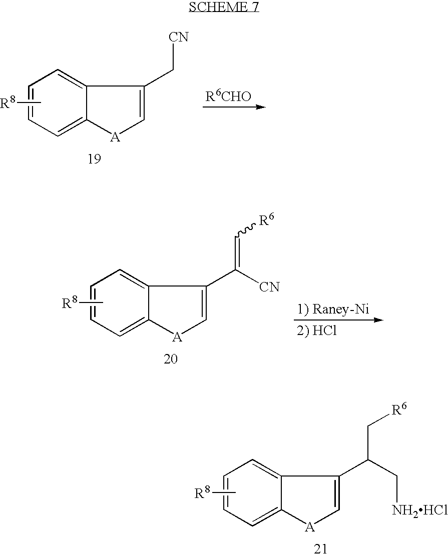

As depicted in Scheme 8 below, the azepine ring found in compounds of formula I (e.g. 23) can be achieved by a Pictet-Spengler reaction and a subsequent rearrangement. Thus, for example, tryptamines 1 (A=NH) can react with a ketone such as 3-halopyruvatcs 6 to afford β-carboline intermediates (22), which are then heated under basic conditions, i.e. with TEA or in pyridine, to give azepines (23) (Kuehne et al. (1985) J. Org. Chem. 50:919-924). Subsequent treatment of 23 with electrophiles, i.e. acyl or sulfonyl chlorides, isocyanates and chloroformates, in the presence of a base, e.g. TEA, affords final products 24. These intermediates 23 and products 24 can be further derivatized to yield additional compounds of formula I, as described in subsequent Schemes. In addition, azepino[4,5-b]benzofurans (24, A=O) and azepino[4,5-b]benzothiophenes (24, A=S) can be prepared in a similar manner from the respective heteroar-3-yl-2-ethylamines 1 (A=O and S).

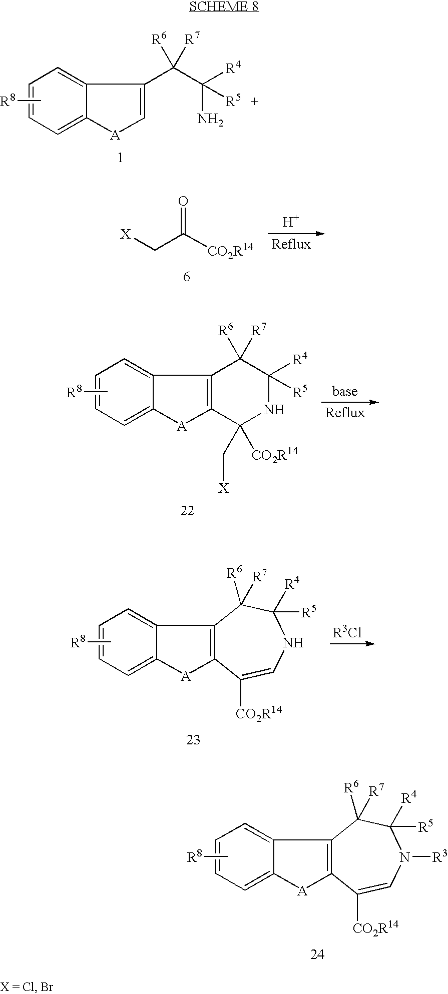

Likewise other haloketones 25 (e.g. R¹=alkyl or aryl) can undergo a similar reaction sequence to afford the corresponding azepines (26), as depicted in Scheme 9 below.

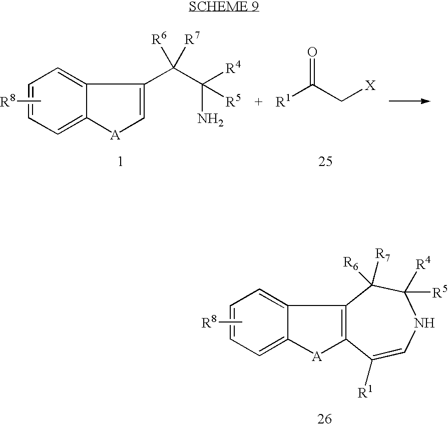

As depicted in Scheme 10 below, hexahydroazepino compounds (25) can be synthesized by reduction of azepines 23. For example, tetrahydroazepino[4,5-b]indoles 23 can be reduced with NaBH₃CN to give hexahydroazepino[4,5-b]indoles 25 (Kuehne et al. (1985) J. Org. Chem. 50:919-924), which can be treated with an electrophile, e.g. acyl chloride, to afford the corresponding azepine product (26).

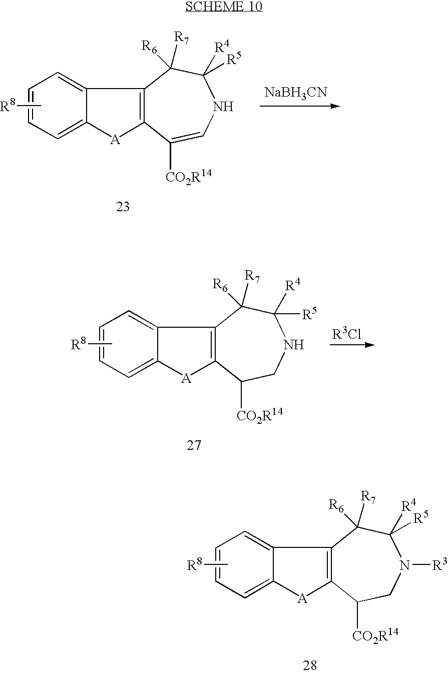

As depicted in Scheme 11 below, 5-esters 27 can be converted to 5-amides (30) via a multi-step reaction sequence. Azepine 27 can be treated with various amines to give the corresponding amides (29), which can then be reacted with an electrophile, e.g. an acyl chloride to afford the corresponding amide (29b). Oxidation of 29b with tert-butyl hypochlorite (Kuehne et al. (1985) J. Org. Chem. 50:919-924) then can yield the azepine product (30).

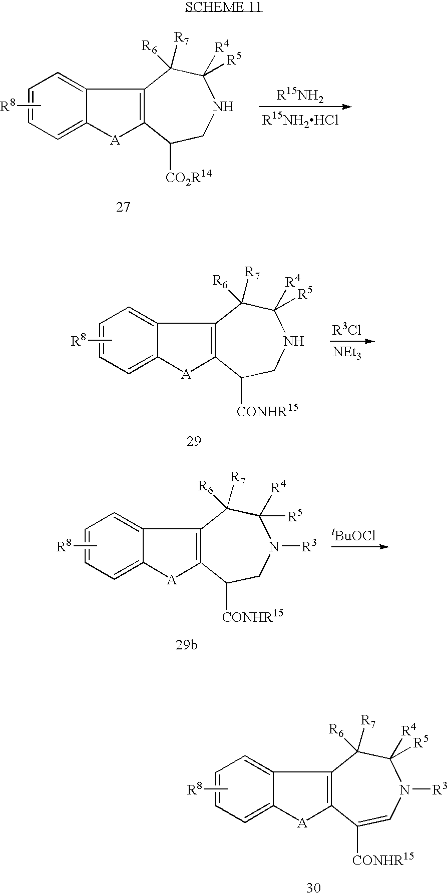

A more general approach for modification of the 5-ester group is depicted in Scheme 12 below. Azepine 28 can be saponified to give the respective acid (31). A nucleophile RYH (i.e. alcohols, phenols, amines, thiols) can be coupled with 31, e.g. using carbonyldiimidazole (CDI), followed by oxidation with tert-butyl hypochlorite to yield azepine (33).

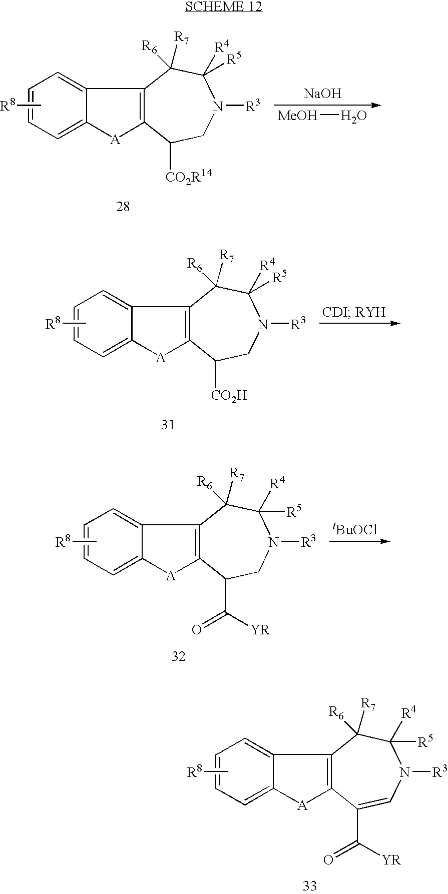

Heterocyclyl groups can be introduced at 5-position from acid 31. For example, as depicted in Scheme 13 below, oxazolines are prepared by formation of amides (34) from respective aminoalcohols and acid 31. The resulting amides 34 then can be cyclized, e.g. via treatment with thionyl chloride followed by strong base, to afford the corresponding heterocycle (36). Halogenation and subsequent dehydrohalogenation of intermediate (35) (not isolated) can occur under the reaction conditions. Similar reactions can be envisaged for other heterocycles, i.e. imidazolines and thiazolines. Also further oxidation would afford the corresponding heteroaromatic product, e.g. oxazole.

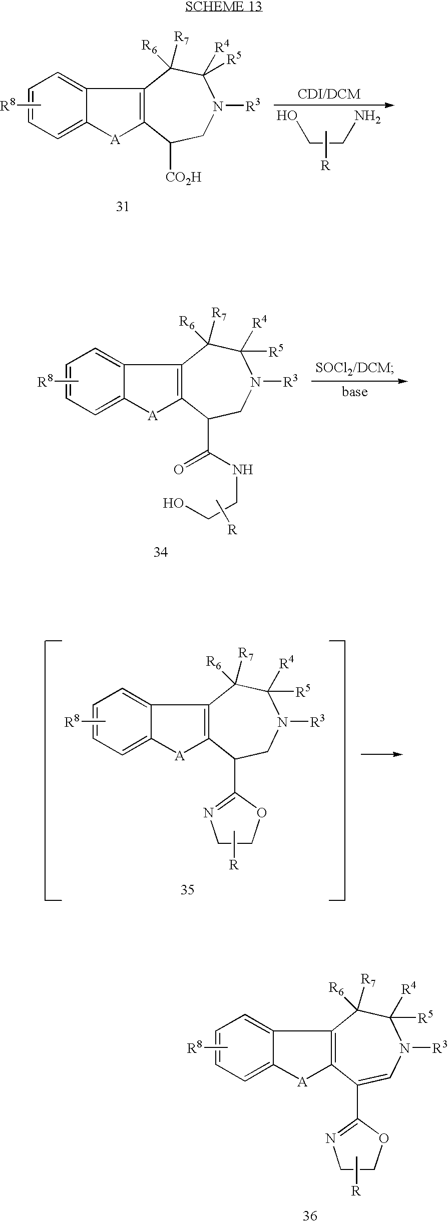

The 5-ester group of 23 can be hydrolyzed to give 5-carboxylic acid (38). However, direct hydrolysis affords 38 in low yield. Accordingly, as depicted in Scheme 14 below, azepine 23 was transformed into the 3-Boc-protected compound (37), which can be hydrolyzed under the standard basic conditions with Boc elimination to afford acid 38.

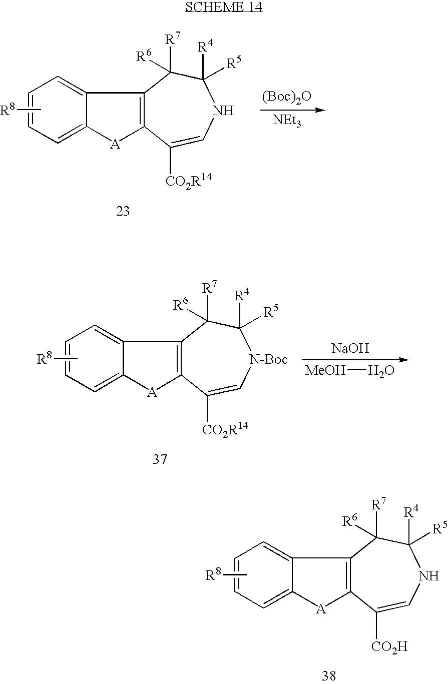

As depicted in Scheme 15 below, azepine 23 can be treated with Lawesson's reagent (Curphey, et al, J. Org. Chem. 2002, 67, 6461-6473) to afford O-alkyl thioester (39), which can be, for example, acylated to yield the azepine product (40).

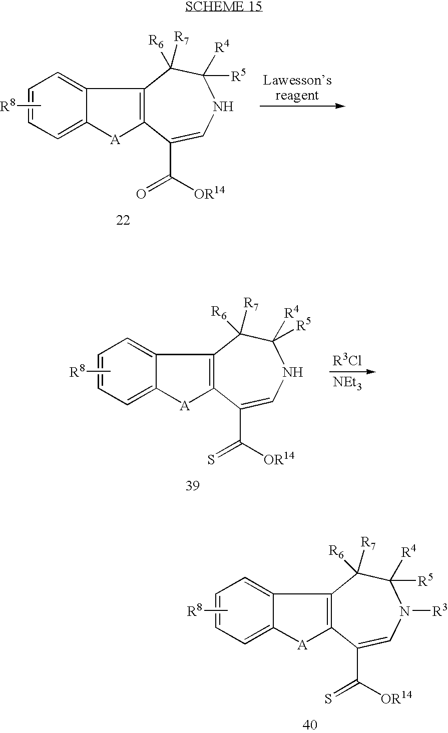

Scheme 16 below depicts the incorporation of 3-alkyl/aryl groups. For example, azepine 23 can be treated with a base, e.g. NaH, and then an alkyl halide (R³X) to yield a 3-alkyl azepine (41). An aryl or heteroaryl group (R³) can be introduced via coupling of 27 with boronic acids (Lam, et al, Tetrahedron Lett. 2001, 42, 3415-3418), followed by oxidation of intermediate (42) to give the corresponding azepine product (43).

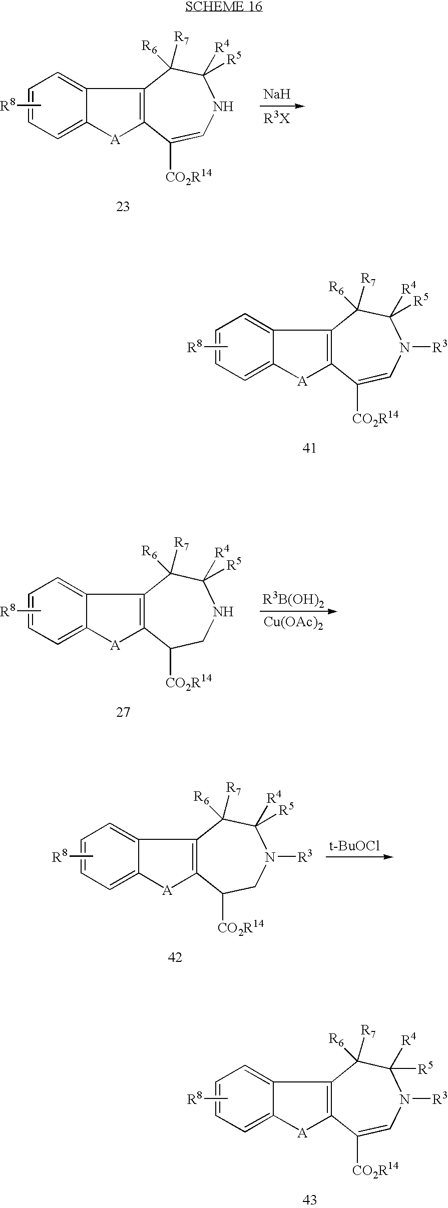

Derivatization of 2-substituted azepines (44) is depicted in Scheme 17 below. Diester (44) can be partially hydrolyzed to give acid (45), which can be transformed into amides (46), e.g. using CDI. Intermediates 46 can be further substituted upon addition of an electrophile, e.g. acyl chloride, to give the corresponding diamides (47).

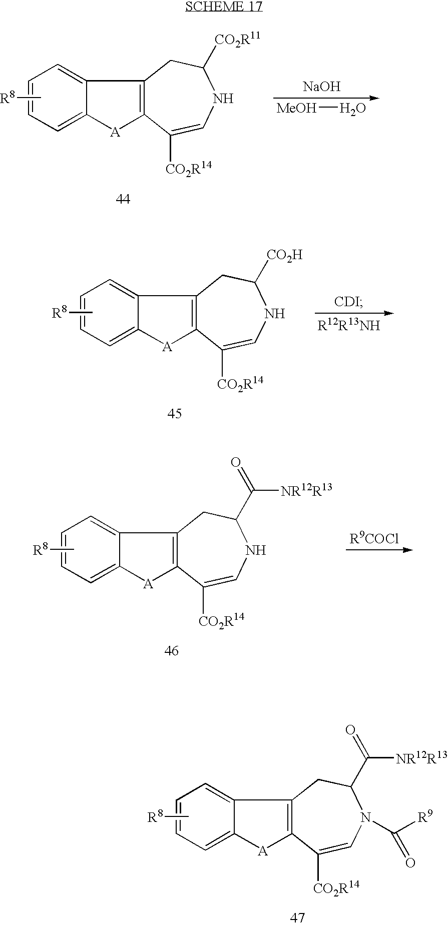

As depicted in Scheme 18 below, alcohol (48) can be derivatized by addition of an electrophile (i.e. acyl chloride, chloroformate or isocyanate). For example, 48 can be esterified in the presence of base to yield diester (49), though a mixture containing diester-amide (50) may result.

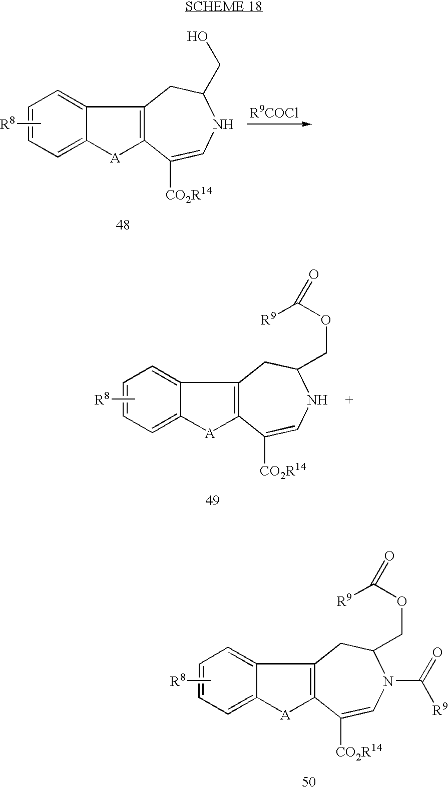

As depicted in Scheme 19 below, 1-oxoazepines (52) can be employed as key intermediates for introduction of other functional groups. For example, azepine (51) can be oxidized, e.g. with DDQ, to yield1-oxoazepine 52, which can be reduced to give the corresponding alcohol (53). Treatment of 53 with trifluoromethanesulfonic anhydride followed by addition of nucleophiles RYH (alcohols, thiols, amines, hydroxylamines and hydrazines) can yield the corresponding azepine products (54).

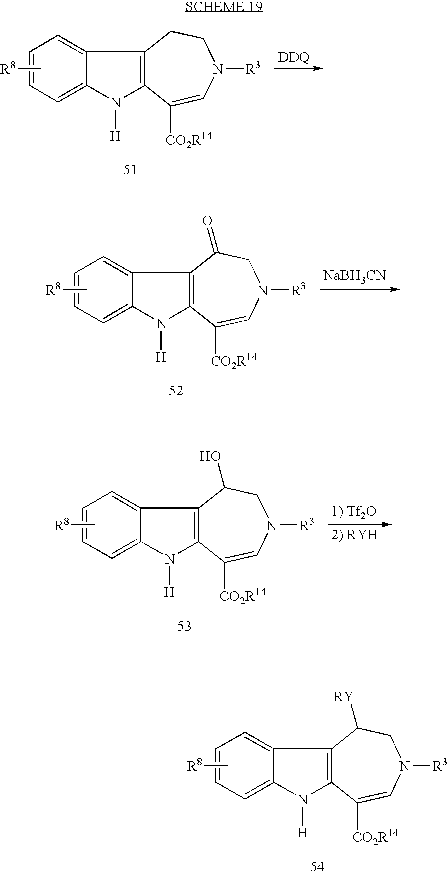

As depicted in Scheme 20 below, 1-oxoazepine 52 can be converteded, e.g. with dimethylphenylsilane in TFA, to the corresponding azepine (55).

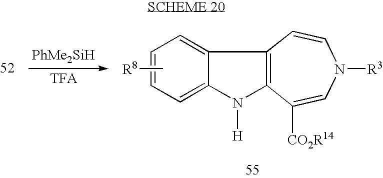

Likewise, as depicted in Scheme 21 below, 1-oxoazepine 52 can be treated with ethylene glycol under acid-catalysis to form cyclic acetal (56). Also 52 can be treated with amines, hydroxylamines and hydrazines to give imines (57, YR=NR¹⁵), oximes (57, YR=NOR¹⁴) and hydrazones (57, YR=NNR¹⁵R¹⁶), respectively. Furthermore 52 can undergo a Wittig or Horner-Wadsworth-Emmons reaction (Maercker (1965) Org. React. 14:270-490; Wadsworth, Jr. (1977) Org. React. 25:73-253) to yield exocyclic alkylidenes (57, e.g. YR=CRR′).

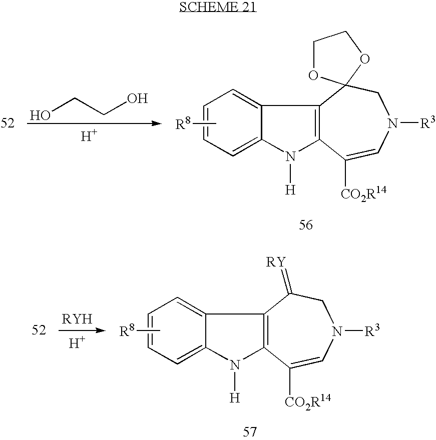

As depicted in Scheme 22 below, substituents on the indole ring can be introduced, i.e. via Suzuki cross-coupling and aryl amination reactions from the corresponding aryl bromides (59). Bromo-substituted indoles 59 can be prepared via direct bromination of indoles (58) with NBS or from commercially available tryptamine. These intermediates 59 can be used in Suzuki cross-coupling reactions (Miyaura, et al, Chem. Rev. 1995, 956, 2457-2483) with boronic acids to afford, for example, aryl-substituted products (60, R⁸=aryl) and in aryl amination reactions (Wolfe, et at, J. Org. Chem. 2000, 65, 1144-1157) to afford amino-substituted products (60, R⁸=NR²⁸R²⁹).

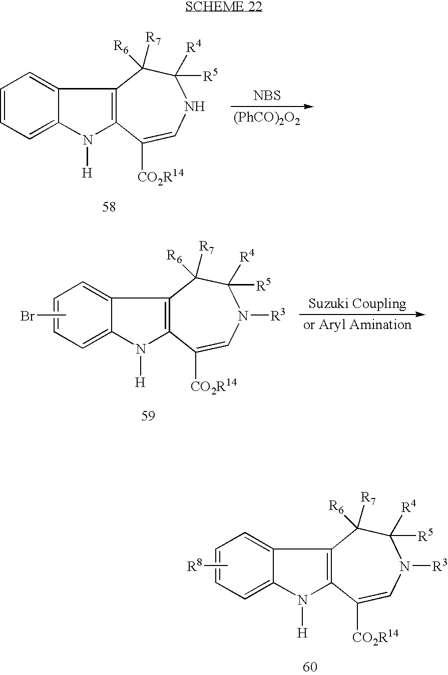

As depicted in Scheme 23 below, other transformations of functional groups can be achieved, for example, on the indole ring of azepine (61). Protective groups, such as alkyl and aryl groups, on oxygen, sulfur or nitrogen containing substituents of azepine 61 can be removed under suitable conditions to yield azepine (62). Treatment of 62 with electrophiles, such as carbamoyl chlorides, can yield the corresponding azepines (63), for which the substituent R⁸ is C(O)NR³²R³³ in this representative example.

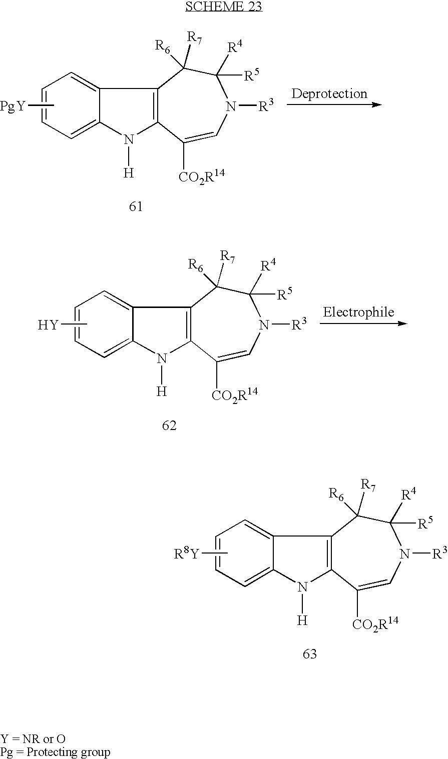

As depicted in Scheme 23 below, other transformations of functional groups can be achieved, for example, on the indole ring of azepine (61). Protective groups, such as alkyl and aryl groups, on oxygen, sulfur or nitrogen containing substituents of azepine 61 can be removed under suitable conditions to yield azepine (62). Treatment of 62 with electrophiles, such as carbamoyl chlorides, can yield the corresponding azepines (63), for which the substituent R⁸ is C(O)NR³²R³³ in this representative example.

As depicted in Scheme 24 below, 3-cyanomethyl-indole-1-carboxylic acid (64) was treated with BOC anhydride in the presence of DMAP/TEA to provide 3-cyanomethyl -indole-1-carboxylic acid tert-butyl ester (65). This product was then dissolved in dimethyl acetamide and then NaOH and iodomethane to obtain 3-(cyano-dimethylmethyl)-indole-1-carboxylic acid tert-butyl ester (66). An aqueous solution of ammonium hydroxide was added in the presence of Raney Ni. The catalyst was filtered off and rinsed with methanol to obtain 3-(2-amino-1,1-dimethyl-ethyl)-indole-1-carboxylic acid tert-butyl ester (67). The product was treated with HCl/dioxane with dichloromethane to yield 2(1-H-indol-3-yl)-2-methyl-propan-1-amine (68). Isopropyl-3-bromo-2-oxopropionale (69a) and Isopropyl-3-chloro-2-oxopropionale (69b) were added to (68) to produce isopropyl 1,1-dimethyl-1,2,3,6-tetrahydroazepino[4,5-b]indole-5-carboxylate.

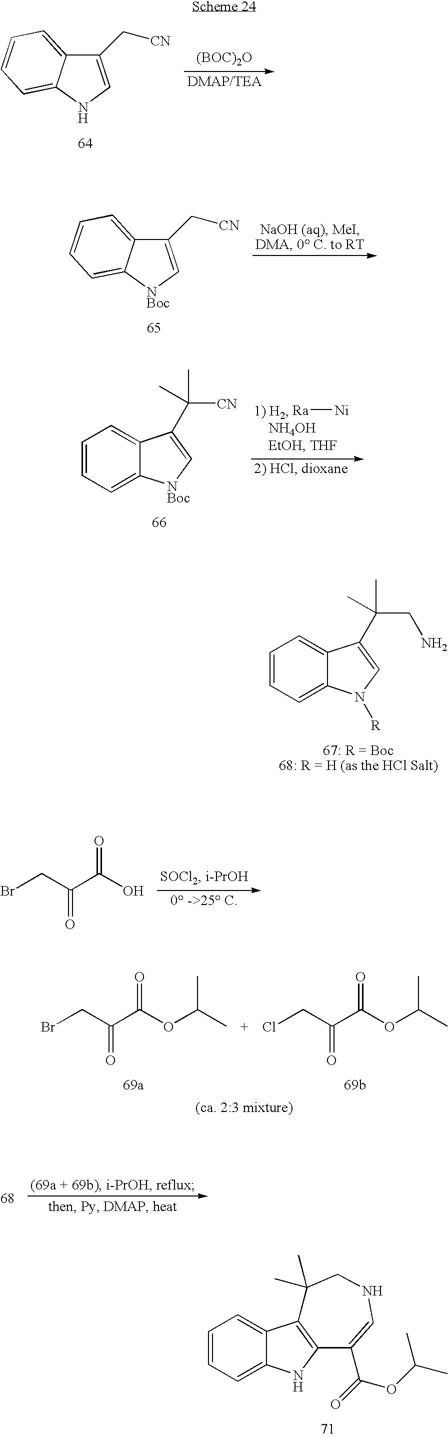

As depicted in Scheme 25, 2(1H-indol-3-yl)acetonitrile (72) was treated with Boc anhydride in the presence of TEA, DMAP and DCM to provide tertiary butyl-3-(cyanomethyl)-1-H-indole-1-carboxylate (73). The carboxylate is then reacted with sodium hydroxide, methyl iodide, water and DMA to obtain tertiary butyl 3-(2-cyanopropan-2-yl)-1H-indole-1-carboxylate (74) which is then reacted with Raney Ni, ammonium hydroxide, methanol and tetrahydrofuran to obtain tertiary butyl 3-(1-amino-2-methylpropane-2-yl)-1H-indole-1-carboxylate (75). The product was treated with 4M hydrochloric acid and dioxane to provide tertiary butyl 3-(2-cyanopropane-2-yl)-1H-indole-1-carboxylate (76). (2,2 dimethyl-1,3-dioxolan-4-yl)methyl-3-bromo-2-oxopropanoate was added to provide (2,2-dimethyl-1,3-dioxolan-4-yl)methyl 1,1-dimethyl-1,2,3,6-tetrahydroazepino[4,5-b]indole-5-carboxylate (77). 1N hydrochloric acid and tetrahydrofuran were added to procide 2,3 dihydroxypropyl 1,1-dimethyl-1,2,3,6-tetrahydroazepino[4,5-h]indole-5-carboxylate (78). 3,4 difluorobenzoyl chloride was added to the resulting product to provide 2,3-dihydroxypropyl-3-(3,4-difluorobenzoyl-1,1-dimethyl-1,2,3,6-tetrahydroazepino[4,5-b]indole-5-carboxylate (79).

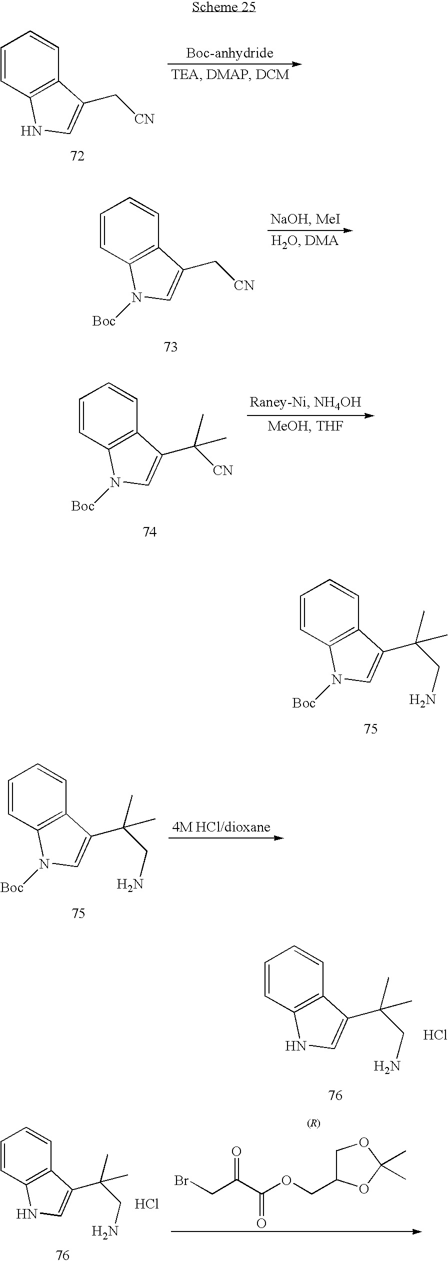

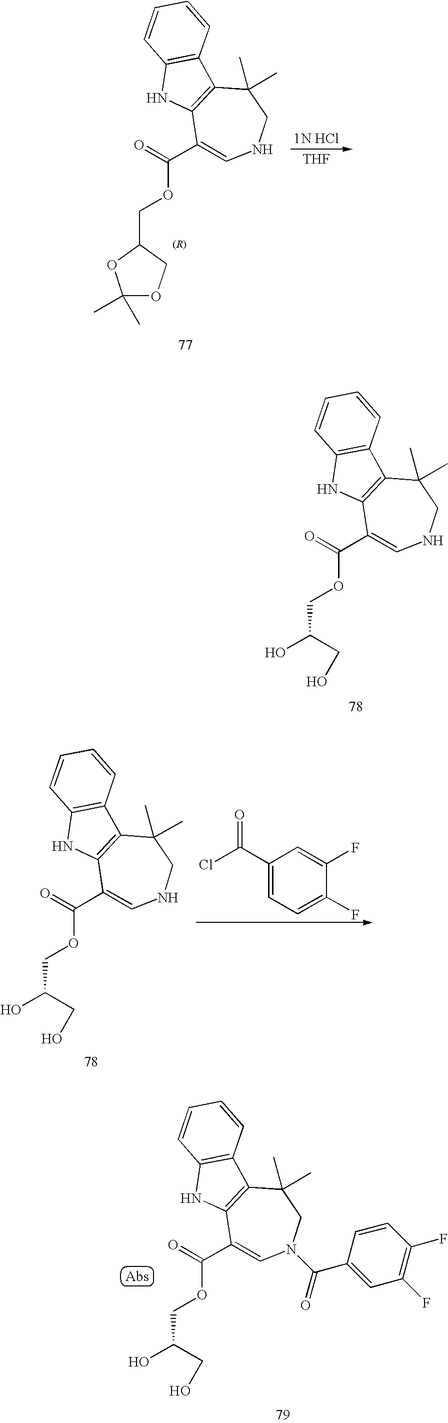

As depicted in Scheme 26, the preparation of 2,3-dihydroxypropyl-3-(3,4-difluorobenzoyl-1,1-dimethyl 1,2,3,6 tetrahydroazepino[4,5-b]indole-5-carboxylate is as follows: 2,2-dimethyl-1,3 dioxolan-4-yl methanol and bromopropionic acid were reacted in the presence of chlorodimethoxymethane to produce (2,2 dimethyl 1,3-dioxolan-4-yl)methyl 3 bromo-2-oxopropionate. 2-(1H-indol-3-yl)-2-methyl-propan-1-amine hydrochloride was treated with (2,2-dimethyl 1,3-dioxolan-4-yl)methyl 3 bromo-2-oxopropanoate to form (2-methyl-1,3-dioxolan-4-yl)methyl1,1-dimethyl-1,2,3,6 tetrahydroazepino[4,5-b]indole-5-carboxylate. This is then treated with difluorobenzoyl chloride to produce (2,2-dimethyl 1,3-dioxolan-4-yl)methyl 3-(3,4-difluorobenzoyl)-1,1-dimethyl-1,2,3,6 tetrahydroazepino[4,5-b]indole-5-carboxylate. This product is treated with 1N hydrochloric acid and tetrahydrofuran to produce 2,3-dihydroxypropyl 3-(3,4-difluorobenzoyl)-1,1-dimethyl-1,2,3,6 tetrahydroazepino[4,5-b]indole-5-carboxylate.

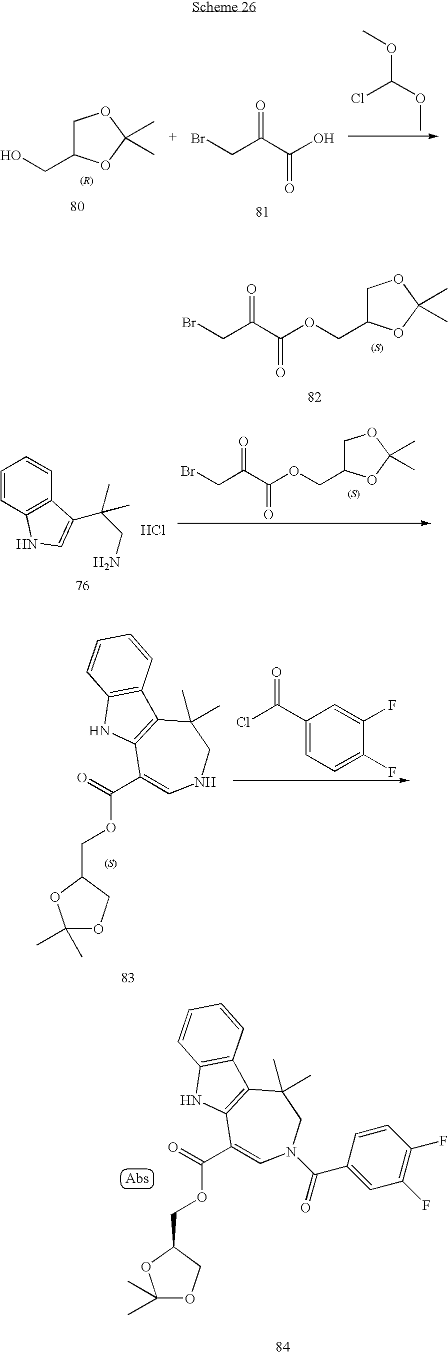

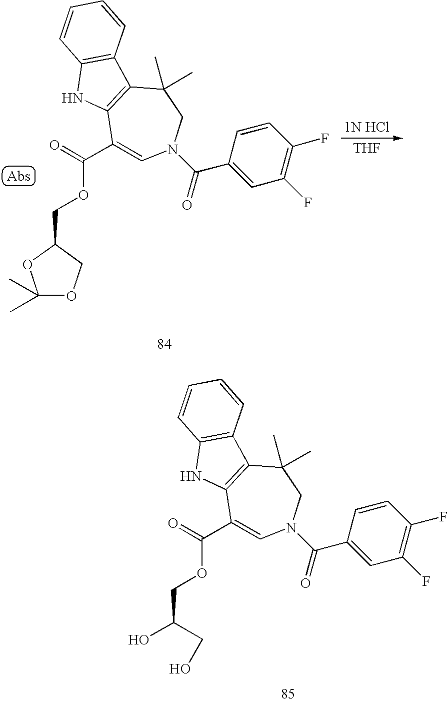

As depicted in Scheme 27, 2-Hydroxy-1-Methylethyl 3-[(3,4-Difluorophenyl)Carbonyl]-1,1-Dimethyl-1,2,3,6-Tetrahydroazepino[4,5-b]Indole-5-Carboxylate is prepared as follows: 3 bromo-2-oxopropanoic acid is treated with tertiary butoxy-propan-2-ol and chlorodimethoxymethane to produce tertiary butoxy propan-2-yl3-bromo-2-oxopropanoate. This product was reacted with 2-(1H-indol-3-yl)-2-methyl-propan-1-amine hydrochloride to yield 1-hydroxy propan-2-yl 1,1-dimethyl -1,2,3,6-Tetrahydroazepino[4,5-b]Indole-5-Carboxylate. This product was then treated with difluorobenzoyl chloride in the presence of DIEA and DCM to produce 1-hydroxypropan-2-yl 3-(3,4-difluorobenzoyl)-1,1-dimethyl-1,2,3,6-Tetrahydroazepino[4,5-b]Indole-5-Carboxylate.

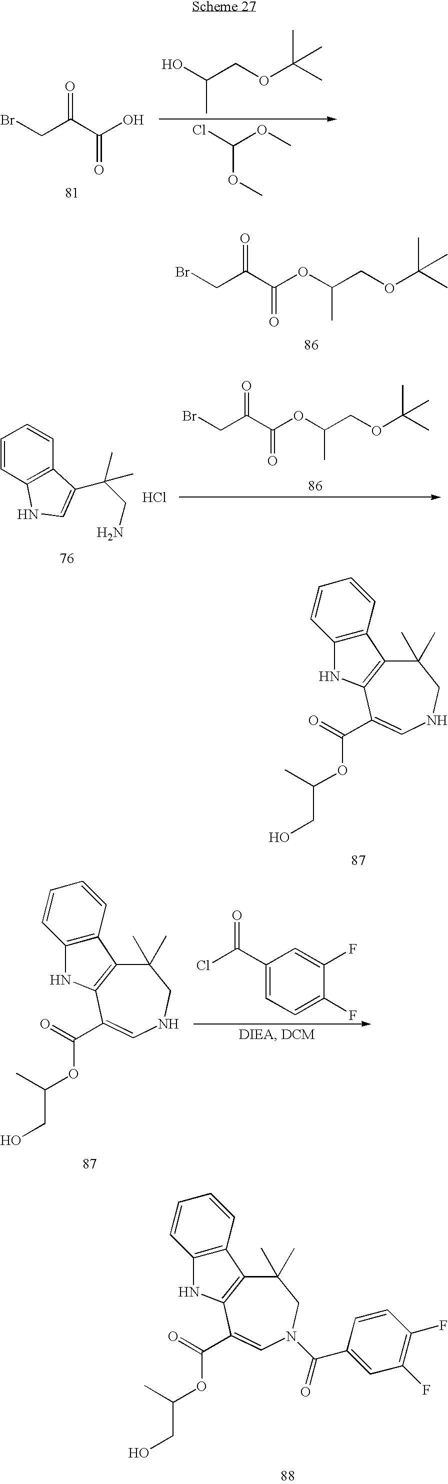

As depicted in Scheme 28, the preparation of 2-[(Phenylmethyl)Oxy]ethyl 3-[(3,4-Difluorophenyl)Carbonyl]-1,1-Dimethyl-1,2,3,6-Tetrahydroazepino[4,5-b]Indole-5-Carboxylate is prepared as follows:

3-bromo-2-oxopropionic acid was reacted with 2 benzyloxy ethanol in the presence of chlorodimethoxymethane to produce 2-(benzyloxy)ethyl 3-bromo-2-oxopropanoate. The product was reacted with 2-(1H-indol-3-yl)-2-methyl-propan-1-amine hydrochloride to yield 2-(benzyloxy)1,1-Dimethyl-1,2,3,6-Tetrahydroazepino[4,5-b]Indole-5-Carboxylate. The product was treated with difluorobenzoyl chloride to produce 2-(benzyloxy)ethyl 3-(3,4-difluorobenzoyl)-1,1-Dimethyl-1,2,3,6-Tetrahydroazepino[4,5-b]Indole-5-Carboxylate.

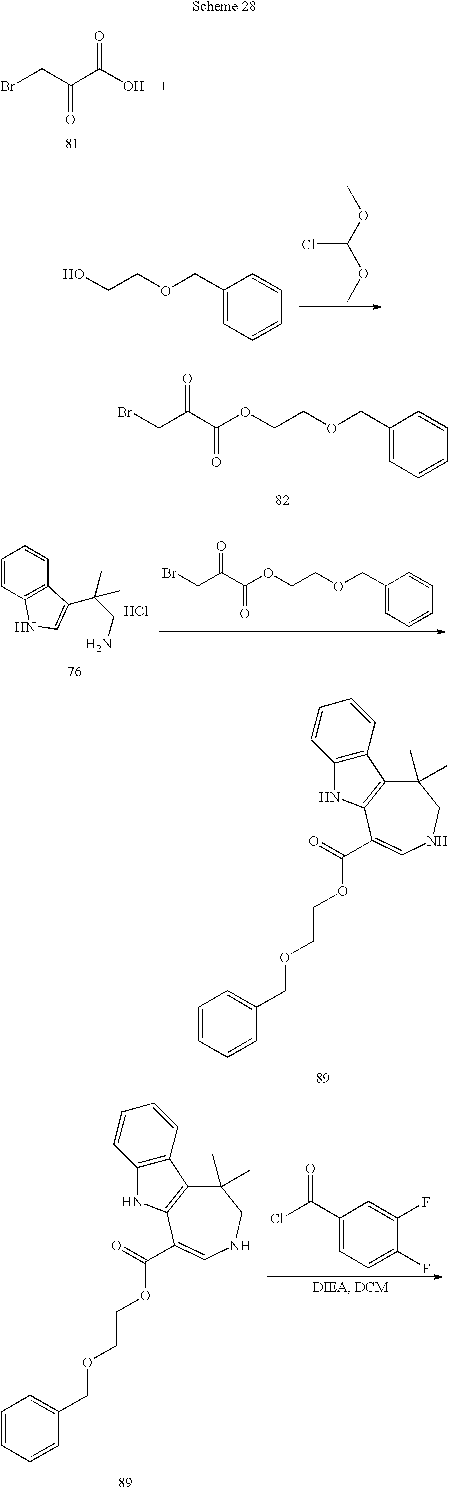

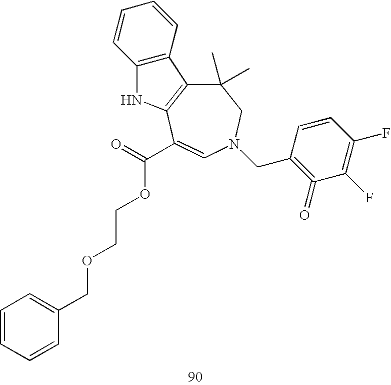

As depicted in scheme 29, 2-Fluoro-1-(Fluoromethyl)Ethyl 3-[(3,4-Difluorophenyl)Carbonyl]-1,1-Dimethyl-1,2,3,6-Tetrahydroazepino[4,5-b]Indole-5-Carboxylate was prepared as follows: 3-bromo-2-oxopropionic acid was reacted with 1,3-difluoropropan-2-ol to produce 1,3-difluoropropan-2-yl-3-chloro-2-oxopropanoate. The product was reacted with 2-(1H-indol-3-yl)-2-methyl-propan-1-amine hydrochloride to yield 1,3-difluoropropan-2-yl 1,1-dimethyl-1,2,3,6-Tetrahydroazepino[4,5-b]Indole-5-Carboxylate. The product was then treated with difluorobenzoyl chloride in the presence of DIEA and DCE to yield 1,3-difluoropropan-2-yl 3-(3,4-difluorobenzoyl)-1,1dimethyl-1,2,3,6-Tetrahydroazepino[4,5-b]Indole-5-Carboxylate.

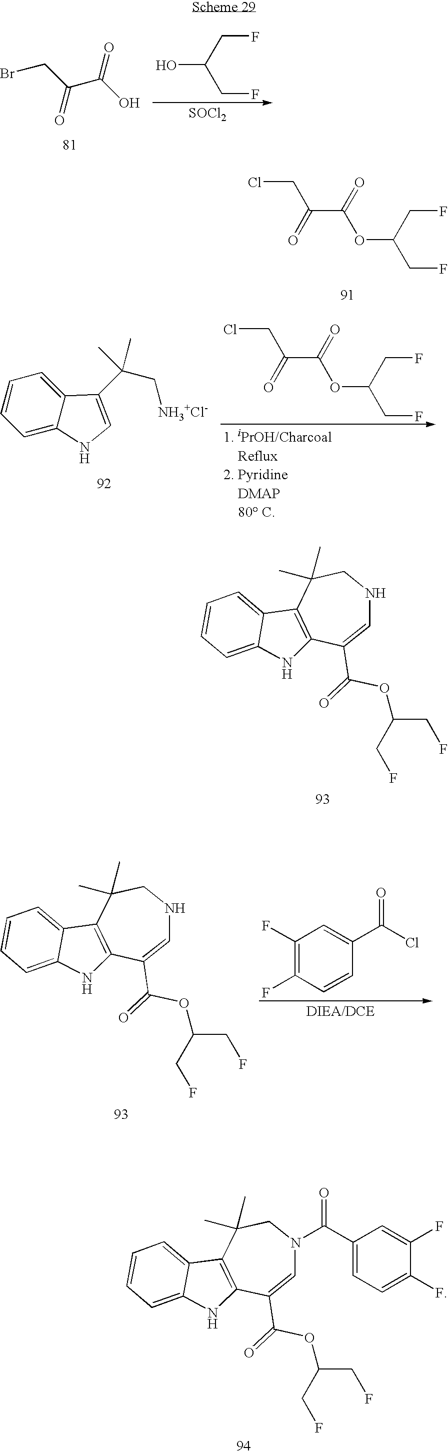

As depicted in Scheme 30, 1-{3-[(3,4-Difluorophenyl)Carbonyl]-1,1-Dimethyl-1,2,3,6-Tetrahydroazepino[4,5-b]Indol-5-yl}Ethanone was prepared as follows: Biacetyl was refluxed with sulfurous dichloride and benzene to produce 1 chlorobutane 2,3-dione. This product was treated with 2-(1H-indol-3-yl)-2-methyl-propan-1-amine hydrochloride to yield 1-(1,1-dimethyl-1,2,3,6-Tetrahydroazepino[4,5-b]Indole-5-Carboxylate. The product was then treated with difluorobenzoyl chloride to obtain 1-(3-(3,4-difluorobenzoyl)-1,1-dimethyl-1,2,3,6-Tetrahydroazepino[4,5-b]Indole-5-Carboxylate.

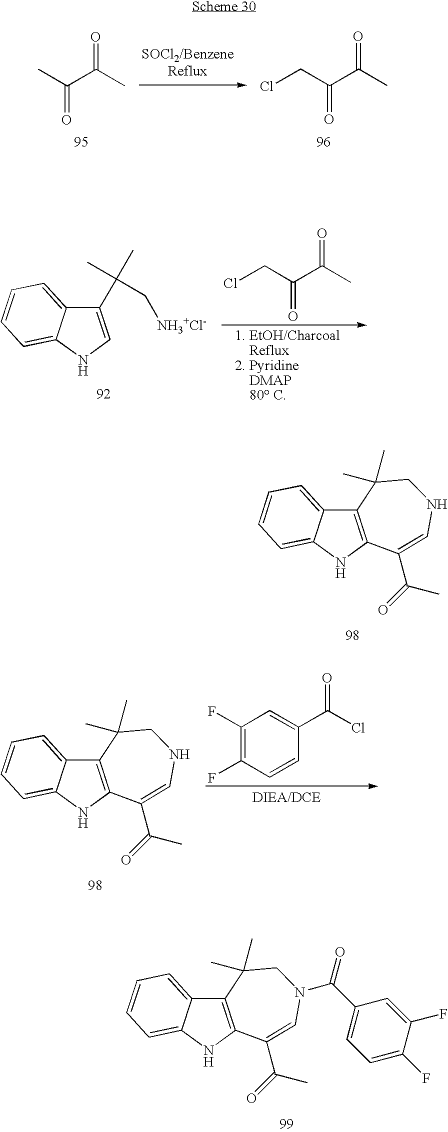

The following examples are provided for illustrative purposes only and are not intended to limit the scope of the invention.

### EXAMPLES

### Example 1

### Preparation of 1-Methylethyl 1,1-Dimethyl-1,2,3,6-Tetrahydroazepino [4,5-b]Indole-5-CarboxylateE

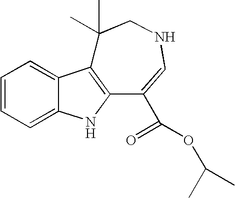

The title compound was prepared as depicted in Scheme 24: ¹H NMR (400 MHz, CDCl₃): δ 10.98 (s, 1H), 7.88 (d, J=8.0 Hz, 1H), 7.78 (d, J=8.0 Hz, 1H), 7.32 (d, J=8.0 Hz, 1H), 7.05 (t, J=6.8 Hz, 1H), 6.99 (td, J=7.8, 1.2 Hz, 1H), 5.29 (br s, 1H), 5.16 (sept., J=6.4 Hz, 1H), 3.27 (br s, 2H), 1.56 (s, 6H), 1.33 (s, 3H), 1.32 (s, 3H); MS (EI) for C₁₈H₂₃N₂O₂, 299.2 (MH⁺).

### Preparation 1

### 3-Cyanomethyl-indole-1-carboxylic acid tert-butyl ester

To a cooled (ice-water bath), stirred solution of 3-indolylacetonitrile (64) (53.4 g, 0.342 mol) in dichloromethane (800 mL) was added triethylamine (84 mL, 0.603 mol) and dimethylaminopyridine (2.64 g, 0.0216 mol). BOC anhydride (88.4 g, 0.405 mol) was melted by slightly heating in warm water bath, and then added slowly to the reaction mixture as a liquid. After the addition was complete, ice bath was removed and stirring continued at room temperature for 2-3 hours, or until no more starting indole was present by TLC (eluted with 25% EtOAc in hexanes). The reaction was washed with 1N HCl (2×400 mL) and brine, then dried with Na₂SO₄, filtered, and concentrated in vacuo to yield 3-cyanomethyl-indole-1-carboxylic acid tert-butyl ester (65) as a pale yellow solid (87.2 g, 100%). ¹H-NMR (400 MHz, CDCl₃): 8.18-8.16 (d, 1H), 7.64 (s, 1H), 7.53-7.51 (dd, 1H), 7.40-7.36 (m, 1H), 7.32-7.26 (m, 1H), 3.78 (s, 2H), 1.68 (s, 9H).

### Preparation 2

### 3-(Cyano-Dimethyl-Methyl)-Indole-1-Carboxylic Acid Tert-butyl Ester

3-Cyanomethyl-indole-1-carboxylic acid tert-butyl ester (65) (40.0 g, 1.0 eq.) dissolved in N,N-dimethylacetamide (400 mL) was cooled in ice-water bath at 0° C., then NaOH (18.728 g, 3.0 eq.) dissolved in H₂O (18.728 mL) was added dropwise to the solution mixture. After stirring for 10 minutes, MeI (66.46 g, 3.0 eq.) was slowly added while the reaction mixture was continued to cool at 0° C. After addition, the mixture was slowly warmed up to room temperature and stirred overnight (16 hours). Precipitate was observed and the reaction was considered complete by LC/MS and TLC (R_{f}=0.37; 10:90 EtOAc/Hex). Water (250 mL) was added to the reaction mixture and the precipitate was collected by filtration, and then rinsed with H₂O several times to remove residual NaOH and DMA. More precipitate was formed in the filtrate, and it was collected by filtration once again as mentioned above. The precipitate was then rinsed with small amount of hexanes, dried overnight under vacuum to yield 3-(cyano-dimethyl-methyl)-indole-1-carboxylic acid tert-butyl ester (66) as an off-white solid (31.8 g 71.7%). ¹H-NMR (400 MHz, CDCl₃): 8.18-8.16 (br d, 1H), 7.83-7.81 (dd, 1H), 7.53 (s, 1H), 7.39-7.28 (m, 2H), 1.85 (s, 6H), 1.68 (s, 9H).

### Preparation 3

### 3-(2-Amino-1,1-Dimethyl-Ethyl)-Indole-1-Carboxylic Acid Tert-Butyl Ester

In a 1-L Parr shaker flask, 3-(cyano-dimethyl-methyl)-indole-1-carboxylic acid tert-butyl ester (66) (19.1 g, 0.067 mol) was dissolved in a 2:1 MeOH/THF solution (375 mL). An aqueous solution of ammonium hydroxide (28-30%) (9.8 mL, 0.067 mol) was added, followed by approximately 19 mL of Raney-Ni (slurry in water). The reaction was carried out in a Parr apparatus with H₂ at 45 psi. at room temperature. The reaction was complete after 3 hours. The catalyst was filtered off and rinsed with MeOH, and the resulting filtrate was concentrated in vacuo. The residual crude oil was taken up into dichloromethane (400 mL), and washed with water (2×250 mL) and brine, then dried with anhydrous Na₂SO₄, filtered, and concentrated in vacuo to yield 3-(2-amino-1,1-dimethyl-ethyl)-indole-1-carboxylic acid tert-butyl ester (67) as a yellow oil. ¹H-NMR (400 MHz, CDCl₃): 8.18 (br s, 1H), 7.72-7.70 (d, 1H), 7.36 (br s, 1H), 7.32-7.28 (m, 1H), 7.23-7.19 (m, 1H), 3.00 (s, 2H), 1.67 (s, 9H), 1.40 (s, 6H), 1.03 (br s, 2H).

### Preparation 4

### 2-(1H-Indol-3-yl)-2-Methyl-Propylamine Hydrochloride

The resulting yellow oil 3-(2-amino-1,1-dimethyl-ethyl)-indole-1-carboxylic acid tert-butyl ester (67) from the previous step was immediately subjected to 4M HCl/dioxane (200 mL) and stirred at room temperature overnight (16 hours). After such time, white precipitate was observed. Small amount of dichloromethane was added to loosen up the solid. The solid was then filtered, rinsed with dichloromethane and dried under vacuum overnight to yield 2-(1H-indol-3-yl)-2-methyl-propylamine hydrochloride (68) as a white solid (16.0 g, >99%). HPLC purity: 98.4% (□=254 nM). ¹H-NMR ¹H-NMR (400 MHz, d₆-DMSO): 11.08 (s, 1H), 7.80 (br s, 3H), 7.73-7.71 (d, 1H), 7.40-7.38 (d, 1H), 7.15-7.14 (d, 1H), 7.11-7.07 (m, 1H), 7.01-6.97 (m, 1H), 3.13-3.12 (d, 2H), 1.44 (s, 6H). Elemental analysis: calcd for Cl₂₁H₁₆N₂.HCl.0.65H₂O: 60.96% C, 7.80% H, 11.85% N, 14.99% Cl; found: 61.13% C, 7.06% H, 11.37% N, 14.51% Cl.

### Preparation 5

### 3-Bromo-2-Oxo-Propionic acid Isopropyl Ester and 3-Chloro-2-Oxo-Propionic Acid Isopropyl Ester

3-Bromo-2-oxo-propionic acid (14.8 g, 88.6 mmol, 1 eq) was dissolved in isopropyl alcohol (50 ml) and the mixture was then cooled in an ice bath. Thionyl chloride (31.6 g, 265 mmol, 19.4 ml, 3 eq) was added dropwise while maintaining the reaction temperature below 5° C. The addition was completed within 30 minutes. Upon completion of the addition of thionyl chloride the reaction was removed from the ice bath and allowed to stir at room temperature for 3 hours. The reaction mixture was concentrated on rotovap to remove the excess of isopropyl alcohol and thionyl chloride. The crude product mixture was then subjected to a vacuum distillation. After distilling off an early fraction (30° C. at 4.3 torr), products 69a and 69b (12.89 g, 88%) were collected as a 3:2 mixture at the temperature between 68-75° C. (4.3 torr). ¹H NMR (400 MHz, CDCl₃): 5.21 (m, 1H), 4.59 (s, 2H, compound 69b), 4.32 (s, 2H, compound 69a), 1.37 (d, 6H) ppm.

### Preparation 6

### 1,1-Dimethyl-1,2,3,6-Tetrahydro-Azepino[4,5-b]Indole-5-Carboxylic Acid Isopropyl Ester

2-(1H-indol-3-yl)-2-methyl-propylamine hydrochloride (68) (3.0 g, 13.4 mmol, 1 eq), isopropyl alcohol (30 ml), a 2:3 mixture of 3-bromo-2-oxo-propionic acid isopropyl ester (69a) and 3-chloro-2-oxo-propionic acid isopropyl ester (69b) (3.2 g, 1.5 eq, based on the MW of 69b) and charcoal (10% by weight, 0.3 g) were added in a round bottom flask. The mixture was heated under nitrogen atmosphere at reflux for 2 hours. The reaction was monitored by LCMS following the formation of intermediates isopropyl 1-(bromomethyl)-4,4-dimethyl-2,3,4,9-tetrahydro-1H-pyrido[3,4-b]indole-1-carboxylate (70a) and isopropyl 1-(chloromethyl)-4,4-dimethyl-2,3,4,9-tetrahydro-1H-pyrido[3,4-b]indole-1-carboxylate (70b) and depletion of starting material (68). The reaction was then cooled to room temperature and pyridine (2.65 g, 2.68 ml, 33.4 mmol, 2.5 eq) was added along with DMAP (260 mg, 9% by weight) (Note: DMAP may not be necessary, however in some cases it was found to shorten reaction times). The reaction was then refluxed overnight and upon completion the reaction was cooled and filtered through celite. The celite was washed by 300 ml of DCM and the filtrate was evaporated to dryness. TLC revealed an Rf=0.85 in DCM with no nearby impurities and at this point either a column or silica plug may be used. If a silica plug is used a 50:50 mixture of DCM /hexane should be considered. The dark brown solid was columned using a biotage system and DCM as the eluent, providing the product as the first fraction collected off the column. After removal of solvents in vacuo, hexane was added to wash a small impurity away, yielding the product (71) as a yellow solid (2.98 g, 75% yield). ¹H NMR (400 MHz, CDCl₃): 10.99 (bs, 1H), 7.85 (d, 1H), 7.78 (m, 1H), 7.32 (d, 1H), 7.03 (m, 1H), 6.99 (m, 1H), 5.28 (bs, 1H), 5.15 (m, 1H), 3.22 (bs, 2H), 1.53 (s, 6H), 1.32 (d, 6H); MS (EI) for C₁₈H₂₂N₂O₂: 335.1 (MH⁺); Anal. Calcd. for C₁₈H₂₂N₂O₂ 0.1H₂O: C, 72.02; H, 7.45; N, 9.33. Found: C, 71.78; H, 7.64; N, 9.23.

### Example 1A

### Preparation of Ethyl 1,2,3,6-tetrahydroazepino[4,5-b]indole-5-carboxylate

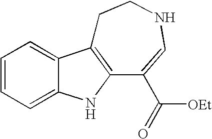

A mixture of tryptamine hydrochloride (1.96 g, 10 mmol), ethyl 3-bromopyruvate (1.67 mL, 1.2 equiv) and decolorizing charcoal (0.5 g) in absolute ethanol was heated to reflux under nitrogen overnight. TEA was added and the reaction mixture was heated to reflux for another 7.5 hours. After cooling, charcoal was removed by filtration and washed with ethanol. The filtrate was concentrated under vacuum and diluted with water (20 mL). It was then extracted by EtOAc (3×30 mL) and the combined organic layers were washed with brine and dried over MgSO₄. Evaporation of solvent and recrystallization from DCM-Hexane gave the title compound (1.17 g). ¹H-NMR (CDCl₃): δ 10.49 (1H, br s), 7.79 (1H, d), 7.43 (1H, d), 7.43 (1H, d), 7.06 (2H, m), 5.27 (1H, br s), 4.29 (2H, q) 3.58 (2H, m), 3.17 (2H, m), 1.36 (3H, t); MS (ES): 257 (MH⁺).

### Example 2

### Preparation of iso-Propyl 1,2,3,6-Tetrahydroazepino[4,5-b]indole-5-carboxylate

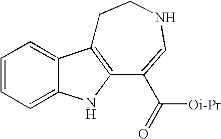

A. 3-Bromopyruvic acid-hydrate (3.34 g, 20 mmol) was placed in a flask and 1,1-dichloromethyl methyl ether (3.7 mL, 20 mmol) was added at 20° C. The mixture was heated to 50° C. with stirring and a clear solution was obtained in 10 minutes. Heating was continued for 2 h. Solvent was removed under high vacuum to give 3-bromopyruvic chloride (6 g, 90% pure by ¹H NMR) and the compound was used without further purification.

B. To iso-propanol was added 3-bromopyruvic chloride (5 g) at −5° C. dropwise and the solution was stirred overnight at 20° C. Evaporation of solvent gave iso-propyl 3-bromopyruvate (3.5 g), which was used in the next step without further purification.

C. A mixture of tryptamine hydrochloride (1.96 g, 10 mmol), iso-propyl 3-bromopyruvate in iso-propanol (1.67 mL, 1.2 equiv) and decolorizing charcoal (0.5 g) was heated to reflux under nitrogen overnight. TEA was added and the reaction mixture was heated to reflux for another 7.5 hours. After cooling, charcoal was removed by filtration and washed with ethanol. The filtrate was concentrated under vacuum and diluted with water (20 mL). It was then extracted by EtOAc (3×30 mL) and the combined organic layers were washed with brine and dried over MgSO₄. Evaporation of solvent and recrystallization from DCM-Hexane gave the title compound; ¹H-NMR (DMSO): δ 10.61 (1H, br s), 7.81 (1H, s), 7.67 (1H, m), 7.28 (2H, m), 6.83 (1H, m), 4.96 (1H, br s), 3.39 (2H, m), 3.27 (1H, m), 2.93 (2H, m), 1.20 (6H, d); MS (ES): 271 (MH⁺).

### Example 3

### Preparation of iso-Propyl 3-Benzoyl-1,2,3,6-tetrahydroazepino[4,5-B]indole-5-carboxylate

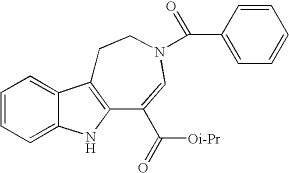

A. To a solution of iso-propyl 1,2,3,6-tetrahydroazepino[4,5-b]indole-5-carboxylate (52 mg, 0.2 mmol) in DCM was added benzoyl chloride (36 μL, 0.2 mmol) and TEA (56 μL, 0.4 mmol) and the mixture was shaken overnight at 20° C. Trisamine resin (50 mg) was added and the suspension was shaken for 2 hours at 20° C. The resin was removed by filtration through a Florisil® cartridge. Evaporation of solvent gave a crude product, which was purified by trituration with methanol to give the title compound; ¹H-NMR (CDCl₃): δ 10.48 (1H, br s), 7.98 (1H, s), 7.47 (2H, m), 7.41 (2H, m), 7.40 (2H, m), 7.30 (1H, m), 7.15 (1H, m), 6.99 (1H, m), 5.04 (1H, m), 4.15 (2H, t), 3.2 (2H, d), 1.10 (6H, d); MS (ES): 375 (MH⁺).

In a similar manner, but replacing benzoyl chloride with the appropriately substituted acyl chloride, chloroformate, isocyanate or sulfonyl chloride, the following compounds were made:

iso-propyl 3-(4-fluorobenzoyl)-1,2,3,6-tetrahydroazepino[4,5-b]indole-5-carboxylate; ¹H-NMR (CDCl₃): δ 10.43 (1H, br s), 7.86 (1H, s), 7.50 (2H, m), 7.41 (1H, d)), 7.26 (1H, d), 6.98-7.15 (4H, m), 5.02 (1H, m), 4.10 (2H, t), 3.2 (2H, d), 1.09 (6H, m); MS (ES): 393 (MH⁺);

iso-propyl 3-(4-anisoyl)-1,2,3,6-tetrahydroazepino[4,5-b]indole-5-carboxylate; ¹H-NMR (CDCl₃): δ 10.45 (1H, br s), 8.27 (1H, s), 7.47 (1H, d), 7.22 (1H, d), 7.03 (1H, m), 6.90 (4H, m), 6.77 (2H, m), 5.07 (1H, m), 3.99 (2H, m), 3.11 (2H, d), 1.21 (6H, d); MS (ES): 421 (MH⁺);

iso-propyl 3-piperonyloyl-1,2,3,6-tetrahydroazepino[4,5-b]indole-5-carboxylate; ¹H-NMR (CDCl₃): δ 10.52 (1H, br s), 8.04 (1H, s), 7.48 (1H, d), 7.33 (1H, d), 7.15 (1H, m), 7.08 (3H, m), 6.82 (1H, 8.5), 6.02 (2H, s), 5.17 (1H, m), 4.17 (2H, d), 3.11 (2H, d), 1.20 (6H, d); MS (ES): 419 (MH⁺);

iso-propyl 3-phenoxylcarbonyl-1,2,3,6-tetrahydroazepino[4,5-b]indole-5-carboxylate; ¹H-NMR (CDCl₃): δ 10.47 (1H, br s), 8.29 (1H, s), 7.38 (1H, d), 7.23-7.31 (3H, m), 7.16 (1H, d), 7.06 (3H, m), 6.97 (1H, m), 5.10 (1H, m), 4.02 (2H, m), 3.13 (2H, d), 1.24 (6H, d); MS (ES): 391 (MH⁺);

iso-propyl 3-(2,4-dichlorophenylcarbamoyl)-1,2,3,6-tetrahydroazepino[4,5-b]indole-5-carboxylate; ¹H-NMR (CDCl₃): δ 10.41 (1H, br s), 8.06 (1H, d), 7.92 (1H, s), 7.31 (1H, d), 7.07-7.21 (4H, m), 6.90 (1H, m), 6.97 (1H, m), 5.07 (1H, m), 3.89 (2H, t), 3.04 (2H, t), 1.18 (6H, d); MS (ES): 458 (MH⁺);

iso-propyl 3-(4-tert-butylbenzenesulfonyl)-1,2,3,6-tetrahydroazepino[4,5-b]indole-5-carboxylate; ¹H-NMR (CDCl₃): δ 10.52 (1H, br s), 8.43 (1H, d), 7.79 (2H, d), 7.56 (2H, d), 7.40 (1H, d), 7.33 (1H, d), 7.15 (1H, m), 7.05 (1H, m), 5.23 (1H, m), 3.84 (2H, t), 3.00 (2H, t), 1.41 (6H, d), 1.33 (9H, s); MS (ES): 467 (MH⁺);

3-(4-chlorobenzoyl)-1,2,3,6-tetrahydroazepino[4,5-b]indole-5-carboxylic acid isopropyl ester; MS (ES): 409 (MH⁺);

3-phenylcarbamoyl-1,2,3,6-tetrahydroazepino[4,5-b]indole-5-carboxylic acid isopropyl ester; MS (ES): 390 (MH⁺);

3-(4-chlorophenylcarbamoyl)-1,2,3,6-tetrahydroazepino[4,5-b]indole-5-carboxylic acid isopropyl ester; MS (ES): 424 (MH⁺);

3-p-tolylcarbamoyl-1,2,3,6-tetrahydroazepino[4,5-b]indole-5-carboxylic acid isopropyl ester; MS (ES): 404 (MH⁺);

3-phenylacetyl-1,2,3,6-tetrahydroazepino[4,5-b]indole-5-carboxylic acid isopropyl ester; MS (ES): 389 (MH⁺);

3-(4-methoxybenzoyl)-1,2,3,6-tetrahydroazepino[4,5-b]indole-5-carboxylic acid isopropyl ester; MS (ES): 405 (MH⁺);

1,6-dihydro-2H-azepino[4,5-b]indole-3,5-dicarboxylic acid 3-(4-chlorophenyl) ester 5-isopropyl ester; MS (ES): 425 (MH⁺);

1,6-dihydro-2H-azepino[4,5-b]indole-3,5-dicarboxylic acid 5-isopropyl ester 3-p-tolyl ester MS (ES): 405 (MH⁺);

3-(4-methoxyphenylcarbamoyl)-1,2,3,6-tetrahydroazepino[4,5-b]indole-5-carboxylic acid isopropyl ester; MS (ES): 420 (MH⁺);

3-nonanoyl-1,2,3,6-tetrahydroazepino[4,5-b]indole-5-carboxylic acid isopropyl ester; MS (ES): 411 (MH⁺);

1-(2-methoxybenzoyl)-1,2,3,6-tetrahydroazepino[4,5-b]indole-5-carboxylic acid isopropyl ester; MS (ES): 405 (MH⁺);

3-(3-phenylpropionyl)-1,2,3,6-tetrahydroazepino[4,5-b]indole-5-carboxylic acid isopropyl ester; MS (ES): 403 (MH⁺);

3-(toluene-4-sulfonyl)-1,2,3,6-tetrahydroazepino[4,5-b]indole-5-carboxylic acid isopropyl ester; MS (ES): 425 (MH⁺);

3-(4-chlorobenzenesulfonyl)-1,2,3,6-tetrahydroazepino[4,5-b]indole-5-carboxylic acid isopropyl ester; MS (ES): 445 (MH⁺);

3-(4-methoxybenzenesulfonyl)-1,2,3,6-tetrahydroazepino[4,5-b]indole-5-carboxylic acid isopropyl ester; MS (ES): 441 (MH⁺);

3-(3,4-dimethoxybenzenesulfonyl)-1,2,3,6-tetrahydroazepino[4,5-b]indole-5-carboxylic acid isopropyl ester; MS (ES): 471 (MH⁺);

3-(4-trifluoromethoxybenzenesulfonyl)-1,2,3,6-tetrahydroazepino[4,5-b]indole-5-carboxylic acid isopropyl ester; MS (ES): 495 (MH⁺);

3-(2,4-dichlorobenzoyl)-1,2,3,6-tetrahydroazepino[4,5-b]indole-5-carboxylic acid isopropyl ester; MS (ES): 443 (MH⁺);

3-(3-methoxybenzoyl)-1,2,3,6-tetrahydroazepino[4,5-b]indole-5-carboxylic acid isopropyl ester; MS (ES): 405 (MH⁺); and

3-(benzo[1,3]dioxole-5-carbonyl)-1,2,3,6-tetrahydroazepino[4,5-b]indole-5-carboxylic acid isopropyl ester; MS (ES): 419 (MH⁺).

### Example 4A

### Preparation of n-Propyl 3-Benzoyl-1,2,3,6-tetrahydroazepino[4,5-b]indole-5-carboxylate

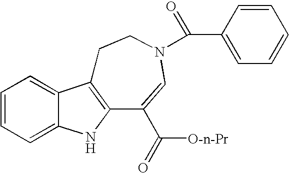 [0677] A. In a similar manner as described in Example 1A, but using n-propyl 3-bromopyruvate and n-propanol, the following compound was prepared:

n-propyl 1,2,3,6-tetrahydroazepino[4,5-b]indole-5-carboxylate; ¹H-NMR (CDCl₃): δ 10.41 (1H, br s), 7.74 (1H, d), 7.35 (1H, s), 7.56 (1H, d), 7.26 (1H, d), 7.09 (1H, m), 5.23 (1H, br s), 4.11 (2H, d), 3.54 (2H, br s), 3.12 (2H, br s), 1.68 (2H, m), 0.95 (3H, t); MS (ES): 271 (MH⁺).
B. The title compound was prepared in a manner similar to that described in Example 2A by using n-propyl 1,2,3,6-tetrahydroazepino[4,5-b]indole-5-carboxylate (compound of step A) and benzoyl chloride; ¹H-NMR (CDCl₃): δ 10.55 (1H, br s), 8.07 (1H, s), 7.52-7.58 (4H, m), 7.47 (2H, m), 7.33 (1H, d), 7.21 (1H, m), 7.12 (1H, m), 4.23 (2H, t), 4.13 (2H, m), 3.28 (2H, m), 1.56 (2H, m), 1.40 (3H, t); MS (ES): 375 (MH⁺).

### Example 4

### Preparation of 1,2,3,6-Tetrahydroazepino[4,5-b]indole-5-carboxylic acid

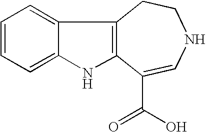

A mixture of 1,2,3,6-tetrahydroazepino[4,5-b]indole-5-carboxylic acid methyl ester (1.21 g, 5 mmol), di-tert-butyl dicarbonate (1.69 g, 1.5 eq.) and diisopropylethylamine (1.3 mL, 1.5 eq.) in benzene was heated to reflux with a Dean-Stark trap for 48 hours. After cooling, the solvent was removed and the crude product was redissolved in DCM and passed through a plug of silica gel, and eluted with DCM. Evaporation of the solvent gave a glue-like product (1,6-dihydro-2H-azepino[4,5-b]indole-3,5-dicarboxylic acid 3-tert-butyl ester 5-methyl ester); ¹H-NMR (CDCl₃): δ 10.52 (1H, br s), 8.40 (1H, s), 7.47 (2H, d), 7.34 (1H, m), 7.15 (1H, dd), 7.07 (1H, dd), 3.97 (2H, t), 3.87 (3H, s), 3.14 (2H, t), 1.57 (9H, s), 1.52 (3H, t).

To a solution of 1,6-dihydro-2H-azepino[4,5-b]indole-3,5-dicarboxylic acid 3-tert-butyl ester 5-methyl ester (0.293 g, 0.62 mmol) in MeOH (4 mL) was added 4 N NaOH (2 mL) and the mixture was heated for 5 hours under nitrogen. After cooling, the reaction mixture was diluted with water and extracted with EtOAc. The aqueous layer was acidified with AcOH. Precipitate was collected by filtration and washed with water and ether and dried under high vacuum to give the title compound (70 mg); ¹H-NMR (DMSO-d₆): δ 11.40 (1H, s), 10.73 (1H, br s), 7.83 (1H, m), 7.73 (2H, d), 7.38 (1H, m), 7.25 (1H, m), 6.88 (2H, m), 3.45 (2H, t), 3.87 (3H, s), 3.2.98 (2H, t), MS (ES): 229 (MH⁺).

### Example 4A

### Preparation of diethyl-1,2,3,6-tetrahydroazepino[4,5-b]indole-2,5-dicarboxylate

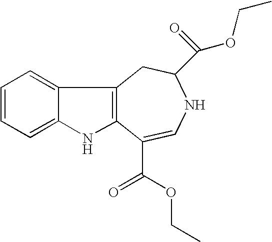

In a manner similar to that described in Example 1A, but replacing tryptamine hydrochloride with the appropriate tryptophan-methyl ester-HCl, the following compounds were prepared:

diethyl 1,2,3,6-tetrahydroazepino[4,5-b]indole-2,5-dicarboxylate; ¹H-NMR (CDCl₃): δ 10.44 (1H, br s), 7.86 (1H, d), 7.48 (1H, m), 7.33 (1H, d), 7.09 (2H, m), 6.10 (1H, d), 4.29 (4H, m), 4.10 (1H, m), 3.84 (1H, d), 2.97 (1H, dd), 1.33 (6H, m); MS (ES): 329 (MH⁺);

### Example 5

Preparation of isopropyl 3-(3,4-difluorobenzoyl)-1,1-dimethyl-1,2,3,6-tetrahydroazepino[4,5-b]indole-5-carboxylate

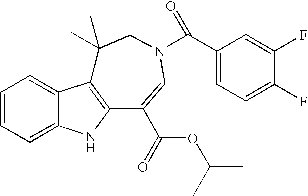

Ethyl 3-(3,4-difluorobenzoyl)-1,1-dimethyl-1,2,3,4,5,6-hexahydro-azepino[4,5-b]indole-5-carboxylate was saponified, converted to the corresponding isopropyl ester using CDI and isopropanol, and then oxidized as to give the title compound; ¹H-NMR (DMSO-d₆): δδ 10.83 (1H, s), 7.76 (1H, d), 7.71 (1H, app t), 7.64 (1H, s), 7.52-7.61 (2H, m), 7.40 (1H, m), 7.08 (1H, app t), 6.98 (1H, app t), 5.05 (1H, sept), 1.52 (6H, s), 1.18 (6H, d); MS (ESI): 439 (MH⁺).

### Example 6

### Preparation of 1-Methylethyl1,1-Dimethyl-1,2,3,6-Tetrahydroazepino[4,5-β]indole-5-Carboxylate Analogs

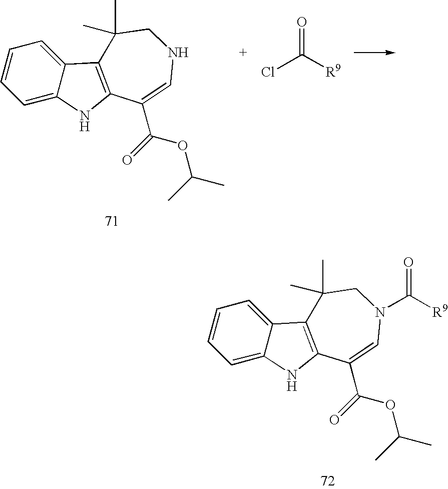

To a solution of 1-methylethyl 1,1-dimethyl-1,2,3,6-tetrahydroazepino[4,5-β]indole-5-carboxylate (1.0 equiv) in DCM was added the corresponding acyl chloride (1.5 equiv) and diisopropyethylamine (1.8 equiv) and the mixture was stirred for 1 h to overnight at room temperature to 50° C. The reaction mixture was concentrated by rotary evaporator and purified by flash column chromatography to give the corresponding product in moderate to good yields.

Using the same or analogous synthetic techniques and/or substituting with alternative reagents, the following compounds of the invention were prepared:

1-Methylethyl3-[(2-chloro-3,6-difluorophenyl)carbonyl]-1,1-dimethyl-1,2,3,6-tetrahydroazepino[4,5-b]indole-5-carboxylate: ¹H NMR (400 MHz, CDCl₃): δ 10.63 (s, 1H), 7.85 (d, 1H), 7.60 (s, 1H), 7.37 (d, 1H), 7.18 (t, 1H), 7.08 (m, 3H), 5.05 (m, 1H), 4.11 (bs, 1H), 1.67 (d, 6H), 1.17 (t, 6H); MS (EI) for C₂₅H₂₃ClF₂N₂O₃: 473.1 (MH⁺).

1-methylethyl 1,1-dimethyl-3-(phenylcarbonyl)-1,2,3,6-tetrahydroazepino[4,5-b]indole-5-carboxylate: ¹H NMR (400 MHz, CDCl₃): δ 10.73 (bs, 1H), 8.12-7.06 (m, 10H), 5.11 (m, 1H), 4.05 (bs, 2H), 1.64 (s, 6H), 1.17 (d, J=6.0 Hz, 6H); MS (EI) for C₂₅H₂₆N₂O₃: 403.3 (MH¹).

1-methylethyl3-[(2-fluorophenyl)carbonyl]-1,1-dimethyl-1,2,3,6-tetrahydroazepino[4,5-b]indole-5-carboxylate: ¹H NMR (400 MHz, CDCl₃): δ 10.71 (bs, 1H), 7.84-7.05 (m, 9H), 5.08 (m 1H), 4.06 (bs, 1H), 1.64 (s, 6H), 1.16 (d, J=6.0 Hz, 6H); MS (EI) for C₂₅H₂₆FN₂O₃: 421.3 (MH⁺).

1-Methylethyl1,1-dimethyl-3-{[2-(trifluoromethyl)phenyl]carbonyl}-1,2,3,6-tetrahydroazepino[4,5-b]indole-5-carboxylate: ¹H NMR (400 MHz, CDCl₃): δ 10.65 (s, 1H), 7.92 (s, 1H), 7.82 (m, 3H), 7.68 (s, 1H), 7.58 (t, 1H), 7.40 (d, 1H), 7.20 (t, 1H), 7.10 (t, 1H), 5.15 (m, 1H), 4.10 (bs, 2H), 1.64 (s, 6H), 1.21 (s, J=6.26 Hz, 6H); MS (EI) for C₂₆H₂₅F₃N₂O₃: 471.2 (MH⁺).

1-Methylethyl1,1-dimethyl-3-{[4-(trifluoromethyl)phenyl]carbonyl}-1,2,3,6-tetrahydroazepino[4,5-b]indole-5-carboxylate: ¹H NMR (400 MHz, CDCl₃): δ 10.68 (s, 1H), 8.28 (d, 1H), 7.82 (m, 2H), 7.71 (m, 3H), 7.40 (d, 1H), 7.19 (t, 1H), 7.09 (t, 1H), 5.11 (m, 1H), 4.11 (bs, 1H), 1.64 (s, 6H), 1.17 (d, J=6.26 Hz, 6H); MS (EI) for C₂₆H₂₅F₃N₂O₃: 471.2 (MH⁺).

1-methylethyl3-[(2-chlorophenyl)carbonyl]-1,1-dimethyl-1,2,3,6-tetrahydroazepino[4,5-b]indole-5-carboxylate: ¹H NMR (400 MHz, DMSO): δ 10.81 (bs, 1H), 7.84-7.45 (m, 7H), 7.05-6.95 (m 2H), 4.96 (m, 1H), 1.64 (s, 6H), 1.09 (d, J=6.0 Hz, 6H); MS (EI) for C₂₅H₂₆ClN₂O₃: 437.3 (MH⁺).

1-methylethyl3-[(2-bromophenyl)carbonyl]-1,1-dimethyl-1,2,3,6-tetrahydroazepino[4,5-b]indole-5-carboxylate: ¹H NMR (400 MHz, DMSO): δ 10.83 (bs, 1H), 7.83-7.07 (m, 9H), 4.96 (m, 1H), 1.70 (s, 6H), 1.10 (d, J=6.0 Hz, 6H); MS (EI) for C₂₅H₂₆BrN₂O₃: 482.3 (MH⁺).

1-methylethyl 1,1-dimethyl-3-[(2-methylphenyl)carbonyl]-1,2,3,6-tetrahydroazepino[4,5-b]indole-5-carboxylate: ¹H NMR (400 MHz, DMSO): δ 10.69 (bs, 1H), 7.76-6.97 (m, 9H), 4.95 (bs, 1H), 4.00 (bs, 2H), 2.22 (s, 3H), 1.54 (s, 6H), 1.11 (bs, 6H); MS (EI) for C₂₆H₂₈N₂O₃: 417.3 (MH⁺).

1-methylethyl 1,1-dimethyl-3-{[2-(methyloxy)phenyl]carbonyl}-1,2,3,6-tetrahydroazepino[4,5-b]indole-5-carboxylate: ¹H NMR (400 MHz, DMSO): δ 10.83 (bs, 1H), 7.75-6.92 (m, 9H), 4.93 (bs, 1H), 3.67 (s, 3H), (s, 3H), 1.52 (s, 6H), 1.06 (s, 6H); MS (EI) for C₂₆H₂₈N₂O₄: 433.3 (MH⁺).

1-methylethyl 1,1-dimethyl-3-({2-[(trifluoromethyl)oxy]phenyl}carbonyl)-1,2,3,6-tetrahydroazepino[4,5-b]indole-5-carboxylate: ¹H NMR (400 MHz, DMSO): δ 10.82 (bs, 1H), 7.74-7.42 (m, 7H), 7.05-6.95 (m, 2H), 4.92 (bs, 1H), 3.98 (bs, 2H), 1.54 (s, 6H), 1.52 (s, 6H), 1.04 (s, 6H); MS (EI) for C₂₆H₂₅F₃N₂O₄: 487.4 (MH⁺).

1-methylethyl 3-{[4-fluoro-3-(trifluoromethyl)phenyl]carbonyl}-1,1-dimethyl-1,2,3,6-tetrahydroazepino[4,5-b]indole-5-carboxylate: ¹H NMR (400 MHz, CDCl₃): δ 10.65 (bs, 1H), 7.92-7.82 (m, 3H), 7.73 (s, 1H), 7.41-7.08 (m, 4H), 5.16 (m, 1H), 4.05 (bs, 2H), 1.64 (s, 6H), 1.21 (d, J=6.4 Hz, 6H); MS (EI) for C₂₆H₂₄F₄N₂O₃: 489.4 (MH⁺).

1-methylethyl 3-{[3-fluoro-4-(trifluoromethyl)phenyl]carbonyl}-1,1-dimethyl-1,2,3,6-tetrahydroazepino[4,5-b]indole-5-carboxylate: ¹H NMR (400 MHz, CDCl₃): δ 10.65 (bs, 1H), 7.84-7.07 (m, 8H), 5.12 (m, 1H), 4.08 (1bs, 2H), 1.64 (s, 6H), 1.20 (d, J=6.4 Hz, 6H); MS (EI) for C₂₆H₂₄F₄N₂O₃: 489.4 (MH⁺).

1-methylethyl 1,1-dimethyl-3-[(4-piperidin-4-ylphenyl)carbonyl]-1,2,3,6-tetrahydroazepino[4,5-b]indole-5-carboxylate: ¹H NMR (400 MHz, DMSO-d₆): δ 10.85 (bs, 1H), 8.74 (bs, 1H), 8.50 (bs, 1H), 7.76-6.98 (m, 9H), 5.02 (m, 1H), 3.98 (bs, 2H), 2.99 (m, 4H), 2.09-1.84 (m, 4H), 1.53 (s, 6H), 1.14 (d, J=6.4 Hz, 6H); MS (EI) for C₂₅H₃₃N₃O₃: 487.2 (MH⁺).

1-methylethyl 1,1-dimethyl-3-[(3-piperidin-4-ylphenyl)carbonyl]-1,2,3,6-tetrahydroazepino[4,5-b]indole-5-carboxylate: ¹H NMR (400 MHz, DMSO-d₆): δ 10.85 (bs, 1H), 8.76 (bs, 1H), 8.48 (bs, 1H), 7.78-6.97 (m, 9H), 5.03 (m, 1H), 3.98 (bs, 2H), 3.71 (m, 1H), 2.95 (m, 4H), 1.99-1.78 (m, 4H), 1.53 (s, 6H), 1.13 (d, J=6.4 Hz, 6H); MS (EI) for C₂₅H33N₃O₃: 487.2 (MH¹).

1-methylethyl3-({4-[(dimethylamino)methyl]phenyl}carbonyl)-1,1-dimethyl -1,2,3,6-tetrahydroazepino[4,5-b]indole-5-carboxylate: ¹H NMR (400 MHz, DMSO-d₆): δ 10.86 (bs, 1H), 7.76-6.98 (m, 9H), 5.02 (m, 1H), 3.98 (bs, 2H), 3.45 (s, 2H), 2.15 (s, 6H), 1.53 (s, 6H), 1.13 (d, J=6.4 Hz, 6H); MS (EI) for C₂₉H₃₃N₃O₃: 460.4 (MH⁺).

1-methylethyl3-({3-[(dimethylamino)methyl]phenyl}carbonyl)-1,1-dimethyl -1,2,3,6-tetrahydroazepino[4,5-b]indole-5-carboxylate: ¹H NMR (400 MHz, DMSO-d₆): δ 10.85 (bs, 1H), 7.74-6.96 (m, 9H), 4.98 (m, 1H), 3.96 (bs, 2H), 3.40 (s, 2H), 2.09 (s, 6H), 1.57 (s, 6H), 1.10 (d, J=6.4 Hz, 6H); MS (EI) for C₂₈H₃₃N₃O₃: 425.4 (MH⁺).

1-Methylethyl3-[(3,4-difluorophenyl)carbonyl]-1,1-dimethyl-9-[(phenylmethyl)oxy]-1,2,3,6-tetrahydroazepino[4,5-b]indole-5-carboxylate: ¹H NMR (400 MHz, CDCl₃): δ 10.57 (s, 1H), 7.73 (s, 1H), 7.49 (d, 2H), 7.39 (t, 1H), 7.32 (m, 7H), 7.21 (t, 1H), 6.96 (m, 1H), 5.13 (m, 1H), 4.06 (bs, 2H), 1.57 (s, 6H), 1.22 (d, 6H); MS (EI) for C₃₂H₃₀F₂N₂O₄: 545.4 (MH⁺).

1-Methylethyl3-[(3,4-difluorophenyl)carbonyl]-9-hydroxy-1,1-dimethyl-1,2,3,6-tetrahydroazepino[4,5-b]indole-5-carboxylate: ¹H NMR (400 MHz, CDCl₃): δ 10.56 (s, 1H), 7.74 (s, 1H), 7.51 (m, 1H), 7.34 (m, 1H), 7.25 (m, 2H), 7.22 (m, 1H), 6.78 (dd, 1H), 5.14 (m, 1H), 4.47 (s, 1H), 4.05 (bs, 2H), 1.57 (d, J=11.5 Hz, 6H), 1.22 (d, J=6.25 Hz, 6H); MS (EI) for C₂₅H₂₄F₂N₂O₄: 455.2 (MH⁺).

1-Methylethyl1,1-dimethyl-3-{[3-(trifluoromethyl)phenyl]carbonyl}-1,2,3,6-tetrahydroazepino[4,5-b]indole-5-carboxylate: ¹H NMR (400 MHz, CDCl₃): δ 10.68 (s, 1H), 7.80 (m, 5H), 7.60 (t, 1H), 7.39 (d, 1H), 7.20 (t, 1H), 7.09 (t, 1H), 5.13 (m, 1H), 4.09 (bs, 2H), 1.65 (s, 6H), 1.17 (d, 6H); MS (EI) for C₂₆H₂₅F₃N₂O₃: 471.1 (MH⁺).

1-Methylethyl1,1-dimethyl-3-{[4-(1H-pyrazol-1-yl)phenyl]carbonyl}-1,2,3,6-tetrahydroazepino[4,5-b]indole-5-carboxylate: ¹H NMR (400 MHz, CDCl₃): δ 10.72 (s, 1H), 8.00 (d, 1H), 7.85 (m, 2H), 7.82 (d, 1H), 7.78 (m, 2H), 7.72 (m, 2H), 7.40 (d, 1H), 7.19 (t, 1H), 7.09 (t, 1H), 5.13 (m, 1H), 4.11 (bs, 2H), 1.64 (s, 6H), 1.20 (d, J=5.86 Hz, 6H); MS (EI) for C₂₈H₂₈N₄O₃: 469.3 (MH⁺).

1-Methylethyl3-[(3-cyanophenyl)carbonyl]-1,1-dimethyl-1,2,3,6-tetrahydroazepino[4,5-b]indole-5-carboxylate: ¹H NMR (400 MHz, CDCl₃): δ 10.65 (s, 1H), 7.92 (s, 1H), 7.82 (m, 3H), 7.69 (s, 1H), 7.58 (t, 1H), 7.40 (d, 1H), 7.21 (t, 1H), 7.10 (t, 1H), 5.15 (m, 1H), 4.12 (bs, 2H), 1.64 (s, 1H), 1.21 (d, 6H); MS (EI) for C₂₆H₂₅N₃O₃: 428.3 (MH⁺).

1-Methylethyl3-[(2,4-dichlorophenyl)carbonyl]-1,1-dimethyl-1,2,3,6-tetrahydroazepino[4,5-b]indole-5-carboxylate: ¹H NMR (400 MHz, CDCl₃): δ 10.65 (s, 1H), 7.84 (d, 1H), 7.51 (d, 2H), 7.37 (q, 3H), 7.17 (t, 1H), 5.06 (m, 1H), 4.07 (bs, 1H), 1.67 (s, 6H), 1.17 (s, 6H); MS (EI) for C₂₅H₂₄Cl₂N₂O₃: 471.0 (M⁺), 473.1 (M+2).

1-Methylethyl 3-[(3,4-dichlorophenyl)carbonyl]-1,1-dimethyl-1,2,3,6-tetrahydroazepino[4,5-b]indole-5-carboxylate: ¹H NMR (400 MHz, CDCl₃): δ 10.67 (s, 1H), 7.82 (d, 1H), 7.74 (m, 2H), 7.52 (d, 1H), 7.41 (m, 2H), 7.20 (t, 1H), 7.09 (t, 1H), 5.16 (m, 1H), 4.11 (bs, 2H), 1.62 (s, 6H), 1.23 (d, 6H); MS (EI) for C₂₅H₂₄Cl₂N₂O₃: 471.2 (M⁺), 473.3 (M+2).

1-Methylethyl3-[(4-chloro-2,5-difluorophenyl)carbonyl]-1,1-dimethyl-1,2,3,6-tetrahydroazepino[4,5-b]indole-5-carboxylate: ¹H NMR (400 MHz, CDCl₃): δ 10.65 (s, 1H), 7.82 (d, 1H), 7.63 (m, 1H), 7.37 (m, 1H), 7.34 (m, 2H), 7.18 (t, 1H), 7.07 (t, 1H), 5.13 (m, 1H), 4.11 (bs, 2H), 1.62 (s, 6H), 1.22 (s, 6H); MS (EI) for C₂₅H₂₃ClF₂N₂O₃: 473.3 (MH⁺).

1-Methylethyl1,1-dimethyl-3-[(1-methyl-1H-1,2,3-benzotriazol-5-yl)carbonyl]-1,2,3,6-tetrahydroazepino[4,5-b]indole-5-carboxylate: ¹H NMR (400 MHz, CDCl₃): δ 10.70 (s, 1H), 8.32 (s, 1H), 7.81 (m, 3H), 7.58 (d, 1H), 7.41 (d, 1H), 7.20 (t, 1H), 7.09 (t, 1H), 5.09 (m, 1H), 4.35 (s, 3H), 4.13 (bs, 2H), 1.66 (s, 6H), 1.12 (d, 6H); MS (EI) for C₂₆H₂₇N₅O₃: 458.2 (MH⁺).

1-Methylethyl 1,1-dimethyl-3-({4-[4-(trifluoromethyl)-1H-pyrazol-1-yl]phenyl}carbonyl)-1,2,3,6-tetrahydroazepino[4,5-b]indole-5-carboxylate: ¹H NMR (400 MHz, CDCl₃): δ 10.72 (s, 1H), 7.82 (m, 2H), 7.74 (m, 3H), 7.60 (dd, 2H), 7.40 (dd, 1H), 7.20 (t, 1H), 7.09 (t, 1H), 6.87 (s, 1H), 5.12 (m, 1H), 1.66 (s, 6H), 1.20 (d, 6H); MS (EI) for C₂₉H₂₇F₃N₄O₃: 458.2 (MH⁺).

1-methylethyl 3-[(3-fluorophenyl)carbonyl]-1,1-dimethyl-1,2,3,6-tetrahydroazepino[4,5-b]indole-5-carboxylate: ¹H NMR (400 MHz, CDCl₃): δ 10.82 (s, 1H), 7.83 (d, J=8.4 Hz, 1H), 7.79 (s, 1H), 7.41 (m, 3H), 7.33 (m, 3H), 7.19 (d, J=8.4 Hz, 1H), 7.08 (t, J=7.2 Hz, 1H), 5.13 (sept., J=6.4 Hz, 1H), 4.11 (br s, 2H), 1.63 (s, 6H), 1.20 (s, 3H), 1.19 (s, 3H); MS (EI) for C₂₅H₂₅FN₂O₃: 421.2 (MH.

1-methylethyl 3-[(2,4-difluorophenyl)carbonyl]-1,1-dimethyl-1,2,3,6-tetrahydroazepino[4,5-b]indole-5-carboxylate: ¹H NMR (400 MHz, CDCl₃): δ 10.62 (s, 1H), 7.82 (d, J=8.4 Hz, 1H), 7.69 (br s, 1H), 7.52 (q, J=7.2 Hz, 1H), 7.37 (d, J=8.0 Hz, 1H), 7.17 (td, J=6.8 Hz, 1H), 7.07 (t, J=7.2 Hz, 1H), 7.02 (t, J=1.6 Hz, 1H), 6.90 (t, J=2.4 Hz, 1H), 5.11 (m, 1H), 4.06 (br s, 2H), 1.62 (s, 6H), 1.20 (s, 3H), 1.19 (s, 3H); MS (EI) for C₂₅H₂₅F₂N₂O₃: 439.2 (MH⁺).

1-methylethyl 3-[(2,3-difluorophenyl)carbonyl]-1,1-dimethyl-1,2,3,6-tetrahydroazepino[4,5-b]indole-5-carboxylate: ¹H NMR (400 MHz, CDCl₃): δ 10.69 (s, 1H), 7.83 (d, J=8.0 Hz, 1H), 7.68 (br s, 1H), 7.37 (d, J=8.0 Hz, 1H), 7.33 (m, 1H), 7.23 (m, 2H), 7.17 (t, J=8.0 Hz, 1H), 7.07 (t, J=7.2 Hz, 1H), 5.10 (br s, 1H), 4.07 (br s, 2H,) 1.63 (s, 6H), 1.18 (s, 6H); MS (EI) for C₂₅H₂₅F₂N₂O₃: 439.2 (MH⁺).

1-methylethyl 3-[(2,6-difluorophenyl)carbonyl]-1,1-dimethyl-1,2,3,6-tetrahydroazepino[4,5-b]indole-5-carboxylate: ¹H NMR (400 MHz, CDCl₃): δ 10.66 (s, 1H), 7.84 (d, J=8.0 Hz, 1H), 7.71 (s, 1H), 7.47 (m, 1H), 7.36 (d, J=8.0 Hz, 1H), 7.17 (t, J=7.6 Hz, 1H), 7.04 (m, 3H), 7.02 (t, J=1.6 Hz, 1H), 5.06 (sept., J=6.4, 1H), 4.13 (br s, 2H), 1.66 (s, 6H), 1.16 (s, 3H), 1.14 (s, 3H); MS (EI) for C₂₅H₂₅F₂N₂O₃: 439.2 (MH⁺).

1-methylethyl 3-[(2,5-difluorophenyl)carbonyl]-1,1-dimethyl-1,2,3,6-tetrahydroazepino[4,5-b]indole-5-carboxylate: ¹H NMR (400 MHz, CDCl₃): δ 10.69 (s, 1H), 7.82 (d, J=8.0 Hz, 1H), 7.68 (br s, 1H), 7.37 (d, J=8.0 Hz, 1H), 7.17 (m, 4H), 7.07 (t, J=7.2 Hz, 1H), 5.10 (br s, 1H), 4.06 (br s, 2H), 1.64 (s, 6H), 1.19 (s, 6H); MS (EI) for C₂H₂₅F₂N₂O₃: 439.2 (MH⁺).

1-methylethyl 1,1-dimethyl-3-[(2,3,4-trifluorophenyl)carbonyl]-1,2,3,6-tetrahydroazepino[4,5-b]indole-5-carboxylate: ¹H NMR (400 MHz, CDCl₃): δ 10.66 (s, 1H), 7.82 (d, J=8.0 Hz, 1H), 7.64 (br s, 1H), 7.37 (m, 1H), 7.18 (t, J=7.6 Hz, 1H), 7.12 (t, J=7.9 Hz, 1H), 7.08 (t, J=7.2, 1H), 5.13 (m, 1H), 4.06 (br s, 2H), 1.63 (s, 6H), 1.23 (s, 3H), 1.21 (s, 3H); MS (EI) for C₂₅H₂₄F₃N₂O₃, 457.2 (MH⁺).

1-methylethyl 1,1-dimethyl-3-[(2,4,6-trifluorophenyl)carbonyl]-1,2,3,6-tetrahydroazepino[4,5-b]indole-5-carboxylate: ¹H NMR (400 MHz, CDCl₃): δ 10.64 (s, 1H), 7.84 (d, J=8.0 Hz, 1H), 7.66 (s, 1H), 7.37 (d, J=8.0 Hz, 1H), 7.66 (s, 1H), 7.37 (d, J=8.0 Hz, 1H), 7.18 (t, J=8.0 Hz, 1H), 7.07 (t, J=8.0 Hz, 1H), 6.79 (t, J=8.0 Hz, 2H), 5.09 (sept., J=6.0 Hz, 1H), 4.09 (br s, 2H), 1.65 (s, 6H), 1.20 (s, 3H), 1.19 (s, 3H); MS (EI) for C₂₅H₂₄F₃N₂O₃, 457.2 (MH⁺).

1-methylethyl 1,1-dimethyl-3-[(2,4,5-trifluorophenyl)carbonyl]-1,2,3,6-tetrahydroazepino[4,5-b]indole-5-carboxylate: ¹HNMR (400 MHz, CDCl₃): δ 10.67 (s, 1H), 7.82 (d, J=8.0 Hz, 1H), 7.65 (br s, 1H), 7.40 (m, 2H), 7.18 (t, J=6.8 Hz, 1H), 7.08 (t, J=6.8 Hz, 1H), 7.01 (m, 1H), 5.14 (sept., J=5.6 Hz, 1H), 4.05 (br S, 2H), 1.62 (s, 6H), 1.23 (s, 3H), 1.22 (s, 3H); MS (EI) for C₂₅H₂₄F₃N₂O₃, 457.2 (MH⁺).

1-methylethyl 3-(1,3-benzodioxol-5-ylcarbonyl)-1,1-dimethyl-1,2,3,6-tetrahydroazepino[4,5-b]indole-5-carboxylate: ¹H NMR (400 MHz, CDCl₃): δ 10.71 (s, 1H), 7.87 (s, 1H), 7.82 (d, J=8.0 Hz, 1H), 7.39 (d, J=8.4 Hz, 1H), 7.14 (m, 4H), 6.81 (d, J=8.0 Hz, 1H), 6.04 (s, 2H), 5.16 (sept., J=6.0 Hz, 1H), 4.06 (br s, 2H), 1.61 (s, 6H), 1.25 (s, 3H), 1.23 (s, 3H).

1-methylethyl 3-[(3-chlorophenyl)carbonyl]-1,1-dimethyl-1,2,3,6-tetrahydroazepino[4,5-b]indole-5-carboxylate: ¹H NMR (400 MHz, CDCl₃): δ 10.70 (s, 1H), 7.83 (d, J=8.0 Hz, 1H), 7.79 (s, 1H), 7.60 (s, 1H), 7.51 (d, J=7.8 Hz, 1H), 7.45 (d, J=8.0 Hz, 1H), 7.38 (m, 2H), 7.19 (d, J=8.0 Hz, 1H), 7.08 (d, J=7.8 Hz, 1H), 5.14 (sept., J=6.4 Hz, 1H), 4.08 (br s, 2H), 1.63 (s, 6H), 1.21 (s, 3H), 1.20 (s, 3H); MS (EI) for C₂₅H₂₆Cl₁N₂O₃, 437.2 (MH⁺).

1-methylethyl 3-[(4-chlorophenyl)carbonyl]-1,1-dimethyl-1,2,3,6-tetrahydroazepino[4,5-b]indole-5-carboxylate: ¹H NMR (400 MHz, CDCl₃): δ 10.69 (s, 1H), 7.82 (dd, J=8.0, 0.8 Hz, 1H), 7.76 (s, 1H), 7.54 (m, 2H), 7.41 (m, 3H), 7.19 (t, J=7.6 Hz, 1H), 7.08 (t, J=7.8 Hz, 1H), 5.14 (sept., J=6.4 Hz, 1H), 4.07 (br s, 2H), 1.62 (s, 6H), 1.22 (s, 3H), 1.21 (s, 3H); MS (EI) for C₂₅H₂₆Cl₁N₂O₃, 437.2 (MH⁺).

1-methylethyl 1,1-dimethyl-3-[(3-methylphenyl)carbonyl]-1,2,3,6-tetrahydroazepino[4,5-b]indole-5-carboxylate: ¹H NMR (400 MHz, CDCl₃): δ 10.73 (s, 1H), 7.86 (s, 1H), 7.83 (d, J=8.0 Hz, 1H), 7.35 (m, 5H), 7.18 (t, J=7.6 Hz, 1H), 7.08 (t, J=7.6 Hz, 1H), 5.12 (sept., J=6.4 Hz, 1H), 4.08 (br s, 2H), 2.38 (s, 3H), 1.64 (s, 6H), 1.19 (s, 3H), 1.17 (s, 3H); MS (EI) for C₂₆H₂₉N₂O₃, 417.2 (MH⁺).

1-methylethyl 1,1-dimethyl-3-[(4-methylphenyl)carbonyl]-1,2,3,6-tetrahydroazepino[4,5-b]indole-5-carboxylate: ¹H NMR (400 MHz, CDCl₃): δ 10.72 (s, 1H), 7.86 (s, 1H), 7.82 (d, J=8.0 Hz, 1H), 7.49 (d, J=8.0 Hz, 2H), 7.39 (dt, J=8.0, 1.2 Hz, 1H), 7.23 (dt, J=7.6, 0.8 Hz, 2H), 7.17 (t, J=7.8 Hz, 1H), 7.08 (t, J=8.4 Hz, 1H), 5.12 (sept., J=6.4 Hz, 1H), 4.09 (br s, 2H), 2.41 (s, 3H), 1.63 (s, 6H), 1.20 (s, 3H), 1.19 (s, 3H); MS (EI) for C₂₆H₂₉N₂O₃, 417.2 (MH⁺).

1-methylethyl 1,1-dimethyl-3-{[3-(methyloxy)phenyl]carbonyl}-1,2,3,6-tetrahydroazepino[4,5-b]indole-5-carboxylate: ¹H NMR (400 MHz, CDCl₃): δ 10.72 (s, 1H), 7.86 (s, 1H), 7.83 (d, J=8.0 Hz, 1H), 7.39 (d, J=8.0 Hz, 1H), 7.32 (t, J=7.8, Hz, 1H), 7.17 (m, 2H), 7.08 (m, 3H), 5.12 (sept., J=6.4 Hz, 1H), 4.01 (br s, 2H), 3.83 (s, 3H), 1.64 (s, 6H), 1.20 (s, 3H), 1.18 (s, 3H); MS (EI) for C₂₆H₂₉N₂O₄, 433.2 (MH⁺).

1-methylethyl 1,1-dimethyl-3-{[4-(methyloxy)phenyl]carbonyl}-1,2,3,6-tetrahydroazepino[4,5-b]indole-5-carboxylate: ¹H NMR (400 MHz, CDCl₃): δ 10.73 (s, 1H), 7.89 (s, 1H), 7.82 (d, J=8.0 Hz, 1H), 7.60 (d., J=8.8 Hz, 1H), 7.39 (d, J=8.0, Hz, 2H), 7.18 (ddd, J=8.0, 7.2, 1.2 Hz, 1H), 7.08 (ddd, J=7.6, 7.2, 1.2 Hz, 1H), 6.92 (d, J=9.2 Hz, 2H), 5.12 (sept., J=6.4 Hz, 1H), 4.09 (br s, 2H), 3.82 (s, 3H), 1.62 (s, 6H), 1.22 (s, 3H), 1.21 (s, 3H); MS (EI) for C₂₆H₂₉N₂O₄, 433.2 (MH⁺).

1-methylethyl 3-[(2,2-difluoro-1,3-benzodioxol-4-yl)carbonyl]-1,1-dimethyl-1,2,3,6-tetrahydroazepino[4,5-b]indole-5-carboxylate: ¹H NMR (400 MHz, CDCl₃): δ 10.71 (s, 1H), 7.84 (dd, J=8.0, 0.8 Hz, 1H), 7.74 (s, 1H), 7.39 (d, J=8.4 Hz, 1H), 7.31 (dd J=7.4, 1.6 Hz, 1H), 7.21 (m, 3H), 7.08 (t, J=7.6 Hz, 1H), 5.15 (sept., J=6.4 Hz, 1H), 4.08 (br s, 2H), 1.64 (s, 6H), 1.19 (s, 3H), 1.17 (s, 3H); MS (EI) for C₂₆H₂₅F₂N₂O₅, 483.2 (MH⁺).

1-methylethyl 3-[(2,2-difluoro-1,3-benzodioxol-5-yl)carbonyl]-1,1-dimethyl-1,2,3,6-tetrahydroazepino[4,5-b]indole-5-carboxylate: ¹H NMR (400 MHz, CDCl₃): δ 10.67 (s, 1H), 7.82 (d, J=8.0 Hz, 1H), 7.76 (s, 1H), 7.39 (m, 2H), 7.36 (dd J=8.2, 2.0 Hz, 1H), 7.19 (t, J=7.4 Hz, 1H), 7.09 (m, 2H), 5.16 (sept., J=6.4 Hz, 1H), 4.08 (br s, 2H), 1.62 (s, 6H), 1.24 (s, 3H), 1.22 (s, 3H); MS (EI) for C₂₆H₂₅F₂N₂O₅, 483.2 (MH⁺).

1-methylethyl 3-{[3,4-bis(methyloxy)phenyl]carbonyl}-1,1-dimethyl-1,2,3,6-tetrahydroazepino[4,5-b]indole-5-carboxylate: ¹H NMR (400 MHz, CDCl₃): δ 10.71 (s, 1H), 7.91 (s, 1H), 7.83 (d, J=8.0 Hz, 1H), 7.39 (d, J=8.4 Hz, 1H), 7.24 (d, J=2.0, Hz, 1H), 7.18 (m, 2H), 7.08 (t, J=7.8 Hz, 1H), 6.84 (d, J=8.0 Hz, 1H), 5.15 (sept., J=6.0 Hz, 1H), 4.09 (br s, 2H), 3.93 (s, 3H), 3.90 (s, 3H), 1.63 (s, 6H), 1.23 (s, 3H), 1.21 (s, 3H); MS (EI) for C₂₇H₃₁N₂O₅, 463.2 (MH⁺).

1-methylethyl 1,1-dimethyl-3-[(5-methylisoxazol-3-yl)carbonyl]-1,2,3,6-tetrahydroazepino[4,5-b]indole-5-carboxylate: ¹H NMR (400 MHz, CDCl₃): δ 10.73 (s, 1H), 8.48 (s, 1H), 7.80 (d, J=8.4 Hz, 1H), 7.37 (dd, J=8.4, 0.8 Hz, 1H), 7.17 (t, J=7.4 Hz, 1H), 7.06 (t, J=7.8, Hz, 1H), 6.46 (s, 1H), 5.22 (sept., J=6.0 Hz, 1H), 4.13 (br s, 2H), 2.52 (s, 3H), 1.57 (s, 6H), 1.36 (s, 3H), 1.35 (s, 3H); MS (EI) for C₂₃H₂₆N₃O₄, 408.2 (MH⁺).

1-methylethyl 3-{[4-fluoro-2-(trifluoromethyl)phenyl]carbonyl}-1,1-dimethyl-1,2,3,6-tetrahydroazepino[4,5-b]indole-5-carboxylate: ¹H NMR (400 MHz, CDCl₃): δ 10.63 (s, 1H), 7.83 (d, J=8.4 Hz, 1H), 7.51 (d, J=7.2 Hz, 1H), 7.48 (s, 1H), 7.37 (s, 1H), 7.34 (m, 2H), 7.17 (t, J=7.2 Hz, 1H), 7.08 (t, J=7.6, Hz, 1H), 5.03 (sept., J=6.0 Hz, 1H), 4.01 (br s, 2H), 1.66 (s, 6H), 1.14 (s, 3H), 1.13 (s, 3H); MS (EI) for C₂₆H₂₅F₄N₂O₃, 489.1 (MH⁺).

1-methylethyl 3-[(2-chloro-4-fluorophenyl)carbonyl]-1,1-dimethyl-1,2,3,6-tetrahydroazepino[4,5-b]indole-5-carboxylate: ¹H NMR (400 MHz, CDCl₃): δ 10.67 (s, 1H), 7.83 (d, J=8.0 Hz, 1H), 7.56 (s, 1H), 7.40 (t, J=6.0 Hz, 1H), 7.36 (t, J=8.0 Hz, 1H), 7.14 (m, 4H), δ 06 (m, 1H), 4.09 (br s, 2H), 1.67 (s, 6H), 1.17 (s, 3H), 1.15 (s, 3H); MS (EI) for C₂₅H₂₅ClFN₂O₃, 455.1 (MH⁺).

1-methylethyl 1,1-dimethyl-3-(4-methylpentanoyl)-1,2,3,6-tetrahydroazepino[4,5-b]indole-5-carboxylate: ¹H NMR (400 MHz, CDCl₃): δ 10.70 (s, 1H), 8.11 (br s, 1H), 7.79 (d, J=8.0 Hz, 1H), 7.36 (d, J=8.0 Hz, 1H), 7.16 (t, J=7.2 Hz, 1H), 7.05 (t, J=8.0 Hz, 1H), 5.25 (sept., J=6.4, 1H), 3.89 (br s, 2H), 2.61 (t, J=8.4 Hz, 2H), 1.62 (m, 1H), 1.58 (s, 3H), 1.55 (s, 3H), 1.40 (s, 3H), 1.38 (s, 3H), 1.35 (m, 1H), 0.94 (s, 3H), 0.93 (s, 3H); MS (EI) for C₂₄H₃₃N₂O₃, 397.1 (MH⁺).

1-methylethyl 3-{[3-(chloromethyl)phenyl]carbonyl}-1,1-dimethyl-1,2,3,6-tetrahydroazepino[4,5-b]indole-5-carboxylate: ¹HNMR (400 MHz, CDCl₃): δ 10.73 (s, 1H), 7.83 (d, J=8.0 Hz, 1H), 7.79 (s, 1H), 7.60 (s, 1H), 7.55 (m, 2H), 7.42 (m, 2H), 7.19 (t, J=7.2 Hz, 1H), 7.09 (t, J=8.0 Hz, 1H), 5.11 (sept., J=6.0, 1H), 4.58 (s, 2H), 4.10 (br s, 2H), 1.64 (s, 6H), 1.19 (s, 3H), 1.18 (s, 3H); MS (EI) for C₂₆H₂₈ClN₂O₃, 451.1 (MH⁺).

1-methylethyl 3-[(3-fluoro-4-methylphenyl)carbonyl]-1,1-dimethyl-1,2,3,6-tetrahydroazepino[4,5-b]indole-5-carboxylate: ¹H NMR (400 MHz, CDCl₃): δ 10.70 (s, 1H), 7.82 (m, 2H), 7.39 (d, J=8.4 Hz, 1H), 7.30 (d, J=9.2 Hz, 1H), 7.25 (m, 2H), 7.18 (t, J=6.8 Hz, 1H), 7.08 (t, J=8.0 Hz, 1H), 5.14 (sept., J=6.4, 1H), 4.09 (br s, 2H), 2.34 (s, 3H), 1.62 (s, 6H), 1.22 (s, 3H), 1.20 (s, 3H); MS (EI) for C₂₆H₂₈FN₂O₃: 435.2 (MH⁺).

1-methylethyl 3-{[2-fluoro-4-(trifluoromethyl)phenyl]carbonyl}-1,1-dimethyl-1,2,3,6-tetrahydroazepino[4,5-b]indole-5-carboxylate: ¹H NMR (400 MHz, CDCl₃): δ 10.66 (s, 1H), 7.84 (d, J=8.0 Hz, 1H), 7.60 (m, 3H), 7.44 (d, J=8.8 Hz, 1H), 7.37 (d, J=8.0 Hz, 1H), 7.18 (t, J=6.8 Hz, 1H), 7.08 (t, J=7.2, Hz, 1H), 5.07 (m, 1H), 4.10 (br s, 2H), 1.66 (s, 6H), 1.14 (s, 3H), 1.13 (s, 3H); MS (EI) for C₂₆H₂₅F₄N₂O₃: 489.2 (MH⁺).

1-methylethyl 3-{[3-chloro-2-fluoro-4-(trifluoromethyl)phenyl]carbonyl}-1,1-dimethyl-1,2,3,6-tetrahydroazepino[4,5-b]indole-5-carboxylate: ¹H NMR (400 MHz, CDCl₃): δ 10.64 (s, 1H), 7.85 (m, 2H), 7.70 (s, 1H), 7.57 (s, 1H), 7.37 (d, J=8.0 Hz, 1H), 7.19 (t, J=6.8 Hz, 1H), 7.08 (t, J=7.2, Hz, 1H), 5.11 (m, 1H), 4.10 (br s, 2H), 1.66 (s, 6H), 1.18 (s, 3H), 1.17 (s, 3H); MS (EI) for C₂₆H₂₄ClF₄N₂O₃: 523.1 (MH⁺).

1-methylethyl 3-{[2-fluoro-3-(trifluoromethyl)phenyl]carbonyl}-1,1-dimethyl-1,2,3,6-tetrahydroazepino[4,5-b]indole-5-carboxylate: ¹H NMR (400 MHz, CDCl₃): δ 10.68 (s, 1H), 7.81 (m, 2H), 7.72 (t, J=6.4 Hz, 1H), 7.62 (s, 1H), 7.40 (m, 2H), 7.18 (t, J=7.2 Hz, 1H), 7.07 (t, J=7.6, Hz, 1H), 5.09 (m, 1H), 4.11 (br s, 2H), 1.66 (s, 6H), 1.14 (s, 3H), 1.13 (s, 3H); MS (EI) for C₂₆H₂₅F₄N₂O₃: 489.2 (MH⁺).

1-methylethyl 3-{[3-fluoro-5-(trifluoromethyl)phenyl]carbonyl}-1,1-dimethyl-1,2,3,6-tetrahydroazepino[4,5-b]indole-5-carboxylate: ¹H NMR (400 MHz, CDCl₃): δ 10.65 (s, 1H), 7.83 (d, J=8.0, 1H), 7.71 (s, 1H), 7.63 (s, 1H), 7.52 (t, J=9.6 Hz, 2H), 7.40 (d, J=8.0 Hz, 1H), 7.20 (t, J=7.6 Hz, 1H), 7.10 (t, J=8.0, Hz, 1H), 5.15 (sept., J=6.4 Hz, 1H), 4.09 (br s, 2H), 1.65 (s, 6H), 1.21 (s, 3H), 1.19 (s, 3H); MS (EI) for C₂₆H₂₅F₄N₂O₃: 489.2 (MH⁺).

1-methylethyl 3-{[3,5-bis(trifluoromethyl)phenyl]carbonyl}-1,1-dimethyl-1,2,3,6-tetrahydroazepino[4,5-b]indole-5-carboxylate: ¹H NMR (400 MHz, CDCl₃): δ 10.64 (s, 1H), 8.06 (s, 2H), 8.05 (s, 1H), 7.84 (d, J=8.0 Hz, 1H), 7.68 (s, 1H), 7.40 (d, J=8.0 Hz, 1H), 7.21 (t, J=7.6 Hz, 1H), 7.10 (t, J=8.0, Hz, 1H), 5.15 (sept., J=6.4 Hz, 1H), 4.11 (br s, 2H), 1.66 (s, 6H), 1.18 (s, 3H), 1.17 (s, 3H); MS (EI) for C₂₇H₂₅F₆N₂O₃: 539.2 (MH⁺).

1-methylethyl 3-{[2,5-bis(trifluoromethyl)phenyl]carbonyl}-1,1-dimethyl-1,2,3,6-tetrahydroazepino[4,5-b]indole-5-carboxylate: ¹H NMR (400 MHz, CDCl₃): δ 10.63 (s, 1H), 7.91 (m, 3H), 7.62 (s, 1H), 7.45 (s, 1H), 7.37 (d, J=8.4 Hz, 1H), 7.19 (t, J=7.6 Hz, 1H), 7.09 (t, J=8.0 Hz, 1H), 5.15 (sept., J=6.4 Hz, 1H), 4.10 (br s, 2H), 1.69 (s, 6H), 1.09 (s, 3H), 1.07 (s, 3H); MS (EI) for C₂₇H₂₅F₆N₂O₃: 539.1 (MH⁺).

1-methylethyl 3-{[2,3-difluoro-4-(trifluoromethyl)phenyl]carbonyl}-1,1-dimethyl-1,2,3,6-tetrahydroazepino[4,5-b]indole-5-carboxylate: ¹H NMR (400 MHz, CDCl₃): δ 10.63 (s, 1H), 7.84 (d, J=7.6 Hz, 1H), 7.53 (m, 2H), 7.37 (m, 2H), 7.19 (t, J=8.0 Hz, 1H), 7.08 (t, J=7.6 Hz, 1H), 5.09 (m, 1H), 4.10 (br s, 2H), 1.66 (s, 6H), 1.18 (s, 3H), 1.16 (s, 3H); MS (EI) for C₂₆H₂₄F₅N₂O₃: 507.0 (MH⁺).

1-methylethyl 3-[(4-fluoro-3-methylphenyl)carbonyl]-1,1-dimethyl-1,2,3,6-tetrahydroazepino[4,5-b]indole-5-carboxylate: ¹H NMR (400 MHz, CDCl₃): δ 10.70 (s, 1H), 7.83 (s, 1H), 7.82 (d, J=7.2 Hz, 1H), 7.49 (dd, J=7.2, 1.6 Hz, 1H), 7.40 (m, 2H), 7.18 (t, J=7.4 Hz, 1H), 7.08 (t, J=7.4 Hz, 1H), 7.05 (t, J=9.2 Hz, 1H), 5.15 (sept., J=6.4 Hz, 1H), 4.08 (br s, 2H), 2.30 (s, 3H), 1.63 (s, 6H), 1.22 (s, 3H), 1.20 (s, 3H); MS (EI) for C₂₆H₂₈FN₂O₃: 435.3 (MH⁺).

1-methylethyl 3-[(3-chloro-2,6-difluorophenyl)carbonyl]-1,1-dimethyl-1,2,3,6-tetrahydroazepino[4,5-b]indole-5-carboxylate: ¹H NMR (400 MHz, CDCl₃): δ 10.64 (s, 1H), 7.84 (s, 1H), 7.84 (d, J=8.0 Hz, 1H), 7.66 (s, 1H), 7.53 (m, 2H), 7.37 (d, J=8.0 Hz, 1H), 7.18 (t, J=7.6 Hz, 1H), 7.08 (t, J=7.8 Hz, 1H), 7.00 (t, J=8.8 Hz, 1H), 5.08 (sept., J=6.0 Hz, 1H), 4.11 (br s, 2H), 1.66 (s, 6H), 1.18 (s, 6H); MS (EI) for C₂₅H₂₄ClF₂N₂O₃: 473.2 (MH⁺).

1-methylethyl 3-[(3-chloro-4-fluorophenyl)carbonyl]-1,1-dimethyl-1,2,3,6-tetrahydroazepino[4,5-b]indole-5-carboxylate: ¹H NMR (400 MHz, CDCl₃): δ 10.67 (s, 1H), 7.83 (d, J=8.0 Hz, 1H), 7.76 (s, 1H), 7.72 (dd, J=6.8, 2.0 Hz, 1H), 7.50 (m, 1H), 7.39 (d, J=8.4 Hz, 1H), 7.21 (t, J=8.4 Hz, 1H), 7.20 (t, J=7.8 Hz, 1H), 7.09 (t, J=8.2 Hz, 1H), 5.16 (sept., J=6.4 Hz, 1H), 4.07 (br s, 2H), 1.63 (s, 6H), 1.24 (s, 3H), 1.22 (s, 3H); MS (EI) for C₂₅H₂₅ClFN₂O₃: 455.2 (MH⁺).

1-methylethyl 3-[(3-bromo-4-fluorophenyl)carbonyl]-1,1-dimethyl-1,2,3,6-tetrahydroazepino[4,5-b]indole-5-carboxylate: ¹H NMR (400 MHz, CDCl₃): δ 10.68 (s, 1H), 7.86 (dd, J=6.6, 2.4 Hz, 1H), 8.10 (m, 1H), 7.84 (m, 1H), 7.77 (s, 1H), 7.56 (m, 1H), 7.40 (d, J=8.4 Hz, 1H), 7.34 (m, 1H), 7.20 (t, J=7.8 Hz, 1H), 7.09 (t, J=7.6 Hz, 1H), 5.16 (sept., J=6.4 Hz, 1H), 4.08 (br s, 2H), 1.63 (s, 6H), 1.24 (s, 3H), 1.23 (s, 3H); MS (EI) for C₂₅H₂₅BrFN₂O₃: 499.2 (MH⁺).

1-methylethyl1,1-dimethyl-3-({3-[(phenylmethyl)oxy]phenyl}carbonyl)-1,2,3,6-tetrahydroazepino[4,5-b]indole-5-carboxylate: ¹H NMR (400 MHz, CDCl₃): δ 10.72 (bs, 1H), 7.84-7.00 (m, 14H), 5.12 (m, 1H), 5.08 (s, 1H), 4.11 (bs, 2H), 1.57 (s, 6H), 1.19 (d, J=6.4 Hz, 6H); MS (EI) for C₃₂H₃₂N₂O₄: 509.3 (MH⁺).

1-Methylethyl-3-(cyclohexylcarbonyl)-1,1-dimethyl-1,2,3,6-tetrahydroazepino[4,5-b]indole-5-carboxylate: ¹H NMR (400 MHz, CDCl₃): δ 10.67 (s, 1H), 8.18 (s, 1H), 7.78 (d, 1H), 7.36 (d, 2H), 7.20 (t, 1H), 7.12 (t, 1H), 5.22 (m, 1H), 3.85 (bs, 2H), 2.68 (m, 1H), 1.84 (s, 2H), 1.47 (m, 9H), 1.39 (d, 6H), 1.22 (m, 5H); MS (EI) for C₂₅H₃₂N₂O₃: 409.2 (MH⁺).

1-Methylethyl-1,1-dimethyl-3-[(1-methylpiperidin-3-yl)carbonyl]-1,2,3,6-tetrahydroazepino[4,5-b]indole-5-carboxylate: ¹H NMR (400 MHz, DMSO): δ 8.01 (bs, 1H), 7.96 (s, 1H), 7.77-6.98 (m, 4H), 5.26 (m, 1H), 3.80-3.40 (m, 4H), 3.40-3.08 (m, 2H), 2.98 (m, 1H), 2.88 (s, 3H), 2.10-1.60 (m 4H), 1.52 (m, 6H), 1.40 (m, 6H); MS (EI) for C₂₅H₃₃N₃O₃: 424.2 (MH⁺).

1-methylethyl 3-acetyl-1,1-dimethyl-1,2,3,6-tetrahydroazepino[4,5-b]indole-5-carboxylate: ¹H NMR (400 MHz, CDCl₃): δ 10.70 (s, 1H), 8.03 (br s, 1H), 7.79 (d, J=8.4 Hz, 1H), 7.36 (dd J=8.0, 0.8 Hz, 1H), 7.16 (t, J=8.2 Hz, 1H), 7.06 (t, J=7.6 Hz, 1H), 5.24 (sept., J=6.4 Hz, 1H), 3.88 (br s, 2H), 2.39 (s, 3H), 1.56 (s, 6H), 1.40 (s, 3H), 1.38 (s, 3H); MS (EI) for C₂₀H₂₅N₂O₃, 314.2 (MH⁺).

1-methylethyl 3-butanoyl-1,1-dimethyl-1,2,3,6-tetrahydroazepino[4,5-b]indole-5-carboxylate: ¹H NMR (400 MHz, CDCl₃): δ10.70 (s, 1H), 8.10 (br s, 1H), 7.79 (d, J=8.0 Hz, 1H), 7.36 (d, J=8.0 Hz, 1H), 7.15 (d, J=7.6 Hz, 1H), 7.05 (d, J=7.6, Hz, 1H), 5.24 (sept., J=6.4 Hz, 1H), 3.90 (br s, 2H), 2.59 (t, J=7.2, Hz, 2H), 1.76 (app. sext., J=7.6, 2H), 1.55 (s, 6H), 1.40 (s, 3H), 1.38 (s, 3H), 1.00 (t, J=7.2 Hz, 3H); MS (EI) for C₂₂H₂₉N₂O₃, 369.2 (MH⁺).

1-methylethyl 1,1-dimethyl-3-pentanoyl-1,2,3,6-tetrahydroazepino[4,5-b]indole-5-carboxylate: ¹H NMR (400 MHz, CDCl₃): δ 10.70 (s, 1H), 8.10 (br s, 1H), 7.79 (d, J=8.0 Hz, 1H), 7.36 (dd, J=8.0, 0.8 Hz, 1H), 7.16 (t, J=6.8 Hz, 1H), 7.05 (t, J=7.2, Hz, 1H), 5.24 (sept., J=6.4 Hz, 1H), 3.90 (br s, 2H), 2.59 (t, J=7.2, Hz, 2H), 1.76 (app. sext., J=7.6, 2H), 1.55 (s, 6H), 1.39 (m, 8H), 1.00 (t, J=7.2 Hz, 3H); MS (EI) for C₂₃H₃₁N₂O₃, 383.2 (MH⁺).

1-methylethyl1,1-dimethyl-3-[(1-methylpiperidin-4-yl)carbonyl]-1,2,3,6-tetrahydroazepino[4,5-b]indole-5-carboxylate ¹H NMR (400 MHz, DMSO-d₆): δ10.91 (bs, 1H), 9.42 (bs, 1H), 7.97 (bs, 1H), 7.72-6.97 (m, 4H), 5.15 (m, 1H), 3.82 (bs, 2H), 3.44 (m, 1H), 3.05 (m, 1H), 2.76 (m, 3H), 2.0-1.75 (m, 4H), 1.44 (s, 6H), 1.37 (d, J=6.4 Hz, 6H); MS (EI) for C₂₅H₃₃N₃O₃: 424.2 (MH⁺).

1-methylethyl 3-(cyclopentylcarbonyl)-1,1-dimethyl-1,2,3,6-tetrahydroazepino[4,5-b]indole-5-carboxylate: ¹H NMR (400 MHz, CDCl₃): δ 10.69 (s, 1H), 8.20 (br s, 1H), 7.78 (d, J=8.4 Hz, 1H), 7.36 (d, J=8.4 Hz, 1H), 7.15 (t, J=8.0 Hz, 1H), 7.05 (t, J=8.0 Hz, 1H), 5.23 (m, 1H), 3.92 (br s, 2H), 3.14 (m, 1H), 1.90 (br m, 4H), 1.77 (br m, 2H), 1.64 (br m, 2H), 1.58-1.54 (m, 6H), 1.40 (s, 3H), 1.38 (s, 3H); MS (EI) for C₂₄H₃₀N₂O₃, 395.2 (MH⁺).

1-methylethyl 3-(2,2-dimethylpropanoyl)-1,1-dimethyl-1,2,3,6-tetrahydroazepino[4,5-b]indole-5-carboxylate: ¹H NMR (400 MHz, CDCl₃): δ 10.66 (s, 1H), 8.36 (s, 1H), 7.78 (d, J=8.0 Hz, 1H), 7.36 (d, J=8.0 Hz, 1H), 7.15 (t, J=7.2 Hz, 1H), 7.05 (t, J=7.2 Hz, 1H), 5.24 (m, 1H), 3.95 (br s, 2H), 1.60-1.30 (m, 21H); MS (EI) for C₂₃H₃₀N₂O₃, 383.2 (MH⁺).

1-methylethyl 3-(2-ethylbutanoyl)-1,1-dimethyl-1,2,3,6-tetrahydroazepino[4,5-b]indole-5-carboxylate: ¹H NMR (400 MHz, CDCl₃): δ 10.69 (s, 1H), 8.18 (br s, 1H), 7.79 (d, J=8.4 Hz, 1H), 7.36 (d, J=8.0 Hz, 1H), 7.15 (t, J=7.6 Hz, 1H), 7.05 (t, J=7.2, Hz, 1H), 5.24 (sept., J=6.0 Hz, 1H), 3.93 (br s, 2H), 2.69 (br s, 1H), 1.76 (m, 2H), 1.58 (m, 2H), 1.55 (s, 6H), 1.40 (s, 3H), 1.39 (s, 3H), 0.93 (t, J=7.6 Hz, 6H); MS (EI) for C₂₄H₃₃N₂O₃, 397.2 (MH⁺).

1-methylethyl 1,1-dimethyl-3-(3-methylbutanoyl)-1,2,3,6-tetrahydroazepino[4,5-b]indole-5-carboxylate: ¹H NMR (400 MHz, CDCl₃): δ 10.69 (s, 1H), 8.09 (br s, 1H), 7.79 (dd, J=8.4, 0.8 Hz, 1H), 7.36 (d, J=8.0 Hz, 1H), 7.15 (t, J=7.8 Hz, 1H), 7.05 (t, J=7.4, Hz, 1H), 5.24 (sept., J=6.4 Hz, 1H), 3.93 (br s, 2H), 2.49 (d, J=7.2 Hz, 2H), 2.22 (sept., J=6.8 Hz, 1H), 1.55 (s, 6H), 1.40 (s, 3H), 1.38 (s, 3H), 1.38 (s, 3H), 1.01 (s, 3H), 1.00 (s, 3H); MS (EI) for C₂₃H₃₁N₂O₃, 383.2 (MH⁺).

1-methylethyl 3-(cycloheptylcarbonyl)-1,1-dimethyl-1,2,3,6-tetrahydroazepino[4,5-b]indole-5-carboxylate: ¹H NMR (400 MHz, CDCl₃): δ 10.70 (s, 1H), 8.14 (br s, 1H), 7.79 (d, J=8.4 Hz, 1H), 7.36 (d, J=8.0 Hz, 1H), 7.15 (t, J=7.2 Hz, 1H), 7.05 (t, J=7.2, Hz, 1H), 5.25 (sept., J=6.0 Hz, 1H), 3.89 (br s, 2H), 2.88 (m, 1H), 1.81 (m, 6H), 1.60 (m, 6H), 1.54 (s, 6H), 1.40 (s, 3H), 1.38 (s, 3H); MS (EI) for C₂₆H₃₅N₂O₃, 423.3 (MH⁺).

1-methylethyl 1,1-dimethyl-3-propanoyl-1,2,3,6-tetrahydroazepino[4,5-b]indole-5-carboxylate: ¹H NMR (400 MHz, CDCl₃): δ 10.70 (s, 1H), 8.11 (br s, 1H), 7.79 (d, J=8.0 Hz, 1H), 7.36 (d, J=8.0 Hz, 1H), 7.16 (t, J=7.6 Hz, 1H), 7.05 (t, J=6.8, Hz, 1H), 5.24 (sept., J=6.4 Hz, 1H), 3.90 (br s, 2H), 2.64 (q, J=7.2 Hz, 2H), 1.55 (s, 6H), 1.40 (s, 3H), 1.39 (s, 3H), 1.25 (t, J=7.2, 3H); MS (EI) for C₂₁H₂₇N₂O₃, 355.3 (MH⁺).

1-methylethyl 3-[4-(dimethylamino)butanoyl]-1,1-dimethyl-1,2,3,6-tetrahydroazepino[4,5-b]indole-5-carboxylate: ¹HNMR (400 MHz, CDCl₃): δ 10.67 (s, 1H), 7.97 (br s, 1H), 7.78 (d, J=8.0 Hz, 1H), 7.37 (d, J=8.4 Hz, 1H), 7.17 (t, J=7.2 Hz, 1H), 7.06 (t, J=7.6, Hz, 1H), 5.24 (sept., J=6.4 Hz, 1H), 3.90 (br s, 2H), 2.93 (t, J=7.2 Hz, 2H), 2.77 (m, 2H), 2.68 (s, 6H), 2.06 (app. quint., J=6.8, 2H), 1.55 (s, 6H), 1.41 (s, 3H), 1.39 (s, 3H); MS (EI) for C₂₄H₃₄N₃O₃, 412.3 (MH⁺).

1-methylethyl1,1-dimethyl-3-[(3s,5s,7s)-tricyclo[3.3.1.1˜3,7˜]dec-1-ylcarbonyl]-1,2,3,6-tetrahydroazepino[4,5-b]indole-5-carboxylate: ¹H NMR (400 MHz, CDCl₃): δ 10.66 (s, 1H), 8.50 (s, 1H), 7.77 (d, J=8.0 Hz, 1H), 7.36 (dd, J=8.0, 0.8 Hz, 1H), 7.15 (t, J=7.8 Hz, 1H), 7.05 (t, J=8.4, Hz, 1H), 5.27 (sept., J=6.0 Hz, 1H), 3.94 (br s, 2H), 2.06 (m, 5H), 1.93 (m, 2H), 1.73 (m, 8H), 1.50 (s, 6H), 1.40 (s, 3H), 1.39 (s, 3H); MS (EI) for C₂₉H₃₇N₂O₃, 461.2 (MH⁺).

### Example 7

### Preparation of 1-Methylethyl 3-[(3-Hydroxyphenyl)Carbonyl]-1,1-Dimethyl-1,2,3,6-Tetrahydroazepino[4,5-b]Indole-5-Carboxylate

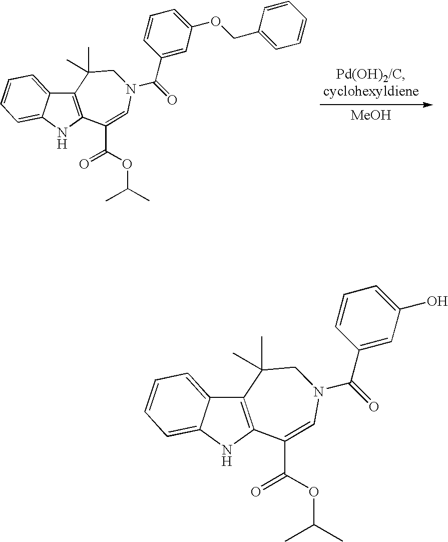

1-methylethyl3-[(3-hydroxyphenyl)carbonyl]-1,1-dimethyl-1,2,3,6-tetrahydroazepino[4,5-b]indole-5-carboxylate: To the solution of 1-methylethyl 1,1-dimethyl-3-({3-[(phenylmethyl)oxy]phenyl}carbonyl)-1,2,3,6-tetrahydroazepino[4,5-b]indole-5-carboxylate (1.075 g, 2.11 mmol) in methanol was added cyclohexyldiene (1.69 g, 21.1 mmol) and Pd(OH)₂/C in a sealed tube. The reaction mixture was heated to 64° C. overnight. After completion, the reaction mixture was filtered and the solvent was evaporated to give the desired product (0.82 g, 93% yield): ¹HNMR (400 MHz, CDCl₃): δ 10.71 (bs, 1H), 7.83-6.99 (m, 9H), 5.92 (s, 1H), 5.13 (m, 1H), 4.05 (bs, 2H), 1.63 (s, 6H), 1.20 (d, J=6.4 Hz, 6H); MS (EI) for C₂₅H₂₆N₂O₄: 419.3 (MH⁺).

### Example 8

### Preparation of 1-Methylethyl 3-[(3-{[2-(Dimethylamino)Ethyl]Oxy}Phenyl) Carbonyl]-1,1-Dimethyl-1,2,3,6-Tetrahydroazepino[4,5-b]Indole-5-Carboxylate

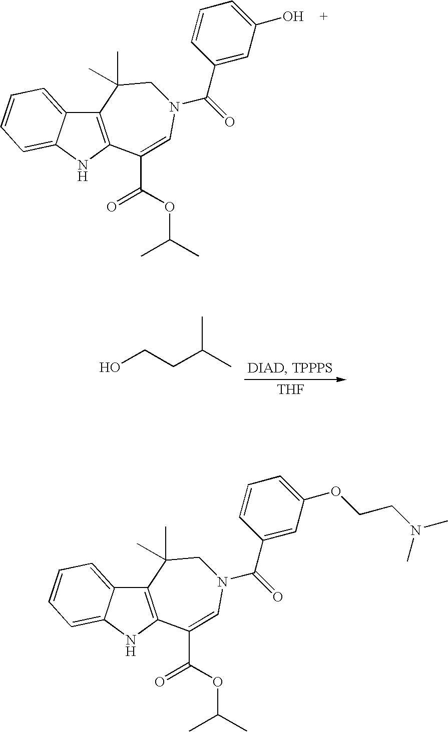

1-methylethyl 3-[(3-([2-(dimethylamino)ethyl]oxy phenyl)carbonyl]-1,1-dimethyl-1,2,3,6-tetrahydroazepino[4,5-b]indole-5-carboxylate: To the solution of 1-methylethyl3-[(3-hydroxyphenyl)carbonyl]-1,1-dimethyl-1,2,3,6-tetrahydroazepino[4,5-b]indole-5-carboxylate (150 mg, 0.36 mmol) in THF were added 2-dimethylaminoethanol (35.14 mg, 0.39 mmol), diisopropyl azodicarboxylate (80 mg, 0.39 mmol), triphenylphosphine polystyrene (358 mg, 0.39 mmol) subsequently. The reaction mixture was stirred at room temperature overnight. After filtration, solvent was evaporated in vacuum and the residue was purified by preperative liquid chromatography using a 10%-90% gradient of ACN/H₂O with 0.05% TFA for 11 minutes. Desired fractions were combined and neutralized by saturated NaHCO₃ and extracted with ethylacetate. The organic layer was dried over Na₂SO₄ and filtered. Removal of the product on a rotary evaporator gave the desired product (26.4 mg, 15% yield): ¹H NMR (400 MHz, CDCl₃): δ 10.72 (bs, 1H), 7.84-7.06 (m, 9H), 5.12 (m, 1H), 4.11 (bs, 2H), 4.08 (t, J=5.6 Hz, 2H), 2.73 (t, J=5.6 Hz, 2H), 2.32 (s, 6H), 1.63 (s, 6H), 1.19 (d, J=6.4 Hz, 6H); MS (EI) for C₂₉H₃₅N₃O₄: 490.2 (MH⁺).

Using the same or analogous synthetic techniques and/or substituting with alternative reagents, the following compounds of the invention were prepared:

1-methylethyl3-[(3-{[3-(dimethylamino)propyl]oxy}phenyl)carbonyl]-1,1-dimethyl-1,2,3,6-tetrahydroazepino[4,5-b]indole-5-carboxylate: ¹H NMR (400 MHz, CDCl₃): δ 10.72 (bs, 1H), 7.86-7.05 (m, 9H), 5.12 (m, 1H), 4.06 (bs, 2H), 4.03 (t, J=6.4 Hz, 2H), 2.43 (t, J=7.2 Hz, 2H), 2.24 (s, 6H), 1.95 (m, 2H), 1.63 (s, 6H), 1.19 (d, J=6.0 Hz, 6H); MS (ET) for C₃₀H₃₇N₃O₄: 504.4 (MH⁺).

1-methylethyl1,1-dimethyl-3-({3-[(2-piperidin-1-ylethyl)oxy]phenyl}carbonyl)-1,2,3,6-tetrahydroazepino[4,5-b]indole-5-carboxylate: ¹H NMR (400 MHz, CDCl₃): δ 10.72 (bs, 1H), 7.84-7.08 (m, 9H), 5.12 (m, 1H), 4.11 (t, J=6.0 Hz, 2H) 4.09 (bs, 2H), 2.76 (t, J=6.0 Hz, 2H), 2.48 (bs, 4H), 1.63 (s, 6H), 1.58 (m, 4H), 1.43 (m, 2H), 1.19 (d, J=6.0 Hz, 6H); MS (I) for C₃₂H₃₉N₃O₄: 530.4 (MH⁺).

1-methylethyl1,1-dimethyl-3-({3-[(2-morpholin-4-ylethyl)oxy]phenyl}carbonyl)-1,2,3,6-tetrahydroazepino[4,5-b]indole-5-carboxylate: ¹H NMR (400 MHz, CDCl₃): δ 10.71 (bs, 1H), 7.84-7.06 (m, 9H), 5.11 (m, 1H), 4.12 (t, J=5.6 Hz, 2H) 4.09 (bs, 2H), 3.72 (t, J=4.8 Hz, 4H), 2.80 (t, J=5.6 Hz, 2H), 2.57 (m, 4H), 1.63 (s, 6H), 1.19 (d, J=6.0 Hz, 6H); MS (EI) for C₃₁H₃₇N₃O₅: 532.4 (MH⁺).

1-methylethyl3-{[3,4-difluoro-5-({[4-(methyloxy)phenyl]methyl}oxy)phenyl]carbonyl}-1,1-dimethyl-1,2,3,6-tetrahydroazepino[4,5-b]indole-5-carboxylate: ¹H NMR (400 MHz, CDCl₃): δ 10.68 (bs, 1H), 7.82-6.68 (m, 11H), 5.17 (m, 1H), 5.04 (s, 2H), 4.04 (bs, 2H), 3.69 (s, 3H), 1.58 (s, 6H), 1.25 (d, J=6.0 Hz, 6H); MS (EI) for C₃₃H₃₂F₂N₂O₅: 575.4 (MH⁺).

1-methylethyl3-({3,4-difluoro-5-[(2-morpholin-4-yl ethyl)oxy]phenyl}carbonyl)-1,1-dimethyl-1,2,3,6-tetrahydroazepino[4,5-b]indole-5-carboxylate: ¹H NMR (400 MHz, CDCl₃): δ 10.64 (s, 1H), 7.83-7.03 (m, 7H), 5.17 (m, 1H), 4.17 (bs, 2H), 4.07 (bs, 2H), 3.69 (bs, 4H), 2.82 (bs, 2H), 2.56 (bs, 4H), 1.56 (s, 6H), 1.25 (d, J=5.6 Hz, 6H); MS (EI) for C₃₁H₃₅F₂N₃O₅: 568.4 (MH⁺).

1-methylethyl3-({3,4-difluoro-5-[(2-piperidin-1-ylethyl)oxy]phenyl}carbonyl)-1,1-dimethyl-1,2,3,6-tetrahydroazepino[4,5-b]indole-5-carboxylate: ¹H NMR (400 MHz, CDCl₃): δ 10.65 (bs, 1H), 7.83-7.00 (m, 7H), 5.17 (m, 1H), 4.16 (t, J=5.6 Hz, 2H), 4.10 (bs, 2H) 2.78 (t, J=5.6 Hz, 2H), 2.47 (m, 4H), 1.62 (s, 6H), 1.55 (m, 4H), 1.42 (m, 2H), 1.25 (d, J=6.0 Hz, 6H); MS (EI) for C₃₂H₃₇F₂N₃O₄: 566.4 (MH⁺).

1-methylethyl1,1-dimethyl-3-({4-[(2-morpholin-4-ylethyl)oxy]phenyl}carbonyl)-1,2,3,6-tetrahydroazepino[4,5-b]indole-5-carboxylate carboxylate: ¹H NMR (400 MHz, CDCl₃): δ 10.72 (bs, 1H), 7.88-6.93 (m, 9H), 5.15 (m, 1H), 4.16 (t, J=5.6 Hz, 2H), 4.10 (bs, 2H) 3.74 (m, 4H), 2.82 (t, J=5.6 Hz, 2H), 2.58 (m, 4H), 1.62 (s, 6H), 1.22 (d, J=6.0 Hz, 6H); MS (EI) for C₃₁H₃₇N₃O₅: 532.4 (MH⁺).

1-methylethyl1,1-dimethyl-3-({4-[(2-piperidin-1-ylethyl)oxy]phenyl}carbonyl)-1,2,3,6-tetrahydroazepino[4,5-b]indole-5-carboxylate: ¹H NMR (400 MHz, CDCl₃): δ 10.73 (bs, 1H), 7.89-6.93 (m, 9H), 5.14 (m, 1H), 4.14 (t, J=5.6 Hz, 2H), 4.13 (bs, 2H) 3.74 (m, 4H), 2.78 (t, J=5.6 Hz, 2H), 2.50 (bs, 4H), 1.62 (m, 10H), 1.46 (m, 2H), 1.21 (d, J=6.0 Hz, 6H); MS (EI) for C₃₂H₃₉N₃O₄: 530.4 (MH⁺).

1-methylethyl 3-[(4-([2-(dimethylamino)ethyl]oxy phenyl)carbonyl]-1,1-dimethyl-1,2,3,6-tetrahydroazepino[4,5-b]indole-5-carboxylate: ¹H NMR (400 MHz, CDCl₃): δ 10.73 (bs, 1H), 7.89-6.93 (m, 9H), 5.14 (m, 1H), 4.11 (t, J=5.6 Hz, 2H), 4.10 (bs, 2H), 2.77 (t, J=5.6 Hz, 2H), 2.36 (s, 6H), 1.62 (s, 6H), 1.22 (d, J=6.0 Hz, 6H); MS (EI) for C₂₉H₃₅N₃O₄: 490.2 (MH⁺).

1-methylethyl3-[(4-{[3-(dimethylamino)propyl]oxy}phenyl)carbonyl]-1,1-dimethyl-1,2,3,6-tetrahydroazepino[4,5-b]indole-5-carboxylate: ¹H NMR (400 MHz, CDCl₃): δ 10.73 (bs, 1H), 7.90-6.92 (m, 9H), 5.14 (m, 1H), 4.10 (bs, 2H), 4.06 (t, J=6.40 Hz, 2H), 2.45 (t, J=6.8 Hz, 2H), 2.25 (s, 5H), 1.97 (dt, J=6.4, 6.8 Hz, 2H), 1.62 (s, 6H), 1.22 (d, J=6.0 Hz, 6H); MS (EI) for C₃₀H₃₇N₃O₄: 504.2 (MH⁺).

1-methylethyl 1,1-dimethyl-3-({4-[(2-pyrrolidin-1-ylethyl)oxy]phenyl}carbonyl)-1,2,3,6-tetrahydroazepino[4,5-b]indole-5-carboxylate: ¹HNMR (400 MHz, CDCl₃): δ 10.73 (bs, 1H), 7.89-6.93 (m, 9H), 5.14 (m, 1H), 4.15 (t, J=5.8 Hz, 2H), 4.15 (bs, 2H), 2.92 (t, J=5.8 Hz, 2H), 2.63 (m, 4H), 1.82 (m, 4H), 1.62 (s, 6H), 1.22 (d, J=6.40 Hz, 6H); MS (EI) for C₃₀H₃₇N₃O₄: 516.2 (MH⁺).

1-methylethyl 1,1-dimethyl-3-({4-[(3-piperidin-1-ylpropyl)oxy]phenyl}carbonyl)-1,2,3,6-tetrahydroazepino[4,5-b]indole-5-carboxylate: ¹H NMR (400 MHz, CDCl₃): δ 10.73 (bs, 1H), 7.90-6.92 (m, 9H), 5.14 (m, 1H), 4.10 (bs, 2H), 4.05 (t, J=6.4 Hz, 2H), 2.47 (t, J=7.4 Hz, 2H), 2.39 (m, 4H), 1.99 (dt, J=6.4, 7.4 Hz, 2H), 1.62 (s, 6H), 1.59 (m, 4H), 1.44 (m, 2H), 1.22 (d, J=6.40 Hz, 6H); MS (EI) for C₃₃H₄₁N₃O₄: 544.2 (MH⁺).

1-methylethyl 1,1-dimethyl-3-({4-[(3-morpholin-4-ylpropyl)oxy]phenyl}carbonyl)-1,2,3,6-tetrahydroazepino[4,5-b]indole-5-carboxylate: ¹H NMR (400 MHz, CDCl₃): δ 10.72 (bs, 1H), 7.89-6.90 (m, 9H), 5.15 (m, 1H), 4.09 (bs, 2H), 4.07 (t, J=6.2 Hz, 2H), 3.72 (m, 4H), 2.52 (t, J=7.4 Hz, 2H), 2.47 (m, 4H), 1.99 (dt, J=6.2, 7.4 Hz, 2H), 1.62 (s, 6H), 1.22 (d, J=6.00 Hz, 6H); MS (EI) for C₃₂H₃₉N₃O₅: 546.2 (MH⁺).

1-methylethyl1,1-dimethyl-3-({3-[(3-morpholin-4-ylpropyl)oxy]phenyl}carbonyl)-1,2,3,6-tetrahydroazepino[4,5-b]indole-5-carboxylate: ¹H NMR (400 MHz, DMSO-d₆): δ 10.84 (s, 1H), 7.76 (d, J=8.0 Hz, 1H), 7.70 (s, 1H), 7.55 (d, J=8.0 Hz, 1H), 7.42 (t, J=8.4 Hz, 1H), 7.14 (m, 1H), 7.08 (m, 2H), 6.98 (t, J=7.2 Hz, 1H), 5.03 (sept, J=6.0 Hz, 1H), 4.03 (t, J=6.0 Hz, 2H), 3.97 (bs, 2H), 3.53 (bt, J=4.4 Hz, 4H), 2.39 (bt, J=7.2 Hz, 2H), 2.33 (bs, 4H), 1.86 (quint, J=7.2 Hz, 2H), 1.52 (s, 6H), 1.15 (d, J=6.4 Hz, 6H); MS (EI) for C₃₂H₃₉N₃O₅: 546.3 (MH⁺).

1-methylethyl1,1-dimethyl-3-({3-[(3-piperidin-1-ylpropyl)oxy]phenyl}carbonyl)-1,2,3,6-tetrahydroazepino[4,5-b]indole-5-carboxylate: ¹H NMR (400 MHz, DMSO-d₆): δ 10.84 (s, 1H), 7-76 (d, J=8.0 Hz, 1H), 7.70 (s, 1H), 7.55 (d, J=8.0 Hz, 1H), 7.41 (t, J=8.4 Hz, 1H), 7.15 (m, 1H), 7.07 (m, 2H), 6.98 (t, J=7.2 Hz, 1H), 5.03 (sept, J=6.0 Hz, 1H), 4.02 (t, J=6.0 Hz, 2H), 3.97 (bs, 2H), 2.34 (bt, J=7.2 Hz, 2H), 2.33 (bs, 4H), 1.84 (quint, J=7.2 Hz, 2H), 1.52 (s, 6H), 1.45 (m, 4H), 1.34 (m, 2H), 1.15 (d, J=6.4 Hz, 6H); MS (EI) for C₃₃H₄₁N₃O₄: 544.3 (MH⁺).

1-methylethyl3-[(3-{[3-(diethylamino)propyl]oxy}phenyl)carbonyl]-1,1-dimethyl-1,2,3,6-tetrahydroazepino[4,5-b]indole-5-carboxylate: ¹H NMR (400 MHz, DMSO-d₆): δ 10.84 (s, 1H), 7.76 (d, J=8.0 Hz, 1H), 7.70 (s, 1H), 7.55 (d, J=8.0 Hz, 1H), 7.42 (t, J=8.4 Hz, 1H), 7.16 (m, 1H), 7.06 (m, 2H), 6.97 (t, J=7.2 Hz, 1H), 5.03 (sept, J=6.0 Hz, 1H), 4.03 (t, J=6.4 Hz, 2H), 3.96 (bs, 2H), 1.80 (bquint, J=6.0 Hz, 2H), 1.52 (s, 6H), 1.22 (m, 4H), 1.15 (d, J=6.4 Hz, 6H), 0.91 (bt, J=6.8 Hz); MS (EI) for C₃₂H₄₁N₃O₄: 532.3 (MH⁺).

### Example 9

### Preparation of N-({3-[(3,4-Difluorophenyl)Carbonyl]-1,1-Dimethyl-1,2,3,4,5,6-Hexahydroazepino[4,5-b]Indol-5-yl}Carbonyl)-β-Alanine

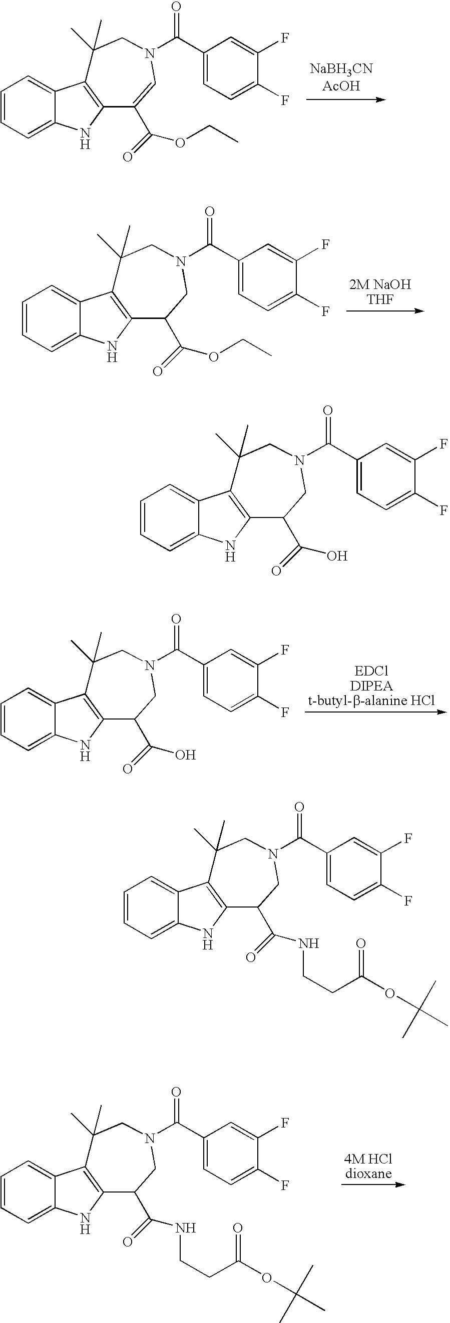

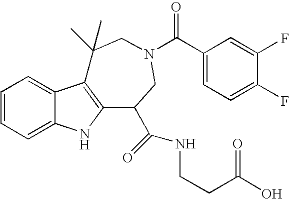

Sodium cyanoborohydride (0.137 g, 2.17 mmol) was added to a solution of ethyl 3-[(3,4-difluorophenyl)carbonyl]-1,1-dimethyl-1,2,3,6-tetrahydroazepino[4,5-b]indole-5-carboxylate (0.461 g, 1.09 mmol) in glacial acetic acid (10 mL) at room temperature for five hours. After the solution cleared, the reaction was quenched with 2M HCl until gas evolution ceased, then poured over ice. The slurry was neutralized to pH 7 with 5M ammonium hydroxide and the aqueous phase washed with CH₂Cl₂ (4×50 mL). The organic layers were combined and washed with brine, then separated and dried over Na₂SO₄, filtered and concentrated in vacuo to give ethyl 3-[(3,4-difluorophenyl)carbonyl]-1,1-dimethyl-1,2,3,4,5,6-hexahydroazepino[4,5-b]indole-5-carboxylate (0.460 g, 99% yield) as a white foam.

The crude residue (0.460 g, 1.09 mmol) was dissolved in THF (5.0 mL) and a 2M solution of NaOH (0.109 mL, 2.18 mmol) was added. The reaction was stirred at room temperature for 24 hours or until no starting material remained then concentrated to a minimum volume. The reaction was acidified by dropwise addition of 1M HCl until a precipitate formed which was collected by vacuum filtration, and dried to give 3-[(3,4-difluorophenyl)carbonyl]-1,1-dimethyl-1,2,3,4,5,6-hexahydroazepino[4,5-b]indole-5-carboxylic acid (0.399 g, 92% yield) as an off-white solid.

EDCI (0.130 g, 0.678 mmol) was added to a solution of the carboxylic acid (0.090 g, 0.226 mmol) and β-alanine t-butyl ester hydrochloride (0.082 g, 0.452 mmol) and diisopropylethylamine (0.112 mL, 0.678 mmol) in anhydrous THF (1.0 mL) at room temperature. After 12 hours, the reaction was concentrated in vacuo and the residue purified on reverse phase HPLC (25 mM ammonium acetate: acetonitrile, 20-90% gradient). The product was collected and lyophilized to give 1,1-dimethylethyl N-({3-[(3,4-difluorophenyl)carbonyl]-1,1-dimethyl-1,2,3,4,5,6-hexahydroazepino[4,5-b]indol-5-yl}carbonyl)-β-alaninate (60 mg, 51% yield) as a white solid. The title compound was obtained by treating a solution of the t-butyl ester (0.042 g, 0.080 mmol) in dioxane (1.0 mL) with 4M HCl in dioxane (0.5 mL) at room temperature for 30 minutes. The corresponding acid was concentrated in vacuo and precipitated from dioxane and ethyl ether to provide the title compound (0.010 g, 26% yield) as an off white solid: ¹H NMR (400 MHz, DMSO-d₆): δ 10.80 (s, 1H), 10.74 (s, 1H), 7.66 (bd, J=8.0 Hz, 1H), 7.52 (m, 2H), 7.26 (m, 2H), 6.98 (t, J=7.2 Hz, 1H), 6.92 (m, 1H), 3.92 (bs, 2H), 3.78 (m, 2H), 3.52 (t, J=6.0 Hz, 2H), 3.18 (m, 1H), 3.06 (bt, J=6.0 Hz, 2H), 1.46 (bs, 6H); MS (EI) for C₂₅H₂₅F₂N₃O₄: 468.3 (MH⁺).

### Example 10

### Preparation of 3-[(3,4-Difluorophenyl)Carbonyl]-1,1-Dimethyl-N-(1-methylethyl)-1,2,3,4,5,6-Hexahydroazepino[4,5-b]Indole-5-Carboxamide

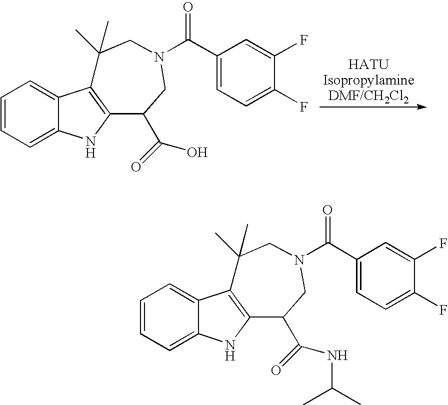

HATU (0.338 g, 0.888 mmol) was added to a solution of 3-[(3,4-difluorophenyl)carbonyl]-1,1-dimethyl-1,2,3,4,5,6-hexahydroazepino[4,5-b]indole-5-carboxylic acid (0.118 g, 0.291 mmol) and isopropylamine (200 mL, 2.37 mmol) in 1:1 anhydrous DMF:CH₂Cl₂. The reaction flask was capped tightly and stirred at room temperature overnight. The reaction was concentrated in vacuo and passed through a short plug of SiO₂ and the residue purified on reverse phase HPLC (25 mM ammonium acetate: acetonitrile, 20-90% gradient). The product was collected and lyophilized to give the title compound (21 mg, 16% yield) as a white solid: ¹H NMR (400 MHz, DMSO-d₆): δ 10.79 (s, 1H), 10.73 (s, 1H), 7.66 (bd, J=8.0 Hz, 1H), 7.52 (m, 2H), 7.26 (m, 2H), 6.98 (t, J=7.2 Hz, 1H), 6.92 (m, 1H), 3.96 (m, 1H), 3.92 (s, 1H), 3.78 (m, 1H), 3.52 (m, 1H), 3.18 (m, 1H), 3.06 (bt, J=6.0 Hz, 1H), 1.46 (bs, 3H), 1.19 (bd, J=6.0 Hz, 3H), 1.17 (bs, 3H), 1.11 (bd, J=6.0 Hz, 3H); MS (EI) for C₂₅H₂₇F₂N₃O₂: 440.2 (MH⁺).

### Example 11

### Preparation of N-({3-[(3,4-Difluorophenyl)Carbonyl]-1,1-Dimethyl-1,2,3,6-Tetrahydroazepino[4,5-b]Indol-5-yl}Carbonyl)-β-Alanine

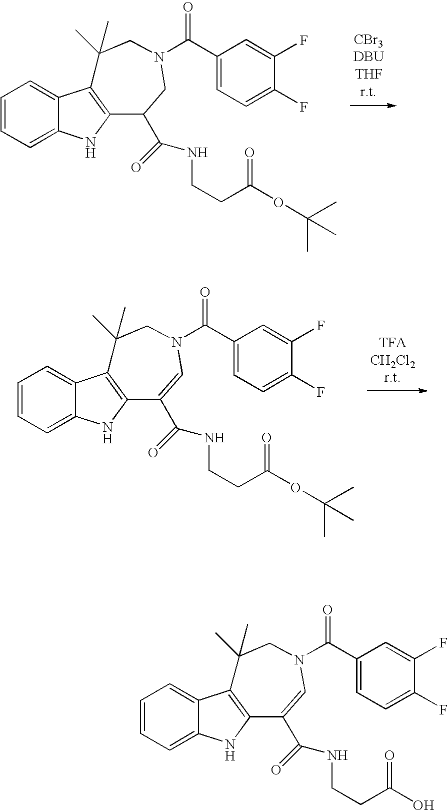

DBU (57 μL, 0.381 mmol) was added to a solution of 1,1-dimethylethyl N-({3-[(3,4-difluorophenyl)carbonyl]-1,1-dimethyl-1,2,3,4,5,6-hexahydroazepino[4,5-b]indol-5-yl}carbonyl)-β-alaninate (40 mg, 0.0761 mmol) and trichlorobromomethane (38 μL, 0.381 mmol) in anhydrous THF (1.0 mL) at room temperature. After 12 hours, the reaction was concentrated in vacuo and purified on reverse phase HPLC (25 mM ammonium acetate: acetonitrile, 20-90% gradient) to provide 1,1-dimethylethyl N-({3-[(3,4-difluorophenyl)carbonyl]-1,1-dimethyl-1,2,3,6-tetrahydroazepino[4,5-b]indol-5-yl}carbonyl)-β-alaninate (22 mg, 55% yield) as a yellow solid.

Trifluoroacetic acid (100 μL) was added to a solution of the t-butyl ester (0.022 g, 0.0420 mmol) in anhydrous CH₂Cl₂ (2.0 mL) at room temperature. After 12 hours, the reaction was diluted with CH₂Cl₂ (50 mL) and poured into water (50 mL). The organic layer was separated, washed with brine (50 mL) and dried over Na₂SO₄, filtered and concentrated in vacuo. The residue was redissolved in a minimal amount of CH₂Cl₂ and precipitated with hexanes to provide the title compound (2 mg, 10% yield) as a yellow solid: ¹H NMR (400 MHz, CDCl₃): δ 10.31 (s, 1H), 7.80 (bd, J=7.6 Hz, 1H), 7.47 (m, 1H), 7.38 (bd, J=8.4 Hz, 1H), 7.30 (m, 1H), 7.17 (m, 1H), 7.09 (t, J=7.2 Hz, 1H), 6.96 (bs, 1H), 6.51 (bs, 1H), 4.03 (bs, 2H), 3.60 (m, 2H), 2.67 (t, J=6.0 Hz, 2H), 1.60 (bs, 6H); MS (EI) for C₂₅H₂₃F₂N₃O₄: 468.1 (MH⁺).

### Example 12

### Preparation of 1-Methylethyl 3-[(3,4-Difluorophenyl)Carbonyl]-8-[(f{[2-(Dimethylamino)Ethyl]amino}Carbonyl)Oxy]-1,1-Dimethyl-1,2,3,6-tetrahydroazepino[4,5-b]Indole-5-Carboxylate

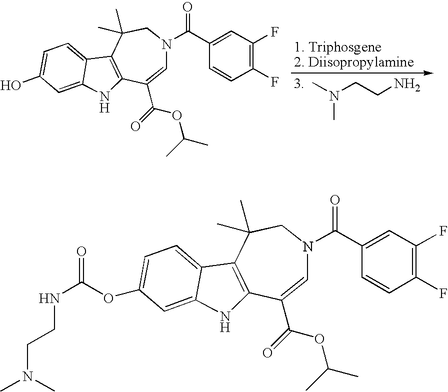

Triphosgene was added to a stirred solution of 1-methylethyl 3-[(3,4-difluorophenyl)carbonyl]-8-hydroxy-1,1-dimethyl-1,2,3,6-tetrahydroazepino 4,5-b]indole-5-carboxylate (100 mg, 0.23 mmol) and diisopropylamine (0.8 mL, 4.6 mmol) in dry dichloromethane (10 mL) at 0° C. under N₂. The resulting mixture was stirred at ambient temperature for 1.5 h and N,N-dimethylene diamine (125 μL, 1.15 mmol) was added and it was allowed to warn to room temperature overnight. The solvent was evaporated and it was directly applied to prep-LC to provide the title compound in (56 mg, 43% yield) as a yellow solid: ¹H NMR (400 MHz, CDCl₃): δ 10.70 (s, 1H), 7.76 (s, 1H), 7.74 (d, 1H), 7.50 (t, 1H), 7.39 (m, 1H), 7.30 (d, 1H), 7.20 (s, 1H), 6.82 (dd, J=8.6, 2.4 Hz, 1H), 5.18 (m, 1H), 4.10 (br, 2H), 3.64 (m, 2H), 3.30 (m, 2H), 2.93 (s, 6H), 1.60 (s, 6H), 1.24 (d, J=6.4 Hz, 6H); MS (EI) for C₃₀H₃₄F₂N₄O₅: 569.48 (MH⁺).

Using the same or analogous synthetic techniques and/or substituting with alternative reagents, the following compounds of the invention were prepared:

1-methylethyl3-[(3,4-difluorophenyl)carbonyl]-1,1-dimethyl-8-{[(methylamino)carbonyl]oxy}-1,2,3,6-tetrahydroazepino[4,5-b]indole-5-carboxylate: ¹H NMR (400 MHz, CDCl₃): δ 10.70 (s, 1H), 7.74 (s, 1H), 7.72 (d, 1H), 7.50 (m, 1H), 7.30 (m, 1H), 7.20 (d, 1H), 7.18 (s, 1H), 6.80 (dd, 1H), 5.18 (m, 1H), 4.10 (br, 2H), 2.90 (s, 3H), 1.60 (s, 6H), 1.23 (d, 6H); MS (EI) for C₂₇H₂₇F₂N₃O₅: 512.35 (MH⁺).

1-methylethyl 8-[({[3-(diethylamino)propyl]amino}carbonyl)oxy]-3-[(3,4-difluorophenyl)carbonyl]-1,1-dimethyl-1,2,3,6-tetrahydroazepino[4,5-b]indole-5-carboxylate: ¹H NMR (400 MHz, CDCl₃): δ 10.62 (s, 1H), 7.74 (s, 1H), 7.72 (d, 1H), 7.50 (m, 1H), 7.39 (m, 1H), 7.20 (d, 10H), 7.18 (s, 1H), 6.82 (dd, 1H), 5.18 (m, 1H), 4.10 (br, 2H), 3.38 (m, 2H), 2.60 (br, 6H), 1.78 (br, 2H), 1.60 (s, 6H), 1.20 (m, 9H), 1.02 (t, 3H); MS (EI) for C₃₃H₄₀F₂N₄O₅: 611.56 (MH⁺).

1-methylethyl3-[(3,4-difluorophenyl)carbonyl]-1,1-dimethyl-8-({[(2-pyrrolidin-1-ylethyl)amino]carbonyl}oxy)-1,2,3,6-tetrahydroazepino[4,5-b]indole-5-carboxylate: ¹HNMR (400 MHz, CDCl₃): δ 10.62 (s, 1H), 7.74 (s, 1H), 7.72 (d, 1H), 7.50 (m, 1H), 7.39 (m, 1H), 7.20 (d, 1H), 7.18 (s, 1H), 6.82 (dd, 1H), 5.18 (m, 1H), 4.10 (br, 2H), 3.58 (m, 2H), 3.00 (br, 4H), 2.00 (br, 6H), 1.60 (s, 6H), 1.23 (d, 6H); MS (EI) for C₃₂H₃₆F₂N₄O₅: 595.3 (MH⁺).

1-methylethyl3-[(3,4-difluorophenyl)carbonyl]-1,1-dimethyl-8-({[(2-piperidin-1-ylethyl)amino]carbonyl}oxy)-1,2,3,6-tetrahydroazepino[4,5-b]indole-5-carboxylate: ¹H NMR (400 MHz, CDCl₃): δ 10.62 (s, 1H), 7.74 (s, 1H), 7.72 (d, 1H), 7.50 (m, 1H), 7.39 (m, 1H), 7.20 (d, 1H), 7.18 (s, 1H), 6.82 (dd, 1H), 5.18 (m, 1H), 4.10 (br, 2H), 3.78 (m, 2H), 3.20 (m, 2H), 2.10 (br, 4H), 1.70 (br, 6H), 1.60 (s, 6H), 1.23 (d, 6H); MS (EI) for C₃₃H₃₅F₂N₄O₅: 609.3 (MH⁺).

1-methylethyl 8-[({[2-(diethylamino)ethyl]amino}carbonyl)oxy]-3-[(3,4-difluorophenyl)carbonyl]-1,1-dimethyl-1,2,3,6-tetrahydroazepino[4,5-b]indole-5-carboxylate: ¹H NMR (400 MHz, CDCl₃): δ 10.70 (s, 1H), 7.76 (s, 1H), 7.74 (d, 1H), 7.50 (m, 1H), 7.39 (m, 1H), 7.30 (m, 1H), 7.20 (s, 1H), 6.82 (dd, 1H), 5.18 (m, 1H), 4.10 (br, 2H), 3.58 (br, 2H), 2.90 (br, 6H), 1.60 (s, 6H), 1.25 (br, 6H), 1.23 (d, 6H); MS (EI) for C₃₂H₃₈F₂N₄O₅: 567.52 (MH⁺).

### Example 13

### Preparation of 1-Methylethyl 3-[(3,4-Difluorophenyl)Carbonyl]-8-({[(1,1-Dimethylethyl)oxy]Carbonyl}oxy)-1,1-Dimethyl-1,2,3,6-Tetrahydroazepino[4,5-b]Indole-5-Carboxylate

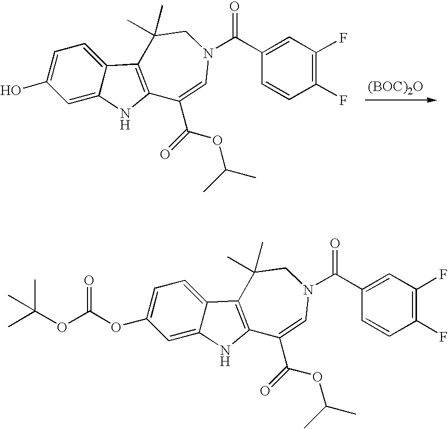

Boc anhydride (76 mg, 0.36 mmol) was added to a stirred solution of 1-methylethyl3-[(3,4-difluorophenyl)carbonyl]-8-hydroxy-1,1-dimethyl-1,2,3,6-tetrahydroazepino[4,5-b]indole-5-carboxylate (102 mg, 0.23 mmol) in dry dichloromethane (10 mL) at ambient temperature. The resulting mixture was stirred for 1.5 h and the solvent was evaporated and it was directly applied to prep-LC to provide the title compound in (52 mg, 40% yield) as a yellow solid: ¹H NMR (400 MHz, CDCl₃): δ 10.70 (s, 1H), 7.76 (s, 1H), 7.74 (d, 1H), 7.50 (m, 1H), 7.39 (m, 1H), 7.30 (d, 1H), 7.20 (s, 1H), 6.90 (dd, 1H), 5.18 (m, 1H), 4.10 (br, 2H), 2.93 (s, 6H), 1.60 (m, 9H), 1.24 (d, 6H); MS (EI) for C₃₀H₃₂F₂N₂O₆: 555.20 (MH⁺).

### Example 14

### Preparation of 1-Methylethyl 3-[(3,4-Difluorophenyl)Carbonyl]-8-{[2-(Dimethylamino)Ethyl]oxy}-1,1-Dimethyl-1,2,3,6-Tetrahydroazepino[4,5-b]Indole-5-Carboxylate

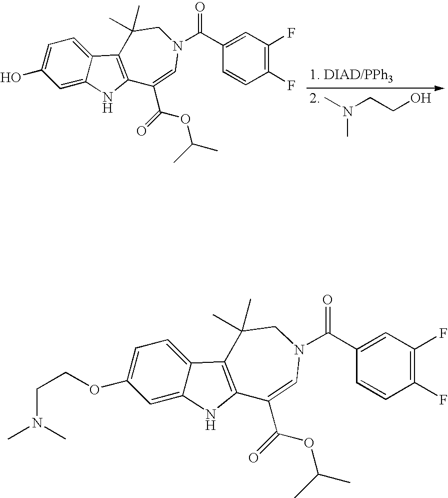

Triphenylphosphine (89.3 mg, 0.34 mmol), dimethylamine ethanol (34.3 μL, 0.34 mmol) and diisoproylazodicarboxylate (66 μL, 0.34 mmol) were added successively to a stirred solution of 1-methylethyl 3-[(3,4-difluorophenyl)carbonyl]-8-hydroxy-1,1-dimethyl-1,2,3,6-tetrahydroazepino[4,5-b]indole-5-carboxylate (100 mg, 0.23 mmol) in dry toluene (10 mL) at 0° C. under N₂. It was allowed to warm to room temperature overnight. The solvent was evaporated and it was directly applied to prep-LC to provide the title compound in (43.0 mg, 36% yield) as a yellow solid: ¹H NMR (400 MHz, CDCl₃): δ 10.50 (s, 1H), 7.74 (s, 1H), 7.72 (d, 1H), 7.50 (m, 1H), 7.39 (m, 1H), 7.20 (d, 1H), 6.83 (s, 1H), 6.78 (dd, 1H), 5.18 (m, 1H), 4.20 (m, 2H), 4.10 (br, 2H), 2.85 (br, 2H), 2.42 (s, 6H), 1.60 (s, 6H), 1.23 (d, 6H); MS (EI) for C₂₉H₃₃F₂N₃O₄: 526.42 (MH⁺).

Using the same or analogous synthetic techniques and/or substituting with alternative reagents, the following compounds of the invention were prepared:

1-methylethyl3-[(3,4-difluorophenyl)carbonyl]-8-{[3-(dimethylamino)propyl]oxy}-1,1-dimethyl-1,2,3,6-tetrahydroazepino[4,5-b]indole-5-carboxylate: ¹HNMR (400 MHz, CDCl₃): δ 10.50 (s, 1H), 7.74 (s, 1H), 7.72 (d, 1H), 7.50 (m, 1H), 7.39 (m, 1H), 7.20 (d, 1H), 6.83 (s, 1H), 6.78 (dd, 1H), 5.18 (m, 1H), 4.10 (m, 2H), 2.60 (m, 2H), 2.38 (s, 6H), 2.10 (m, 2H), 1.92 (br, 2H), 1.60 (s, 6H), 1.23 (d, 6H); MS (EI) for C₃₀H₃₅F₂N₃O₄: 540.44 (MH⁺).

1-methylethyl8-{[2-(diethylamino)ethyl]oxy}-3-[(3,4-difluorophenyl)carbonyl]-1,1-dimethyl-1,2,3,6-tetrahydroazepino[4,5-b]indole-5-carboxylate: ¹H NMR (400 MHz, CDCl₃): δ 10.50 (s, 1H), 7.74 (s, 1H), 7.72 (d, 1H), 7.50 (m, 1H), 7.39 (m, 1H), 7.20 (d, 1H), 6.83 (s, 1H), 6.78 (dd, 1H), 5.18 (m, 1H), 4.20 (m, 3H), 2.70 (m, 2H), 2.60 (m, 3H), 2.00 (m, 2H), 1.60 (s, 6H), 1.23 (d, 6H), 1.02 (t, 6H); MS (EI) for C₃₁H₃₇F₂N₃O₄: 554.45 (MH⁺).

1-methylethyl8-{[3-(diethylamino)propyl]oxy}-3-[(3,4-difluorophenyl)carbonyl]-1,1-dimethyl-1,2,3,6-tetrahydroazepino[4,5-b]indole-5-carboxylate: ¹H NMR (400 MHz, CDCl₃): δ 10.50 (s, 1H), 7.74 (s, 1H), 7.72 (d, 1H), 7.50 (m, 1H), 7.39 (m, 1H), 7.20 (d, 1H), 6.83 (s, 1H), 6.78 (dd, 1H), 5.18 (m, 1H), 4.18 (m, 2H), 4.10 (br, 2H), 2.98 (m, 2H), 2.70 (m, 4H), 1.60 (s, 6H), 1.23 (m, 8H), 1.02 (t, 6H); MS (EI) for C₃₂H₃₉F₂N₃O₄: 568.48 (MH⁺).

1-methylethyl3-[(3,4-difluorophenyl)carbonyl]-1,1-dimethyl-8-{[2-(methyloxy)ethyl]oxy}-1,2,3,6-tetrahydroazepino[4,5-b]indole-5-carboxylate: ¹H NMR (400 MHz, CDCl₃): δ 10.50 (s, 1H), 7.74 (s, 1H), 7.72 (d, 1H), 7.50 (m, 1H), 7.39 (m, 1H), 7.20 (d, 1H), 6.83 (s, 1H), 6.78 (dd, 1H), 5.18 (m, 1H), 4.18 (m, 2H), 4.10 (br, 2H), 3.80 (m, 2H), 3.42 (s, 3H), 1.60 (s, 6H), 1.23 (d, 6H); MS (EI) for C₂₈H₃₀F₂N₂O₅: 513.45 (MH⁺).

### Example 15

### Preparation of 1-Methylethyl 1,1-Dimethyl-3-{[3-(Morpholin-4-ylmethyl)Phenyl]Carbonyl}-1,2,3,6-Tetrahydroazepino[4,5-b]Indole-5-Carboxylate

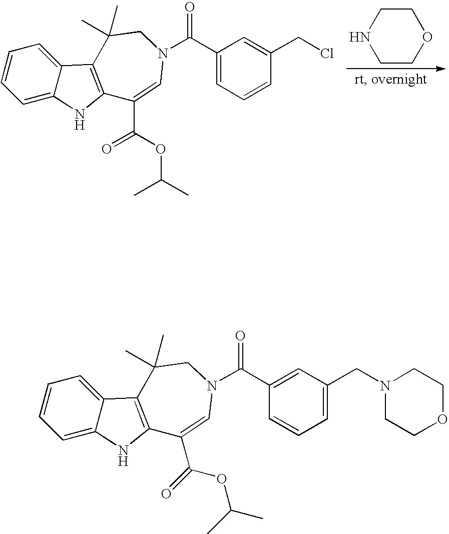

Morpholine (290 μL, 3.32 mmol) was added to a stirred solution of 2(1H-indole-3-yl)-2-methylpropan-1-amine hydrochloride (100 mg, 0.23 mmol) in acetonitrile: dichloromethane (5 mL, 5:1 ratio) at room temperature. The reaction was stirred overnight. The solvent was evaporated and the residue was directly applied to prep-LC to provide the title compound in (41.5 mg, 36% yield) as a yellow solid: ¹H NMR (400 MHz, CDCl₃): δ 10.62 (s, 1H), 7.82 (d, 1H), 7.80 (s, 1H), 7.40 (m, 5H), 7.20 (m, 1H), 7.15 (m, 1H), 5.18 (m, 1H), 4.10 (br, 2H), 3.62 (s, 4H), 3.50 (s, 2H), 2.40 (s, 4H), 1.60 (s, 6H), 1.23 (d, 6H); MS (EI) for C₃₀H₃₅N₃O₄: 502.3 (MH⁺).

Using the same or analogous synthetic techniques and/or substituting with alternative reagents, the following compounds of the invention were prepared:

1-methylethyl3-({3-[(diethylamino)methyl]phenyl}carbonyl)-1,1-dimethyl-1,2,3,6-tetrahydroazepino[4,5-b]indole-5-carboxylate: ¹H NMR (400 MHz, CDCl₃): δ 10.62 (s, 1H), 7.82 (d, 1H), 7.80 (s, 1H), 7.40 (m, 5H), 7.20 (m, 1H), 7.15 (m, 1H), 5.18 (m, 1H), 4.10 (br, 4H), 2.80 (br, 4H), 1.80 (br, 6H), 1.60 (s, 6H), 1.23 (d, 6H); MS (EI) for C₃₀H₃₇N₃O₃: 488.84 (MH⁺).

1-methylethyl3-{[2-fluoro-5-(morpholin-4-ylmethyl)phenyl]carbonyl}-1,1-dimethyl-1,2,3,6-tetrahydroazepino[4,5-b]indole-5-carboxylate: ¹H NMR (400 MHz, CDCl₃): δ 10.62 (s, 1H), 7.82 (d, 1H), 7.80 (s, 1H), 7.65 (d, 1H), 7.45 (m, 1H), 7.40 (d, 1H), 7.20 (t, 1H), 7.10 (m, 2H), 5.18 (m, 1H), 4.10 (br, 2H), 3.70 (m, 4H), 3.60 (s, 2H), 2.40 (m, 4H), 1.60 (s, 6H), 1.23 (d, 6H); MS (EI) for C₃₀H₃₄FN₃O₄: 520.30 (MH⁺).

1-methylethyl3-{[4-fluoro-3-(morpholin-4-ylmethyl)phenyl]carbonyl}-1,1-dimethyl-1,2,3,6-tetrahydroazepino[4,5-b]indole-5-carboxylate: ¹H NMR (400 MHz, CDCl₃): δ 10.62 (s, 1H), 7.82 (d, 1H), 7.70 (br, 1H), 7.42 (m, 2H), 7.38 (d, 1H), 7.18 (m, 1H), 7.10 (m, 2H), 5.18 (m, 1H), 4.10 (br, 2H), 3.70 (m, 4H), 3.60 (s, 2H), 2.40 (m, 4H), 1.60 (s, 6H), 1.23 (d, 6H); MS (EI) for C₃₀H₃₄FN3O₄: 520.30 (MH⁺).

1-methylethyl 3-{[2-fluoro-5-(piperidin-1-ylmethyl)phenyl]carbonyl}-1,1-dimethyl-1,2,3,6-tetrahydroazepino[4,5-b]indole-5-carboxylate: ¹H NMR (400 MHz, CDCl₃): δ 10.62 (s, 1H), 7.82 (d, 1H), 7.80 (s, 1H), 7.65 (d, 1H), 7.45 (m, 1H), 7.40 (d, 1H), 7.20 (t, 1H), 7.10 (m, 2H), 5.18 (m, 1H), 4.10 (br, 2H), 3.50 (s, 2H), 2.40 (s, 4H), 1.60 (s, 6H), 1.45 (m, 4H), 1.40 (m, 2H), 1.20 (d, 6H); MS (EI) for C₃₁H₃₆FN₃O₃: 518.30 (MH⁺).

1-methylethyl3-{[4-fluoro-3-(piperidin-1-ylmethyl)phenyl]carbonyl}-1,1-dimethyl-1,2,3,6-tetrahydroazepino[4,5-b]indole-5-carboxylate: ¹H NMR (400 MHz, CDCl₃): δ 10.62 (s, 1H), 7.82 (d, 1H), 7.80 (s, 1H), 7.65 (d, 1H), 7.45 (m, 1H), 7.40 (d, 1H), 7.20 (t, 1H), 7.10 (m, 2H), 5.18 (m, 1H), 4.10 (br, 2H), 3.50 (s, 2H), 2.40 (s, 4H), 1.60 (s, 6H), 1.45 (m, 4H), 1.40 (m, 2H), 1.20 (d, 6H); MS (EI) for C₃₁H₃₆FN₃O₃: 518.30 (MH⁺).

### Example 16

### Preparation of 1-Methylethyl 1,1-Dimethyl-3-{[3-(Pyrrolidin-1-ylmethyl)Phenyl]Carbonyl}-1,2,3,6-Tetrahydroazepino[4,5-b]Indole-5-Carboxylate

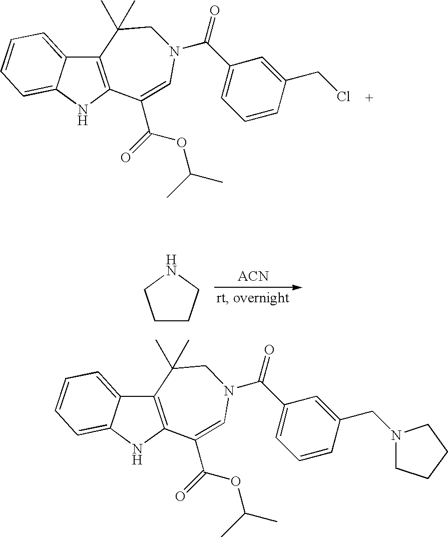

1-methylethyl 3-{[3-(chloromethyl)phenyl]carbonyl}-1,1-dimethyl-1,2,3,6-tetrahydroazepino[4,5-b]indole-5-carboxylate (60.0 mg, 0.133 mmol) was dissolved in 2 mL ACN. Pyrrolidine (142 mg, 2.00 mmol) was added to the solution and allowed to stir overnight at room temperature. The sample was purified by preparative liquid chromatography using a 20%-55% gradient of ACN/H₂O with 0.05% TFA for 8 minutes. Desired fractions were combined and made basic by saturated NaHCO₃ and diluted with ethyl acetate. Organic layer was extracted with water and brine, then dried over Na₂SO₃ and filtered. Yellow solution was reduced to dryness and lyophilized overnight in ACN/H₂O to form the bright yellow powder (25.9 mg, 40% yield) of the title compound: ¹H NMR (400 MHz, CDCl₃): δ10.71 (s, 1H), 7.83 (d, J=8.0 Hz, 1H), 7.78 (s, 1H), 7.53 (s, 1H), 7.46 (m, 2H), 7.39 (d, J=8.0 Hz, 1H), 7.19 (d, J=8.0 Hz, 1H), 7.09 (d, J=8.0 Hz, 1H), 5.11 (sept., J=6.4, 1H), 4.05 (br s, 2H), 3.75 (br s, 2H), 2.57 (br s, 4H), 1.84 (br s, 4H), 1.64 (s, 6H), 1.18 (s, 3H) 1.17 (s, 3H); MS (EI) for C₃₀H₃₆N₃O₃, 486.2 (MH⁺).

Using the same or analogous synthetic techniques and/or substituting with alternative reagents, the following compounds of the invention were prepared:

1-methylethyl 1,1-dimethyl-3-{[3-(piperidin-1-ylmethyl)phenyl]carbonyl}-1,2,3,6-tetrahydroazepino[4,5-b]indole-5-carboxylate: ¹H NMR (400 MHz, CDCl₃): δ 10.72 (s, 1H), 7.83 (d, J=8.0 Hz, 1H), 7.79 (s, 1H), 7.57 (br s, 1H), 7.51 (s, 1H), 7.41 (m, 3H), 7.18 (t, J=7.2 Hz, 1H), 7.08 (t, J=7.2 Hz, 1H), 5.09 (sept., J=6.0, 1H), 4.07 (br s, 2H), 3.56 (br s, 2H), 2.41 (br s, 4H), 1.64 (s, 6H), 1.43 (br s, 4H), 1.26 (s, 2H), 1.17 (s, 3H) 1.16 (s, 3H); MS (EI) for C₃₁H₃₈N₃O₃, 500.2 (MH⁺).

1-methylethyl 1,1-dimethyl-3-({3-[(4-methylpiperazin-1-yl)methyl]phenyl}carbonyl)-1,2,3,6-tetrahydroazepino[4,5-b]indole-5-carboxylate: ¹H NMR (400 MHz, CDCl₃): δ 10.70 (s, 1H), 7.83 (d, J=8.0 Hz, 1H), 7.79 (s, 1H), 7.54 (s, 1H), 7.46 (m, 2H), 7.40 (d, J=7.6 Hz, 2H), 7.18 (t, J=6.8 Hz, 1H), 7.08 (t, J=7.2 Hz, 1H), 5.10 (sept., J=6.0, 1H), 4.06 (br s, 2H), 3.54 (br s, 2H), 2.53 (br s, 8H), 2.36 (s, 3H), 1.64 (s, 6H), 1.17 (s, 3H) 1.16 (s, 3H); MS (EI) for C₃₁H₃₉N₄O₃, 515.3 (MH⁺).

1-methylethyl 3-({3-[(4-ethylpiperazin-1-yl)methyl]phenyl}carbonyl)-1,1-dimethyl-1,2,3,6-tetrahydroazepino[4,5-b]indole-5-carboxylate: ¹H NMR (400 MHz, CDCl₃): δ 10.70 (s, 1H), 7.83 (d, J=8.0 Hz, 1H), 7.80 (s, 1H), 7.55 (s, 1H), 7.49 (d, J=7.6 Hz, 2H), 7.43 (d, J=7.6 Hz, 2H), 7.38 (m, 3H), 7.18 (t, J=7.2 Hz, 1H), 7.08 (t, J=8.0 Hz, 1H), 5.10 (sept., J=6.4, 1H), 4.06 (br s, 2H), 3.54 (s, 2H), 2.51 (br s, 8H), 2.43 (q, J=7.2, 2H), 1.64 (s, 6H), 1.17 (s, 3H), 1.16 (s, 3H), 1.09 (t, J=7.2, 3H); MS (EI) for C₃₂H₄₁N₄O₃, 529.3 (MH⁺).

1-methylethyl 3-({3-[(4-acetylpiperazin-1-yl)methyl]phenyl}carbonyl)-1,1-dimethyl-1,2,3,6-tetrahydroazepino[4,5-b]indole-5-carboxylate: ¹H NMR (400 MHz, CDCl₃): δ 10.67 (s, 1H), 7.83 (d, J=8.0 Hz, 1H), 7.79 (s, 1H), 7.55 (s, 1H), 7.50 (d, J=7.6 Hz, 2H), 7.45 (d, J=7.0 Hz, 1H), 7.39 (m, 3H), 7.19 (t, J=7.2 Hz, 1H), 7.09 (t, J=7.4 Hz, 1H), 5.13 (sept., J=6.4, 1H), 4.09 (br s, 2H), 3.59 (t, J=3.6 Hz, 2H), 3.54 (s, 2H), 3.42 (t, J=3.6 Hz, 2H), 2.41 (m, 4H), 2.07 (m, 4H), 2.07 (s, 3H), 1.64 (s, 6H), 1.18 (s, 3H), 1.16 (s, 3H); MS (EI) for C₃₂H₃₉N₄O₄, 543.2 (MH⁺).

1-methylethyl 1,1-dimethyl-3-[(3-{[4-(methylsulfonyl)piperazin-1-yl]methyl}phenyl)carbonyl]-1,2,3,6-tetrahydroazepino[4,5-b]indole-5-carboxylate: ¹HNMR (400 MHz, CDCl₃): δ 10.69 (s, 1H), 7.83 (d, J=8.0 Hz, 1H), 7.76 (s, 1H), 7.54 (s, 1H), 7.47 (d, J=7.2 Hz, 1H), 7.40 (t, J=7.2 Hz, 1H), 7.19 (t, J=6.8 Hz, 1H), 7.09 (t, J=8.0 Hz, 1H), 5.11 (sept., J=6.0 Hz, 1H), 4.10 (br s, 2H), 3.58 (s, 2H), 3.17 (m, 4H), 2.67 (s, 3H), 2.53 (t, J=4.4 Hz, 4H), 1.65 (s, 6H), 1.18 (s, 3H), 1.16 (s, 3H); MS (EI) for C₃₁H₃₉N₄O₅S: 579.4 (MH⁺).

1-methylethyl 3-{[3-(azepan-1-ylmethyl)phenyl]carbonyl}-1,1-dimethyl-1,2,3,6-tetrahydroazepino[4,5-b]indole-5-carboxylate: ¹H NMR (400 MHz, CDCl₃): δ 10.66 (s, 1H), 8.02 (br s, 1H), 7.83 (d, J=8.0 Hz, 1H), 7.77 (s, 1H), 7.73 (br s, 1H), 7.55 (s, 2H), 7.39 (d, J=8.0 Hz, 1H), 7.20 (t, J=6.8 Hz, 1H), 7.09 (t, J=7.2 Hz, 1H), 5.30 (s, 2H), 5.13 (m, 1H), 4.21 (br m, 4H), 3.52 (br s, 2H), 2.82 (br s, 2H), 2.02 (br s, 2H), 1.80 (br s, 2H), 1.64 (s, 8H), 1.23 (s, 3H), 1.19 (s, 3H); MS (EI) for C₃₂H₄₀N₃O₃: 514.3 (MH⁺).

1-methylethyl 1,1-dimethyl-3-({3-[(4-methyl-1,4-diazepan-1-yl)methyl]phenyl}carbonyl)-1,2,3,6-tetrahydroazepino[4,5-b]indole-5-carboxylate: ¹H NMR (400 MHz, CDCl₃): δ 10.69 (s, 1H), 7.83 (d, J=8.0 Hz, 1H), 7.77 (s, 1H), 7.56 (s, 1H), 7.48 (t, J=7.2 Hz, 1H), 7.40 (t, J=7.6 Hz, 1H), 7.19 (t, J=6.8 Hz, 1H), 7.09 (t, J=6.8 Hz, 1H), 5.31 (s, 2H), 5.11 (sept., J=6.0 Hz, 1H), 4.10 (br s, 2H), 3.70 (s, 2H), 2.94 (br s, 2H), 2.84 (br s, 2H), 2.72 (t, J=6.0 Hz, 2H), 2.42 (s, 3H), 2.03 (m, 2H), 1.65 (s, 6H), 1.18 (s, 3H), 1.17 (s, 3H); MS (EI) for C₃₂H₄₁N₄O₃: 529.4 (MH⁺).

1-methylethyl 3-({3-[(4-{[(1,1-dimethylethyl)oxy]carbonyl}piperazin-1-yl)methyl]phenyl}carbonyl)-1,1-dimethyl-1,2,3,6-tetrahydroazepino[4,5-b]indole-5-carboxylate: ¹H NMR (400 MHz, CDCl₃): δ 10.71 (s, 1H), 7.83 (d, J=8.0 Hz, 1H), 7.79 (s, 1H), 7.51 (m, 2H), 7.51 (m, 2H), 7.41 (m, 3H), 7.18 (t, J=6.8 Hz, 1H), 7.09 (t, J=7.2 Hz, 1H), 5.10 (sept. J=6.0 Hz, 1H), 4.09 (br s, 2H), 3.53 (s, 2H), 3.40 (m, 4H), 2.36 (m, 4H), 2.36 (m, 4H), 1.64 (s, 6H), 1.45 (s, 9H), 1.17 (s, 3H), 1.16 (s, 3H); MS (EI) for C₃₅H₄₅N₄O₅: 601.4 (MH⁺).

1-methylethyl 3-{[3-(azocan-1-ylmethyl)phenyl]carbonyl}-1,1-dimethyl-1,2,3,6-tetrahydroazepino[4,5-b]indole-5-carboxylate: ¹H NMR (400 MHz, CDCl_): δ 10.71 (s, 1H), 7.83 (d, J=8.4 Hz, 1H), 7.58 (s, 2H), 7.38 (d, J=8.4 Hz, 3H), 7.18 (t, J=7.6 Hz, 1H), 7.08 (t, J=7.2 Hz, 1H), 5.10 (sept. J=6.0 Hz, 1H), 4.10 (br s, 2H), 3.62 (s, 2H), 2.52 (s, 2H), 1.64 (s, 6H), 1.59 (s, 2H), 1.51 (s, 8H), 1.18 (s, 3H), 1.16 (s, 3H); MS (EI) for C₃₄H₄₃N₄O₄: 528.3 (MH⁺).

1-methylethyl 3-({3-[(4-acetyl-1,4-diazepan-1-yl)methyl]phenyl}carbonyl)-1,1-dimethyl-1,2,3,6-tetrahydroazepino[4,5-b]indole-5-carboxylate: ¹H NMR (400 MHz, CDCl₃): δ 10.68 (s, 1H), 7.83 (d, J=8.0 Hz, 1H), 7.79 (s, 1H), 7.55 (s, 2H), 7.44 (br s, 1H), 7.39 (br s, 1H), 7.39 (d, J=8.4 Hz, 2H), 7.19 (t, J=8.0 Hz, 1H), 7.09 (t, J=7.2 Hz, 1H), 5.11 (m, 1H), 4.08 (br s, 2H), 3.60 (m, 4H), 2.64 (br m, 2H), 2.08 (s, 3H), 1.64 (s, 6H), 1.17 (m, 6H); MS (EI) for C₃₃H₄₂N₃O₃: 557.3 (MH⁺).

### Example 17

### Preparation of 1-Methylethyl 1,1-Dimethyl-3-[(3-{[(1-Methylpiperidin-4-yl)Oxy]Methyl}phenyl)Carbonyl]-1,2,3,6-Tetrahydroazepino[4,5-b]Indole-5-Carboxylate

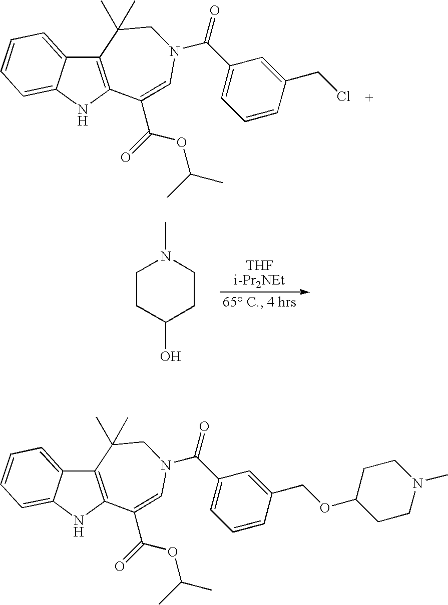

1-methylethyl 3-{[3-(chloromethyl)phenyl]carbonyl}-1,1-dimethyl-1,2,3,6-tetrahydroazepino[4,5-b]indole-5-carboxylate (30.0 mg, 0.0666 mmol) was dissolved in 2 mL THF. N,N′-diisopropylethylamine (194 mg, 0.150 mmol) and 4-hydroxy-N-methylpiperidine (115 mg, 0.998 mmol) were added to the stirring solution. The reaction mixture was brought to 65° C. and allowed to stir for 4 hours. The sample was purified by preparative liquid chromatography using a 20%-55% gradient of ACN/H₂O with 0.05% TFA for 8 minutes. Desired fractions were combined and made basic by saturated NaHCO₃ and diluted with ethyl acetate. Organic layer was extracted with water and brine, then dried over Na₂SO₃ and filtered. Yellow solution was reduced to dryness and lyophilized overnight in ACN/H₂O to form the yellow powder (10.5 mg, 30% yield) of the title compound: ¹H NMR (400 MHz, CDCl₃): δ 10.64 (s, 1H), 7.97 (m, 1H), 7.80 (m, 1H), 7.67 (m, 3H), 7.55 (t, J=8.0 Hz, 1H), 7.19 (m, 2H), 7.08 (t, J=8.0 Hz, 1H), 5.13 (m, 1H), 4.06 (br s, 2H), 3.70 (br s, 4H), 2.05 (m, 4H), 1.66 (s, 3H), 1.62 (s, 6H) 1.21 (m, 6H); MS (EI) for C₃₂H₄₀N₃O₄, 530.3 (MH⁺).

### Example 18

### Preparation of 1-Methylethyl 1,1-Dimethyl-3-[(3-{[4-(Phenylsulfonyl)piperazin-1-yl]Methyl}phenyl)Carbonyl]-1,2,3,6-Tetrahydroazepino[4,5-b]Indole-5-Carboxylate

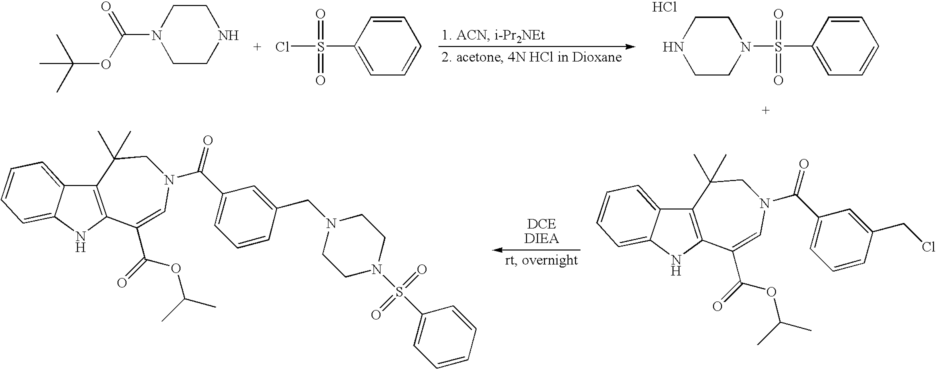

Piperazine-1-carboxylic acid tert-butyl ester (1.00 g, 5.37 mmol) was dissolved in 10 μL ACN. To the solution, N,N′-diisopropylethylamine (2.08 g, 16.1 mmol) and benzenesulfonyl chloride (948 mg, 5.37 mmol) were added and allowed to stir for 1 h. When TLC indicated complete disappearance of sulfonly chloride, solution was evaporated to dryness. The white powder was dissolved in 20 mL of acetone and diluted with 20 mL of 4N HCl in dioxane. 20 minutes later the white precipitate of N-phenylsulfonylpiperazine hydrochloride that formed was filtered and washed with cold acetone.

1-methylethyl 3-{[3-(chloromethyl)phenyl]carbonyl}-1,1-dimethyl-1,2,3,6-tetrahydroazepino[4,5-b]indole-5-carboxylate (80.0 mg, 0.177 mmol) was dissolved in 4 mL DCE. N,N′-diisopropylethylamine (344 mg, 2.66 mmol) and N-phenylsulfonylpiperazine hydrochloride (699 mg, 2.66 mmol) were added to the stirring solution. The mixture was allowed to react at room temperature overnight. The sample was filtered through a Millipore Millex-GN filter, then purified by preparative liquid chromatography using a 50%-100% gradient of ACN/H₂O with 0.05% TFA for 8 minutes. Desired fractions were combined and made basic by saturated NaHCO₃ and diluted with ethyl acetate. Organic layer was extracted with water and brine, then dried over Na₂SO₃ and filtered. The yellow solution was reduced to dryness and lyophilized overnight in ACN/H₂O to form the yellow powder (114 mg, 38% yield) of the title compound: ¹H NMR (400 MHz, CDCl₃): δ 10.67 (s, 1H), 7.83 (d, J=8.0 Hz, 1H), 7.73 (s, 3H), 7.60 (m, 3H), 7.52 (m, 3H), 7.37 (m, 4H), 7.19 (t, J=7.2 Hz, 1H), 7.09 (t, J=8.0 Hz, 1H), 5.11 (sept. J=6.4 Hz, 1H), 4.05 (br s, 2H), 3.52 (s, 2H), 3.01 (br s, 4H), 2.51 (t, J=4.4 Hz, 4H), 1.62 (s, 6H), 1.17 (s, 3H), 1.16 (s, 3H); MS (EI) for C₃₆H₄₁N₄O₅S: 641.3 (MH⁺).

Using the same or analogous synthetic techniques and/or substituting with alternative reagents, the following compounds of the invention were prepared:

1-methylethyl 3-{[3-({4-[(4-fluorophenyl)sulfonyl]piperazin-1-yl}methyl)phenyl]carbonyl}-1,1-dimethyl-1,2,3,6-tetrahydroazepino[4,5-b]indole-5-carboxylate: ¹H NMR (400 MHz, CDCl₃): δ 10.67 (s, 1H), 7.83 (d, J=8.0 Hz, 1H), 7.73 (m, 3H), 7.47 (m, 2H), 7.38 (m, 3H), 7.19 (m, 2H), 7.09 (t, J=6.8 Hz, 1H), 5.10 (sept. J=6.4 Hz, 1H), 4.08 (br s, 2H), 3.52 (s, 2H), 2.99 (br s, 4H), 2.51 (t, J=4.0 Hz, 4H), 1.62 (s, 6H), 1.17 (s, 3H), 1.15 (s, 3H); MS (EI) for C₃₆H₄₀FN₄O₅S: 659.3 (MH⁺).

1-methylethyl 3-[(3-{[4-(ethylsulfonyl)piperazin-1-yl]methyl}phenyl)carbonyl]-1,1-dimethyl-1,2,3,6-tetrahydroazepino[4,5-b]indole-5-carboxylate: ¹H NMR (400 MHz, CDCl₃): δ 10.68 (s, 1H), 7.83 (d, J=7.6 Hz, 1H), 7.76 (s, 1H), 7.55 (s, 1H), 7.48 (m, 1H), 7.40 (m, 2H), 7.31 (s, 1H), 7.19 (t, J=8.0 Hz, 1H), 7.09 (t, J=7.2 Hz, 1H), 5.12 (sept. J=6.4 Hz, 1H), 4.17 (br s, 2H), 3.60 (s, 2M), 3.27 (br s, 4H), 2.88 (q, J=6.8 Hz, 2H), 2.53 (m, 4H), 1.64 (s, 6H), 1.57 (s, 3H), 1.18 (s, 3H), 1.16 (s, 3H); MS (EI) for C₃₂H₄₁N₄O₅S: 593.3 (MH⁺).

1-methylethyl 3-[(3-([4-(cyclopropylcarbonyl)piperazin-1-yl]methyl)phenyl)carbonyl]-1,1-dimethyl-1,2,3,6-tetrahydroazepino[4,5-b]indole-5-carboxylate: ¹H NMR (400 MHz, CDCl₃): δ 10.70 (s, 1H), 7.83 (d, J=8.0 Hz, 1H), 7.79 (s, 1H), 7.55 (s, 1H), 7.51 (d, J=7.2 Hz, 1H), 7.45 (d, J=7.6 Hz, 1H), 7.40 (m, 2H), 7.19 (t, J=7.2 Hz, 1H), 7.09 (t, J=7.2 Hz, 1H), 5.11 (sept. J=6.4 Hz, 1H), 4.08 (br s, 2H), 3.63 (d, J=14.4 Hz, 4H), 3.55 (s, 2H), 2.43 (d, J=23.6 Hz, 4H), 1.70 (m, 4H), 1.64 (s, 6H), 1.17 (s, 3H), 1.16 (s, 3H), 0.98 (s, 2H), 0.75 (s, 2H); MS (EI) for C₃₄H₄₁N₄O₄: 564.4 (MH⁺).

1-methylethyl 1,1-dimethyl-3-[(3-{[4-(2-methylpropanoyl)piperazin 1-yl]methyl}phenyl)carbonyl]-1,2,3,6-tetrahydroazepino[4,5-b]indole-5-carboxylate: ¹H NMR (400 MHz, CDCl₃): δ 10.70 (s, 1H), 7.83 (d, J=8.0 Hz, 1H), 7.79 (s, 1H), 7.55 (s, 1H), 7.50 (d, J=7.6 Hz, 1H), 7.45 (d, J=8.0 Hz, 1H), 7.39 (m, 2H), 7.19 (t, J=7.6 Hz, 1H), 7.09 (t, J=7.6 Hz, 1H), 5.11 (sept. J=6.4 Hz, 1H), 4.06 (br s, 2H), 3.60 (br s, 4H), 3.54 (s, 2H), 3.48 (t, J=4.8 Hz, 2H), 2.76 (q, J=6.4 Hz, 1H), 2.41 (t, J=4.4 Hz, 4H), 1.64 (s, 6H), 1.18 (s, 3H), 1.16 (s, 3H), 1.12 (s, 3H), 1.11 (s, 3H); MS (EI) for C₃₄H₄₃N₄O₄: 571.4 (MH⁺).

1-methylethyl 1,1-dimethyl-3-[(3-{[4-(phenylcarbonyl)piperazin-1-yl]methyl}phenyl)carbonyl]-1,2,3,6-tetrahydroazepino[4,5-b]indole-5-carboxylate: ¹H NMR (400 MHz, CDCl₃): δ 10.69 (s, 1H), 7.83 (d, J=8.0 Hz, 1H), 7.78 (s, 1H), 7.52 (m, 3H), 7.40 (m, 7H), 7.19 (t, J=8.0 Hz, 1H), 7.09 (t, J=7.2 Hz, 1H), 5.09 (m, 1H), 4.06 (br s, 2H), 3.55 (s, 2H), 3.40 (br s, 4H), 2.37 (br s, 4H), 1.64 (s, 6H), 1.17 (s, 3H), 1.16 (s, 3H); MS (EI) for C₃₇H₄₁N₄O₄: 605.4 (MH⁺).

### Example 19

### Preparation of 1-Methylethyl 1,1-Dimethyl-3-{[3-(piperazin-1-ylmethyl)Phenyl]carbonyl}-1,2,3,6-Tetrahydroazepino[4,5-b]Indole-5-Carboxylate

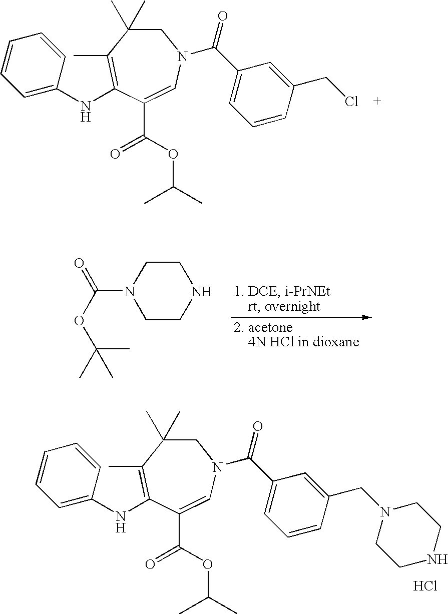

1-methylethyl 3-{[3-(chloromethyl)phenyl]carbonyl}-1,1-dimethyl-1,2,3,6-tetrahydroazepino[4,5-b]indole-5-carboxylate (80.0 mg, 0.1774 mmol) was dissolved in 3 mL DCE. N,N′-diisopropylethylamine (344 mg, 2.6610 mmol) and piperazine-1-carboxylic acid tert-butyl ester (496 mg, 2.66 mmol) were added and allowed to stir overnight at room temperature. When TLC indicated starting material was no longer present, the sample was evaporated to dryness and dissolved in 20 mL acetone. 20 mL 4N HCl in dioxane was added to the stirring solution and allowed to react at room temperature for 2 hrs. The light yellow precipitate was filtered and washed with cold acetone producing 100.1 mg (98%) of the title compound: ¹H NMR (400 MHz, CDCl₃): δ 9.30 (s, 1H), 7.79 (d, J=7.6 Hz, 1H), 7.74 (s, 1H), 7.69 (s, 1H), 7.62 (m, 3H), 7.42 (d, J=8.0 Hz, 1H), 7.12 (t, J=6.8 Hz, 1H), 7.01 (t, J=8.0 Hz, 1H), 5.13 (m, 1H), 3.54 (s, 4H), 3.30 (s, 4H), 1.62 (s, 6H), 1.20 (s, 3H), 1.18 (s, 3H), MS (EI) for C₃₀H₃₇N₄O₃: 501.2 (MH⁺).

### Example 20

### Preparation of 1-Methylethyl 1,1-Dimethyl-3-{[3-({4-[(Phenylamino)Carbonyl]piperazin-1-yl}Methyl)Phenyl]Carbonyl}-1,2,3,6-Tetrahydroazepino[4,5-b]Indole-5-Carboxylate

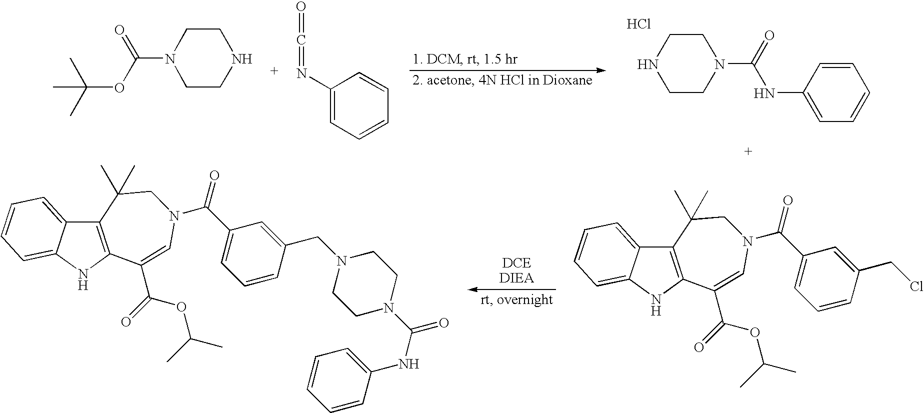

Piperazine-1-carboxylic acid tert-butyl ester (2.00 g, 10.7 mmol) was dissolved in 10 mL DCM. Phenylisocyanate (1.66 g, 14.0 mmol) was added and reaction was allowed to stir for 1.5 h. When LCMS indicated desired mass, solution was diluted with 50 mL ethyl acetate and extracted with 2×50 mL water and 1×50 mL brine. Organic layer was dried over Na₂SO₃ and filtered. The sample was evaporated to a colorless oil, which was diluted in 20 mL acetone and 20 mL 4N HCl in dioxane. 30 minutes later the white precipitate of piperazine-1-carboxylic acid phenylamine hydrochloride that formed was filtered and washed with cold acetone.

1-methylethyl 3-{[3-(chloromethyl)phenyl]carbonyl}-1,1-dimethyl-1,2,3,6-tetrahydroazepino[4,5-b]indole-5-carboxylate (100 mg, 0.221 mmol) was dissolved in 3 mL DCE. N,N′-diisopropylethylamine (860 mg, 6.65 mmol) and piperazine-1-carboxylic acid phenylamine hydrochloride (804 mg, 3.32 mmol) were added to the solution. The reaction mixture was allowed to stir at room temperature overnight. The sample was filtered through a Millipore Millex-GN filter, then purified by preparative liquid chromatography using a 20%-80% gradient of ACN/H₂O with 0.05% TFA for 15 minutes. Desired fractions were combined and made basic by saturated NaHCO₃ and diluted with ethyl acetate. Organic layer was extracted with water and brine, then dried over Na₂SO₃ and filtered. The yellow solution was reduced to dryness and lyophilized overnight in ACN/H₂O to form the yellow powder (137.3 mg, 28% yield) of the title compound: ¹H NMR (400 MHz, CDCl₃): δ 10.67 (s, 1H), 7.83 (d, J=8.4 Hz, 1H), 7.77 (s, 1H), 7.40 (m, 8H), 7.18 (m, 2H), 7.07 (m, 2H), 5.11 (sept., J=6.0 Hz, 1H), 3.51 (br s, 4H), 2.53 (br s, 4H), 1.65 (s, 6H), 1.18 (s, 3H), 1.17 (s, 3H); MS (EI) for C₃₇H₄₁N₅O₄: 620.4 (MH⁺).

Using the same or analogous synthetic techniques and/or substituting with alternative reagents, the following compounds of the invention were prepared:

1-methylethyl 3-{[3-({4-[(ethylamino)carbonyl]piperazin-1-yl}methyl)phenyl]carbonyl}-1,1-dimethyl-1,2,3,6-tetrahydroazepino[4,5-b]indole-5-carboxylate: ¹H NMR (400 MHz, CDCl₃): δ 10.69 (s, 1H), 7.83 (d, J=8.0 Hz, 1H), 7.78 (s, 1H), 7.53 (s, 1H), 7.50 (d, J=7.6 Hz, 1H), 7.45 (d, J=7.6 Hz, 1H), 7.39 (m, 2H), 7.19 (t, J=7.2 Hz, 1H), 7.09 (d, J=6.8 Hz, 1H), 5.11 (sept., J=6.4 Hz, 1H), 4.04 (br s, 2H), 3.55 (s, 2H), 3.33 (m, 4H), 2.70 (m, 2H), 2.41 (m, 4H), 1.64 (s, 6H), 1.18 (s, 3H), 1.16 (s, 3H), 1.14 (s, 3H); MS (EI) for C₃₃H₄₁N₅O₄: 572.4 (MH⁺).

### Example 24

### Preparation of 2-{[(3,4-Difluorophenyl)Carbonyl]oxy}-1-Methylethyl 3-[(3,4-Difluorophenyl)Carbonyl]-1,1-Dimethyl-1,2,3,6-Tetrahydroazepino[4,5-b]Indole-5-Carboxylate

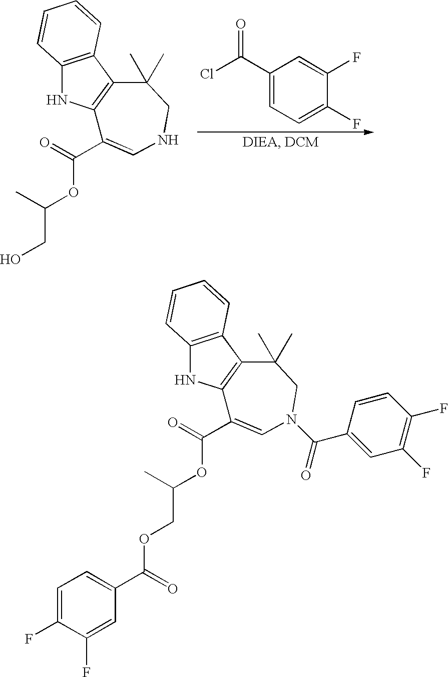

### Example 25

### Preparation of 2-Hydroxyethyl 3-[(3,4-Difluorophenyl)Carbonyl]-1,1-Dimethyl-1,2,3,6-Tetrahydroazepino[4,5-b]Indole-5-Carboxylate

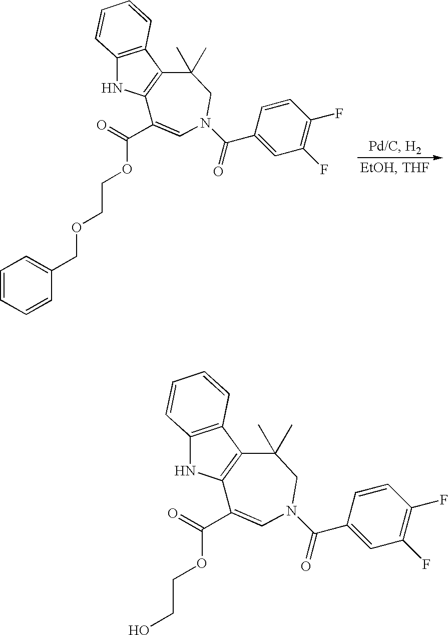

### Example 26

### Preparation of (E)-Isopopyl 1,1-dimethyl-3-(4-(3-morpholino propoxy)benzoyl)-1,2,3,6-tetrahydroazepino[4,5-b]indole-5-carboxylate

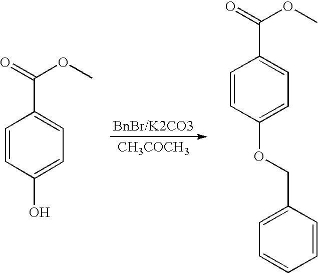

To a solution of methyl 4-hydroxybenzoate (5 g, 32.86 mmol) in acetone (50 mL) was added benzyl bromide (5.63 g, 32.86 mmol) and potassium carbonate (9.08 g, 65.72 mmol). The reaction mixture was heated to reflux overnight with stirring. After 12 h the reaction was concentrated on a rotary evaporator. The residue was dissolved in ethyl acetate and washed with water. The organics were dried over sodium sulfate, filtered, and concentrated to afford 7.29 g of the product. ¹H NMR (400 MHz, CDCl₃): 8.01-6.98 (m, 9H), 5.12 (s, 1H), 3.89 (s, 3H).

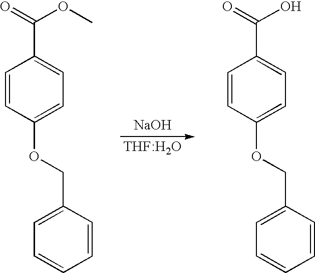

To a solution of methyl 4-(benzyloxy)benzoate (7.29 g, 30.09 mmol) in THF was added an aqueous solution of sodium hydroxide (12.0 g, 30 0 mmol/50 mL). The reaction mixture was heated to 64° C. for 4 h. After completion of the reaction (as monitored by LC/MS), the reaction mixture was neutralized with 3N aqueous HCl. The product (6.79 g) was collected by filtration.

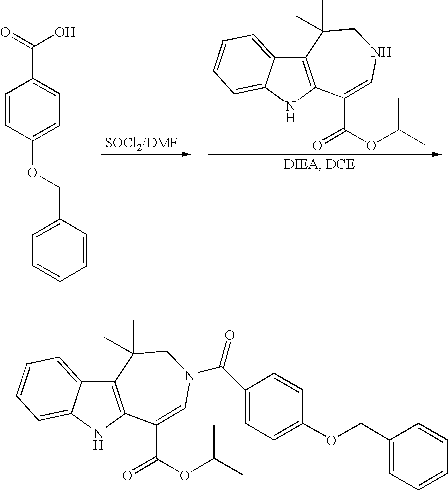

To a round bottom flask containing thionyl chloride (15 mL) was added 4-(benzyloxy)benzoic acid (1.44 g, 6.3 mmol) at room temperature. Then the reaction mixture was heated to reflux for 2 h. After removing excess thionyl chloride on a rotary evaporator, the residue was added to a solution of (E)-isopropyl 1,1-dimethyl-1,2,3,6-tetrahydroazepino[4,5-b]indole-5-carboxylate (large scale preparation in DC document) in DCE (30 mL) and 2 equivalents of diisopropyethyl amine was added to the above reaction mixture. The reaction mixture was then stirred overnight at room temperature. After aqueous workup, the product (1.4 g) was isolated by purification using silica gel column chromatography with 10% acetate/hexane as eluent: ¹H NMR (400 MHz, CDCl₃): δ 10.73 (bs, 1H), 7.89-6.90 (m, 13H), 5.13 (m, 3H), 4.09 (bs, 2H), 1.62 (s, 6H), 1.22 (d, J=6.00 Hz, 6H).

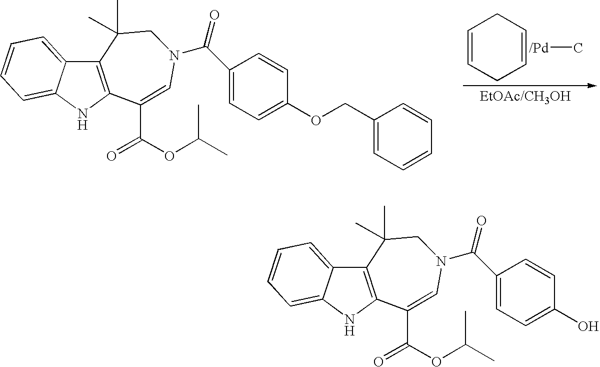

To a solution of 1-methylethyl 1,1-dimethyl-3-({4-[(phenylmethyl)oxy]phenyl}carbonyl)-1,2,3,6-tetrahydroazepino[4,5-b]indole-5-carboxylate (0.24 g, 0.47 mmol) in methanol was added 1,4-cyclohexadiene (0.378 g, 4.72 mmol) and Pd(OH)₂/C (120 mg) in a sealed tube. The reaction mixture was heated to 64° C. for 12 h. The reaction mixture was filtered and the solvent was evaporated to give the desired crude product (0.180 g, 91% yield). The material was used as such in the subsequent reaction. ¹H NMR (400 MHz, CDCl₃): δ 10.71 (bs, 1H), 7.83-6.99 (m, 9H), 5.92 (s, 1H), 5.13 (m, 1H), 4.05 (bs, 2H), 1.63 (s, 6H), 1.20 (d, J=6.4 Hz, 6H).

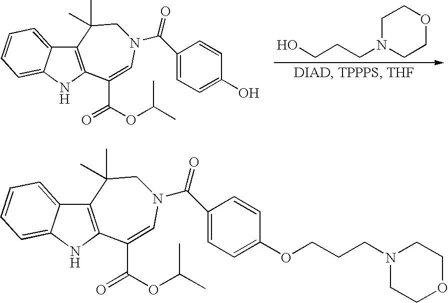

To the solution of 1-methylethyl 3-[(4-hydroxyphenyl)carbonyl]-1,1-dimethyl-1,2,3,6-tetrahydroazepino[4,5-b]indole-5-carboxylate (90 mg, 0.215 mmol) in THF (5 mL) was added 3-morpholinopropanol (62 mg, 0.43 mmol), diisopropyl azodicarboxylate (87 mg, 0.43 mmol), and triphenylphosphine polystyrene (90 mg, 0.43 mmol). The reaction mixture was stirred at room temperature overnight. After filtration, the solvent was removed on a rotary evaporator. The crude material was purified by preperative liquid chromatography using a 10%-90% gradient of ACN/H₂O with 0.05% TFA for 11 minutes. Desired fractions were combined and neutralized by partitioning between saturated aqueous NaHCO₃ and ethylacetate. The organic layer was dried over Na₂SO₄, and filtered. Concentration on a rotary evaporator gave the desired product (52 mg, 44% yield). ¹H NMR (400 MHz, CDCl₃): δ 10.72 (bs, 1H), 7.89-6.90 (m, 9H), 5.15 (m, 1H), 4.09 (bs, 2H), 4.07 (t, J=6.2 Hz, 2H), 3.72 (m, 4H), 2.52 (t, J=7.4 Hz, 2H), 2.47 (m, 4H), 1.99 (dt, J=6.2, 7.4 Hz, 2H), 1.62 (s, 6H), 1.22 (d, J=6.00 Hz, 6H); MS (EI) for C₃₂H₃₉N₃O₅: 546.2 (MH⁺).

### Example 27

### Preparation of 1-{3-[(3,4-Difluorophenyl)Carbonyl]-1,1-Dimethyl-1,2,3,6-Tetrahydroazepino[4,5-b]Indol-5-yl}Ethanone

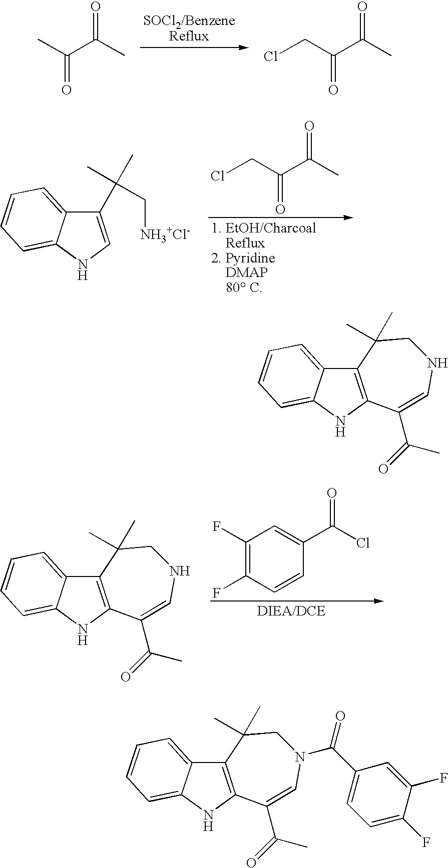

### Example 28

### Preparation of 1-Methylethyl3-({[3-(Dimethylamino)Propyl]amino}Carbonyl)-1,1-Dimethyl-1,2,3,6-Tetrahydroazepino[4,5-b]Indole-5-Carboxylate

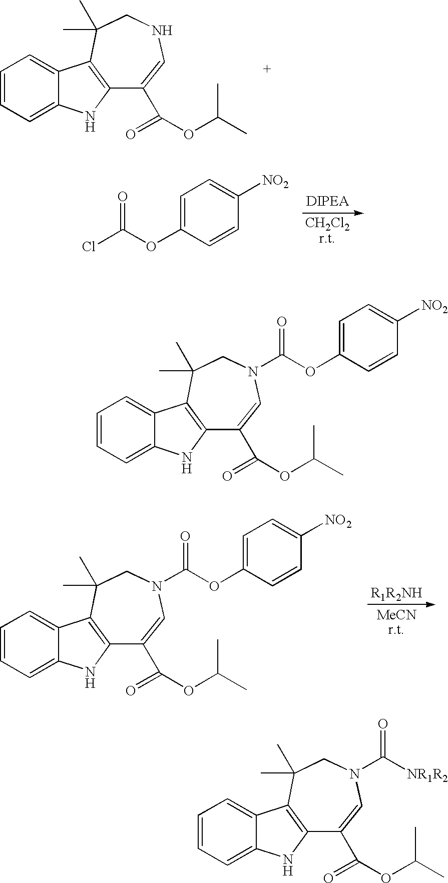

p-Nitrophenyl chloroformate (0.658 g, 3.26 mmol) was added portion wise to a solution of 1-methylethyl 1,1-dimethyl-1,2,3,6-tetrahydroazepino[4,5-b]indole-5-carboxylate (0.927 g, 3.11 mmol) and diisopropylethylamine (1.03 mL, 6.22 mmol) in anhydrous CH₂Cl₂ (10 mL) at room temperature. After 12 hours, the reaction was concentrated in vacuo and the residue purified on SiO₂ (10% ethyl acetate:hexanes) to give 5-(1-methylethyl) 3-(4-nitrophenyl) 1,1-dimethyl-1,6-dihydroazepino[4,5-b]indole-3,5(2H)-dicarboxylate (1.24 g, 86% yield) as a light yellow solid. A solution of 5-(1′-methylethyl) 3-(4-nitrophenyl) 1,1-dimethyl-1,6-dihydroazepino[4,5-b]indole-3,5(2H)-dicarboxylate (0.101 g, 0.218 mmol) and N,N′-dimethylpropanediamine (110 μL, 0.872 mmol) in anhydrous acetonitrile (1.0 mL) was stirred at room temperature under nitrogen atmosphere. After one hour or disappearance of starting material, the reaction was concentrated in vacuo and purified on reverse phase HPLC (25 mM ammonium acetate: acetonitrile, 20-90% gradient). The product was collected and lyophilized to give the title compound (82 mg, 89% yield) as a yellow solid: ¹H NMR (400 MHz, DMSO-d₆): δ 8.12 (s, 1H), 7.78 (t, J=5.2 Hz, 1H), 7.69 (d, J=8.4 Hz, 1H), 7.51 (d, J=8.0 Hz, 1H), 7.01 (dt, J=7.2, 1.2 Hz, 1H), 6.96 (dt, J=7.2, 1.2 Hz, 1H), 5.12 (sept, J=6.0 Hz, 1H), 3.17 (m, 2H), 2.25 (t, J=7.2 Hz, 2H), 2.12 (s, 6H), 1.61 (quint, J=6.8 Hz, 2H), 1.44 (bs, 6H), 1.33 (d, J=6.0 Hz, 6H); MS (EI) for C₂₄H₃₄N₄O₃: 427.3 (MH⁺).

Using the same or analogous synthetic techniques and/or substituting with alternative reagents, the following compounds of the invention were prepared:

1-methylethyl 1,1-dimethyl-3-(piperidin-1-ylcarbonyl)-1,2,3,6-tetrahydroazepino[4,5-b]indole-5-carboxylate: ¹H NMR (400 MHz, DMSO-d₆): δ 10.85 (bs, 1H), 7.74-6.93 (m, 5H), 5.13 (m, 1H), 3.98 (bs, 2H), 3.71 (bs, 2H), 3.25 (m, 4H), 1.60-1.50 (m, 6H), 1.43 (s, 6H), 1.32 (d, J=6.4 Hz, 6H); MS (ET) for C₂₄H₃₁N₃O₃: 410.1 (MH¹).

1-methylethyl 1,1-dimethyl-3-({[4-(4-methylpiperzin-1-yl)phenyl]amino}carbonyl-1,2,3,6-tetrahydroazepinoe[4,5-b]indole-5-carboxylate: ¹H NMR (400 MHz, DMSO-d₆): δ 10.95 (s, 1H), 9.49 (s, 1H), 8.10 (s, 1H), 7.71 (d, J=8.0 Hz, 1H), 7.54 (d, J=7.6 Hz, 1H), 7.33 (m, 2H), 7.04 (dt, J=7.2, 1.2 Hz, 1H), 6.94 (dt, J=7.2, 1.2 Hz, 1H), 6.90 (m, 2H), 5.14 (sept, J=6.4 Hz, 1H), 3.07 (m, 4H), 2.44 (m, 4H), 2.21 (s, 3H), 1.90 (s, 2H), 1.47 (bs, 6H), 1.33 (d, J=6.4 Hz, 6H); MS (EI) for C₃₀H₃₇N₅O₃: 516.3 (MH⁺).

1-methylethyl 3-({[2-(dimethylamino)ethyl]amino}carbonyl)-1,1-dimethyl-1,2,3,6-tetrahydroazepino[4,5-b]indole-5-carboxylate: ¹H NMR (400 MHz, DMSO-d₆): δ 10.91 (s, 1H), 8.13 (s, 1H), 7.69 (d, J=8.4 Hz, 1H), 7.52 (d, J=8.4 Hz, 1H), 7.02 (t, J=7.6 Hz, 1H), 6.93 (t, J=8.8 Hz, 1H), 6.90 (m, 2H), 5.12 (sept, J=6.4 Hz, 1H), 3.30 (m, 4H), 2.57 (m, 2H), 2.33 (bs, 2H), 1.45 (bs, 6H), 1.33 (d, J=6.4 Hz, 6H); MS (EI) for C₂₃H₃₂N₄O₃: 413.3 (MH⁺).

1-methylethyl1,1-dimethyl-3-{[(3-morpholin-4-ylpropyl)amino]carbonyl}-1,2,3,6-tetrahydroazepino[4,5-b]indole-5-carboxylate: ¹H NMR (400 MHz, DMSO-d₆): δ 10.90 (s, 1H), 8.11 (s, 1H), 7.69 (d, J=8.4 Hz, 1H), 7.51 (d, J=8.4 Hz, 1H), 7.01 (t, J=7.6 Hz, 1H), 6.96 (t, J=8.8 Hz, 1H), 5.12 (sept, J=6.4 Hz, 1H), 3.74 (bs, 2H), 3.55 (bs, 4H), 3.19 (m, 3H), 2.31 (m, 5H), 1.63 (m, 2H), 1.44 (bs, 6H), 1.33 (d, J=6.4 Hz, 6H); MS (EI) for C₂₆H₃₆N₄O₄: 469.3 (MH⁺).

1-methylethyl1,1-dimethyl-3-{[(2-morpholin-4-ylethyl)amino]carbonyl}-1,2,3,6-tetrahydroazepino[4,5-b]indole-5-carboxylate: ¹H NMR (400 MHz, DMSO-d₆): δ 10.92 (s, 1H), 8.11 (s, 1H), 7.70 (d, J=8.0 Hz, 1H), 7.61 (bt, J=5.2 Hz, 1H), 7.52 (d, J=8.0 Hz, 1H), 7.02 (t, J=7.2 Hz, 1H), 6.93 (t, J=7.6 Hz, 1H), 5.13 (sept, J=6.4 Hz, 1H), 3.73 (bs, 2H), 3.55 (m, 4H), 3.26 (m, 2H), 2.43 (m, 6H), 1.45 (bs, 6H), 1.33 (d, J=6.0 Hz, 6H); MS (EI) for C₂₅H₃₄N₄O₄: 455.4 (MH⁺).

1-methylethyl1,1-dimethyl-3-[(propylamino)carbonyl]-1,2,3,6-tetrahydroazepino[4,5-b]indole-5-carboxylate: ¹H NMR (400 MHz, DMSO-d₆): δ 10.91 (s, 1H), 8.14 (s, 1H), 7.70 (m, 2H), 7.51 (d, J=8.0 Hz, 1H), 7.01 (dt, J=7.2, 1.2 Hz, 1H), 6.93 (dt, J=7.2, 1.2 Hz, 1H), 5.11 (sept, J=6.0 Hz, 1H), 3.73 (bs, 2H), 3.11 (m, 2H), 1.48 (m, 2H), 1.44 (bs, 6H), 1.33 (d, J=6.0 Hz, 6H), 0.86 (t, J=7.2 Hz, 2H); MS (EI) for C₂₂H₂₉N₃O₃: 384.4 (MH⁺).

1-methylethyl3-({[2-(diethylamino)ethyl](ethyl)amino}carbonyl)-1,1-dimethyl-1,2,3,6-tetrahydroazepino[4,5-b]indole-5-carboxylate: ¹H NMR (400 MHz, DMSO-d₆): δ 10.86 (s, 1H), 7.73 (s, 1H), 7.70 (d, J=7.6 Hz, 1H), 7.53 (d, J=7.6 Hz, 1H), 7.04 (t, J=8.0 Hz, 1H), 6.95 (t, J=8.0 Hz, 1H), 5.14 (sept, J=6.4 Hz, 1H), 3.70 (bs, 2H), 3.22 (m, 4H), 2.55 (bt, J=6.8 Hz, 2H), 2.43 (q, J=6.8 Hz, 4H), 1.43 (bs, 6H), 1.31 (d, J=6.4 Hz, 6H), 1.12 (t, J=6.8 Hz, 3H), 0.91 (t, J=6.8 Hz, 6H); MS (EI) for C₂₇H₄₀N₄O₃: 469.2 (MH⁺).

1-methylethyl1,1-dimethyl-3-[(4-pyrrolidin-1-ylpiperidin-1-yl)carbonyl]-1,2,3,6-tetrahydroazepino[4,5-b]indole-5-carboxylate: ¹H NMR (400 MHz, DMSO-d₆): δ 10.84 (s, 1H), 7.73 (s, 1H), 7.70 (d, J=8.4 Hz, 1H), 7.52 (d, J=8.4 Hz, 1H), 7.04 (t, J=8.4 Hz, 1H), 6.93 (t, J=8.4 Hz, 1H), 5.13 (sept, J=6.4 Hz, 1H), 3.71 (bs, 2H), 3.64 (bd, J=12.4 Hz, 2H), 2.95 (bt, J=12.4 Hz, 4H), 2.55 (m, 2H), 2.41 (m, 3H), 1.68 (bs, 4H), 1.43 (bs, 6H), 1.39 (m, 2H), 1.32 (d, J=6.4 Hz, 6H); MS (EI) for C₂₈H₃₈N₄O₃: 479.4 (MH⁺).

1-methylethyl1,1-dimethyl-3-({4-[(1-methylpiperidin-4-yl)methyl]piperazin-1-yl}carbonyl)-1,2,3,6-tetrahydroazepino[4,5-b]indole-5-carboxylate: ¹H NMR (400 MHz, DMSO-d₆): δ 10.85 (s, 1H), 7.75 (s, 1H), 7.70 (d, J=8.0 Hz, 1H), 7.52 (d, J=8.4 Hz, 1H), 7.03 (dt, J=7.2, 0.8 Hz, 1H), 6.94 (dt, J=6.8, 0.8 Hz, 1H), 5.13 (sept, J=6.0 Hz, 1H), 3.70 (bs, 2H), 3.26 (bs, 4H), 2.70 (m, 2H), 2.34 (bs, 4H), 2.11 (s, 3H), 2.10 (m, 2H), 1.78 (bt, J=11.6 Hz, 2H), 1.61 (bd, J=11.2 Hz, 2H), 1.43 (bs, 6H), 1.33 (d, J=6.4 Hz, 6H), 1.06 (m, 3H); MS (EI) for C₃₀H₄₃N₅O₃: 522.5 (MH⁺).

1-methylethyl3-{[4-(diethylamino)piperidin-1-yl]carbonyl}-1,1-dimethyl-1,2,3,6-tetrahydroazepino[4,5-b]indole-5-carboxylate: ¹H NMR (400 MHz, DMSO-d₆): δ 10.85 (s, 1H), 7.77 (s, 1H), 7.70 (d, J=8.4 Hz, 1H), 7.52 (d, J=8.0 Hz, 1H), 7.03 (dt, J=7.2, 1.2 Hz, 1H), 6.95 (dt, J=6.8, 0.8 Hz, 1H), 5.14 (sept, J=6.4 Hz, 1H), 3.71 (bd, J=11.6 Hz, 4H), 3.26 (bs, 4H), 2.84 (bt, J=11.6 Hz, 4H), 2.68 (m, 2H), 2.45 (q, J=7.2 Hz, 4H), 1.69 (bd, J=10.8 Hz, 2H), 1.43 (bs, 6H), 1.38 (m, 1H), 1.32 (d, J=6.4 Hz, 6H), 0.94 (t, J=7.2 Hz, 6H); MS (EI) for C₂₈H₄₀N₄O₃: 481.3 (MH⁺).

1-methylethyl1,1-dimethyl-3-{[4-(2-oxo-2-pyrrolidin-1-ylethyl)piperazin-1-yl]carbonyl}-1,2,3,6-tetrahydroazepino[4,5-b]indole-5-carboxylate: ¹H NMR (400 MHz, DMSO-d₆): δ 10.86 (s, 1H), 7.75 (s, 1H), 7.71 (d, J=8.0 Hz, 1H), 7.52 (d, J=8.0 Hz, 1H), 7.04 (t, J=8.2 Hz, 1H), 6.95 (t, J=8.2 Hz, 1H), 5.13 (sept, J=6.0 Hz, 1H), 3.71 (bs, 2H), 3.52-3.19 (m, 14H), 3.14 (bs, 2H), 1.83 (m, 2H), 1.74 (m, 2H), 1.43 (bs, 6H), 1.34 (d, J=6.0 Hz, 6H); MS (EI) for C₂₉H₃₉N₅O₄: 522.3 (MH⁺).

1-methylethyl 3-({4-[2-(diethylamino)ethyl]piperazin-1-yl}carbonyl)-1,1-dimethyl-1,2,3,6-tetrahydroazepino[4,5-b]indole-5-carboxylate: ¹H NMR (400 MHz, DMSO-d₆): δ 10.83 (s, 1H), 7.73 (s, 1H), 7.68 (d, J=8.4 Hz, 1H), 7.50 (d, J=8.0 Hz, 1H), 7.01 (t, J=8.0 Hz, 1H), 6.92 (t, J=8.4 Hz, 1H), 5.11 (sept, J=6.4 Hz, 1H), 3.68 (bs, 2H), 3.24 (bs, 4H), 2.46-2.32 (m, 12H), 1.40 (bs, 6H), 1.31 (d, J=6.4 Hz, 6H), 0.89 (t, J=6.8 Hz, 6H); MS (EI) for C₂₉H₄₃N₅O₃: 510.3 (MH⁺).

1-methylethyl3-{[3-(dimethylamino)piperidin-1-yl]carbonyl}-1,1-dimethyl-1,2,3,6-tetrahydroazepino[4,5-b]indole-5-carboxylate: ¹H NMR (400 MHz, DMSO-d₆): δ 10.85 (s, 1H), 7.72 (s, 1H), 7.71 (d, J=8.4 Hz, 1H), 7.55 (d, J=8.0 Hz, 1H), 7.06 (t, J=8.0 Hz, 1H), 6.95 (t, J=8.4 Hz, 1H), 5.11 (sept, J=6.0 Hz, 1H), 3.91 (bs, 2H), 3.66 (m, 1H), 3.59 (m, 1H), 3.50 (m, 1H), 2.80 (m, 2H), 2.21 (m, 1H), 2.12 (s, 6H), 1.85 (m, 1H), 1.75 (m, 1H), 1.43 (m, 7H), 1.31 (t, J=6.4 Hz, 6H); MS (EI) for C₂₆H₃₆N₄O₃: 453.3 (MH⁺).

1-methylethyl1,1-dimethyl-3-{[4-(4-methylpiperazin-1-yl)piperidin-1-yl]carbonyl}-1,2,3,6-tetrahydroazepino[4,5-b]indole-5-carboxylate: ¹H NMR (400 MHz, DMSO-d₆): δ 10.84 (s, 1H), 7.74 (s, 1H), 7.70 (d, J=8.0 Hz, 1H), 7.50 (d, J=8.0 Hz, 1H), 7.04 (t, J=8.0 Hz, 1H), 6.96 (t, J=8.4 Hz, 1H), 5.14 (sept, J=6.0 Hz, 1H), 3.72 (bs, 2H), 3.68 (bs, 2H), 3.40 (bs, 2H), 2.85 (m, 3), 2.45 (m, 4H), 2.27 (m, 3H), 2.12 (s, 3H), 1.77 (m, 2H), 1.42 (bs, 6H), 1.31 (d, J=6.0 Hz, 6H); MS (EI) for C₂₉H₄₁N₅O₃: 508.3 (MH⁺).

1-methylethyl1,1-dimethyl-3-(morpholin-4-ylcarbonyl)-1,2,3,6-tetrahydroazepino[4,5-b]indole-5-carboxylate: ¹H NMR (400 MHz, DMSO-d₆): δ 10.83 (s, 1H), 7.76 (s, 1H), 7.69 (d, J=8.0 Hz, 1H), 7.51 (d, J=7.6 Hz, 1H), 7.01 (dt, J=7.2, 1.2 Hz, 1H), 6.93 (dt, J=6.8, 1.2 Hz, 1H), 5.11 (sept, J=6.0 Hz, 1H), 3.71 (bs, 2H), 3.59 (m, 4H), 3.26 (m, 4H), 1.42 (bs, 6H), 1.32 (d, J=6.4 Hz, 6H); MS (EI) for C₂₃H₂₉N₃O₄: 412.2 (MH⁺).

1-methylethyl3-({3-[(dimethylamino)methyl]piperidin-1-yl}carbonyl)-1,1-dimethyl-1,2,3,6-tetrahydroazepino[4,5-b]indole-5-carboxylate: ¹H NMR (400 MHz, DMSO-d₆): δ 10.86 (s, 1H), 7.75 (s, 1H), 7.71 (d, J=8.0 Hz, 1H), 7.53 (d, J=8.0 Hz, 1H), 7.04 (dt, J=7.2, 1.2 Hz, 1H), 6.94 (dt, J=6.8, 1.2 Hz, 1H), 5.10 (sept, J=6.4 Hz, 1H), 3.87 (bs, 2H), 3.72 (m, 1H), 3.59 (m, 1H), 2.83 (m, 1H), 2.54 (m, 1H), 2.06 (m, 1H), 2.02 (s, 6H), 1.96 (m, 1H), 1.70 (m, 3H), 1.44 (s, 3H), 1.42 (s, 3H), 1.32 (d, J=6.0 Hz, 6H), 1.11 (m, 2H); MS (EI) for C₂₇H₃₈N₄O₃: 467.3 (MH⁺).

1-methylethyl3-({(3S)-3-[(dimethylamino)methyl]piperidin-1-yl}carbonyl)-1,1-dimethyl-1,2,3,6-tetrahydroazepino[4,5-b]indole-5-carboxylate: ¹H NMR (400 MHz, DMSO-d₆): δ 10.85 (s, 1H), 7.74 (s, 1H), 7.69 (d, J=8.0 Hz, 1H), 7.51 (d, J=8.4 Hz, 1H), 7.03 (t, J=7.2 Hz, 1H), 6.94 (t, J=8.0 Hz, 1H), 5.09 (sept, J=6.4 Hz, 1H), 3.85 (bs, 2H), 3.72 (m, 1H), 3.57 (m, 2H), 2.83 (m, 2H), 2.55 (m, 1H), 2.07 (m, 6H), 1.71 (m, 3H), 1.43 (s, 3H), 1.41 (s, 3H), 1.31 (d, J=6.0 Hz, 6H), 1.11 (m, 2H); MS (EI) for C₂₇H₃₈N₄O₃: 467.3 (MH⁺).

1-methylethyl3-({(3R)-3-[(dimethylamino)methyl]piperidin-1-yl}carbonyl)-1,1-dimethyl-1,2,3,6-tetrahydroazepino[4,5-b]indole-5-carboxylate: ¹H NMR (400 MHz, DMSO-d₆): δ 10.85 (s, 1H), 7.75 (s, 1H), 7.70 (d, J=8.4 Hz, 1H), 7.53 (d, J=8.4 Hz, 1H), 7.04 (dt, J=7.2, 1.2 Hz, 1H), 6.95 (dt, J=8.0, 1.2 Hz, 1H), 5.10 (sept, J=6.0 Hz, 1H), 3.88 (bs, 2H), 3.72 (m, 1H), 3.59 (m, 1H), 2.83 (m, 2H), 2.53 (m, 1H), 1.99 (m, 7H), 1.73 (m, 3H), 1.44 (s, 3H), 1.42 (s, 3H), 1.31 (d, J=6.0 Hz, 6H), 1.09 (m, 2H); MS (EI) for C₂₇H₃₅N₄O₃: 467.3 (MH⁺).

1-methylethyl1,1-dimethyl-3-{[3-(morpholin-4-ylmethyl)piperidin-1-yl]carbonyl}-1,2,3,6-tetrahydroazepino[4,5-b]indole-5-carboxylate: ¹H NMR (400 MHz, DMSO-d₆): δ 10.84 (s, 1H), 7.72 (s, 1H), 7.70 (d, J=8.4 Hz, 1H), 7.52 (d, J=8.4 Hz, 1H), 7.01 (dt, J=7.2, 1.2 Hz, 1H), 6.94 (dt, J=8.0, 1.2 Hz, 1H), 5.09 (sept, J=6.0 Hz, 1H), 3.80-3.57 (m, 4H), 3.26 (m, 3H), 2.81 (m, 2H), 2.53 (m, 1H), 2.29-2.03 (m, 6H), 1.68 (m, 3H), 1.44 (s, 3H), 1.42 (s, 3H), 1.31 (d, J=6.0 Hz, 6H), 1.09 (m, 2H); MS (EI) for C₂₉H₄₀N₄O₄: 509.4 (MH⁺).

1-methylethyl3-{[3-({[3-(dimethylamino)propyl]oxy}methyl)piperidin-1-yl]carbonyl}-1,1-dimethyl-1,2,3,6-tetrahydroazepino[4,5-b]indole-5-carboxylate: ¹H NMR (400 MHz, CDCl₃): δ 10.68 (s, 1H), 7.76 (d, J=8.0 Hz, 1H), 7.68 (s, 1H), 7.52 (d, J=8.0 Hz, 1H), 7.15 (t, J=7.2 Hz, 1H), 7.05 (t, J=8.0 Hz, 1H), 5.22 (sept, J=6.4 Hz, 1H), 3.97 (m, 2H), 3.67 (m, 4H), 3.52 (d, J=13.2 Hz, 2H), 3.44 (dd, J=13.6, 5.6 Hz, 1H), 3.26 (m, 2H), 3.20 (s, 3H), 3.19 (s, 3H), 3.03 (m, 2H), 2.92 (m, 1H), 2.15 (m, 1H), 1.98 (m, 2H), 1.54 (s, 3H), 1.51 (s, 3H), 1.46 (m, 2H), 1.37 (d, J=6.4 Hz, 6H); MS (EI) for C₃₀H₄₄N₄O₄: 525.3 (MH⁺).

1-methylethyl1,1-dimethyl-3-{[(3R)-3-(morpholin-4-ylmethyl)piperidin-1-yl]carbonyl}-1,2,3,6-tetrahydroazepino[4,5-b]indole-5-carboxylate: ¹H NMR (400 MHz, DMSO-d₆): δ 10.85 (s, 1H), 7.73 (s, 1H), 7.70 (d, J=8.0 Hz, 1H), 7.54 (d, J=8.0 Hz, 1H), 7.03 (t, J=7.2 Hz, 1H), 6.95 (t, J=8.0 Hz, 1H), 5.10 (sept, J=6.4 Hz, 1H), 3.88 (bs, 2H), 3.76 (m, 2H), 3.62 (m, 1H), 3.29 (m, 3H), 2.81 (m, 1H), 2.55 (m, 1H), 2.24 (m, 2H), 2.12-2.07 (m, 3H), 1.73-1.62 (m, 4H), 1.44 (s, 3H), 1.42 (s, 3H), 1.32 (d, J=6.4 Hz, 6H), 1.10 (m, 2H); MS (EI) for C₂₉H₄₀N₄O₄: 509.4 (MH⁺).

1-methylethyl1,1-dimethyl-3-{[(3R)-3-(piperidin-1-ylmethyl)piperidin-1-yl]carbonyl}-1,2,3,6-tetrahydroazepino[4,5-b]indole-5-carboxylate: ¹H NMR (400 MHz, DMSO-d₆): δ 10.85 (s, 1H), 7.75 (s, 1H), 7.70 (d, J=8.0 Hz, 1H), 7.53 (d, J=8.0 Hz, 1H), 7.02 (t, J=7.2 Hz, 1H), 6.94 (t, J=8.0 Hz, 1H), 5.10 (sept, J=6.4 Hz, 1H), 3.86-3.61 (m, 6H), 2.80 (m, 1H), 2.54 (m, 1H), 2.21 (m, 2H), 2.02 (m, 4H), 1.65 (m, 4H), 1.45 (s, 3H), 1.42 (s, 3H), 1.41 (m, 1H), 1.32 (d, J=6.4 Hz, 6H), 1.17 (m, 4H); MS (EI) for C₃₀H₄₂N₄O₃: 507.5 (MH⁺).

1-methylethyl1,1-dimethyl-3-{[4-(phenylmethyl)-1,4-diazepan-1-yl]carbonyl}-1,2,3,6-tetrahydroazepino[4,5-b]indole-5-carboxylate: ¹H NMR (400 MHz, DMSO-d₆): δ 10.83 (s, 1H), 7.72 (s, 1H), 7.71 (d, J=8.0 Hz, 1H), 7.52 (d, J=8.0 Hz, 1H), 7.22 (m, 5H), 7.04 (t, J=7.2 Hz, 1H), 6.95 (t, J=8.0 Hz, 1H), 5.11 (sept, J=6.4 Hz, 1H), 3.72 (bs, 2H), 3.57 (s, 2H), 3.38 (m, 4H), 2.67 (m, 2H), 2.54 (m, 2H), 1.79 (m, 2H), 1.46 (s, 6H), 1.32 (d, J=6.4 Hz, 6H); MS (EI) for C₃₁H₃₈N4O₃: 515.4 (MH⁺).

1-methylethyl3-[(3′R)-1,3′-bipiperidin-1′-ylcarbonyl]-1,1-dimethyl-1,2,3,6-tetrahydroazepino[4,5-b]indole-5-carboxylate: ¹H NMR (400 MHz, DMSO-d₆): δ 10.86 (s, 1H), 7.71 (s, 1H), 7.71 (d, J=8.0 Hz, 1H), 7.53 (d, J=8.0 Hz, 1H), 7.04 (t, J=7.2 Hz, 1H), 6.95 (t, J=8.0 Hz, 1H), 5.13 (sept, J=6.4 Hz, 1H), 3.91 (bs, 2H), 3.68 (m, 2H), 3.60-3.45 (m, 3H), 2.80 (m, 3H), 2.41 (m, 4H), 1.75 (m, 3H), 1.44 (s, 3H), 1.42 (s, 3H), 1.41 (m, 3H), 1.32 (d, J=6.4 Hz, 6H); MS (EI) for C₂₉H₄₀N₄O₃: 493.4 (MH⁺).

1-methylethyl1,1-dimethyl-3-(pyrrolidin-1-ylcarbonyl)-1,2,3,6-tetrahydroazepino[4,5-b]indole-5-carboxylate: ¹H NMR (400 MHz, DMSO-d₆): δ 10.81 (bs, 1H), 7.74-6.92 (m, 5H), 5.08 (m, 1H), 3.67 (bs, 2H), 3.71 (bs, 2H), 3.29 (s, 4H), 1.60-1.50 (m, 6H), 1.43 (s, 6H), 1.32 (d, J=6.4 Hz, 6H), MS (EI) for C₂₄H₃₁N₃O₃: 410.1 (MH⁺).

1-methylethyl1,1-dimethyl-3-[(4-methylpiperazin-1-yl)carbonyl]-1,2,3,6-tetrahydroazepino[4,5-b]indole-5-carboxylate: ¹H NMR (400 MHz, DMSO-d₆): δ 10.85 (bs, 1H), 7.75-6.93 (m, 5H), 5.13 (m, 1H), 3.71 (bs, 2H), 3.71 (bs, 2H), 3.27 (bs, 4H), 2.32 (bs, 4H), 2.17 (s, 3H), 1.43 (s, 6H), 1.34 (d, J=6.4 Hz, 6H); MS (EI) for C₂₄H₃₂N₄O₃: 425.4 (MH⁺).

1-methylethyl3-[(4-ethylpiperazin-1-yl)carbonyl]-1,1-dimethyl-1,2,3,6-tetrahydroazepino[4,5-b]indole-5-carboxylate: ¹H NMR (400 MHz, DMSO-d₆): δ 10.83 (bs, 1H), 7.74-6.93 (m, 5H), 5.11 (m, 1H), 3.69 (bs, 2H), 3.25 (bs, 4H), 2.36 (bs, 4H), 2.32 (bs, 4H), 2.30 (q, J=7.6 Hz, 2H), 1.41 (s, 6H), 1.31 (d, J=6.4 Hz, 6H), 0.955 (t, J=7.6 Hz, 3H); MS (EI) for C₂₅H₃₄N₄O₃: 439.4 (MH⁺).

1-methylethyl1,1-dimethyl-3-(piperazin-1-ylcarbonyl)-1,2,3,6-tetrahydroazepino[4,5-b]indole-5-carboxylate: ¹H NMR (400 MHz, DMSO-d₆): δ 10.83 (bs, 1H), 7.73-6.92 (m, 5H), 5.11 (m, 1H), 3.69 (bs, 2H), 3.16 (bs, 4H), 2.67 (bs, 4H), 1.41 (s, 6H), 1.31 (d, J=6.4 Hz, 6H); MS (EI) for C₂₃H₃₀N₄O₃: 425.4 (MH⁺).

1-methylethyl 1,1-dimethyl-3-{[4-(1-methylethyl)piperazin-1-yl]carbonyl}-1,2,3,6-tetrahydroazepino[4,5-b]indole-5-carboxylate: ¹H NMR (400 MHz, DMSO-d₆): δ 10.84 (bs, 1H), 7.75-6.92 (m, 5H), 5.11 (m, 1H), 3.69 (bs, 2H), 3.23 (bs, 4H), 2.64 (m, 1H), 2.43 (bs, 4H), 1.41 (s, 6H), 1.31 (d, J=6.4 Hz, 6H), 0.92 (d, J=6.8 Hz, 6H); MS (EI) for C₂₆H₃₆N₄O₃: 453.4 (MH⁺).

1-methylethyl 1,1-dimethyl-3-[(4-propylpiperazin-1-yl)carbonyl]-1,2,3,6-tetrahydroazepino[4,5-b]indole-5-carboxylate: ¹H NMR (400 MHz, DMSO-d₆): δ 10.83 (bs, 1H), 7.73-6.92 (m, 5H), 5.11 (m, 1H), 3.68 (bs, 2H), 3.25 (bs, 4H), 2.35 (bs, 4H), 2.20 (t, J=7.6 Hz, 2H), 1.41 (s, 6H), 1.38 (m, 2H), 1.31 (d, J=6.4 Hz, 6H), 0.81 (t, J=7.6 Hz, 3H); MS (EI) for C₂₆H₃₆N₄O₃: 453.2 (MH⁺).

1-methylethyl 9-fluoro-1,1-dimethyl-3-[(4-methylpiperazin-1-yl)carbonyl]-1,2,3,6-tetrahydroazepino[4,5-b]indole-5-carboxylate: ¹H NMR (400 MHz, DMSO-d₆): δ 10.96 (bs, 1H), 7.76 (s, 1H), 7.52-6.87 (m, 3H), 5.11 (m, 1H), 3.68 (bs, 2H), 3.27 (bs, 4H), 2.37 (bs, 4H), 2.20 (s, 3H), 1.38 (s, 6H), 1.31 (d, J=6.4 Hz, 6H); MS (EI) for C₂₄H₃₁FN₄O₃: 443.4 (MH⁺).

1-Methylethyl3-(azepan-1-ylcarbonyl)-1,1-dimethyl-1,2,3,6-tetrahydroazepino[4,5-b]indole-5-carboxylate: ¹H NMR (400 MHz, CDCl₃): δ 10.74 (s, 1H), 7.79 (d, 2H), 7.39 (d, 1H), 7.18 (t, 1H), 7.06 (t, 1H), 5.21 (m, 1H), 3.82 (bs, 2H), 3.43 (m, 4H), 1.79 (s, 4H), 1.62 (s, 4H), 1.58 (s, 6H), 1.38 (d, 6H); MS (EI) for C25H₃₃N₃O₃: 424.2 (MH⁺).

1-Methylethyl 1,1-dimethyl-3-{[(1S,4S)-5-methyl-2,5-diazabicyclo[2.2.1]hept-2-yl]carbonyl}-1,2,3,6-tetrahydroazepino[4,5-b]indole-5-carboxylate: ¹H NMR (400 MHz, CDCl₃): δ 10.68 (s, 1H), 7.80 (m, 2H), 7.36 (d, 1H), 7.15 (t, 1H), 7.05 (t, 1H), 5.23 (m, 1H), 4.28 (bs, 2H), 3.41 (m, 4H), 2.95 (d, 1H), 2.72 (d, 1H), 2.38 (s, 1H), 1.89 (d, 1 μM), 1.68 (d, 1H), 1.53 (d, J=15.64 Hz, 6H), 1.36 (t, 6H); MS (EI) for C₂₅H₃₂N₄O₃: 437.3 (MH⁺).

1-Methylethyl1,1-dimethyl-3-[(4-methyl-1,4-diazepan-1-yl)carbonyl]-1,2,3,6-tetrahydroazepino[4,5-b]indole-5-carboxylate: ¹H NMR (400 MHz, CDCl₃): δ 10.68 (s, 1H), 7.77 (d, J=8.21 Hz, 1H), 7.69 (s, 1H), 7.37 (d, J=8.21 Hz, 1H), 7.15 (t, 1H), 7.06 (t, 1H), 5.23 (m, 1H), 3.84 (bs, 2H), 3.67 (s, 2H), 3.43 (t, 2H), 2.93 (s, 2H), 2.72 (s, 2H), 2.47 (s, 3H), 2.08 (s, 2H), 1.54 (s, 6H), 1.36 (d, 6H); MS (EI) for C₂₅H₃₄N₄O₃: 439.2 (MH⁺).

1-Methylethyl3-[(cyclopentylamino)carbonyl]-1,1-dimethyl-1,2,3,6-tetrahydroazepino[4,5-b]indole-5-carboxylate: ¹H NMR (400 MHz, CDCl₃): δ 10.69 (s, 1H), 8.00 (s, 1H), 7.78 (d, 1H), 7.35 (d, 1H), 7.14 (t, 1H), 7.05 (t, 1H), 5.22 (m, 1H), 4.76 (d, 1H), 4.18 (m, 1H), 3.82 (bs, 2H), 2.04 (m, 2H), 1.67 (m, 2H), 1.54 (s, 6H), 1.45 (m, 2H), 1.38 (d, 6H); MS (EI) for C₂₄H₃₁N₃O₃: 410.3 (MH⁺).

1-Methylethyl 3-[(cyclohexylamino)carbonyl]-1,1-dimethyl-1,2,3,6-tetrahydroazepino[4,5-b]indole-5-carboxylate: ¹H NMR (400 MHz, CDCl₃): δ 10.69 (s, 1H), 8.02 (s, 1H), 7.78 (d, 1H), 7.36 (d, 1H), 7.15 (t, 1H), 7.05 (t, 1H), 5.22 (m, 1H), 4.71 (d, 1H), 3.79 (bs, 2H), 3.74 (m, 1H), 2.00 (m, 2H), 1.72 (m, 2H), 1.63 (m, 1H), 1.55 (s, 6H), 1.43 (m, 1H), 1.38 (d, 6H), 1.21 (m, 4H); MS (EI) for C₂₅H₃₃N₃O₃: 424.4 (MH⁺).

1-Methylethyl 3-[(cycloheptylamino)carbonyl]-1,1-dimethyl-1,2,3,6-tetrahydroazepino[4,5-b]indole-5-carboxylate: ¹H NMR (400 MHz, CDCl₃): δ 10.70 (s, 1H), 8.02 (s, 1H), 7.78 (d, 1H), 7.36 (d, 1H), 7.15 (t, 1H), 7.05 (t, 1H), 5.22 (m, 1H), 4.77 (d, 1H), 3.93 (m, 1H), 3.80 (bs, 2H), 1.98 (m, 2H), 1.64 (m, 4H), 1.55 (d, 6H), 1.53 (m, 6H), 1.38 (d, 6H); MS (EI) for C₂₆H₃₅N₃O₃: 438.4 (MH⁺).

1-Methylethyl 1,1-dimethyl-3-{[(phenylmethyl)amino]carbonyl}-1,2,3,6-tetrahydroazepino[4,5-b]indole-5-carboxylate: ¹HNMR (400 MHz, CDCl₃): δ 10.66 (s, 1H), 8.30 (s, 1H), 7.78 (d, 1H), 7.32 (m, 6H), 7.15 (t, 1H), 7.05 (t, 1H), 5.19 (m, 2H), 4.54 (d, J=5.48 Hz, 2H), 3.86 (bs, 2H), 1.56 (s, 6H), 1.36 (d, J=6.26 Hz, 6H); MS (EI) for C₂₆H₂₉N₃O₃: 432.4 (MH⁺).

1-Methylethyl 3-[(diethylamino)carbonyl]-1,1-dimethyl-1,2,3,6-tetrahydroazepino[4,5-b]indole-5-carboxylate: ¹H NMR (400 MHz, CDCl₃): δ 10.73 (s, 1H), 7.78 (m, 2H), 7.36 (d, 1H), 7.14 (t, 1H), 7.05 (t, 1H), 5.23 (m, 1H), 3.82 (bs, 2H), 3.26 (q, 4H), 1.52 (s, 6H), 1.35 (d, J=6.41 Hz, 6H), 1.18 (t, 6H); MS (EI) for C₂₃H₃₁N₃O₃: 398.2 (MH⁺).

1-Methylethyl 1,1-dimethyl-3-{[(3S)-piperidin-3-ylamino]carbonyl}-1,2,3,6-tetrahydroazepino[4,5-b]indole-5-carboxylate: ¹H NMR (400 MHz, CDCl₃): δ 10.74 (s, 1H), 8.12 (s, 1H), 7.77 (d, 1H), 7.35 (d, 1H), 7.13 (t, 1H), 7.04 (t, 1H), 6.31 (bs, 1H), 5.20 (m, 1H), 4.07 (bs, 1H), 3.81 (bs, 2H), 2.97 (m, 3H), 2.88 (m, 2H), 2.77 (m, 1H), 1.74 (m, 2H), 1.55 (d, 6H), 1.37 (d, 6H); MS (EI) for C₂₄H₃₂N₄O₃: 425.2 (MH⁺).

1-Methylethyl 1,1-dimethyl-3-[(piperidin-3-ylamino)carbonyl]-1,2,3,6-tetrahydroazepino[4,5-b]indole-5-carboxylate: ¹H NMR (400 MHz, CDCl₃): δ 10.74 (s, 1H), 8.12 (s, 1H), 7.77 (d, 1H), 7.35 (d, 1H), 7.13 (t, 1H), 7.04 (t, 1H), 6.31 (bs, 1H), 5.20 (m, 1H), 4.07 (bs, 1H), 3.81 (bs, 2H), 2.97 (m, 3H), 2.88 (m, 2H), 2.77 (m, 1H), 1.74 (m, 2H), 1.55 (d, 6H), 1.37 (d, 6H); MS (EI) for C₂₄H₃₂N₄O₃: 425.3 (MH⁺).

### Example 29

### Preparation of 1-Methylethyl 8-Fluoro-1,1-Dimethyl-3-[(4-Methylpiperazin-1-yl)Carbonyl]-1,2,3,6-Tetrahydroazepino[4,5-b]Indole-5-Carboxylate

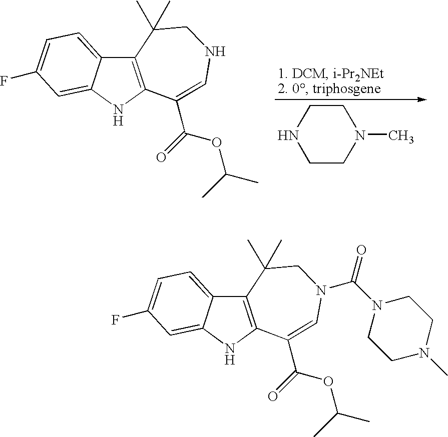

8-fluoro-1,1-dimethyl-3-[(4-methylpiperazin-1-yl)carbonyl]-1,2,3,6-tetrahydroazepino[4,5-b]indole-5-carboxylic acid tert-butyl ester (100 mg, 0.316 mmol) was dissolved in 10 mL dry DCM. N,N′-diisopropylethylamine (77.9 mg, 0.603 mmol) was added and solution was brought to 0° C. under N₂. Triphosgene (165 mg, 0.556 mmol) was added slowly and the mixture was allowed to stir for 1.5 h at 0° C. N-methylpiperazine (158 mg, 1.58 mmol) was added; reaction mixture was brought to room temperature, and allowed to stir overnight. When TLC indicated starting material was no longer present, the sample was purified by preparative liquid chromatography using a 40%-100% gradient of ACN/H₂O with 0.05% TFA for 10 minutes. Desired fractions were combined and made basic by saturated NaHCO₃ and diluted with ethyl acetate. The organic layer was extracted with water and brine, then dried over Na₂SO₃ and filtered. The yellow solution was reduced to dryness and lyophilized overnight in ACN/H₂O to form the bright yellow powder (26.5 mg, 19% yield) of the title compound: ¹H NMR (400 MHz, CDCl₃): δ 10.73 (s, 1H), 7.77 (s, 1H), 7.67 (m, 1H), 7.02 (dd, J=9.2, 2.4 Hz, 1H), 6.82 (td, J=9.2, 2.4 Hz, 1H), 5.23 (sept., J=6.4, 1H), 3.81 (br s, 2H), 3.47 (m, 4H), 2.47 (m, 4H), 2.35 (s, 3H), 1.50 (s, 6H) 1.38 (s, 3H) 1.36 (s, 3H); MS (EI) for C₂₄H₃₂FN₄O₃, 443.2 (MH⁺).

Using the same or analogous synthetic techniques and/or substituting with alternative reagents, the following compounds of the invention were prepared:

1-methylethyl 3-[(4-ethylpiperazin-1-yl)carbonyl]-8-fluoro-1,1-dimethyl-1,2,3,6-tetrahydroazepino[4,5-b]indole-5-carboxylate: ¹HNMR (400 MHz, CDCl₃): δ 10.74 (s, 1H), 7.78 (s, 1H), 7.66 (m, 1H), 7.02 (dd, J=9.2, 2.4 Hz, 1H), 6.82 (td, J=9.2, 2.4 Hz, 1H), 5.23 (sept., J=6.4, 1H), 3.80 (br s, 2H), 3.47 (m, 4H), 3.11 (q, J=6.8, 2H), 2.45 (m, 4H), 1.50 (s, 6H), 1.37 (s, 3H) 1.35 (s, 3H), 1.09 (t, J=7.2, 3H); MS (EI) for C₂₅H₃₄FN₄O₃, 457.1 (MH⁺).

All of the above compounds can be converted to pharmaceutically acceptable salts such as HCl salts, by dissolving the compound in a suitable solvent such as dioxane or methanol, followed by the addition of anhydrous HCl in dioxane. The product can be isolated by filtration, or concentration of the solution or mixture to dryness.

### Example 30

### Time Resolved Fluorescence Resonance Energy Transfer (TR-FRET) Assay

The TR-FRET assay was performed by incubating 8 nM of GST-farnesoid X receptor-LBD (comprising glutathione-5-transferase fused in frame to the farnesoid X receptor ligand binding domain, (amino acids 244-471 of the human farnesoid X receptor)), 8 nM of Europium-labeled anti-GST antibody (Wallac/PE Life Sciences Cat#AD0064), 16 nM biotin-SRC-1 peptide [5′-biotin-CPSSHSSLTERHKILHRLLQEGSPS-CONH2], 20 nM APC-SA [allophycocyanin conjugated streptavidin] (Wallac/PE Life Sciences, Cat# AD0059A) in FRET assay buffer (20 mM KH₂PO₄/K₂HPO₄ (pH 7.3), 150 mM NaCl, 2 mM CHAPS, 2 mM EDTA, 1 mM DTT) in the presence of the test compound(s) for 2-4 hours at room temperature in a 384 well assay plate. Data was collected using an LJL Analyst using the standard operating instructions and conditions with readings at emission wavelengths of 615 nm and 665 nm after a delay of 65 □s and an excitation wavelength of 330 nm.

### Example 31

### Co-Transfection Assay

The basic co-transfection protocol for measuring the farnesoid X receptor activity is as follows. CV-1 African Green Monkey Kidney cells were plated 24 hours before transfection to achieve approximately 70-80 percent confluency. Cells were transfected with the following expression vectors, CMX-farnesoid X receptor (full length human farnesoid X receptor), CMX-RXRα (fall length human RXR), Luc12 ((ECREx7-Tk-Luciferase) luciferase reporter gene construct. (See WO 00/76523, Venkateswaran et al., (2000) J. Biol. Chem. 275 14700-14707). A CMX-β-Galactosidase expression vector was used as a transfection control. The transfection reagent used was DOTAP (Boebringer Mannheim). Cells were incubated with the DOTAP/DNA mixture for 5 hours after which the cells were harvested and plated onto either 96 well or 384 well plates containing the appropriate concentration of test compound. The assay was allowed to continue for an additional 18-20 hours, after which the cells were lysed, with lysis buffer (1% triton X 100, 10% glycerol, 5 mM Dithiothreitol, 1 mM EGTA, 25 mM Tricine, pH 7.8) and the luciferase activity measured in the presence of Luciferase assay buffer (0.73 mM ATP, 22.3 mm Tricine, 0.11 mM EGTA, 0.55 mM Luciferin, 0.15 mM Coenzyme A, 0.5 mM HEPES, 10 mM Magnesium sulphatc) on a standard luminometer plate reader (PE Biosystems, NorthStar Reader), using recommended operating instructions and conditions.

### Example 32

### Formulation and Experimental Design

### A. Solution Formulation

Test article was administered intravenously at 3 mg/kg formulated in carrier dosage vehicle suitable for IV administration of the test article. Oral solution (or suspension) doses of 3, 10, 30, 100, 300 and 1000 mg/kg were administered using a suitable carrier dosage vehicle. The compound was also administered at 10 mg/kg as a solid in gelatin capsules. Experimental groups were comprised of five animals for each dose group. Blood was collected (100 μL) in heparinized tubes via a jugular catheter at 0.02, 0.08, 0.25, 0.5, 1, 2, 4, 6, 8, 10, 12, 24, 32, 48 and 72 hours post-dosing for the IV groups. Samples were similarly collected at 0.08, 0.25, 0.5, 1, 2, 4, 6, 8, 10, 12, 24, 32, 48 and 72 hours post-dosing for the PO groups. The plasma obtained was stored at −80° C. and a volume of 50 μL was used for analysis.

### B. Solid Dosage

Torpac size 9 porcine gelatin mini capsules were used to orally dose test article in solid form at 3 or 10 mg/kg. Capsules wore filled with powdered compound based on body weight. Capsules were administered directly into the rat's stomach with the use of a stainless steel dosing device similar to an oral gavage needle. Pilot studies with empty capsules revealed that capsules dissolve in less than 7 minutes in the stomach.

### Bioanalytical Analysis

The concentration of test article in plasma and tissue samples was determined by HPLC/MS/MS analysis using sample preparation and analytical conditions appropriate for the test article quantification by this method. A non-compartmental model was applied to calculate pbarmnacokinetic (PK) parameters for all routes of administration using WinNonlini 3.1 software (Pharsight Co., Mountain View, Calif.).

The compounds of the present invention exhibited greatly enhanced and improved pharmacokinetic properties.

### Example 33

### Kinetic Solubility Assay

The kinetic solubility of test compounds in buffer was evaluated using a 96 well filtration plate format. A 500 μM assay solution in PBS, pH7.4 (or other assay buffer, as needed) was generated form a DMSO stock solution (up to 10 mM). Samples were transferred to a 96 Millipore MultiScreen HTS 96-well Filter plate (Cat# MSSLBPC10) mixed by shaking for 1.5 hours and processed by filtration prior to quantitation by HPLC-UV. Amiodarone and testosterone were used as reference controls. In-house historical data shows that the solubility of amiodarone is between 3-5 μM and testosterone is approximately 330 μM. An Agilent Chemstation using a Waters 4×23 mm threaded cartridge YMC/AQ S-5 120A C18 column was used for separation of analytes at a mobile phase flow rate of 2.2 mL/min. The mobile phase was 0.1% TFA in water (solvent A) and 0.1% TFA in acetonitrile (solvent B). The column was maintained at 37° C. and detection of analytes was achieved by UV signal quantification at 220 nm and 254 nm following a 10 μL injection volume.

The compounds demonstrated kinetic solubility in the range of for example, about 500 μM or less, 400 μM or less, 300 μM or less, 200 μM or less, 100 μM or less. In an advantageous embodiment, the kinetic solubility is about 50 μM or less, 20 μM or less, 10 μM or less, 5 μM or less, 2.5 μM or less, or 1 μM or less.

### Example 34

### In Vivo Studies

### General Methods

Young adult male mice (˜8 weeks of age) were purchased from conventional vendors and group housed (3-4/cage) with ad libitum access to chow and water, in a temperature- and light-controlled vivarium (lights on at 06:00 hours, off at 18:00 hours). Compounds were administered daily by oral gavage in the morning (˜08:00 hrs), in a final volume of 0.1 ml/mouse, with the first dose of compound delivered on study day 0. Compounds were solubilized by gentle mixing in PEG400:Tween80 (4:1) for at least several hours and usually overnight prior to initiation of dosing. When necessary, solutions were sonicated briefly to ensure complete compound solubilization.

Blood samples (˜0.15 ml/mouse) were obtained from the retro-orbital sinus of non-fasted isoflurane-anesthetized mice, 3 hours after drug dosing. Blood samples were collected into heparin-coated tubes, and plasma was recovered following centrifugation. Plasma total cholesterol and triglyceride levels were determined by calorimetric enzymatic commercially available assays that were adapted to 96-well plate formats. Plasma HDL cholesterol (HDL-C) was determined by removing non-HDL-C from the plasma with a precipitating reagent, and then determining the plasma cholesterol levels in the remaining HDL-C fraction. Plasma triglycerides concentrations, determined from a blood sample obtained in the 24 hours prior to the first dose, were used to group the mice such that the pre-study triglyceride levels between groups were equivalent prior to initiation of the dosing regimen.

Representative data from these experiments are shown in FIGS. 1, 2 and 3 for the effect of Compound A (ethyl 3-(3,4-difluorobenzoyl)-1-methyl-1,2,3,6-tetrahydroazepino[4,5-b]indole-5-carboxylate), Compound B (Ethyl 3-(3,4-difluorobenzoyl)-1,1-dimethyl-1,2,3,6-tetrahydro-azepino[4,5-b]indole-5-carboxylate) and Compound C (isopropyl 3-(3,4-difluorobenzoyl)-1,1-dimethyl-1,2,3,6-tetrahydroazepino[4,5-b]indole-5-carboxylate).

### Compound Effects in Normolipidemic Mice

Male C57BL/6 mice (Harlan Sprague Dawley, San Diego, Calif.) consumed standard laboratory chow (˜4.5% fat w/w) ad libitum and were treated with Compound A (FIG. 1A) or Compound B (FIG. 1B) daily by oral gavage at doses of 0.1, 1.0 or 10 mg/kg/day for seven days (n=6/group).

FIG. 1 shows plasma triglyceride levels in the C57BL/6 mice either treated with Compound A (FIG. 1A) or Compound B (FIG. 1B) daily by oral gavage at doses of 0.1 (filled triangles), 1.0 (Upside down filled triangles) or 10 mg/kg/day (Diamonds) for seven days (n=6/group) compared to vehicle alone (filled squares).

Surprisingly across this dose range, both compounds significantly reduced plasma triglycerides ˜25-30% on study day 7 (*p<0.05 vs. vehicle-treated controls within treatment day). Even at the lowest dose tested (0.1 mg/kg) the compounds unexpectedly exhibit the ability to dramatically reduce plasma triglyceride levels.

### Compound Effects in Diet-Induced Hyperlipidemic LDLR^{-/-} Mice

Male LDLR^{-/-} mice (JAX Mice, Bar Harbor, Me.) consumed a purified “Western” diet (˜21% fat, 0.02% cholesterol w/w) ad libitum, for two weeks prior to and during treatment with Compound C daily by oral gavage at a dose of 10 mg/kg/day for 7 days (n=9-10/group).

The results, (FIG. 2) show that by study day 7, the compound had reduced plasma triglyceride concentrations to the level observed prior to introduction of the high-fat chow. Surprisingly, Compound C also significantly lowered plasma total cholesterol levels ˜40% by study day 7, despite continued consumption of the high-fat, cholesterol-supplemented chow (FIG. 2B, *p<0.05 vs. vehicle-treated controls within treatment day).

In a separate study utilizing the same mouse model (n=112-16/group) subjected to the dietary lead-in for 8 weeks, LDLR^{-/-} mice were treated with Compound B at a dose of 10 mg/kg/day for 6 weeks. Surprisingly Compound B also lowered both plasma triglyceride and cholesterol concentrations with a time course similar to that observed with Compound C (FIGS. 3A and B). Daily dosing with Compound C resulted in a sustained normalization of plasma lipid profiles throughout the 6 weeks of study, despite continued consumption of the “Western” diet.

The data demonstrate in sum that the claimed compounds exhibit unexpectedly high potency and efficacy in modulating plasma triglyceride and cholesterol in both normal animals and animal models of hyperlipidemia. Accordingly such compounds show great potential for the development of therapeutic agents and specific utility for use in the various methods disclosed herein.

### Results of Examples 30 and 31

Both the farnesoid X receptor/ECREx7 co-transfection assay (Example 31) and the TR-FRET assay (Example 30) can be used to establish the EC₅₀/IC₅₀ values for potency and percent activity or inhibition for efficacy. Efficacy defines the activity of a compound relative to a high control (chenodeoxycholic acid, CDCA) or a low control (DMSO/vehicle). The dose response curves are generated from an 8 point curve with concentrations differing by ½ LOG units. Each point represents the average of 4 wells of data from a 384 well plate. A curve for the data is generated by using the equation:

Y=Bottom+(Top−Bottom)/(1+10̂((Log EC50−X)*HillSlope))

The EC₅₀/IC₅₀ is therefore defined as the concentration at which an agonist or antagonist elicits a response that is half way between the Top (maximum) and Bottom (baseline) values. The EC₅₀/IC₅₀ values represented are the averages of at least 3 independent experiments. The determination of the relative efficacy or % control for an agonist is by comparison to the maximum response achieved by chenodeoxycholic acid that is measured individually in each dose response experiment.

For the antagonist assay, CDCA is added to each well of a 384 well plate to elicit a response. The % inhibition for each antagonist is therefore a measurement of the inhibition of the activity of CDCA. In this example, 100% inhibition would indicate that the activity of CDCA has been reduced to baseline levels, defined as the activity of the assay in the presence of DMSO only.

The compounds of the invention demonstrated the ability to bind to FXR when tested in this assay. Preferably, the compound binds to the FXR with a binding affinity, for example, of about 50 μM or less, 20 μM or less, 10M or less, 5 μM or less, 2.5 μM or less or 1M or less. In an advantageous embodiment, the IC₅₀ of the binding compounds is about 0.5 μM or less, about 0.3 μM or less, about 0.1 μM or less, about 0.08 μM or less, about 0.06 μM or less, about 0.05 μM or less, about 0.04 μM or less, 0.03 μM or less, preferably, about 0.03 μM or less.

For the antagonist assay, CDCA is added to each well of a 384 well plate to elicit a response. The % inhibition for each antagonist is therefore a measurement of the inhibition of the activity of CDCA. In this example, 100% inhibition would indicate that the activity of CDCA has been reduced to baseline levels, defied as the activity of the assay in the presence of DMSO only.

Most of the compounds disclosed herein and tested exhibited activity in at least one of the above assays (EC₅₀ or IC₅₀ less than 10 uM). Most showed activity at below 1 μM. For example, the compounds exhibited agonist activity with less than 1 μM EC₅₀ and greater than 100% efficacy as measured via the co-transfection assay. The compounds exhibited agonist activity with less than 250 nM EC₅₀ and greater than 100% efficacy as measured via one or more of the in vitro assays described herein as shown in Table I. IC₅₀ and kinetic solubility data are represented as follows: A=0.001-0.01 μM, B=0.01-0.1 μM, C=0.1-1.0 μM, and D=1.0-10 μM, E=>10 μM. The % efficacy is represented as follows: A=>100%; B=80-100%; C=60-80%; D=40-60%; E=<40%.

TABLE I

| | | | | | Kinetic |
|---|---|---|---|---|---|
| Cpd | | | EC50 | % | Solubility |
| # | IUPAC Name | Structure | μM | Efficacy | μM |
| 1 | 1-methylethyl-3- (cyclohexylcarbonyl)-1,1- dimethyl-1,2,3,6- tetrahydroazepino [4,5- b]indole-5-carboxylate | 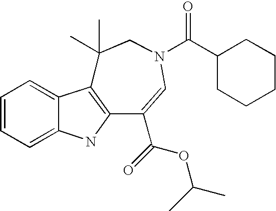 | B | A | A |
| 2 | 1-methylethyl 1,1-dimethyl- 3-[(1-methylpiperidin-3- yl)carbonyl]-1,2,3,6- tetrahydroazepino[4,5- b]-indole-5-carboxylate | 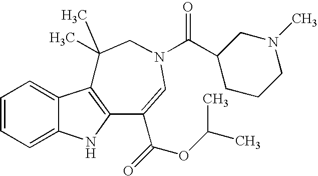 | C | A | E |
| 3 | 1-methylethyl 3-acetyl-1,1- dimethyl-1,2,3,6- tetrahydroazepino[4,5- b[indole-5-carboxylate | 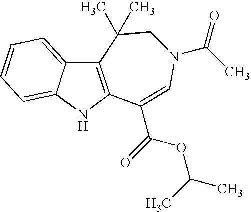 | D | A | A |
| 4 | 1-methylethyl 3-butanoyl- 1,1-dimethyl-1,2,3,6- tetrahydroazepino[4,5- b]indole-5-carboxylate | 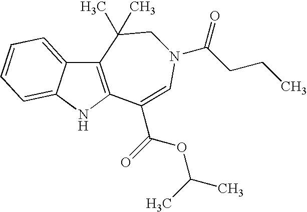 | C | A | A |
| 5 | 1-methylethyl 1,1-dimethyl- 3-pentanoyl-1,2,3,6- tetrahydroazepino[4,5- b]indole-5-carboxylate | 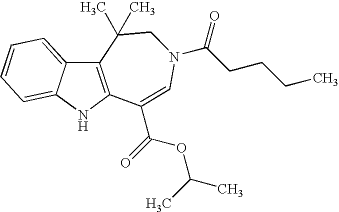 | B | A | A |
| 6 | 1-methylethyl 1,1-dimethyl- 3-[(1-methylpiperidin-4- yl)carbonyl]-1,2,3,6- tetrahydroazepino[4,5- b]indole-5-carboxylate | 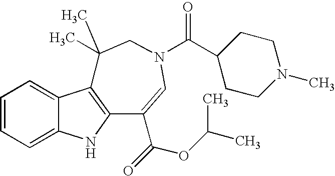 | D | A | C |
| 7 | 1-methylethyl 1,1-dimethyl- 3-(piperidin-1-ylcarbonyl)- 1,2,3,6-tctrahydroazepino [4,5-b]indole-5-carboxylate | 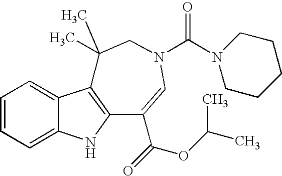 | A | A | A |
| 8 | 1-methylethyl 3- (cyclopentylcarbonyl)-1,1- dimethyl-1,2,3,6- tetrahydroazepino[4,5- b[indole-5-carboxylate | 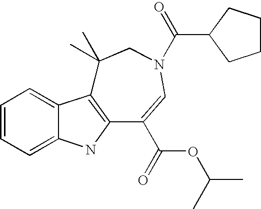 | B | A | A |
| 9 | 1-methylethyl 3-(2,2- dimethyl propanoyl)-1,1- dimethyl-1,2,3,6- tetrahydroazepino[4,5- b]indole-5-carboxylate | 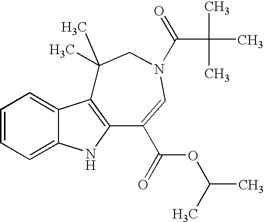 | C | A | A |
| 10 | 1-methylethyl 3-(2- ethylbutanoyl)-1,1-dimethyl- 1,2,3,6- tetrahydroazepino[4,5- b]indole-5-carboxylate | 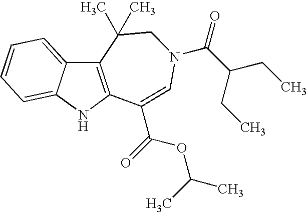 | B | A | A |
| 11 | 1-methylethyl 1,1-dimethyl- 3-(3-methylbutanoyl)- 1,2,3,6- tetrahydroazepino[4,5- b]indole-5-carboxylate | 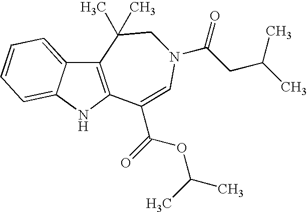 | B | A | A |
| 12 | 1-methylethyl 3- (cycloheptylcarbonyl)-1,1- dimethyl-1,2,3,6- tetrahydroazepino[4,5- b]indole-5-carboxylate | 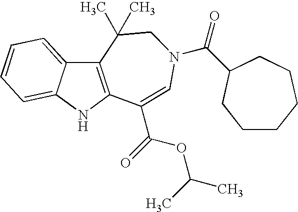 | B | A | A |
| 13 | 1-methylethyl 3-({[3- (dimethylamino)propyl]amino} carbonyl)-1,1-dimethyl- 1,2,3,6-tetrahydroazepino [4,5-b]indole-5-carboxylate | 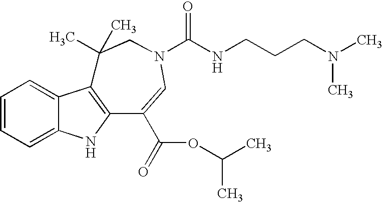 | D | E | |
| 14 | 1-methylethyl 1,1-dimethyl- 3-({[4-(4-methylpiperazin-1- yl)phenyl]amino} carbonyl)- 1,2,3,6-tetrahydroazepino [4,5-b]indole-5-carboxylate | 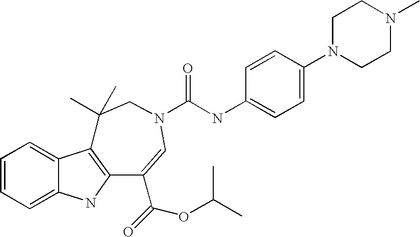 | D | | |
| 15 | 1-methylethyl 1,1-dimethyl- 3-(pyrrolidin-1-ylcarbonyl)- 1,2,3,6-tetrahydroazepino [4,5-b]indole-5-carboxylate | 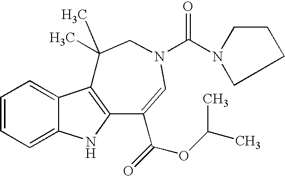 | B | A | D |
| 16 | 1-methylethyl 1,1-dimethyl- 3-[(4-methylpiperazin-1- yl)carbonyl]-1,2,3,6- tetrahydroazepino[4,5- b]indole-5-carboxylate | 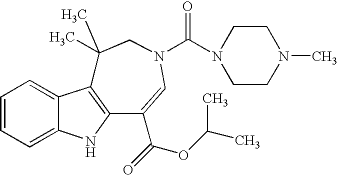 | C | A | C |
| 17 | 1-methylethyl 3-({[2- (dimethylamino) ethyl]amino}carbonyl)-1,1- dimethyl-1,2,3,6- tetrahydroazepino[4,5- b]indole-5-carboxylate | 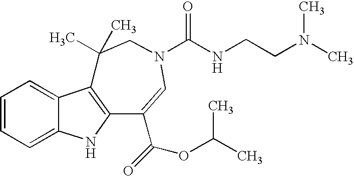 | D | | |
| 18 | 1-methylethyl 1,1-dimethyl- 3-{[(3-morpholin-4- ylpropyl)amino]carbonyl}- 1,2,3,6- tetrahydroazepino[4,5- b[indole-5-carboxylate | 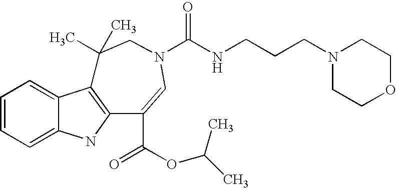 | D | | |
| 19 | 1-methylethyl 1,1-dimethyl- 3-propanoyl-1,2,3,6- tctrahydroazcpino[4,5- b[indole-5-carboxylate | 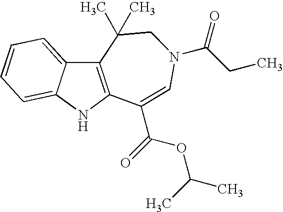 | C | A | E |
| 20 | 1-methylethyl 3-[4- (dimethylamino) butanoyl]- 1,1-dimethyl-1,2,3,6- tetrahydroazepino[4,5- b[indole-5-carboxylate | 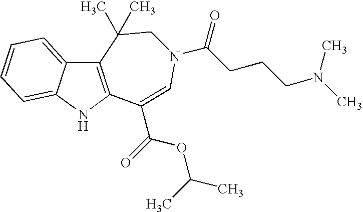 | D | | |
| 21 | 1-(1,1-dimethyl-1,2,3,6- tetrahydro azepino[4,5- b[indol-5-yl)ethanone | 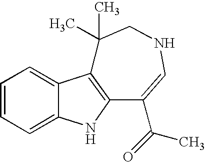 | D | | |
| 22 | 1-methylethyl 1,1-dimethyl- 3-{[(2-morpholin-4- ylethyl)amino]carbonyl}- 1,2,3,6- tetrahydroazepino[4,5- b]indole-5-carboxylate | 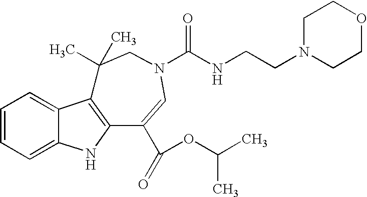 | D | | |
| 23 | 1-methylethyl 1,1-dimethyl- 3-[(propylamino)carbonyl]- 1,2,3,6- tetrahydroazepino[4,5- b]indole-5-carboxylate | 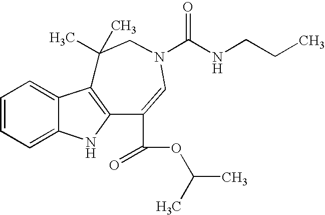 | C | A | D |
| 24 | 1-methylethyl 1,1-dimethyl- 3-[(3s,5s,7s)- tricyclo[3.3.1.1~3,7~]dec-1- ylcarbonyl]-1,2,3,6- tetrahydroazepino [4,5- b]indole-5-carboxylate | 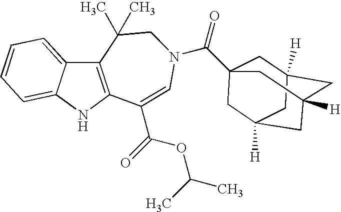 | B | A | A |
| 25 | 1-methylethyl 3-[(4- ethylpiperazin-1- yl)carbonyl]-1,1-dimethyl- 1,2,3,6- tetrahydroazepino[4,5- b]indole-5-carboxylate | 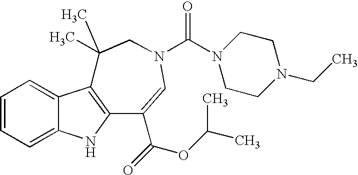 | B | A | E |
| 26 | 1-methylethyl 1,1-dimethyl- 3-(piperazin-1-ylcarbonyl)- 1,2,3,6-tetrahydroazepino [4,5-b]indole-5-carboxylate | 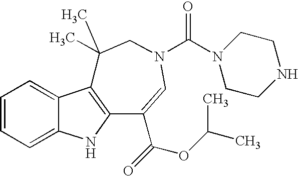 | D | A | E |
| 27 | 1-methylethyl 3-({[2- (diethylamino)ethyl](ethyl) amino}carbonyl)-1,1- dimethyl-1,2,3,6-tetrahydro azepino [4,5-b]indole-5- carboxylate | 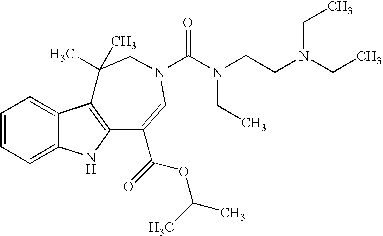 | D | | |
| 28 | 1-methylethyl 1,1-dimethyl- 3[(4-pyrrolidin-1- ylpiperidin-1-yl)carbonyl]- 1,2,3,6- tetrahydroazepino[4,5- b]indole-5-carboxylate | 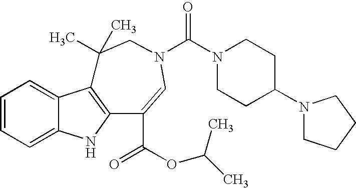 | D | B | E |
| 29 | 1-methylethyl 1,1-dimethyl- 3-({4-[(1-methylpiperidin-4- yl)methyl]piperazin-1- yl}carbonyl)-1,2,3,6- tetrahydroazepino [4,5- b]indole-5-carboxylate | 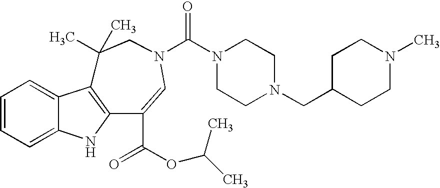 | D | D | E |
| 30 | 1-methylethyl 1,1-dimethyl- 3-{[4-(1- methylethyl)piperazin-1- yl)carbonyl}-1,2,3,6- tetrahydroazepino[4,5- b]indole-5-carboxylate | 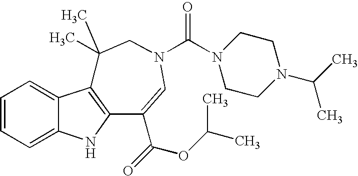 | C | A | E |
| 31 | 1-methylethyl 1,1-dimethyl- 3-[(4-propylpiperazin-1- yl)carbonyl]-1,2,3,6- tetrahydroazepino[4,5- b[indole-5-carboxylate | 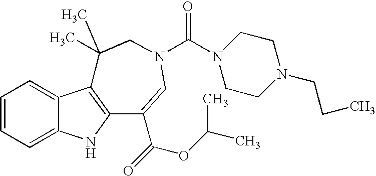 | C | A | D |
| 32 | 1-methylethyl 9-fluoro-1,1- dimethyl-3-[(4- methylpiperazin-1- yl)carbonyl]-1,2,3,6- tetrahydroazepino[4,5- b]indole-5-carboxylate | 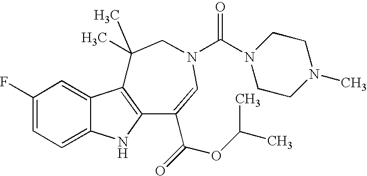 | C | A | A |
| 33 | 1-methylethyl 3-{[4-0 (diethylamino) piperidin-1- yl]carbonyl}-1,1-dimethyl- 1,2,3,6- tetrahydroazepino[4,5- b]indole-5-carboxylate | 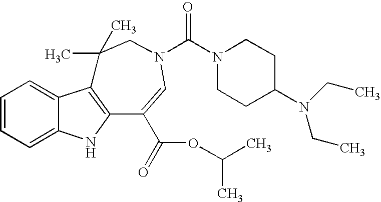 | D | C | E |
| 34 | 1-methylethyl 1,1-dimethyl- 3-(4-methylpentanoyl)- 1,2,3,6-tetrahydro azepino [4,5-b]indole-5-carboxylate | 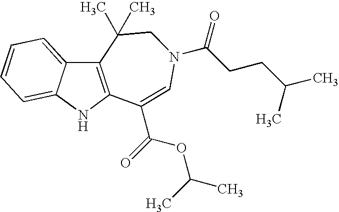 | B | A | E |
| 35 | 1-methylethyl 8-fluoro-1,1- dimethyl-3-[(4- methylpiperazin-1- yl)carbonyl]-1,2,3,6- tetrahydroazepino[4,5- b]indole-5-carboxylate | 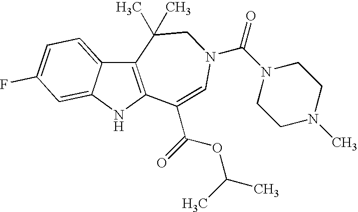 | B | A | E |
| 36 | 1-methylethyl 3-[(4- ethylpiperazin-1- yl)carbonyl]-8-fluoro-1,1- dimethyl-1,2,3,6- tetrahydroazepino[4,5- b]indole-5-carboxylate | 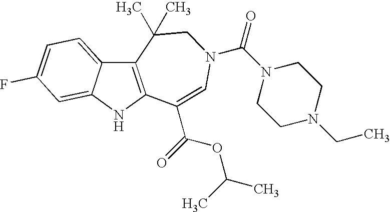 | B | A | E |
| 37 | 1-methylethyl 1,1-dimethyl- 3-{[4-(2-oxo-2-pyrrolidin-1- ylethyl)piperazin-1- yl]carbonyl}-1,2,3,6- tetrahydroazepino [4,5- b]indole-5-carboxylate | 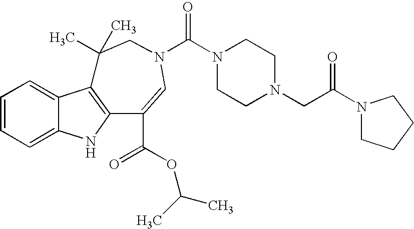 | C | A | E |
| 38 | 1-methylethyl 3-({4-[2- (diethylamino) ethyl]piperazin-1- yl}carbonyl)-1,1-dimethyl- 1,2,3,6- tetrahydroazepino[4,5- b]indole-5-carboxylate | 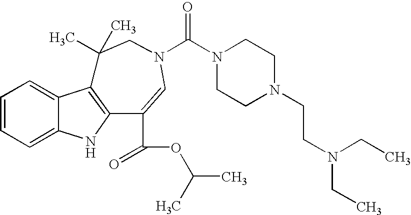 | D | C | E |
| 39 | 1-methylethyl 3-{[3- (dimethylamino) piperidin-1- yl]carbonyl}-1,1-dimethyl- 1,2,3,6- tetrahydroazepino[4,5- b]indole-5-carboxylate | 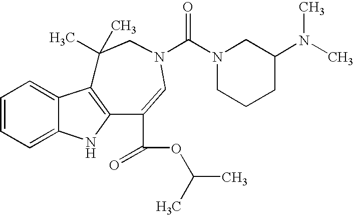 | C | A | E |
| 40 | 1-methylethyl 3-(azepan-1- ylcarbonyl)-1,1-dimethyl- 1,2,3,6-tetrahydroazepino [4,5-b]indole-5-carboxylate | 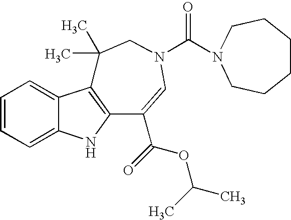 | A | A | A |
| 41 | 1-methylethyl 1,1-dimethyl- 3{[4-(4-methylpiperazin-1- yl)piperidin-1-yl]carbonyl}- 1,2,3,6-tetrahydroazepino [4,5-b]indole-5-carboxylate | 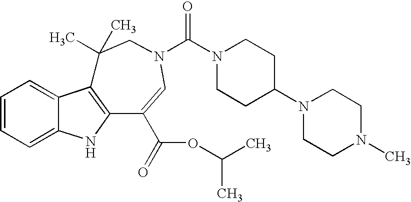 | D | D | E |
| 42 | 1-methylethyl 1,1-dimethyl- 3-{[(1S,4S)-5-methyl-2,5- diazabicyclo[2.2.1]hept-2- yl]carbonyl}-1,2,3,6- tetrahydroazepino [4,5- b]indole-5-carboxylate | 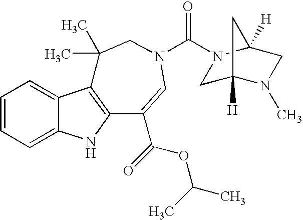 | C | A | E |
| 43 | 1-methyl 1,1-dimethyl- 3-[(4-methyl-1,4-diazepan-1- yl)carbonyl]-1,2,3,6- tetrahydroazepino[4,5- b]indole-5-carboxylate | 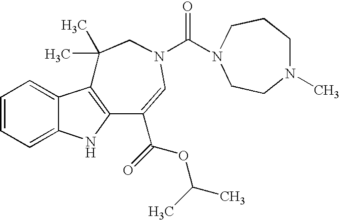 | C | A | E |
| 44 | 1-methylethyl 3- [(cyclopentylamino) carbonyl]-1,1-dimethyl- 1,2,3,6- tetrahydroazepino[4,5- b]indole-5-carboxylate | 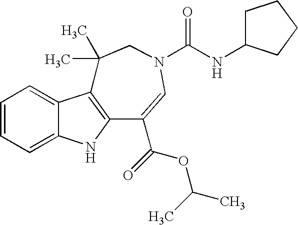 | B | A | A |
| 45 | 1-methylethyl 3- [(cyclohexylamino) carbonyl]-1,1-dimethyl- 1,2,3,6- tetrahydroazepino[4,5- b]indole-5-carboxylate | 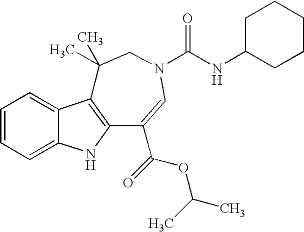 | B | A | D |
| 46 | 1-methylethyl 3- [(cycloheptylamino) carbonyl]-1,1-dimethyl- 1,2,3,6- tetrahydroazepino[4,5- b]indole-5-carboxylate | 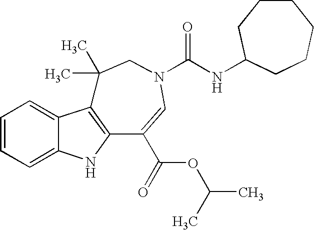 | B | A | A |
| 47 | 1-methylethyl 1,1-dimethyl- 3-(morpholin-4-ylcarbonyl)- 1,2,3,6- tetrahydrazepino[4,5- b]indole-5-carboxylate | 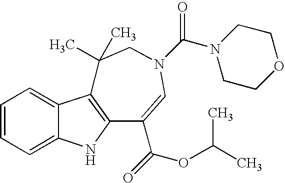 | B | A | E |
| 48 | 1-methylethyl 3-({3- [(dimethylamino) methyl]piperidin-1- yl}carbonyl)-1,1-dimethyl- 1,2,3,6- tetrahydroazepino[4,5- b]indole-5-carboxylate | 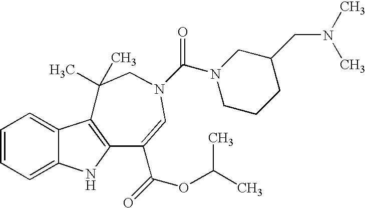 | D | A | E |
| 49 | 1-methylethyl 1,1-dimethyl- 3- {[(phenylmethyl)amino] carbonyl}-1,2,3,6- tetrahydroazepino[4,5- b]indole-5-carboxylate | 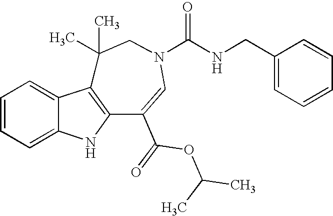 | D | B | A |
| 50 | 1-methylethyl 3-({(3S)-3- [(dimethylamino)methyl] piperidin-1-yl}carbonyl)-1,1- dimethyl-1,2,3,6- tetrahydroazepino[4,5- b]indole-5-carboxylate | 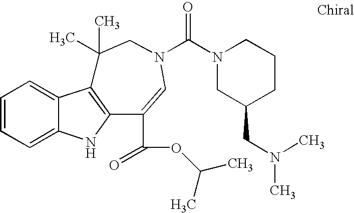 | D | C | E |
| 51 | 1-methylethyl 3-({(3R)-3- [(dimethylamino) methyl]piperidin-1- yl}carbonyl)-1,1-dimethyl- 1,2,3,6- tetrahydroazepino[4,5- b]indole-5-carboxylate | 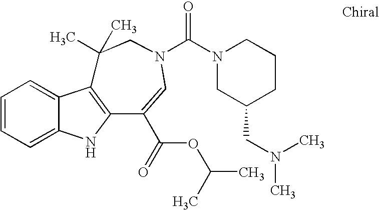 | D | A | E |
| 52 | 1-methylethyl 3- [(diethylamino) carbonyl]- 1,1-dimethyl-1,2,3,6- tetrahydroazepino[4,5- b]indole-5-carboxylate | 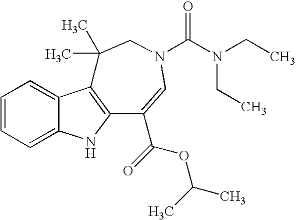 | B | A | A |
| 53 | 1-methylethyl 1,1-dimethyl- 3{[3-(morpholin-4- ylmethyl)piperidin-1- yl]carbonyl}-1,2,3,6- tetrahydroazepino [4,5- b]indole-5-carboxylate | 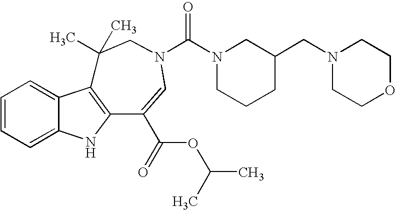 | C | A | E |
| 54 | 1-mcthylethyl 1,1-dimethyl- 3-{[(3S)-piperidin-3- ylamino]carbonyl}-1,2,3,6- tetrahydroazepino[4,5- b]indole-5-carboxylate | 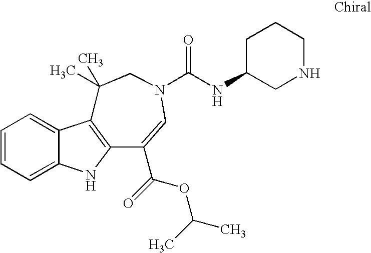 | D | D | E |
| 55 | 1-methylethyl 3-{[3-({[3- (dimethylamino) propyl]oxy}methyl)piperidin- 1-yl]carbonyl}-1,1- dimethyl-1,2,3,6- tetrahydroazepino[4,5- b]indole-5-carboxylate | 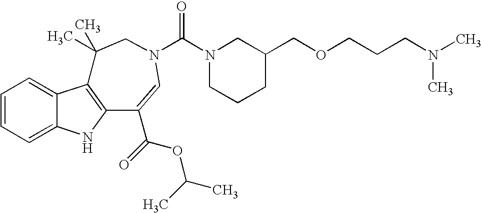 | D | | |
| 56 | 1-methylethyl 1,1-dimethyl- 3-[(piperidin-3- ylamino)carbonyl]-1,2,3,6- tetrahydro azepino[4,5- b]indole-5-carboxylate | 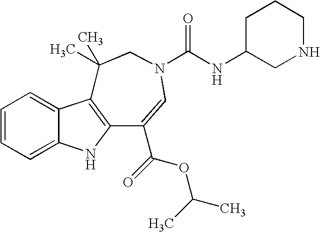 | D | E | E |
| 57 | 1-methylethyl 1,1-dimethyl- 3-{[(3R)-3-(morpholin-4- ylmethyl)piperidin-1- yl]carbonyl}-1,2,3,6- tetrahydroazepino [4,5- b]indole-5-carboxylate | 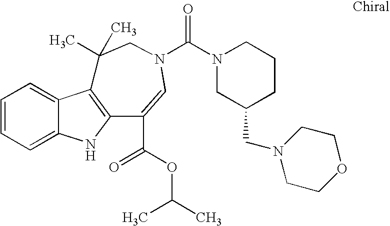 | D | A | E |
| 58 | 1-methylethyl 1,1-dimethyl- 3-{[(3R)-3-(piperidin-1- ylmethyl)piperidin-1- yl]carbonyl}-1,2,3,6- tetrahydroazepino [4,5- b]indole-6-carboxylate | 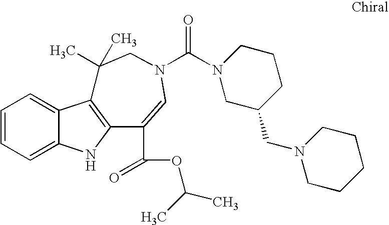 | D | B | E |
| 59 | 1-methylethyl 1,1-dimethyl- 3-{[4-(phenylmethyl)-1,4- diazepan-1-yl]carbonyl}- 1,2,3,6-tetrahydroazepino [4,5-b]indole-5-carboxylate | 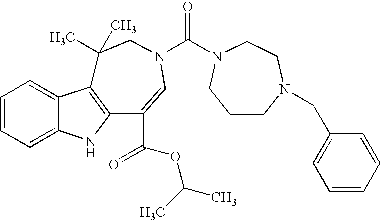 | B | A | A |
| 60 | 1-methylethyl 3-[(3′R)-1,3′- bipiperidin-1′-ylcarbonyl]- 1,1-dimethyl-1,2,3,6- tetrahydroazepino[4,5- b]indole-5-carboxylate | 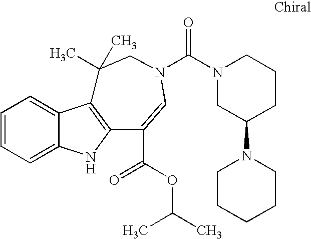 | D | C | E |
| 61 | 1-methylethyl 3-[(2-chloro- 3,6- difluorophenyl)carbonyl]- 1,1-dimethyl-1,2,3,6- tetrahydroazepino[4,5- b]indole-5-carboxylate | 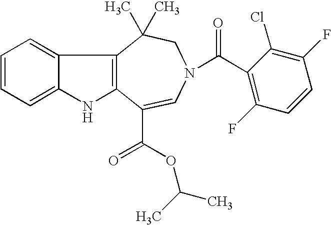 | B | A | A |
| 62 | 1-methylethyl 1,1-dimethyl- 3-(phenylcarbonyl)-1,2,3,6- tetrahydroazepino[4,5- b]indole-5-carboxylate | 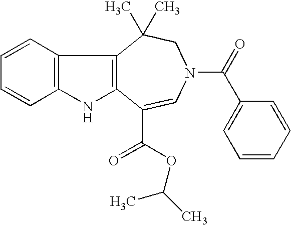 | A | A | A |
| 63 | 1-methylethyl 3-[(2- fluorophenyl)carbonyl]-1,1- dimethyl-1,2,3,6- tetrahydroazepino[4,5- b]indole-5-carboxylate | 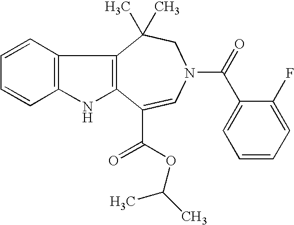 | B | A | A |
| 64 | 1-methylethyl 1,1-dimethyl- 3-{[2- (trifluoromethyl)phenyl]- carbonyl}-1,2,3,6- tetrahydroazepino[4,5- b]indole-5-carboxylate | 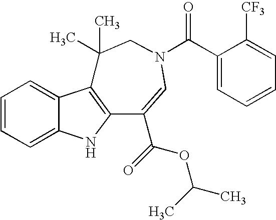 | B | A | D |
| 65 | 1-methylethyl 1,1-dimethyl- 3-{[2- (trifluoromethyl)phenyl]- carbonyl}-1,2,3,6- tetrahydroazepino[4,5- b]indole-5-carboxylate | 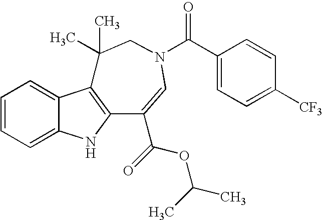 | B | A | A |
| 66 | 1-methylethyl 3-[(2- chlorophenyl)carbonyl]-1,1- dimethyl-1,2,3,6- tetrahydroazepino[4,5- b]indole-5-carboxylate | 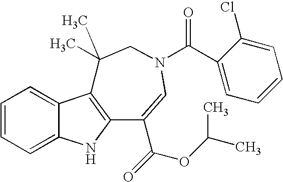 | A | A | A |
| 67 | 1-methylethyl 3-[(2- bromophenyl)carbonyl]-1,1- dimethyl-1,2,3,6- tetrahydroazepino[4,5- b]indole-5-carboxylate | 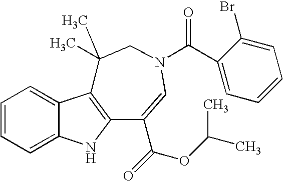 | B | A | D |
| 68 | 1-methylethyl 1,1-dimethyl- 3-[(2- methylphenyl)carbonyl]- 1,2,3,6- tetrahydroazepino[4,5- b]indole-5-carboxylate | 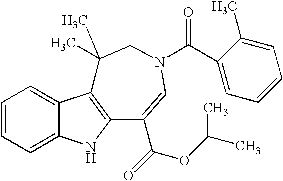 | B | A | A |
| 69 | 1-methylethyl 1,1-dimethyl- 3-{[2- (methyloxy)phenyl]carbonyl}- 1,2,3,6- tetrahydroazepino[4,5- b]indole-5-carboxylate | 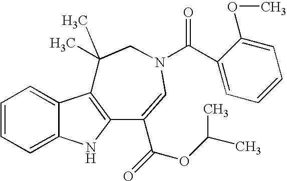 | B | A | A |
| 70 | 1-methylethyl 1,1-dimethyl- 3-({2- [(trifluoromethyl)oxy]phenyl} carbonyl)-1,2,3,6- tetrahydroazepino[4,5- b]indole-5-carboxylate | 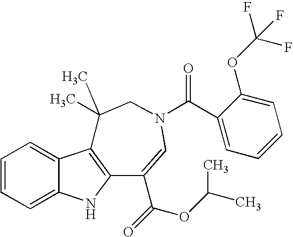 | C | A | D |
| 71 | 1-methylethyl 3-[(2- fluorophenyl)carbonyl]-1,1- dimethyl-1,2,3,6- tetrahydroazepino[4,5- b]indole-5-carboxylate | 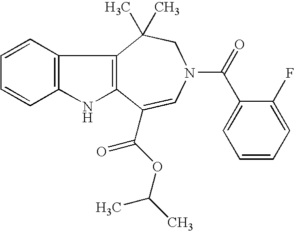 | B | A | A |
| 72 | 1-methylethyl 3-[(3- fluorophenyl)carbonyl]-1,1- dimethyl-1,2,3,6- tetrahydroazepino[4,5- b]indole-5-carboxylate | 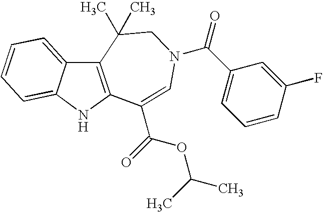 | A | A | D |
| 73 | 1-methylethyl 3-[(2,4- difluorophenyl)carbonyl]- 1,1-dimethyl-1,2,3,6- tetrahydroazepino[4,5- b]indole-5-carboxylate | 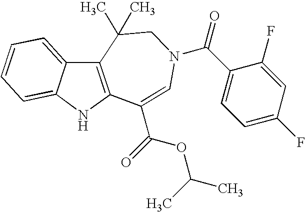 | A | A | A |
| 74 | 1-methylethyl 3-[(2,3- difluorophenyl)carbonyl]- 1,1-dimethyl-1,2,3,6- tetrahydroazepino[4,5- b]indole-5-carboxylate | 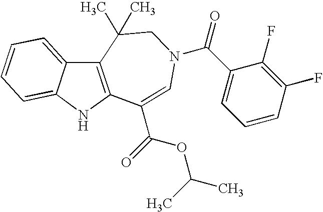 | A | A | A |
| 75 | 1-methylethyl 3-[(2,6- difluorophenyl)carbonyl]- 1,1-dimethyl-1,2,3,6- tetrahydroazepino[4,5- b]indole-5-carboxylate | 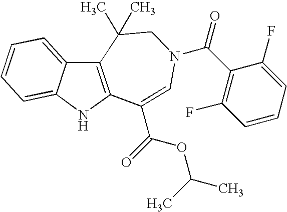 | A | A | A |
| 76 | 1-methylethyl 2-[(2,5- difluorophenyl)carbonyl]- 1,1-dimethyl-1,2,3,6- tetrahydroazepino[4,5- b]indole-5-carboxylate | 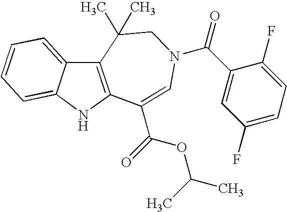 | B | A | D |
| 77 | 1-methylethyl 1,1-dimethyl- 3-[(2,3,4- trifluorophenyl)carbonyl]- 1,2,3,6- tetrahydroazepino[4,5- b]indole-5-carboxylate | 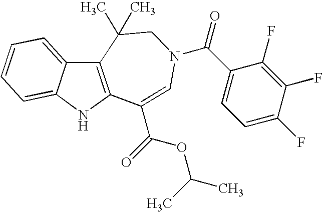 | A | A | A |
| 78 | 1-methylethyl 1,1-dimethyl- 3-[(2,4,6- trifluorophenyl)carbonyl]- 1,2,3,6- tetrahydroazepino[4,5- b]indole-5-carboxylate | 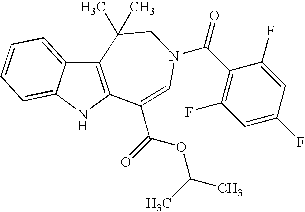 | B | A | A |
| 79 | 1-methylethyl 1,1-dimethyl- 3-[(2,4,5- trifluorophenyl)carbonyl]- 1,2,3,6- tetrahydroazepino[4,5- b]indole-5-carboxylate | 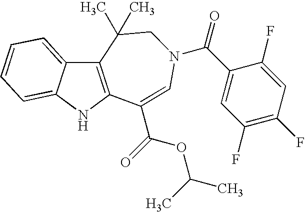 | A | A | A |
| 80 | 1-methylethyl 3-(1,3- benzodioxol-5-ylcarbonyl)- 1,1-dimethyl-1,2,3,6- tetrahydroazepino[4,5- b]indole-5-carboxylate | 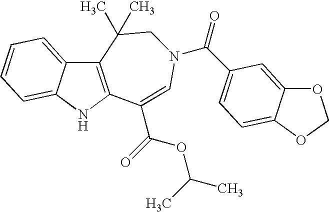 | B | A | D |
| 81 | 1-methylethyl 3-[(3- chlorophenyl)carbonyl]-1,1- dimethyl-1,2,3,6- tetrahydroazepino[4,5- b]indole-5-carboxylate | 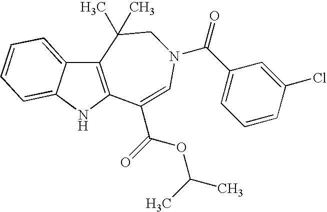 | B | A | D |
| 82 | 1-methylethyl 3[(4- chlorophenyl)carbonyl]-1,1- dimethyl-1,2,3,6- tetrahydroazepino[4,5- b]indole-5-carboxylate | 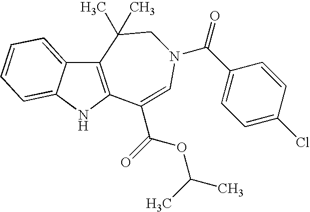 | B | A | D |
| 83 | 1-methylethyl 3-{[4-fluoro- 3- (trifluoromethyl)phenyl]car- bonyl}-1,1-dimethyl-1,2,3,6- tetrahydroazepino[4,5- b]indole-5-carboxylate | 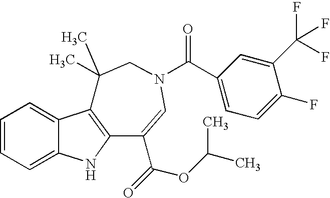 | B | A | D |
| 84 | 1-methylethyl 3-{[3-fluoro- 4- (trifluoromethyl)phenyl]car- bonyl}-1,1-dimethyl-1,2,3,6- tetrahydroazepino[4,5- b]indole-5-carboxylate | 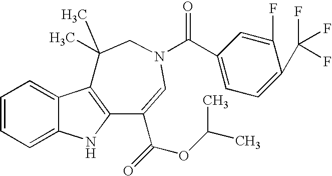 | B | A | A |
| 85 | N-({3-[(3,4- difluorophenyl)carbonyl]- 1,1-dimethyl-1,2,3,4,5,6- hexahydroazepino[4,5- b]indol-5-yl}carbonyl)-beta- alanine | 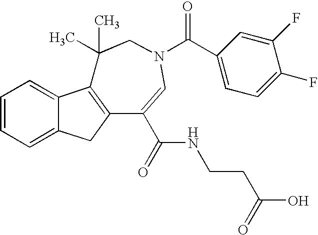 | D | D | |
| 86 | 1-methylethyl 1,1-dimethyl- 3-[(3- methylphenyl)carbonyl]- 1,2,3,6- tetrahydroazepino[4,5- b]indole-5-carboxylate | 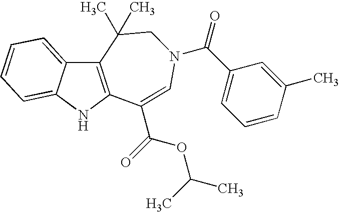 | A | A | D |
| 87 | 1-methylethyl 1,1-dimethyl- 3-[(4- methylphenyl)carbonyl]- 1,2,3,6- tetrahydroazepino[4,5- b]indole-5-carboxylate | 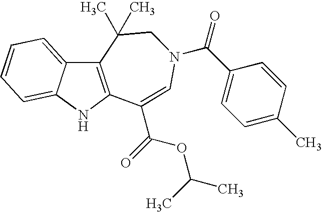 | B | A | |
| 88 | 1-methylethyl 1,1-dimethyl- 3-{[3- (methyloxy)phenyl]carbonyl}- 1,2,3,6- tetrahydroazepino[4,5- b]indole-5-carboxylate | 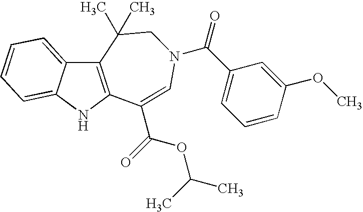 | B | A | |
| 89 | 1-methylethyl 1,1-dimethyl- 3-{[4- (methyloxy)phenyl)carbonyl}- 1,2,3,6- tetrahydroazepino[4,5- b]indole-5-carboxylate | 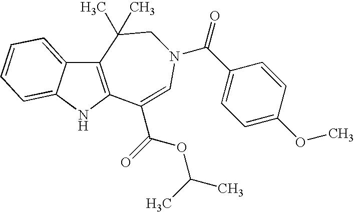 | A | A | A |
| 90 | 3-[(3,4- difluorophenyl)carbonyl]- 1,1-dimethyl-N-(1- methylethyl)-1,2,3,4,5,6- hexahydroazepino[4,5- b]indole-5-carboxamide | 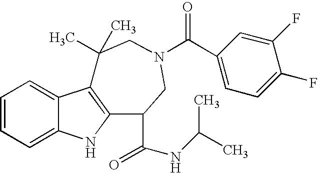 | D | A | E |
| 91 | 1-methylethyl 3-[(2,2- difluoro-1,3-benzodioxol-4- yl)carbonyl]-1,1-dimethyl- 1,2,3,6- tetrahydroazepino[4,5- b]indole-5-carboxylate | 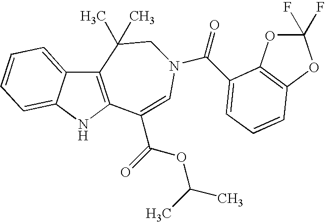 | B | A | A |
| 92 | 1-methylethyl 3-[(2,2- difluoro-1,3-benzodioxol-5- yl)carbonyl]-1,1-dimethyl- 1,2,3,6- tetrahydroazepino[4,5- b]indole-5-carboxylate | 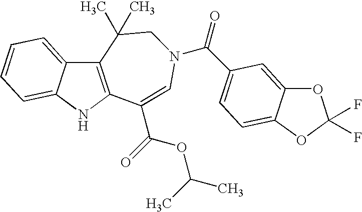 | C | A | |
| 93 | 1-methylethyl 3-[(3,4- difluorophenyl)carbonyl]-8- [({[2- (dimethylamino)ethyl]amino} carbonyl)oxy]-1,1- dimethyl-1,2,3,6- tetrahydroazepino[4,5- b]indole-5-carboxylate | 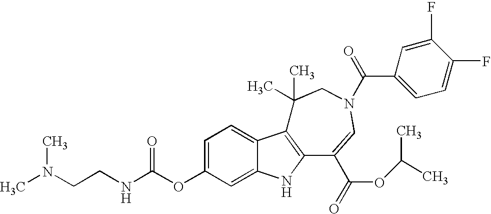 | B | A | E |
| 94 | 1-methylethyl 1,1-dimethyl- 3-[(4-piperidin-4- ylphenyl)carbonyl]-1,2,3,6- tetrahydroazepino[4,5- b]indole-5-carboxylate | 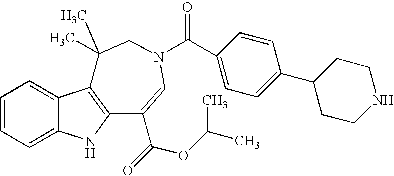 | E | | |
| 95 | 1-methylethyl 1,1-dimethyl- 3-[(3-piperidin-4- ylphenyl)carbonyl]-1,2,3,6- tetrahydroazepino[4,5- b]indole-5-carboxylate | 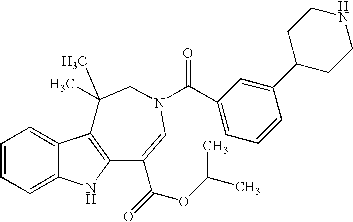 | E | | |
| 96 | 1-methylethyl 3-[(3,4- difluorophenyl)carbonyl]-8- ({[(1,1- dimethylethyl)oxy]carbonyl} oxy)-1,1-dimethyl-1,2,3,6- tetrahydroazepino[4,5- b]indole-5-carboxylate | 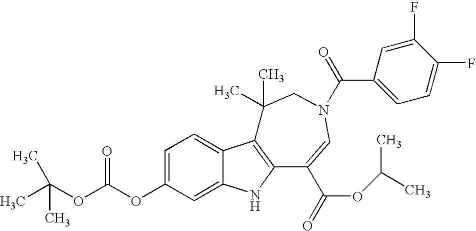 | B | A | D |
| 97 | 1-methylethyl 8-[({[2- (diethylamino)ethyl]amino} carbonyl)oxy]-3-[(3,4- difluorophenyl)carbonyl]- 1,1-dimethyl-1,2,3,6- tetrahydroazepino[4,5- b]indole-5-carboxylate | 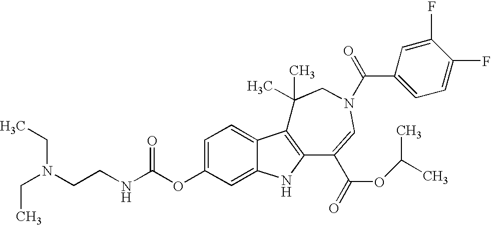 | B | A | |
| 98 | 1-methylethyl 3-[(3,4- difluorophenyl)carbonyl]-8- {[2- (dimethylamino)ethyl]oxy}- 1,1-dimethyl-1,2,3,6- tetrahydroazepino[4,5- b]indole-5-carboxylate | 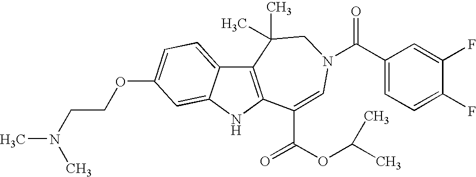 | C | E | |
| 99 | 1-methylethyl 3-[(3,4- difluorophenyl)carbonyl]-8- {[3- (dimethylamino)propyl]oxy}- 1,1-dimethyl-1,2,3,6- tetrahydroazepino[4,5- b]indole-5-carboxylate | 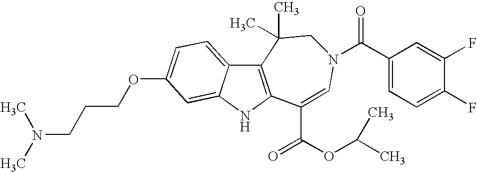 | C | D | |
| 100 | 1-methylethyl 3-{[3,4- bis(methyloxy)phenyl]car- bonyl}-1,1-dimethyl-1,2,3,6- tetrahydroazepino[4,5- b]indole-5-carboxylate | 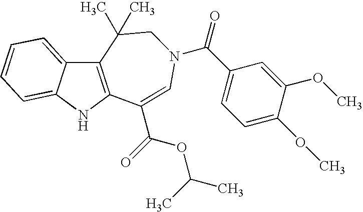 | B | A | A |
| 101 | N-({3-[(3,4- difluorophenyl)carbonyl]- 1,1-dimethyl-1,2,3,6- tetrahydroazepino[4,5- b]indol-5-yl}carbonyl)-beta- alanine | 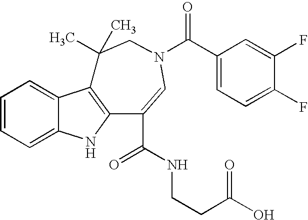 | E | | |
| 102 | 1-methylethyl 3-[(3,4- difluorophenyl)carbonyl]- 1,1-dimethyl-8- {[(methylamino)carbonyl] oxy}-1,2,3,6- tetrahydroazepino[4,5- b]indole-5-carboxylate | 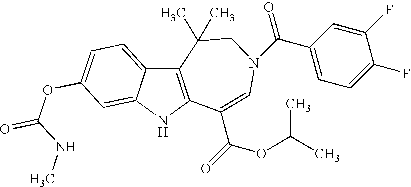 | B | E | A |
| 103 | 1-methylethyl 8-{[2- (diethylamino)ethyl]oxy}-3- [(3,4- difluorophenyl)carbonyl]- 1,1-dimethyl-1,2,3,6- tetrahydroazepino[4,5- b]indole-5-carboxylate | 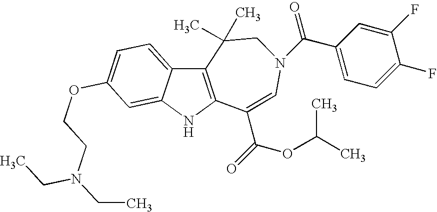 | C | E | |
| 104 | 1-methylethyl 8-{[3- (diethylamino)propyl]oxy}- 3-[3,4- difluorophenyl)carbonyl]- 1,1-dimethyl-1,2,3,6- tetrahydroazepino[4,5- b]indole-5-carboxylate | 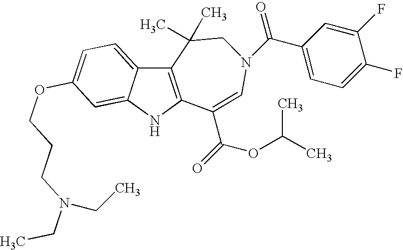 | C | C | |
| 105 | 1-methylethyl 3-[(3,4- difluorophenyl)carbonyl]- 1,1-dimethyl-8-{[2- (methyloxy)ethyl]oxy}- 1,2,3,6- tetrahydroazepino[4,5- b]indole-5-carboxylate | 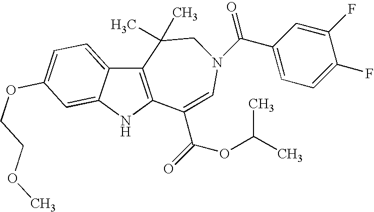 | C | B | A |
| 106 | 1-{3-[(3,4- difluorophenyl)carbonyl]- 1,1-dimethyl-1,2,3,6- tetrahydroazepino[4,5- b]indol-5-yl}ethanone | 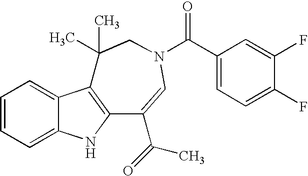 | C | A | A |
| 107 | 1-methylethyl 3-({4- [(dimethylamino)methyl] phenyl}carbonyl)-1,1-dimethyl- 1,2,3,6- tetrahydroazepino[4,5- b]indole-5-carboxylate | 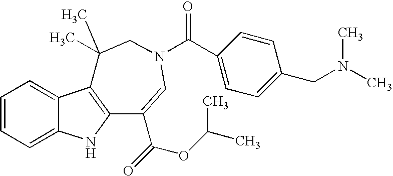 | D | A | |
| 108 | 1-methylethyl 3-({3- [(dimethylamino)methyl] phenyl}-carbonyl)-1,1-dimethyl- 1,2,3,6- tetrahydroazepino[4,5- b]indole-5-carboxylate | 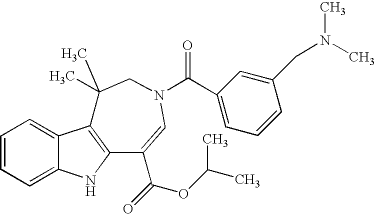 | C | A | |
| 109 | 1-methylethyl 1,1-dimethyl- 3-[(5-methylisoxazol-3- yl)carbonyl]-1,2,3,6- tetrahydroazepino[4,5- b]indole-5-carboxylate | 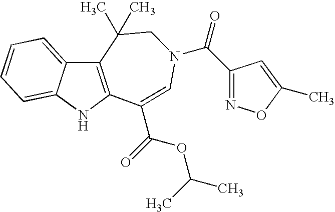 | C | A | A |
| 110 | 1-methylethyl 3-{[4-fluoro- 2- (trifluoromethyl)phenyl]car- bonyl}-1,1-dimethyl-1,2,3,6- tetrahydroazepino[4,5- b]indole-5-carboxylate | 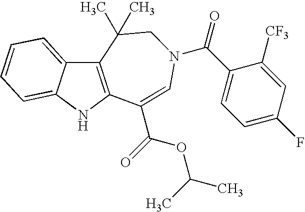 | B | A | A |
| 111 | 1-methylethyl 3-[(2-chloro- 4-fluorophenyl)carbonyl]- 1,1-dimethyl-1,2,3,6- tetrahydroazepino[4,5- b]indole-5-carboxylate | 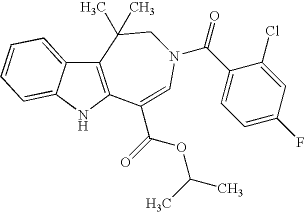 | B | A | A |
| 112 | 1-methylethyl 8-[({[3- (diethylamino)propyl]amino} carbonyl)oxy]-3-[(3,4- difluorophenyl)carbonyl]- 1,1-dimethyl-1,2,3,6- tetrahydroazepino[4,5- b]indole-5-carboxylate | 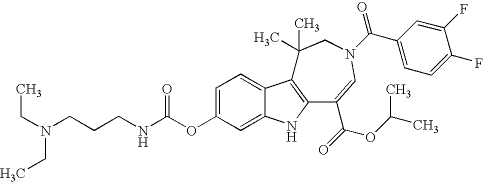 | D | A | |
| 113 | 1-methylethyl 3-{[3- (chloromethyl)phenyl]car- bonyl}-1,1-dimethyl-1,2,3,6- tetrahydroazepino[4,5- b]indole-5-carboxylate | 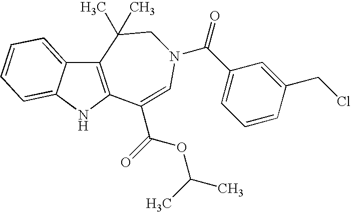 | B | A | A |
| 114 | 1-methylethyl 3-({3- [(diethylamino)methyl]phenyl} carbonyl)-1,1-dimethyl- 1,2,3,6- tetrahydroazepino[4,5- b]indole-5-carboxylate | 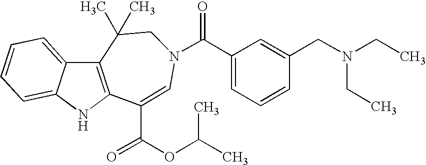 | C | A | |
| 115 | 1-methylethyl 3-[(3,4- difluorophenyl)carbonyl]- 1,1-dimethyl-9- [(phenylmethyl)oxy]-1,2,3,6- tetrahydroazepino[4,5- b]indole-5-carboxylate | 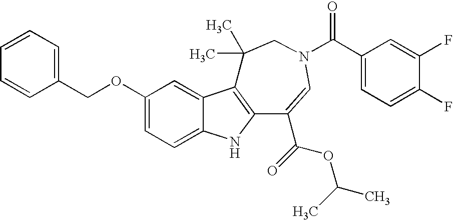 | B | A | |
| 116 | 1-methylethyl 3-[(3,4- difluorophenyl)carbonyl]-9- hydroxy-1,1-dimethyl- 1,2,3,6- tetrahydroazepino[4,5- b]indole-5-carboxylate | 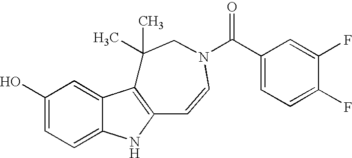 | C | A | |
| 117 | 1-methylethyl 1,1-dimethyl- 3-{[3-(pyrrolidin-1- ylmethyl)phenyl]carbonyl}- 1,2,3,6- tetrahydroazepino[4,5- b]indole-5-carboxylate | 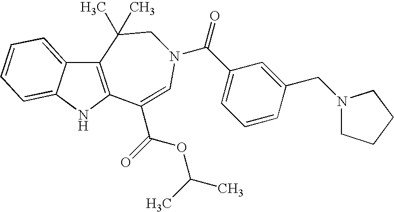 | C | A | |
| 118 | 1-methylethyl 1,1-dimethyl- 3-{[3-(piperidin-1- ylmethyl)phenyl]carbonyl}- 1,2,3,6- tetrahydroazepino[4,5- b]indole-5-carboxylate | 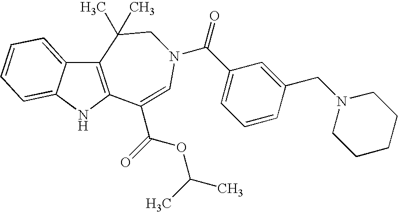 | C | A | |
| 119 | 1-methylethyl 1,1-dimethyl- 3-({3-[(4-methylpiperazin-1- yl)methyl]phenyl}carbonyl)- 1,2,3,6- tetrahydroazepino[4,5- b]indole-5-carboxylate | 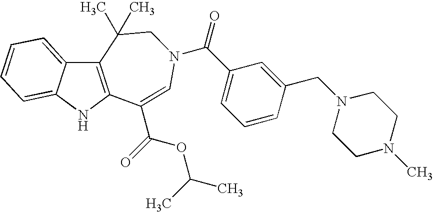 | C | A | A |
| 120 | 1-methylethyl 3-({3-[(4- ethylpiperazin-1- yl)methyl]phenyl}carbonyl)- 1,1-dimethyl-1,2,3,6- tetrahydroazepino[4,5- b]indole-5-carboxylate | 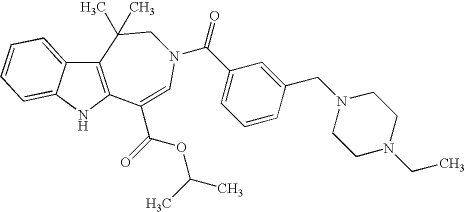 | C | A | A |
| 121 | 2-chloro-1-{3-[(3,4- difluorophenyl)carbonyl]- 1,1-dimethyl-1,2,3,6- tetrahydroazepino[4,5- b]indol-5-yl}ethanone | 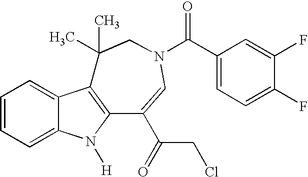 | C | E | A |
| 122 | (2,2-dimethyl-1,3-dioxolan- 4-yl)methyl 3-[(3,4- difluorophenyl)carbonyl]- 1,1-dimethyl-1,2,3,6- tetrahydroazepino[4,5- b]indole-5-carboxylate | 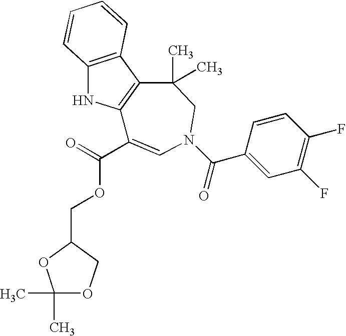 | C | A | A |
| 123 | methyl 3-[(3,4- difluorophenyl)carbonyl]- 1,1-dimethyl-1,2,3,6- tetrahydroazepino[4,5- b]indole-5-carboxylate | 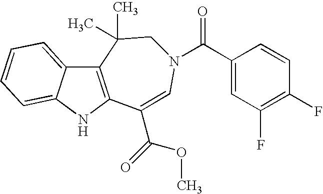 | C | A | A |
| 124 | 2,3-dihydroxypropyl 3-[(3,4- difluorophenyl)carbonyl]- 1,1-dimethyl-1,2,3,6- tetrahydroazepino[4,5- b]indole-5-carboxylate | 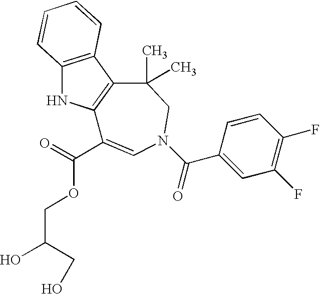 | B | A | |
| 125 | 1-methylethyl 1,1-dimethyl- 3-{[3-(morpholin-4- ylmethyl)phenyl]carbonyl}- 1,2,3,6- tetrahydroazepino[4,5- b]indole-5-carboxylate | 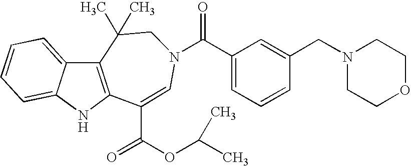 | B | A | A |
| 126 | 1-methylethyl 3-[(3,4- difluorophenyl)carbonyl]- 1,1-dimethyl-8-({[(2- pyrrolidin-1- ylethyl)amino]carbonyl}oxy)- 1,2,3,6- tetrahydroazepino[4,5- b]indole-5-carboxylate | 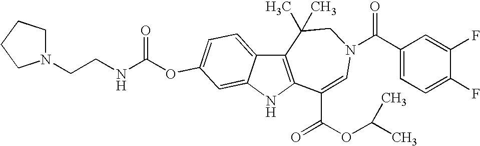 | B | A | |
| 127 | 1-methylethyl 1,1-dimethyl- 3-({3- [(phenylmethyl)oxy]phenyl} carbonyl)-1,2,3,6- tetrahydroazepino[4,5- b]indole-5-carboxylate | 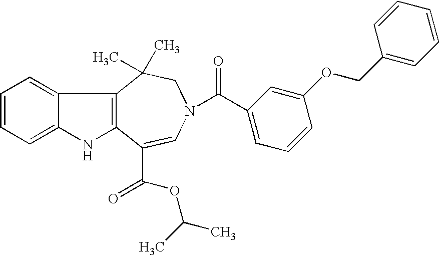 | B | A | D |
| 128 | 1-methylethyl 1,1-dimethyl- 3-{[3- (trifluoromethyl)phenyl]car- bonyl}-1,2,3,6- tetrahydroazepino[4,5- b]indole-carboxylate | 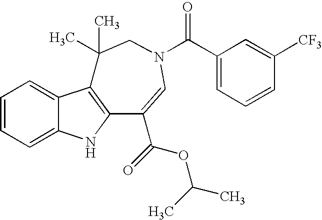 | B | A | |
| 129 | (2R)-2,3-dihydroxypropyl 3- [(3,4- difluorophenyl)carbonyl]- 1,1-dimethyl-1,2,3,6- tetrahydroazepino[4,5- b]indole-5-carboxylate | 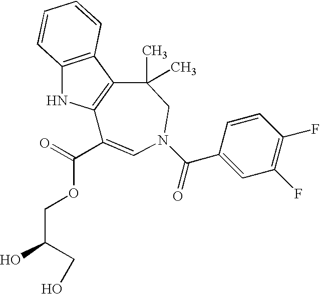 | C | A | |
| 130 | 1-methylethyl 3-[(3-fluoro-4- methylphenyl)carbonyl]-1,1- dimethyl-1,2,3,6- tetrahydroazepino[4,5- b]indole-5-carboxylate | 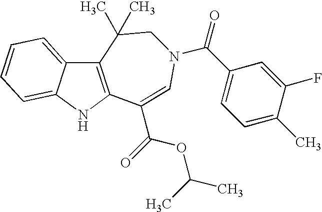 | B | A | D |
| 131 | 1-methylethyl 3-{[2-fluoro- 4- (trifluoromethyl)phenyl]car- bonyl}-1,1-dimethyl-1,2,3,6- tetrahydroazepino[4,5- b]indole-5-carboxylate | 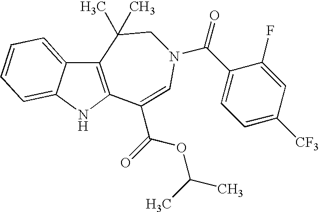 | C | A | E |
| 132 | 1-methylethyl 3-{[3-chloro- 2-fluoro-4- (trifluoromethyl)phenyl]car- bonyl}-1,1-dimethyl-1,2,3,6- tetrahydroazepino[4,5- b]indole-5-carboxylate | 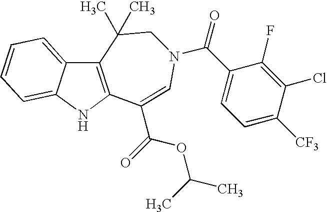 | C | A | D |
| 133 | 1-methylethyl 3-{[2-fluoro- 3- (trifluoromethyl)phenyl]car- bonyl}-1,1-dimethyl-1,2,3,6- tetrahydroazepino[4,5- b]indole-5-carboxylate | 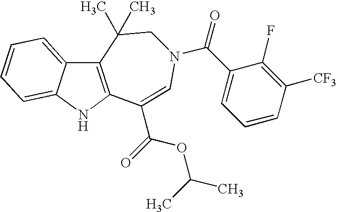 | B | A | A |
| 134 | 1-methylethyl 3-{[3-fluoro- 5- (trifluoromethyl)phenyl]car- bonyl}-1,1-dimethyl-1,2,3,6- tetrahydroazepino[4,5- b]indole-5-carboxylate | 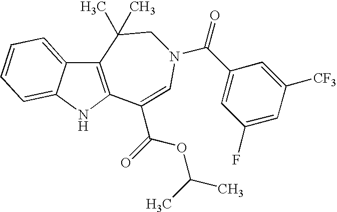 | B | A | D |
| 135 | 1-methylethyl 3-{[3,5- bis(trifluoromethyl)phenyl] carbonyl}-1,1-dimethyl- 1,2,3,6- tetrahydroazepino[4,5- b]indole-5-carboxylate | 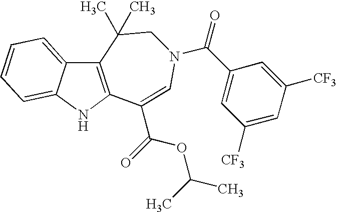 | B | A | D |
| 136 | 2-fluoro-1- (fluoromethyl)ethyl 3-[(3,4- difluorophenyl)carbonyl]- 1,1-dimethyl-1,2,3,6- tetrahydroazepino[4,5- b]indole-5-carboxylate | 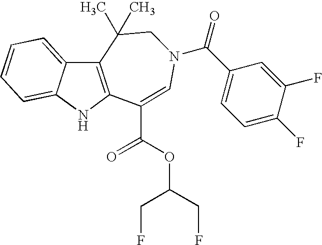 | A | A | A |
| 137 | 1-methylethyl 3-{[2,5- bis(trifluoromethyl)phenyl] carbonyl}-1,1-dimethyl- 1,2,3,6- tetrahydroazepino[4,5- b]indole-5-carboxylate | 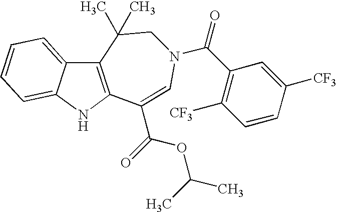 | C | A | C |
| 138 | 1-methylethyl 3-{[2,3- difluoro-4- (trifluoromethyl)phenyl]car- bonyl}-1,1-dimethyl-1,2,3,6- tetrahydroazepino[4,5- b]indole-5-carboxylate | 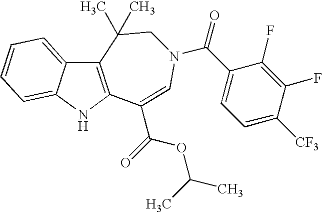 | B | A | C |
| 139 | 1-methylethyl 3-[(3- hydroxyphenyl)carbonyl]- 1,1-dimethyl-1,2,3,6- tetrahydroazepino[4,5- b]indole-5-carboxylate | 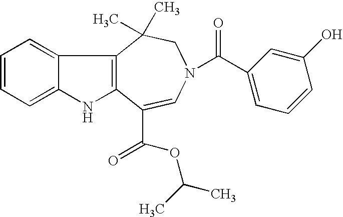 | B | A | D |
| 140 | 1-methylethyl 3-[(3-{[2- (dimethylamino)ethyl]oxy} phenyl)carbonyl]-1,1- dimethyl-1,2,3,6- tetrahydroazepino[4,5- b]indole-5-carboxylate | 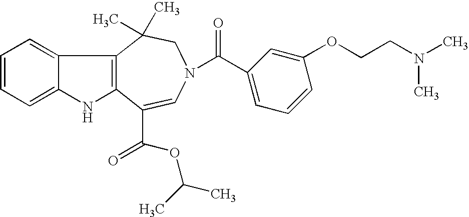 | C | A | E |
| 141 | 1-methylethyl 1,1-dimethyl- 3-{[4-(1H-pyrazol-1- yl)phenyl]carbonyl}-1,2,3,6- tetrahydroazepino[4,5- b]indole-5-carboxylate | 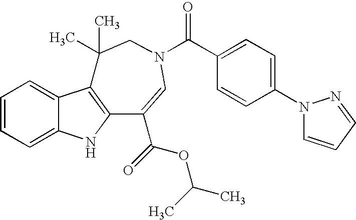 | B | A | A |
| 142 | 1-methylethyl 3-[(3- cyanophenyl)carbonyl]-1,1- dimethyl-1,2,3,6- tetrahydroazepino[4,5- b]indole-5-carboxylate | 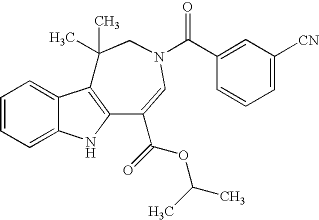 | B | A | A |
| 143 | 1-methylethyl 3-[(2,4- dichlorophenyl)carbonyl]- 1,1-dimethyl-1,2,3,6- tetrahydroazepino[4,5- b]indole-5-carboxylate | 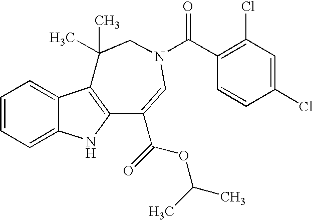 | B | A | A |
| 144 | 1-methylethyl 3-[(4-fluoro-3- methylphenyl)carbonyl]-1,1- dimethyl-1,2,3,6- tetrahydroazepino[4,5- b]indole-5-carboxylate | 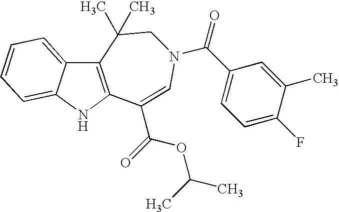 | A | A | A |
| 145 | 1-methylethyl 3-[(3-chloro- 2,6- difluorophenyl)carbonyl]- 1,1-dimethyl-1,2,3,6- tetrahydroazepino[4,5- b]indole-5-carboxylate | 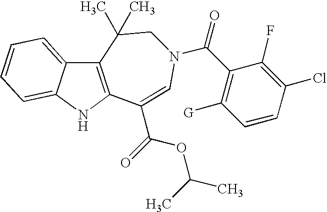 | C | A | |
| 146 | 1-methylethyl 3-({3-[(4- acetylpiperazin-1- yl)methyl]phenyl}carbonyl)- 1,1-dimethyl-1,2,3,6- tetrahydroazepino[4,5- b]indole-5-carboxylate | 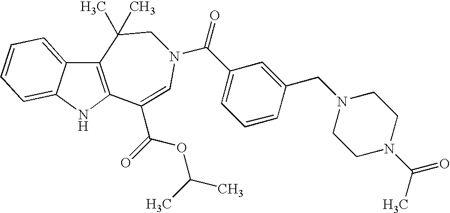 | C | A | D |
| 147 | 1-methylethyl 3-[(3-chloro- 4-fluorophenyl)carbonyl]- 1,1-dimethyl-1,2,3,6- tetrahydroazepino[4,5- b]indole-5-carboxylate | 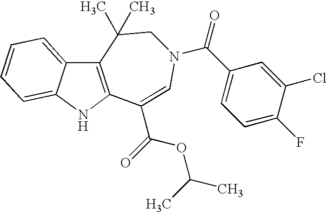 | A | A | A |
| 148 | 1-methylethyl 3-[(3,4- dichlorophenyl)carbonyl]- 1,1-dimethyl-1,2,3,6- tetrahydroazepino[4,5- b]indole-5-carboxylate | 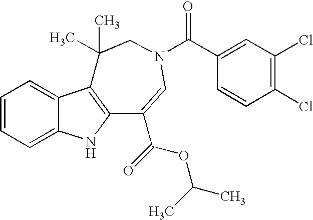 | B | A | A |
| 149 | 1-methylethyl 3-[(4-chloro- 2,5- difluorophenyl)carbonyl]- 1,1-dimethyl-1,2,3,6- tetrahydroazepino[4,5- ]indole-5-carboxylate | 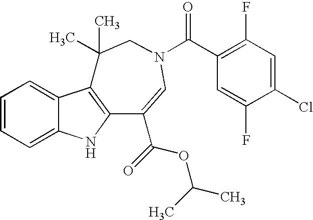 | B | A | A |
| 150 | 1-methylethyl 3-[(3-{[3- (dimethylamino)propyl]oxy} phenyl)carbonyl]-1,1- dimethyl-1,2,3,6- tetrahydroazepino[4,5- b]indole-4-carboxylate | 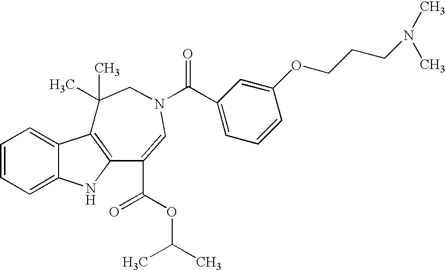 | D | A | |
| 151 | 1-methylethyl 1,1-dimethyl- 3-[(3-{[4- (methylsulfonyl)piperazin-1- yl]methyl}phenyl)carbonyl]- 1,2,3,6- tetrahydroazepino[4,5- b]indole-5-carboxylate | 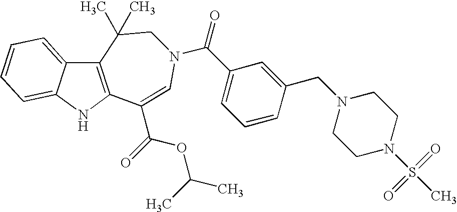 | C | A | A |
| 152 | 1-methylethyl 3-{[3-(azepan- 1- ylmethyl)phenyl]carbonyl}- 1,1-dimethyl-1,2,3,6- tetrahydroazepino[4,5- b]indole-5-carboxylate | 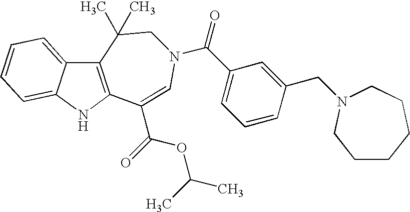 | B | A | A |
| 153 | 1-methylethyl 1,1-dimethyl- 3-({3-[(4-methyl-1,4- diazepan-1- yl)methyl]phenyl}carbonyl)- 1,2,3,6- tetrahydroazepino[4,5- b]indole-5-carboxylate | 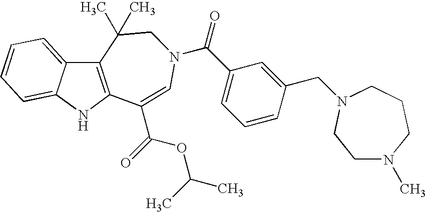 | D | A | |
| 154 | 1-methylethyl 3-[(3-bromo- 4-fluorophenyl)carbonyl]- 1,1-dimethyl-1,2,3,6- tetrahydroazepino[4,5- b]indole-5-carboxylate | 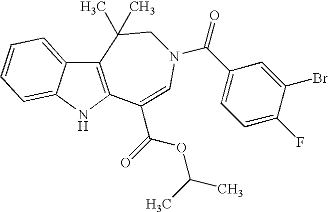 | B | A | A |
| 155 | 1-methylethyl 3-{[2-fluoro- 5-(morpholin-4- ylmethyl)phenyl]carbonyl}- 1,1-dimethyl-1,2,3,6- tetrahydroazepino[4,5- b]indole-5-carboxylate | 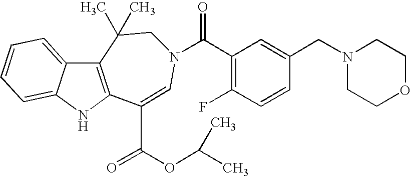 | C | A | A |
| 156 | 1-methylethyl 3-{[4-fluoro- 3-(morpholin-4- ylmethyl)phenyl]carbonyl}- 1,1-dimethyl-1,2,3,6- tetrahydroazepino[4,5- b]indole-4-carboxylate | 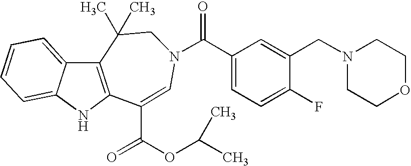 | C | A | A |
| 157 | 1-methylethyl 1,1-dimethyl- 3-[(1-methyl-1H-1,2,3- benzotriazol-5-yl)carbonyl]- 1,2,3,6- tetrahydroazepino[4,5- b]indole-5-carboxylate | 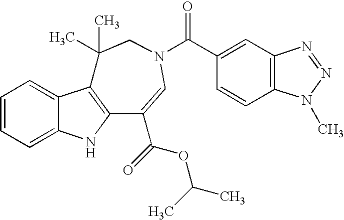 | B | A | A |
| 158 | 1-methyethyl 1,1-dimethyl- 3-({4-[4-(trifluoromethyl)- 1H-pyrazol-1- yl]phenyl}carbonyl)-1,2,3,6- tetrahydroazepino[4,5- b]indole-5-carboxylate | 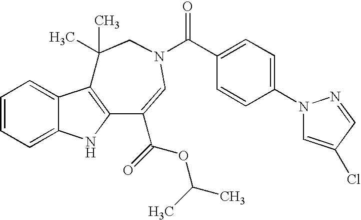 | B | A | C |
| 159 | 1-methylethyl 1,1-dimethyl- 3-({3-[(2-piperidin-1- ylethyl)oxy]phenyl}carbonyl)- 1,2,3,6- tetrahydroazepino[4,5- b]indole-5-carboxylate | 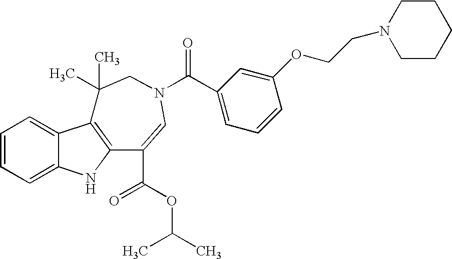 | C | A | D |
| 160 | 1-methylethyl 1,1-dimethyl- 3-({3-[(2-morpholin-4- ylethyl)oxy]phenyl}carbonyl)- 1,2,3,6- tetrahydroazepino[4,5- b]indole-5-carboxylate | 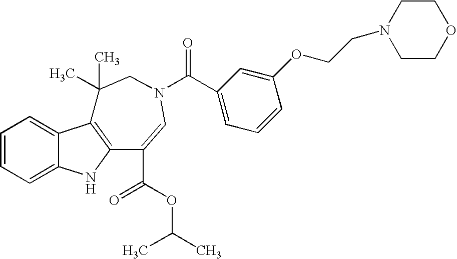 | C | A | A |
| 161 | 3-[(3,4- difluorophenyl)carbonyl]-N- [(2,3-dihydroxypropyl)oxy]- 1,1-dimethyl-1,2,3,6- tetrahydroazepino[4,5- b]indole-5-carboxamide | 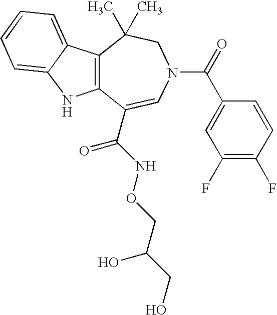 | E | | |
| 162 | 3-[(3,4- difluorophenyl)carbonyl]-N- (2,3-dihydroxypropyl)-1,1- dimethyl-1,2,3,6- tetrahydroazepino[4,5- b]indole-5-carboxamide | 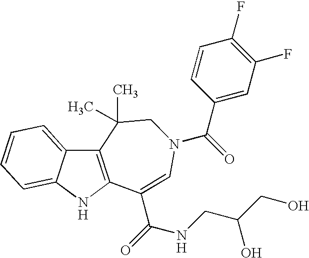 | E | | |
| 163 | 3-[(3,4- difluorophenyl)carbonyl]-N- [2-hydroxy-1- (hydroxymethyl)ethyl]-1,1- dimethyl-1,2,3,6- tetrahydroazepino[4,5- b]indole-5-carboxamide | 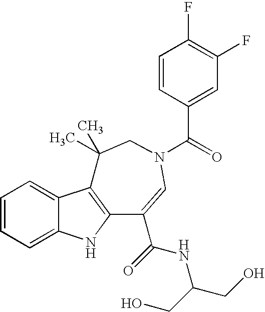 | D | A | |
| 164 | 1-methylethyl 3-{[2-fluoro- 5-(piperidin-1- ylmethyl)phenyl]carbonyl}- 1,1-dimethyl-1,2,3,6- tetrahydroazepino[4,5- b]indole-5-carboxylate | 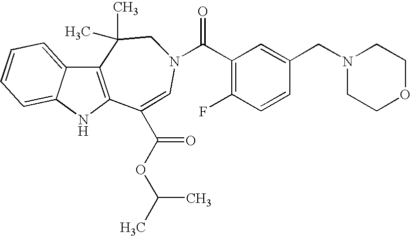 | C | A | A |
| 165 | 1-methylethyl 3-{[4-fluoro- 3-(piperidin-1- ylmethyl)phenyl]carbonyl}- 1,1-dimethyl-1,2,3,6- tetrahydroazepino[4,5- b]indole-5-carboxylate | 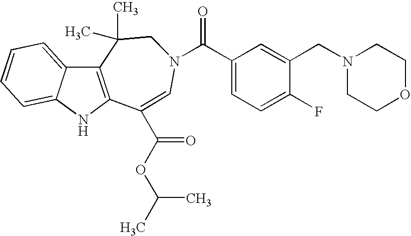 | C | A | A |
| 166 | 1-methyethyl 3-({3-[(4- {[(1,1-dimethylethyl)oxy]carbonyl} piperazin-1- yl)methyl]phenyl}carbonyl)- 1,1-dimethyl-1,2,3,6- tetrahydroazepino[4,5- b]indole-5-carboxylate | 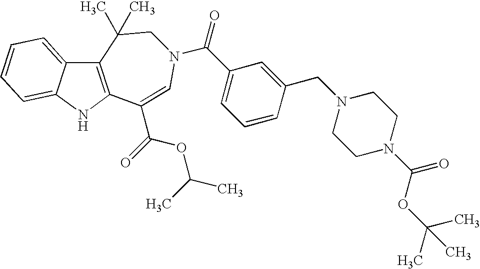 | C | A | A |
| 167 | 1-methylethyl 1,1-dimethyl- 3-[(3-{[4- (phenylsulfonyl)piperazin-1- yl]methyl}phenyl)carbonyl]- 1,2,3,6- tetrahydroazepino[4,5- b]indole-5-carboxylate | 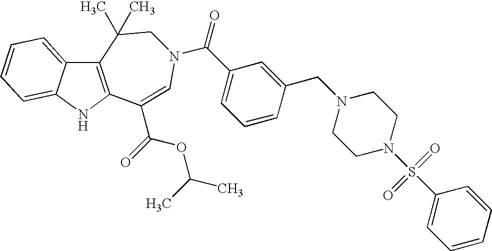 | C | A | A |
| 168 | 1-methylethyl 3-{[3-({4-[(4- fluorophenyl)sulfonyl]piper- azin-1- yl}methyl)phenyl]carbonyl}- 1,1-dimethyl-1,2,3,6- tetrahydroazepino[4,5- b]indole-5-carboxylate | 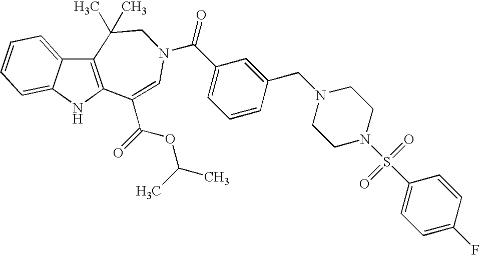 | C | A | A |
| 169 | 1-methylethyl 3-{[3,4- difluoro-5-({[4- (methyloxy)phenyl]methyl} oxy)phenyl]carbonyl}-1,1- dimethyl-1,2,3,6- tetrahydroazepino[4,5- b]indole-5-carboxylate | 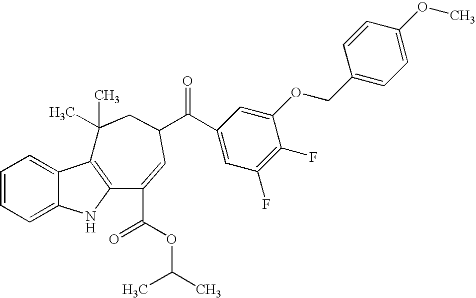 | C | A | D |
| 170 | 1-methylethyl 3-[(3,4- difluorophenyl)carbonyl]- 1,1-dimethyl-8-({[(2- piperidin-1- ylethyl)amino]carbonyl}oxy)- 1,2,3,6- tetrahydroazepino[4,5- b]indole-5-carboxylate | 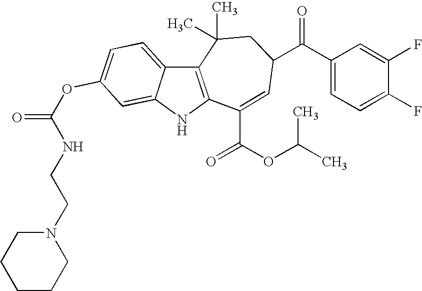 | B | A | E |
| 171 | (2S)-2,3-dihydroxypropyl-3- [(3,4- difluorophenyl)carbonyl]- 1,1-dimethyl-1,2,3,6- tetrahydroazepino[4,5- b]indole-5-carboxylate | 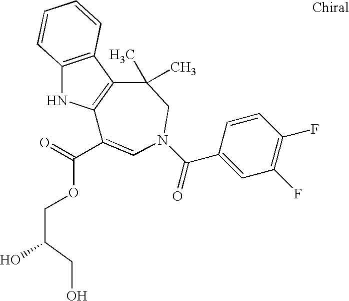 | B | A | E |
| 172 | 1-methylethyl 3-[(3-{[4- (ethylsulfonyl)piperazin-1- yl]methyl}phenyl)carbonyl]- 1,1-dimethyl-1,2,3,6- tetrahydroazepino[4,5- b]indole-5-carboxylate | 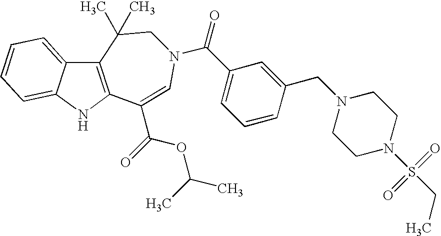 | C | A | |
| 173 | 1-methylethyl 3-[(3-{[4- (cyclopropylcarbonyl)piper- azin-1- yl]methyl}phenyl)carbonyl]- 1,1-dimethyl-1,2,3,6- tetrahydroazepino[4,5- b]indole-5-carboxylate | 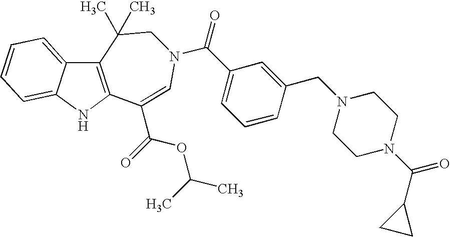 | C | A | A |
| 174 | 1-methylethyl 1,1-dimethyl- 3-[(3-{[4-(2- methylpropanoyl)piperazin- 1- yl]methyl}phenyl)carbonyl]- 1,2,3,6- tetrahydroazepino[4,5- b]indole-5-carboxylate | 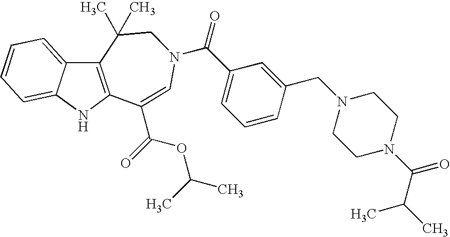 | C | A | A |
| 175 | 1-methylethyl 1,1-dimethyl- 3-[(3-{[4- (phenylcarbonyl)piperazin-1- yl]methyl}phenyl)carbonyl]- 1,2,3,6- tetrahydroazepino[4,5- b]indole-5-carboxylate | 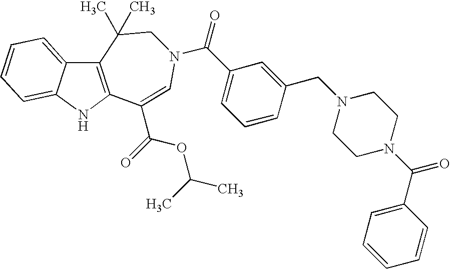 | C | A | |
| 176 | 1-methylethyl 3-{[3-(azocan- 1- ylmethyl)phenyl]carbonyl}- 1,1-dimethyl-1,2,3,6- tetrahydroazepino[4,5- b]indole-5-carboxylate | 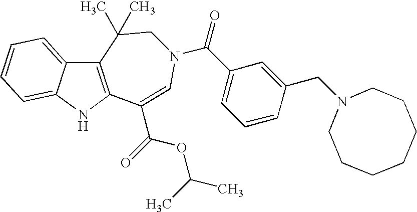 | C | A | A |
| 177 | 1-methylethyl 3-({3-[(4- acetyl-1,4-diazepan-1- yl)methyl]phenyl}carbonyl)- 1,1-dimethyl-1,2,3,6- tetrahydroazepino[4,5- b]indole-5-carboxylate | 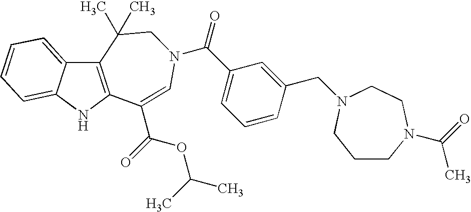 | C | A | D |
| 178 | 1-methylethyl 1,1-dimethyl- 3-{[3-(piperazin-1- ylmethyl)phenyl]carbonyl}- 1,2,3,6- tetrahydroazepino[4,5- b]indole-5-carboxylate | 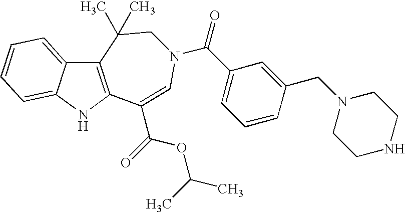 | E | | |
| 179 | 1-methylethyl 3-({3,4- difluoro-5-[(2-morpholin-4- ylethyl)oxy]phenyl}carbonyl)- 1,1-dimethyl-1,2,3,6- tetrahydroazepino[4,5- b]indole-5-carboxylate | 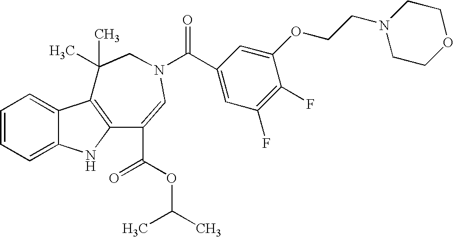 | B | A | A |
| 180 | 1-methylethyl 3-({3,4- difluoro-5-[(2-piperidin-1- ylethyl)oxy]phenyl}carbonyl)- 1,1-dimethyl-1,2,3,6- tetrahydroazepino[4,5- b]indole-5-carboxylate | 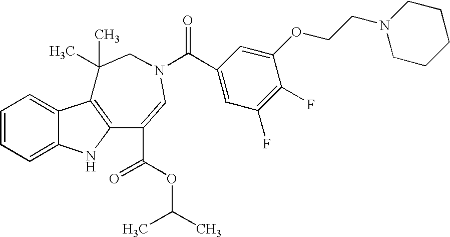 | B | A | A |
| 181 | 1-methylethyl 1,1-dimethyl- 3-({4-[(2-morpholin-4- ylethyl)oxy]phenyl}carbonyl)- 1,2,3,6- tetrahydroazepino[4,5- b]indole-5-carboxylate | 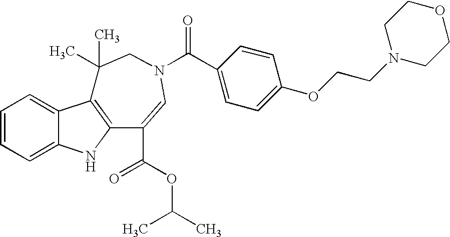 | C | A | D |
| 182 | 1-methylethyl 1,1-dimethyl- 3-({4-[(2-piperidin-1- ylethyl)oxy]phenyl}carbonyl)- 1,2,3,6- tetrahydroazepino[4,5- b]indole-5-carboxylate | 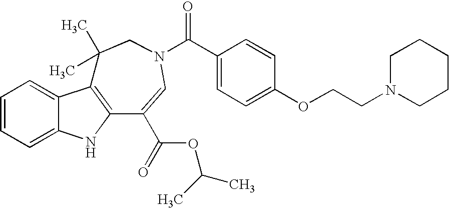 | B | A | A |
| 183 | 1-methylethyl 1,1-dimethyl- 3-({3-[(3-morpholin-4- ylpropyl)oxy]phenyl}car- bonyl)-1,2,3,6- tetrahydroazepino[4,5- b]indole-5-carboxylate | 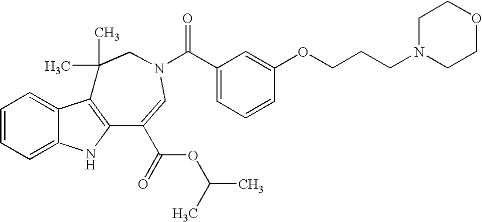 | C | A | A |
| 184 | 1-methylethyl 1,1-dimethyl- 3-{[3-({4- [(phenylamino-carbonyl]pip- erazin-1- yl}methyl)phenyl]carbonyl}- 1,2,3,6- tetrahydroazepino[4,5- b]indole-5-carboxylate | 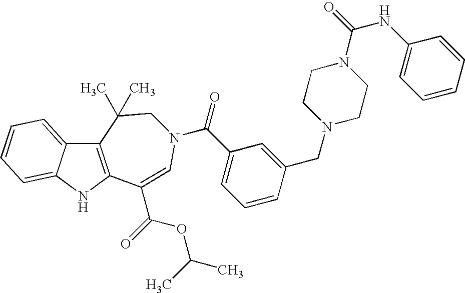 | B | A | A |
| 185 | 1-methylethyl 3-{[3-({4- [(ethylamino)carbonyl]piper- azin-1- yl}methyl)phenyl]carbonyl}- 1,1-dimethyl-1,2,3,6- tetrahydroazepino[4,5- b]indole-5-carboxylate | 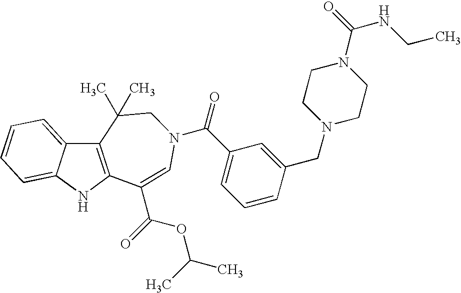 | C | A | D |
| 186 | 1-methylethyl 1,1-dimethyl- 3-({3-[(3-piperidin-1- ylpropyl)oxy]phenyl}carbon- yl)-1,2,3,6- tetrahydroazepino[4,5- b]indole-5-carboxylate | 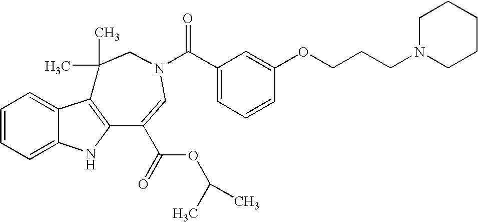 | D | | |
| 187 | 1-methylethyl 3-[(4-{[2- (dimethylamino)ethyl]oxy} phenyl)carbonyl]-1,1- dimethyl-1,2,3,6- tetrahydroazepino[4,5- b]indole-5-carboxylate | 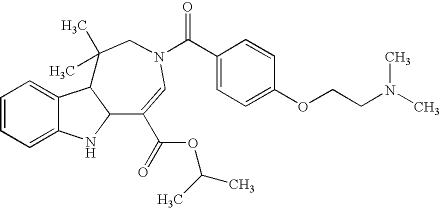 | C | A | |
| 188 | 1-methylethyl 3-[(3-{[3- (diethylamino)propyl]oxy} phenyl)carbonyl]-1,1- dimethyl-1,2,3,6- tetrahydroazepino[4,5- b]indole-5-carboxylate | 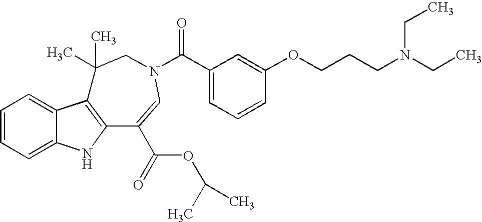 | D | A | |
| 189 | 1-methylethyl 3-[(4-{[3- (dimethylamino)propyl]oxy} phenyl)carbonyl]-1,1- dimethyl-1,2,3,6- tetrahydroazepino[4,5- b]indole-5-carboxylate | 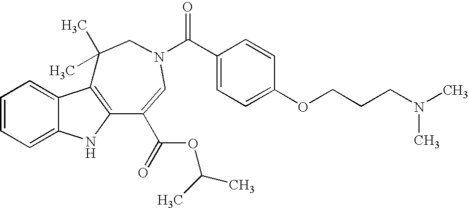 | C | A | |
| 190 | 1-methylethyl 1,1-dimethyl- 3-({4-[(2-pyrrolidin-1- ylethyl)oxy]phenyl}carbonyl)- 1,2,3,6- tetrahydroazepino[4,5- b]indole-5-carboxylate | 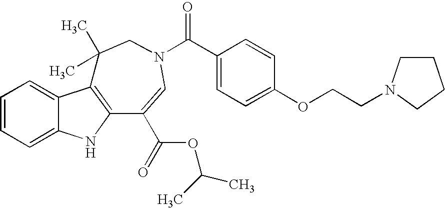 | D | A | |
| 191 | 1-methylethyl 1,1-dimethyl- 3-({4-[(3-piperidin-1- ylpropyl)oxy]phenyl}car- bonyl)-1,2,3,6- tetrahydroazepinop[4,5- b]indole-5-carboxylate | 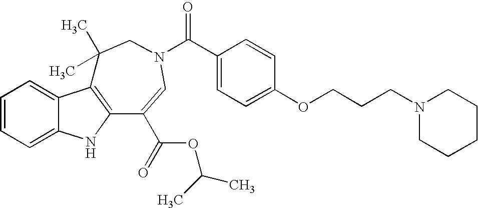 | D | A | |
| 192 | 1-methylethyl 1,1-dimethyl- 3-({4-[(3-morpholin-4- ylpropyl)oxy]phenyl}car- bonyl)-1,2,3,6- tetrahydroazepino[4,5- b]indole-5-carboxylate | 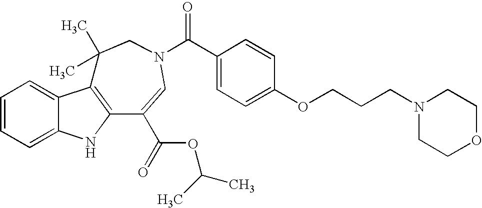 | B | A | A |
| 193 | 2-hydroxy-1-methylethyl 3- [(3,4- difluorophenyl)carbonyl]- 1,1-dimethyl-1,2,3,6- tetrahydroazepino[4,5- b]indole-5-carboxylate | 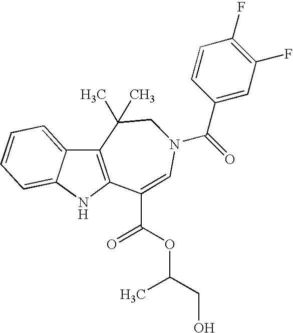 | B | A | D |
| 194 | 2-{[(3,4- difluorophenyl)carbonyl]oxy}- 1-methylethyl 3-[(3,4- difluorophenyl)carbonyl]- 1,1-dimethyl-1,2,3,6- tetrahydroazepino[4,5- b]indole-5-carboxylate | 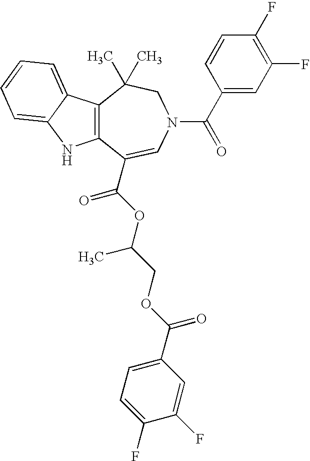 | B | A | A |
| 195 | 2-[(phenylmethyl)oxy]ethyl 3-[(3,4- difluorophenyl)carbonyl]- 1,1-dimethyl-1,2,3,6- tetrahydroazepino[4,5- b]indole-5-carboxylate | 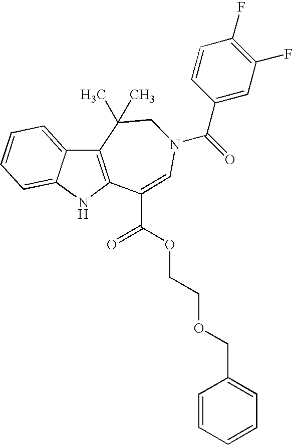 | C | A | A |
| 196 | 2-hydroxyethyl 3-[(3,4- difluorophenyl)carbonyl]- 1,1-dimethyl-1,2,3,6- tetrahydroazepino[4,5- b]indole-5-carboxylate | 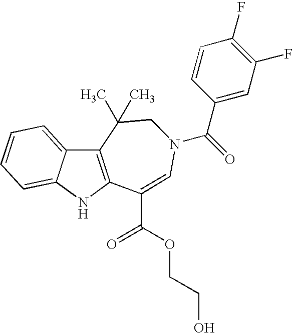 | B | A | D |

All of the U.S. patents, U.S. patent application publications, U.S. patent applications, foreign patents, foreign patent applications and non-patent publications referred to in this specification and/or listed in the Application Data Sheet, are incorporated herein by reference, in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A compound of formula (I): 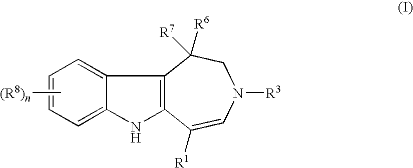
or a pharmaceutically acceptable derivative thereof, wherein:
R¹ is —C(J)R¹¹, —C(J)OR¹¹, or —C(J)N(R¹⁰)(R¹¹);
J is direct bond, O or NR¹⁰;
n is 0 to 4;
R³ is hydrogen, —C(O)R⁹, or CON(R¹¹)(R¹²);
R⁶ or R⁷ is independently optionally substituted alkyl, optionally substituted cycloalkyl or optionally substituted cycloalkylalkyl;
R⁸ is selected from the group consisting of hydroxy, optionally substituted alkyl, optionally substituted alkenyl, optionally substituted alkynyl, halo, haloalkyl, haloalkoxy, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, optionally substituted heteroaralkyl, —OC(O)N(R¹⁵)(R¹⁶), —OC(O)R¹¹, or —OR²⁰;
R⁹ is selected from the group consisting of optionally substituted alkyl, optionally substituted alkenyl, optionally substituted alkynyl, optionally substituted aryl, optionally substituted aralkyl, optionally substituted cycloalkyl, optionally substituted cycloalkylalkyl, optionally substituted heteroaryl, optionally substituted heteroaralkyl, optionally substituted heterocyclylalkyl, optionally substituted heterocyclyl, OR¹⁰ and N(R¹²)(R¹³);
R¹⁰ is independently hydrogen, optionally substituted alkyl, optionally substituted alkenyl or optionally substituted alkynyl; optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, optionally substituted heteroaralkyl;
each R¹¹ is independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, optionally substituted heteroaralkyl, —OR¹⁴ and —N(R¹⁵)(R¹⁶);
R¹² and R¹³ are each independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, and optionally substituted heteroaralkyl; or R¹² and R¹³, together with the nitrogen atom to which they are attached, form an optionally substituted heterocyclyl or an optionally substituted heteroaryl;
R¹⁰, R¹¹ R¹² and R¹³ are selected as in (a) or (b) as follows: (a)R¹⁰, R¹¹ R¹² and R¹³ each independently hydrogen, optionally substituted alkyl, optionally substituted alkenyl, optionally substituted alkynyl, optionally substituted cycloalkyl, optionally substituted cycloalkylalkyl, optionally substituted aryl, optionally substituted aralkyl, optionally substituted heterocyclyl, optionally substituted heterocyclylalkyl, optionally substituted heteroaryl, or optionally substituted heteroaralkyl; or (b) R¹⁰, R¹¹, R¹² and R¹³ together with the atoms to which they are attached, form an optionally substituted heterocyclic ring or an optionally substituted heteroaryl ring; and the others of R¹⁰, R¹¹, R¹² and R¹³, are selected as in (a), above.
each R¹⁴ is independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, optionally substituted heteroaralkyl, —OR¹⁸, —SR¹⁸ and —N(R²⁰)(R²¹);
R¹⁵ and R¹⁶ are each independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, optionally substituted heteroaralkyl, —OR¹⁸, —SR¹⁸ and —N(R²⁰)(R²¹);
or R¹⁵ and R¹⁶, together with the nitrogen atom to which they are attached, form an optionally substituted heterocyclyl ring or an optionally substituted heteroaryl ring;
R¹⁷ is hydrogen, optionally substituted alkyl, optionally substituted alkenyl or optionally substituted alkynyl;
each R¹⁸ is independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, or optionally substituted heteroaralkyl;
R¹⁹ is alkylene or direct bond;
R²⁰ and R²¹ are each independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, or optionally substituted heteroaralkyl; or
R²⁰ and R²¹, together with the nitrogen atom to which they are attached, form an optionally substituted heterocyclyl or an optionally substituted heteroaryl;
each R²² independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, optionally substituted heteroaralkyl, —R¹⁹—OR²³, —R¹⁹—N(R²³)(R²⁴), —R¹⁹—C(J)R²³, —R¹⁹—C(J)OR²³, and —R¹⁹—C(J)N(R²³)(R²⁴);
each R²³ and R²⁴ is independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl, optionally substituted heteroaralkyl, —R¹⁹—OR²⁵, —R¹⁹—N(R²⁵)(R²⁶), R¹⁹—C(J)R²⁵, —R¹⁹—C(J)OR²⁵, and —R¹⁹—C(J)N(R²⁵)(R²⁶);
or R²³ and R²⁴, together with the nitrogen atom to which they are attached, form an optionally substituted heterocyclyl or an optionally substituted heteroaryl;
each R²⁵ and R²⁶ is independently selected from the group consisting of hydrogen, optionally substituted alkyl, optionally substituted alkenyl, optionally substituted alkynyl, optionally substituted cycloalkyl, optionally substituted cycloalkylalkyl, optionally substituted heterocyclyl, optionally substituted heterocyclylalkyl, optionally substituted aryl, optionally substituted aralkyl, optionally substituted heteroaryl and optionally substituted heteroaralkyl;
each R¹-R²⁶, when substituted, are substituted with one or more substituents, each independently selected from Q¹.
where Q¹ is halo, pseudohalo, hydroxy, oxo, thia, nitrile, nitro, formyl, mercapto, amino, hydroxyalkyl, hydroxyalkylaryloxy, hydroxyaryl, hydroxyalkylaryl, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, diaryl, hydroxyaryl, alkylaryl, heteroaryl, aralkyl, aralkenyl, aralkynyl, alkylaralkyl, heteroarylalkyl, trialkylsilyl, dialkylarylsilyl, alkyldiarylsilyl, triarylsilyl, alkylidene, arylalkylidene, alkylcarbonyl, alkylarylcarbonyl, arylcarbonyl, heterocyclylcarbonyl, heteroarylcarbonyl, heteroarylalkoxycarbonyl, alkoxycarbonyl, alkoxycarbonylalkyl, alkoxycarbonylaryloxy, aryloxycarbonyl, aryloxycarbonylalkyl, heterocyclylcarbonylalkylaryl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, haloalkoxy, alkoxyaryloxy, alkylaryloxy, diaryloxy, alkylaryloxyalkyl, alkyldiaryloxy, perfluoroalkoxy, alkenyloxy, alkynyloxy, aryloxyalkaoxy, aralkoxyaryloxy, alkylarylcycloalkyloxy, heterocycloxy, alkoxyalkyl, alkoxyalkoxyalkyl, alkylheteroaryloxy, alkylcycloalkoxy, cycloalkyloxy, heterocyclyloxy, aralkoxy, haloaryloxy, heteroaryloxy, alkylheteroaryloxy, alkoxycarbonylheterocycloxy, alkylcarbonylaryloxy, alkylcarbonyloxy, arylcarbonyloxy, aralkylcarbonyloxy, alkoxycarbonyloxy, aryloxycarbonyloxy, alkoxyaryloxy, aralkoxycarbonyloxy, ureido, alkylureido, arylureido, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, haloalkylarylamino, arylamino, diarylamino, alkylarylamino, aralkylamino, alkylcarbonylamino, aralkylcarbonylamino, haloalkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylenedioxyalkyl, dialkylalkylenedioxyalkyl, alkylsulfonylamino, arylsulfonylamino, azido, dialkylphosphonyl, alkylarylphosphonyl, diarylphosphonyl, alkylthio, arylthio, perfluoroalkylthio, hydroxycarbonylalkylthio, thiocyano, isothiocyano, alkylsulfinyl, alkylsulfonyl, arylsulfinyl, arylsulfonyl, aminosulfonyl, alkylaminosulfonyl, dialkylaminosulfonyl, arylaminosulfonyl, diarylaminosulfonyl or alkylarylaminosulfonyl; or two Q¹ groups, which substitute atoms in a 1, 2 or 1,3 arrangement, together form alkylenedioxy (i.e., —O—(CH₂)_{z}—O—), thioalkylenoxy (i.e., —S—(CH₂)_{z}—O—) or alkylenedithioxy (i.e., —S—(CH₂)_{z}—S—) where z is 1 or 2; and
each Q¹ is independently unsubstituted or substituted with one or more substituents, in one embodiment one to three or four substituents, each independently selected from Q², where Q² is halo, pseudohalo, hydroxy, oxo, thia, nitrile, nitro, formyl, mercapto, amino, hydroxyalkyl, hydroxyaryl, hydroxycarbonyl, alkyl, haloalkyl, polyhaloalkyl, aminoalkyl, diaminoalkyl, alkenyl containing 1 to 2 double bonds, alkynyl containing 1 to 2 triple bonds, cycloalkyl, heterocyclyl, aryl, heteroaryl, aralkyl, aralkenyl, aralkynyl, alkoxycarbonyl, aryloxycarbonyl, aralkoxycarbonyl, arylcarbonylalkyl, aminocarbonyl, alkoxy, aryloxy, aralkoxy, alkylenedioxy, amino, aminoalkyl, dialkylamino, arylamino, diarylamino, alkylamino, dialkylamino, haloalkylamino, arylamino, diarylamino, alkylarylamino, aralkylamino, alkoxycarbonylamino, arylcarbonylamino, alkylthio or arylthio;
provided the compound is not a compound in Table 2.

2. The compound of claim 1 wherein R¹ is —C(J)OR¹¹; J is O; R³ is COR⁹; R⁹ is optionally substituted alkyl, optionally substituted aryl, optionally substituted aralkyl, optionally substituted heteroaryl, optionally substituted heteroaralkyl, optionally substituted heterocyclyl or optionally substituted heterocyclylalkyl; R⁶ or R⁷ is optionally substituted alkyl; and n is 0-3.

3.-7. (canceled)

8. The compound of claim 1 wherein R¹ is —C(J)OR¹¹; J is O; R³ is CON(R¹¹)(R¹²); R¹¹ is hydrogen or optionally substituted alkyl; R¹² is selected from the group consisting of optionally substituted alkyl, optionally substituted aryl, optionally substituted aralkyl, optionally substituted cycloalkyl, optionally substituted cycloalkylalkyl, optionally substituted heterocyclyl and optionally substituted heterocyclylalkyl; R⁶ or R⁷ is optionally substituted alkyl; and n is 0.

9.-10. (canceled)

11. The compound of claim 8 wherein when R³ is CON(R¹¹)(R¹²); R¹¹ is hydrogen and R¹² is selected from the group consisting of methyl, ethyl, propyl, isopropyl, butyl, isobutyl, dimethylaminoethyl, dimethylaminopropyl, diethylaminoethyl, diethylamino, dimethylamino, cyclopntyl, cyclohexyl, cycloheptyl, phenyl, 2-morpholin-4-ylethyl, 3-morpholin-4-ylpropyl, 3-morpholin-4-ylpropyl)amino, and piperidinyl.

12. The compound of claim 1 wherein R¹¹ and R¹² together to which they are attached form optionally substituted heterocyclyl or optionally substituted heterocyclylalkyl selected from the group consisting of pyrrolidin-1-yl, 4-pyrrolidin-1-yl, piperidin-1-yl, 4-methylpiperazin-1-yl, 4-ethylpiperazin-1-yl, 4-piperazin-1-yl, 4-propylpiperazin-1-yl, piperidin-3-yl, piperidinyl, (1S,4S)-5-methyl-2,5-diazabicyclo[2.2.1]hept-2-yl and azepanyl.

13.-14. (canceled)

15. The compound of claim 2 wherein Q¹ is selected from the group consisting of hydroxy, cyano, 2-methyl; 3-methyl; methylpiperazinyl, 3-chloromethyl, 3,4-difluoro; 3-methyl, 4-methyl; 2-methyloxy; 3-methyloxy; 4-methyloxy; 3-fluoro-4-methyl; 4-fluoro-3-methyl; 2-trifluoromethyloxy; 2-chloro; 3-chloro; 4-chloro; 2,4-dichloro; 2-chloro-3,6-difluoro, 3-chloro-2,6-difluoro, 2-fluoro; 3-fluoro; 2-bromo; 3-trifluoromethyl; 2,3-difluoro; 2,4-difluoro; 2,5-difluoro; 2,6-difluoro; 3,4-difluoro; 3,6-difluoro; 3,4-difluoro; 2,3-difluoro-4-trifluoromethyl; 2-fluoro-4-trifluoromethyl; 2-fluoro-3-trifluoromethyl; 3-fluoro-5-trifluoromethyl; 2,5-bistrifluoromethyl; 3,5-bistrifluoromethyl; 3-chloro-2-fluoro-4-trifluoromethy; 3-fluoro-4-trifluoromethyl; 4-fluoro-3-trifluoromethyl; 4-fluoro-2-trifluoromethyl; 2-chloro-4-fluoro; 3-chloro-4-fluoro; 2-trifluoromethyl; 4-trifluoromethyl; 2,3,4-trifluoro; 2,4,6-trifluoro; 2,4,5-trifluoro; 3,4-bis(methyloxy); 3-phenylmethyloxy; methyloxyphenylmethyloxy, 4-piperidin-4-yl, 3-piperidin-4-yl, 3-piperidin-4-ylmethyl, piperidin-4-ylmethyl, dimethylaminomethyl, diethylaminomethyl, dimethylaminoethyloxy, dimethylaminopropyloxy, diethylaminopropyloxy, 4-methylsulfonylpiperazin-1-yl, 3-azepan-1-ylmethyl, 4-methyl-1,4-diazepan-1-yl, 3-pyrrolidin-1-ylethyl, 4-methylpiperazin-1-ylmethyl; 4-ethylpiperazin-1-ylmethyl; 3-piperazin-1-ylmethyl; morpholin-4-ylmethyl; 3-morpholin-4-ylmethyl; 2-morpholin-4-ylethyloxy; 2-piperidin-1-ylethyloxy; 3-morpholin-4-ylpropyloxy 1H-pyrazol-1-yl, 4-trifluoromethyl-1H-pyrazol-1-yl, 4-acetylpiperazin-1-ylmethyl; methylbenzotriazolyl, dimethylethyloxycarbonylpiperazin-1-ylmethyl, 4-phenylsulfonylpiperazin-1-ylmethyl, 4-fluorophenylsulfonylpiperazin-1-yl, 4-ethylsulfonylpiperazin-1-ylmethyl, 4-cyclopropyl carbonylpiperazin-1-ylmethyl, 2-methylpropanoylpiperazin-1-ylmethyl, 4-phenylcarbonyl piperazin-1-ylmethyl, 3-azocan-1-ylmethyl, 4-acetyl-1,4-diazepan-1-yl, 4-phenylamino carbonylpiperazin-1-ylmethyl; 4-ethylaminocarbonylpiperazin-1-ylmethyl; 3-piperidin-1-ylpropyloxy, 2-pyrrolidin-1-ylethyloxy; 3-piperidin-1-ylpropyloxy; and 3-morpholin-4-ylpropyloxy.

16.-19. (canceled)

20. The compound of claim 1 wherein R⁹ is selected from the group consisting of methyl, ethyl, propyl, isopropyl, butyl, isobutyl, pentyl, cyclopentyl, cyclohexyl, cycloheptyl; dimethylaminopropyl, 4-methylpentyl; (3s,5s,7s)-tricyclo{3.3.1.1˜3,7˜]dec-1-yl; 1S,4S)-5-methyl-2,5-diazabicyclo[2.2.1]hept-2-yl]; phenyl, isoxazolyl, piperidinyl, piperazinyl, pyrrolidinyl, morpholinyl, benzodioxolyl, and benzotriazolyl

21.-23. (canceled)

24. The compound of claim 1 wherein n is 0.

25. (canceled)

26. The compound of claim 1 wherein R¹ is —C(J)OR¹¹ and R¹¹ is selected from the group consisting of 2,2-dimethyl-1,3-dioxolan-4-yl; 2-piperidin-1-ylethylaminocarbonyl; 2,3-dihydroxypropyl or 2-fluoro-1-(fluoromethyl)ethyl, hydroxyethyl, phenylmethyloxyethyl, 3,4-difluorophenylcarbonyloxy-1-methylethyl, and 2-hydroxy-1-methylethyl.

27. The compound of claim 1 wherein R¹ is C(J)N(R¹⁰)(R¹¹) and R¹¹ is optionally substituted alkyl, selected from the group consisting of isopropyl; beta-alanine, 2,3-dihydroxypropyl; and 2-hydroxy-1-(hydroxymethyl)ethyl.

28.-29. (canceled)

30. A compound having the formula Ia: 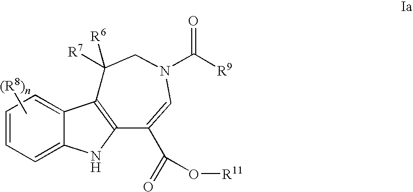
wherein each R⁶, R⁷ or R¹¹ is optionally substituted alkyl; n is 0; R⁹ is optionally substituted alkyl, optionally substituted heterocyclyl or optionally substituted heterocyclylalkyl.

31. The compound of claim 30 wherein R⁹ is optionally substituted with one or more Q¹.

32. The compound of claim 30 selected from the group consisting of:
1-methylethyl 1,1-dimethyl-3-[(1-methylpiperidin-3-yl)carbonyl]-1,2,3,6-tetrahydroazepino[4,5-b]indole-5-carboxylate;
1-methylethyl 1,1-dimethyl-3-[(1-methylpiperidin-4-yl)carbonyl]-1,2,3,6-tetrahydroazepino[4,5-b]indole-5-carboxylate; and
1-methylethyl 3-[4-(dimethylamino)butanoyl]-1,1-dimethyl-1,2,3,6-tetrahydroazepino[4,5-b]indole-5-carboxylate.

33. (canceled)

34. (canceled)

35. (canceled)

36. The compound of claim 30 selected from the group consisting of:
1-methylethyl 3-(cyclohexylcarbonyl)-1,1-dimethyl-1,2,3,6-tetrahydroazepino[4,5-b]indole-5-carboxylate;
1-methylethyl 3-acetyl-1,1-dimethyl-1,2,3,6-tetrahydroazepino[4,5-b]indole-5-carboxylate;
1-methylethyl 3-butanoyl-1,1-dimethyl-1,2,3,6-tetrahydroazepino[4,5-b]indole-5-carboxylate;
1-methylethyl 1,1-dimethyl-3-pentanoyl-1,2,3,6-tetrahydroazepino[4,5-b]indole-5-carboxylate;
1-methylethyl 3-(cyclopentylcarbonyl)-1,1-dimethyl-1,2,3,6-tetrahydroazepino[4,5-b]indole-5-carboxylate;
1-methylethyl 3-(2,2-dimethyl propanoyl)-1,1-dimethyl-1,2,3,6-tetrahydroazepino[4,5-b]indole-5-carboxylate;
1-methylethyl 3-(2-ethylbutanoyl)-1,1-dimethyl-1,2,3,6-tetrahydroazepino[4,5-b]indole-5-carboxylate;
1-methylethyl 1,1-dimethyl-3-(3-methylbutanoyl)-1,2,3,6-tetrahydroazepino[4,5-b]indole-5-carboxylate;
1-methylethyl 3-(cycloheptylcarbonyl)-1,1-dimethyl-1,2,3,6-tetrahydroazepino[4,5-b]indole-5-carboxylate;
1-methylethyl 1,1-dimethyl-3-propanoyl-1,2,3,6-tetrahydroazepino[4,5-b]indole-5-carboxylate;
1-methylethyl 1,1-dimethyl-3-[(3s,5s,7s)-tricyclo[3.3.1.1˜3,7˜]dec-1-ylcarbonyl]-1,2,3,6-tetrahydroazepino[4,5-b]indole-5-carboxylate; and
1-methylethyl 1,1-dimethyl-3-(4-methylpentanoyl)-1,2,3,6-tetrahydroazepino[4,5-b]indole-5-carboxylate.

37. A compound having the formula Ib 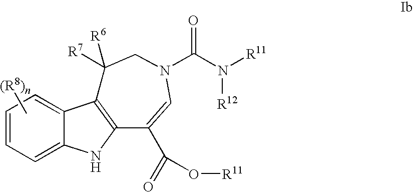
wherein each R⁶ and R⁷ is independently optionally substituted alkyl, n is 0-3; R⁸ is optionally substituted alkyl or halo; R¹¹ is hydrogen or optionally substituted alkyl; R¹² is optionally substituted alkyl, optionally substituted aryl, optionally substituted aralkyl, optionally substituted cycloalkyl, optionally substituted cycloalkylalkyl, optionally substituted heterocyclyl, or optionally substituted heterocyclylalkyl.

38. The compound of claim 37 wherein R¹² is methyl, ethyl, propyl, isopropyl, butyl, isobutyl, dimethylaminoethyl, dimethylaminopropyl, diethylaminoethyl, diethylamino, dimethylamino, 2-morpholin-4-ylethyl, 3-morpholin-4-ylpropyl, 3-morpholin-4-ylpropyl)amino, or piperidinyl.

39. The compound of claim 37 wherein R¹¹ and R¹² together to which they are attached form optionally substituted heterocylcyl or optionally substituted heterocyclylalkyl.

40. The compound of claim 37 wherein R¹¹ and R¹² is selected from the group consisting of pyrrolidin-1-yl, 4-pyrrolidin-1-yl, piperidin-1-yl, 4-methylpiperazin-1-yl, 4-ethylpiperazin-1-yl, 4-piperazin-1-yl, 4-propylpiperazin-1-yl, piperidin-3-yl, piperidinyl, (1S,4S)-5-methyl-2,5-diazabicyclo[2.2.1]hept-2-yl and azepanyl.

41. The compound of claim 37 wherein R¹¹ and R¹² together is optionally substituted with one or more Q¹ selected from the group consisting of optionally substituted alkyl, optionally substituted aryl, optionally substituted heterocyclyl and optionally substituted heterocyclylalkyl.

42. The compound of claim 41 wherein Q¹ is methyl, ethyl, propyl, diethylamino, dimethylamino, diethylaminomethyl, diethylaminoethyl, dimethylaminopropyloxymethyl, phenyl, phenylmethyl, pyrrolidinyl, piperazinyl, piperidinyl, methylpiperidinyl, methylpiperazinyl, 2-oxo-2-pyrrolidin-1ylethyl, and morpholino-4-methyl.

43. The compound of claim 42 selected from the group consisting of:
1-methylethyl 1,1-dimethyl-3-{[(1S,4S)-5-methyl-2,5-diazabicyclo[2.2.1]hept-2-yl]carbonyl}-1,2,3,6-tetrahydroazepino[4,5-b]indole-5-carboxylate;
1-methylethyl 1,1-dimethyl-3-[(4-pyrrolidin-1-ylpiperidin-1-yl)carbonyl]-1,2,3,6-tetrahydroazepino[4,5-b]indole-5-carboxylate;
1-methylethyl 1,1-dimethyl-3-(piperidin-1-ylcarbonyl)-1,2,3,6-tetrahydroazepino[4,5-b]indole-5-carboxylate;
1-methylethyl 3-({[3-(dimethylamino)propyl]amino}carbonyl)-1,1-dimethyl-1,2,3,6-tetrahydroazepino[4,5-b]indole-5-carboxylate;
1-methylethyl 1,1-dimethyl-3-({[4-(4-methylpiperazin-1-yl)phenyl]amino}carbonyl)-1,2,3,6-tetrahydroazepino[4,5-b]indole-5-carboxylate;
1-methylethyl 1,1-dimethyl-3-(pyrrolidin-1-ylcarbonyl)-1,2,3,6-tetrahydroazepino[4,5-b]indole-5-carboxylate;
1-methylethyl 1,1-dimethyl-3-[(4-methylpiperazin-1-yl)carbonyl]-1,2,3,6-tetrahydroazepino[4,5-b]indole-5-carboxylate;
1-methylethyl 3-({[2-(dimethylamino) ethyl]amino}carbonyl)-1,1-dimethyl-1,2,3,6-tetrahydroazepino[4,5-b]indole-5-carboxylate;
1-methylethyl 1,1-dimethyl-3-{[(3-morpholin-4-ylpropyl)amino]carbonyl}-1,2,3,6-tetrahydroazepino[4,5-b]indole-5-carboxylate;
1-methylethyl 1,1-dimethyl-3-{[(2-morpholin-4-ylethyl)amino]carbonyl}-1,2,3,6-tetrahydroazepino[4,5-b]indole-5-carboxylate;
1-methylethyl 3-[(4-ethylpiperazin-1-yl)carbonyl]-1,1-dimethyl-1,2,3,6-tetrahydroazepino[4,5-b]indole-5-carboxylate;
1-methylethyl 1,1-dimethyl-3-(piperazin-1-ylcarbonyl)-1,2,3,6-tetrahydroazepino[4,5-b]indole-5-carboxylate;
1-methylethyl 3-({[2-(diethylamino)ethyl](ethyl)amino}carbonyl)-1,1-dimethyl-1,2,3,6-tetrahydroazepino[4,5-b]indole-5-carboxylate;
1-methylethyl 1,1-dimethyl-3-({4-[(1-methylpiperidin-4-yl)methyl]piperazin-1-yl}carbonyl)-1,2,3,6-tetrahydroazepino[4,5-b]indole-5-carboxylate;
1-methylethyl 1,1-dimethyl-3-{[4-(1-methylethyl)piperazin-1-yl]carbonyl}-1,2,3,6-tetrahydroazepino[4,5-b]indole-5-carboxylate;
1-methylethyl 1,1-dimethyl-3-[(4-propylpiperazin-1-yl)carbonyl]-1,2,3,6-tetrahydroazepino[4,5-b]indole-5-carboxylate;
1-methylethyl 9-fluoro-1,1-dimethyl-3-[(4-methylpiperazin-1-yl)carbonyl]-1,2,3,6-tetrahydroazepino[4,5-b]indole-5-carboxylate;
1-methylethyl 3-{[4-(diethylamino)piperidin-1-yl]carbonyl}-1,1-dimethyl-1,2,3,6-tetrahydroazepino[4,5-b]indole-5-carboxylate;
1-methylethyl 8-fluoro-1,1-dimethyl-3-[(4-methylpiperazin-1-yl)carbonyl]-1,2,3,6-tetrahydroazepino[4,5-b]indole-5-carboxylate;
1-methylethyl 3-[(4-ethylpiperazin-1-yl)carbonyl]-8-fluoro-1,1-dimethyl-1,2,3,6-tetrahydroazepino[4,5-b]indole-5-carboxylate;
1-methylethyl 1,1-dimethyl-3-{[4-(2-oxo-2-pyrrolidin-1-ylethyl)piperazin-1-yl]carbonyl}-1,2,3,6-tetrahydroazepino[4,5-b]indole-5-carboxylate;
1-methylethyl 3-({4-[2-(diethylamino)ethyl]piperazin-1-yl}carbonyl)-1,1-dimethyl-1,2,3,6-tetrahydroazepino[4,5-b]indole-5-carboxylate;
1-methylethyl 3-{[3-(dimethylamino)piperidin-1-yl]carbonyl}-1,1-dimethyl-1,2,3,6-tetrahydroazepino[4,5-b]indole-5-carboxylate;
1-methylethyl 3-(azepan-1-ylcarbonyl)-1,1-dimethyl-1,2,3,6-tetrahydroazepino[4,5-b]indole-5-carboxylate;
1-methylethyl 1,1-dimethyl-3-{[4-(4-methylpiperazin-1-yl)piperidin-1-yl]carbonyl}-1,2,3,6-tetrahydroazepino[4,5-b]indole-5-carboxylate;
1-methylethyl 1,1-dimethyl-3-[(4-methyl-1,4-diazepan-1-yl)carbonyl]-1,2,3,6-tetrahydroazepino[4,5-b]indole-5-carboxylate;
1-methylethyl 1,1-dimethyl-3-(morpholin-4-ylcarbonyl)-1,2,3,6-tetrahydroazepino[4,5-b]indole-5-carboxylate;
1-methylethyl 3-({3-[(dimethylamino) methyl]piperidin-1-yl}carbonyl)-1,1-dimethyl-1,2,3,6-tetrahydroazepino[4,5-b]indole-5-carboxylate;
1-methylethyl 3-({(3S)-3-[(dimethylamino)methyl]piperidin-1-yl}carbonyl)-1,1-dimethyl-1,2,3,6-tetrahydroazepino[4,5-b]indole-5-carboxylate;
1-methylethyl 3-({(3R)-3-[(dimethylamino)methyl]piperidin-1-yl}carbonyl)-1,1-dimethyl-1,2,3,6-tetrahydroazepino[4,5-b]indole-5-carboxylate;
1-methylethyl 3-[(diethylamino)carbonyl]-1,1-dimethyl-1,2,3,6-tetrahydroazepino[4,5-b]indole-5-carboxylate;
1-methylethyl 1,1-dimethyl-3-{[3-(morpholin-4-ylmethyl)piperidin-1-yl]carbonyl}-1,2,3,6-tetrahydroazepino[4,5-b]indole-5-carboxylate;
1-methylethyl 1,1-dimethyl-3-{[(3S)-piperidin-3-ylamino]carbonyl}-1,2,3,6-tetrahydroazepino[4,5-b]indole-5-carboxylate;
1-methylethyl 3-{[3-({[3-(dimethylamino)propyl]oxy}methyl)piperidin-1-yl]carbonyl}-1,1-dimethyl-1,2,3,6-tetrahydroazepino[4,5-b]indole-5-carboxylate;
1-methylethyl 1,1-dimethyl-3-[(piperidin-3-ylamino)carbonyl]-1,2,3,6-tetrahydroazepino[4,5-b]indole-5-carboxylate;
1-methylethyl 1,1-dimethyl-3-{[(3R)-3-(morpholin-4-ylmethyl)piperidin-1-yl]carbonyl}-1,2,3,6-tetrahydroazepino[4,5-b]indole-5-carboxylate;
1-methylethyl 1,1-dimethyl-3-{[(3R)-3-(piperidin-1-ylmethyl)piperidin-1-yl]carbonyl}-1,2,3,6-tetrahydroazepino[4,5-b]indole-5-carboxylate;
1-methylethyl 1,1-dimethyl-3-{[4-(phenylmethyl)-1,4-diazepan-1-yl]carbonyl}-1,2,3,6-tetrahydroazepino[4,5-b]indole-5-carboxylate; and
1-methylethyl 3-[(3′R)-1,3′-bipiperidin-1′-ylcarbonyl]-1,1-dimethyl-1,2,3,6-tetrahydroazepino[4,5-b]indole-5-carboxylate.

44. The compound of claim 37 wherein each R⁶ and R⁷ is independently optionally substituted alkyl; n is 0; R¹¹ is independently hydrogen or optionally substituted alkyl; R¹² is optionally substituted alkyl, optionally substituted cycloalkyl, optionally substituted cycloalkylalkyl, optionally substituted aryl or optionally substituted aralkyl

45. The compound of claim 44 selected from the group consisting of:
1-methylethyl 1,1-dimethyl-3-[(propylamino)carbonyl]-1,2,3,6-tetrahydroazepino[4,5-b]indole-5-carboxylate;
1-methylethyl 3-[(cyclopentylamino)carbonyl]-1,1-dimethyl-1,2,3,6-tetrahydroazepino[4,5-b]indole-5-carboxylate;
1-methylethyl 3-[(cyclohexylamino)carbonyl]-1,1-dimethyl-1,2,3,6-tetrahydroazepino[4,5-b]indole-5-carboxylate;
1-methylethyl 3-[(cycloheptylamino)carbonyl]-1,1-dimethyl-1,2,3,6-tetrahydroazepino[4,5-b]indole-5-carboxylate; and
1-methylethyl 1,1-dimethyl-3-{[(phenylmethyl)amino]carbonyl}-1,2,3,6-tetrahydroazepino[4,5-b]indole-5-carboxylate.

46. A compound having the formula Ic 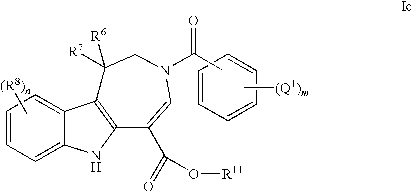
wherein each R⁶ and R⁷ is independently optionally substituted alkyl, n is 0-3; R⁸ is optionally substituted alkyl or halo; R¹¹ is independently optionally substituted alkyl, and Q¹ is independently hydroxy, halogen, haloalkyl, haloalkoxy, optionally substituted alkyl, alkoxy, cyano, optionally substituted aryl, optionally substituted aralkyl, optionally substituted heterocyclyl, or optionally substituted heterocyclylalkyl; and m is 0-3.

47. (canceled)

48. The compound of claim 46 selected from the group consisting of:
1-methylethyl 3-[(2-chloro-3,6-difluorophenyl)carbonyl]-1,1-dimethyl-1,2,3,6-tetrahydro azepino[4,5-b]indole-5-carboxylate;
1-methylethyl 1,1-dimethyl-3-(phenylcarbonyl)-1,2,3,6-tetrahydroazepino[4,5-b]indole-5-carboxylate;
1-methylethyl 3-[(2-fluorophenyl)carbonyl]-1,1-dimethyl-1,2,3,6-tetrahydroazepino[4,5-b]indole-5-carboxylate;
1-methylethyl 1,1-dimethyl-3-{[2-(trifluoromethyl)phenyl]carbonyl}-1,2,3,6-tetrahydro azepino[4,5-b]indole-5-carboxylate;
1-methylethyl 1,1-dimethyl-3-{[4-(trifluoromethyl)phenyl]carbonyl}-1,2,3,6-tetrahydro azepino[4,5-b]indole-5-carboxylate;
1-methylethyl 3-[(2-chlorophenyl)carbonyl]-1,1-dimethyl-1,2,3,6-tetrahydroazepino[4,5-b]indole-5-carboxylate;
1-methylethyl 3-[(2-bromophenyl)carbonyl]-1,1-dimethyl-1,2,3,6-tetrahydroazepino[4,5-b]indole-5-carboxylate;
1-methylethyl 1,1-dimethyl-3-[(2-methylphenyl)carbonyl]-1,2,3,6-tetrahydroazepino[4,5-b]indole-5-carboxylate;
1-methylethyl 1,1-dimethyl-3-{[2-(methyloxy)phenyl]carbonyl}-1,2,3,6-tetrahydroazepino[4,5-b]indole-5-carboxylate;
1-methylethyl 1,1-dimethyl-3-({2-[(trifluoromethyl)oxy]phenyl}carbonyl)-1,2,3,6-tetrahydroazepino[4,5-b]indole-5-carboxylate;
1-methylethyl 3-[(2-fluorophenyl)carbonyl]-1,1-dimethyl-1,2,3,6-tetrahydroazepino[4,5-b]indole-5-carboxylate;
1-methylethyl 3-[(3-fluorophenyl)carbonyl]-1,1-dimethyl-1,2,3,6-tetrahydroazepino[4,5-b]indole-5-carboxylate;
1-methylethyl 3-[(2,4-difluorophenyl)carbonyl]-1,1-dimethyl-1,2,3,6-tetrahydro azepino[4,5-b]indole-5-carboxylate;
1-methylethyl 3-[(2,3-difluorophenyl)carbonyl]-1,1-dimethyl-1,2,3,6-tetrahydro azepino[4,5-b]indole-5-carboxylate;
1-methylethyl 3-[(2,6-difluorophenyl)carbonyl]-1,1-dimethyl-1,2,3,6-tetrahydro azepino[4,5-b]indole-5-carboxylate;
1-methylethyl 3-[(2,5-difluorophenyl)carbonyl]-1,1-dimethyl-1,2,3,6-tetrahydroazepino[4,5-b]indole-5-carboxylate;
1-methylethyl 1,1-dimethyl-3-[(2,3,4-trifluorophenyl)carbonyl]-1,2,3,6-tetrahydro azepino[4,5-b]indole-5-carboxylate;
1-methylethyl 1,1-dimethyl-3-[(2,4,6-trifluorophenyl)carbonyl]-1,2,3,6-tetrahydro azepino[4,5-b]indole-5-carboxylate;
1-methylethyl 1,1-dimethyl-3-[(2,4,5-trifluorophenyl)carbonyl]-1,2,3,6-tetrahydro azepino[4,5-b]indole-5-carboxylate;
1-methylethyl 3-[(3-chlorophenyl)carbonyl]-1,1-dimethyl-1,2,3,6-tetrahydroazepino[4,5-b]indole-5-carboxylate;
1-methylethyl 3-[(4-chlorophenyl)carbonyl]-1,1-dimethyl-1,2,3,6-tetrahydroazepino[4,5-b]indole-5-carboxylate;
1-methylethyl 3-{[4-fluoro-3-(trifluoromethyl)phenyl]carbonyl}-1,1-dimethyl-1,2,3,6-tetrahydroazepino[4,5-b]indole-5-carboxylate;
1-methylethyl 3-{[3-fluoro-4-(trifluoromethyl)phenyl]carbonyl}-1,1-dimethyl-1,2,3,6-tetrahydroazepino[4,5-b]indole-5-carboxylate;
1-methylethyl 1,1-dimethyl-3-[(3-methylphenyl)carbonyl]-1,2,3,6-tetrahydroazepino[4,5-b]indole-5-carboxylate;
1-methylethyl 1,1-dimethyl-3-[(4-methylphenyl)carbonyl]-1,2,3,6-tetrahydroazepino[4,5-b]indole-5-carboxylate;
1-methylethyl 1,1-dimethyl-3-{[3-(methyloxy)phenyl]carbonyl}-1,2,3,6-tetrahydro azepino[4,5-b]indole-5-carboxylate;
1-methylethyl 1,1-dimethyl-3-{[4-(methyloxy)phenyl]carbonyl}-1,2,3,6-tetrahydro azepino[4,5-b]indole-5-carboxylate;
3-[(3,4-difluorophenyl)carbonyl]-1,1-dimethyl-N-(1-methylethyl)-1,2,3,4,5,6-hexahydro azepino[4,5-b]indole-5-carboxamide;
1-methylethyl 3-{[3,4-bis(methyloxy)phenyl]carbonyl}-1,1-dimethyl-1,2,3,6-tetrahydro azepino[4,5-b]indole-5-carboxylate;
1-{3-[(3,4-difluorophenyl)carbonyl]-1,1-dimethyl-1,2,3,6-tetrahydroazepino[4,5-b]indol-5-yl}ethanone;
1-methylethyl 1,1-dimethyl-3-[(5-methylisoxazol-3-yl)carbonyl]-1,2,3,6-tetrahydro azepino[4,5-b]indole-5-carboxylate;
1-methylethyl 3-{[4-fluoro-2-(trifluoromethyl)phenyl]carbonyl}-1,1-dimethyl-1,2,3,6-tetrahydroazepino[4,5-b]indole-5-carboxylate;
1-methylethyl 3-[(2-chloro-4-fluorophenyl)carbonyl]-1,1-dimethyl-1,2,3,6-tetrahydro azepino[4,5-b]indole-5-carboxylate;
1-methylethyl 3-{[3-(chloromethyl)phenyl]carbonyl}-1,1-dimethyl-1,2,3,6-tetrahydro azepino[4,5-b]indole-5-carboxylate;
2-chloro-1-{3-[(3,4-difluorophenyl)carbonyl]-1,1-dimethyl-1,2,3,6-tetrahydroazepino[4,5-b]indol-5-yl}ethanone;
methyl 3-[(3,4-difluorophenyl)carbonyl]-1,1-dimethyl-1,2,3,6-tetrahydroazepino[4,5-b]indole-5-carboxylate;
1-methylethyl 1,1-dimethyl-3-({3-[(phenylmethyl)oxy]phenyl}carbonyl)-1,2,3,6-tetrahydroazepino[4,5-b]indole-5-carboxylate;
1-methylethyl 1,1-dimethyl-3-{[3-(trifluoromethyl)phenyl]carbonyl}-1,2,3,6-tetrahydro azepino[4,5-b]indole-5-carboxylate;
1-methylethyl 3-[(3-fluoro-4-methylphenyl)carbonyl]-1,1-dimethyl-1,2,3,6-tetrahydro azepino[4,5-b]indole-5-carboxylate;
1-methylethyl 3-{[2-fluoro-4-(trifluoromethyl)phenyl]carbonyl}-1,1-dimethyl-1,2,3,6-tetrahydroazepino[4,5-b]indole-5-carboxylate;
1-methylethyl 3-{[3-chloro-2-fluoro-4-(trifluoromethyl)phenyl]carbonyl}-1,1-dimethyl-1,2,3,6-tetrahydroazepino[4,5-b]indole-5-carboxylate;
1-methylethyl 3-{[2-fluoro-3-(trifluoromethyl)phenyl]carbonyl}-1,1-dimethyl-1,2,3,6-tetrahydroazepino[4,5-b]indole-5-carboxylate;
1-methylethyl 3-{[3-fluoro-5-(trifluoromethyl)phenyl]carbonyl}-1,1-dimethyl-1,2,3,6-tetrahydroazepino[4,5-b]indole-5-carboxylate;
1-methylethyl 3-{[3,5-bis(trifluoromethyl)phenyl]carbonyl}-1,1-dimethyl-1,2,3,6-tetrahydroazepino[4,5-b]indole-5-carboxylate;
1-methylethyl 3-{[2,5-bis(trifluoromethyl)phenyl]carbonyl}-1,1-dimethyl-1,2,3,6-tetrahydroazepino[4,5-b]indole-5-carboxylate;
1-methylethyl 3-{[2,3-difluoro-4-(trifluoromethyl)phenyl]carbonyl}-1,1-dimethyl-1,2,3,6-tetrahydroazepino[4,5-b]indole-5-carboxylate;
1-methylethyl 3-[(3-hydroxyphenyl)carbonyl]-1,1-dimethyl-1,2,3,6-tetrahydro azepino[4,5-b]indole-5-carboxylate;
1-methylethyl 3-[(3-cyanophenyl)carbonyl]-1,1-dimethyl-1,2,3,6-tetrahydroazepino[4,5-b]indole-5-carboxylate;
1-methylethyl 3-[(2,4-dichlorophenyl)carbonyl]-1,1-dimethyl-1,2,3,6-tetrahydro azepino[4,5-b]indole-5-carboxylate;
1-methylethyl 3-[(4-fluoro-3-methylphenyl)carbonyl]-1,1-dimethyl-1,2,3,6-tetrahydro azepino[4,5-b]indole-5-carboxylate;
1-methylethyl 3-[(3-chloro-2,6-difluorophenyl)carbonyl]-1,1-dimethyl-1,2,3,6-tetrahydro azepino[4,5-b]indole-5-carboxylate;
1-methylethyl 3-[(3-chloro-4-fluorophenyl)carbonyl]-1,1-dimethyl-1,2,3,6-tetrahydro azepino[4,5-b]indole-5-carboxylate;
1-methylethyl 3-[(3,4-dichlorophenyl)carbonyl]-1,1-dimethyl-1,2,3,6-tetrahydro azepino[4,5-b]indole-5-carboxylate;
1-methylethyl 3-[(4-chloro-2,5-difluorophenyl)carbonyl]-1,1-dimethyl-1,2,3,6-tetrahydro azepino[4,5-b]indole-5-carboxylate;
1-methylethyl 3-[(3-bromo-4-fluorophenyl)carbonyl]-1,1-dimethyl-1,2,3,6-tetrahydro azepino[4,5-b]indole-5-carboxylate; and
1-methylethyl 3-{[3,4-difluoro-5-({[4-(methyloxy)phenyl]methyl}oxy)phenyl]carbonyl}-1,1-dimethyl-1,2,3,6-tetrahydroazepino[4,5-b]indole-5-carboxylate.

49.-51. (canceled)

52. The compound of claim 30 selected from the group consisting of:
1-methylethyl 3-(1,3-benzodioxol-5-ylcarbonyl)-1,1-dimethyl-1,2,3,6-tetrahydro azepino[4,5-b]indole-5-carboxylate;
1-methylethyl 3-[(2,2-difluoro-1,3-benzodioxol-4-yl)carbonyl]-1,1-dimethyl-1,2,3,6-tetrahydroazepino[4,5-b]indole-5-carboxylate;
1-methylethyl 3-[(2,2-difluoro-1,3-benzodioxol-5-yl)carbonyl]-1,1-dimethyl-1,2,3,6-tetrahydroazepino[4,5-b]indole-5-carboxylate; and
1-methylethyl 1,1-dimethyl-3-[(5-methylisoxazol-3-yl)carbonyl]-1,2,3,6-tetrahydro azepino[4,5-b]indole-5-carboxylate.

53. The compound of claim 46 wherein each R⁶ and R⁷ is independently optionally substituted alkyl; n is 0; R¹⁰ is independently optionally substituted alkyl, Q¹ is independently optionally substituted alkyl, halogen, optionally substituted aryl, optionally substituted aralkyl, optionally substituted heterocyclyl, or optionally substituted heterocyclylalkyl; m is 0-3.

54. The compound of claim 53 wherein Q¹ is 3,4-difluoro; 4-piperidin-4-yl, 3-piperidin-4-yl, 3-piperidin-4-ylmethyl, piperidin-4-ylmethyl, dimethylaminomethyl, diethylaminomethyl, dimethylaminoethyloxy, dimethylaminopropyloxy, diethylaminopropyloxy, 4-methylsulfonylpiperazin-1-yl, 3-azepan-1-ylmethyl, 4-methyl-1,4-diazepan-1-yl, 3-pyrrolidin-1-ylethyl, 4-methylpiperazin-1-ylmethyl; 4-ethylpiperazin-1-ylmethyl; 3-piperazin-1-ylmethyl; morpholin-4-ylmethyl; 3-morpholin-4-ylmethyl; 2-morpholin-4-ylethyloxy; 2-piperidin-1-ylethyloxy; 3-morpholin-4-ylpropyloxy 1H-pyrazol-1-yl, 4-trifluoromethyl-1H-pyrazol-1-yl, 4-acetylpiperazin-1-ylmethyl; methylbenzotriazolyl, dimethylethyloxycarbonylpiperazin-1-ylmethyl, 4-phenylsulfonylpiperazin-1-ylmethyl, 4-fluorophenylsulfonylpiperazin-1-yl, 4-ethylsulfonylpiperazin-1-ylmethyl, 4-cyclopropyl carbonylpiperazin-1-ylmethyl, 2-methylpropanoylpiperazin-1-ylmethyl, 4-phenylcarbonyl piperazin-1-ylmethyl, 3-azocan-1-ylmethyl, 4-acetyl-1,4-diazepan-1-yl, 4-phenylamino carbonylpiperazin-1-ylmethyl; 4-ethylaminocarbonylpiperazin-1-ylmethyl; 3-piperidin-1-ylpropyloxy, 2-pyrrolidin-1-ylethyloxy; 3-piperidin-1-ylpropyloxy; or 3-morpholin-4-ylpropyloxy.

55. The compound of claim 53 selected from the group consisting of:
1-methylethyl 1,1-dimethyl-3-[(4-piperidin-4-ylphenyl)carbonyl]-1,2,3,6-tetrahydroazepino[4,5-b]indole-5-carboxylate;
1-methylethyl 1,1-dimethyl-3-[(3-piperidin-4-ylphenyl)carbonyl]-1,2,3,6-tetrahydroazepino[4,5-b]indole-5-carboxylate;
1-methylethyl 3-({4-[(dimethylamino)methyl]phenyl}carbonyl)-1,1-dimethyl-1,2,3,6-tetrahydroazepino[4,5-b]indole-5-carboxylate;
1-methylethyl 3-({3-[(dimethylamino)methyl]phenyl}carbonyl)-1,1-dimethyl-1,2,3,6-tetrahydroazepino[4,5-b]indole-5-carboxylate;
1-methylethyl 3-({3-[(diethylamino)methyl]phenyl}carbonyl)-1,1-dimethyl-1,2,3,6-tetrahydroazepino[4,5-b]indole-5-carboxylate;
1-methylethyl 1,1-dimethyl-3-{[3-(pyrrolidin-1-ylmethyl)phenyl]carbonyl}-1,2,3,6-tetrahydroazepino[4,5-b]indole-5-carboxylate;
1-methylethyl 1,1-dimethyl-3-{[3-(piperidin-1-ylmethyl)phenyl]carbonyl}-1,2,3,6-tetrahydroazepino[4,5-b]indole-5-carboxylate;
1-methylethyl 1,1-dimethyl-3-({3-[(4-methylpiperazin-1-yl)methyl]phenyl}carbonyl)-1,2,3,6-tetrahydroazepino[4,5-b]indole-5-carboxylate;
1-methylethyl 3-({3-[(4-ethylpiperazin-1-yl)methyl]phenyl}carbonyl)-1,1-dimethyl-1,2,3,6-tetrahydroazepino[4,5-b]indole-5-carboxylate;
1-methylethyl 1,1-dimethyl-3-{[3-(morpholin-4-ylmethyl)phenyl]carbonyl}-1,2,3,6-tetrahydroazepino[4,5-b]indole-5-carboxylate;
1-methylethyl 3-[(3-{[2-(dimethylamino)ethyl]oxy}phenyl)carbonyl]-1,1-dimethyl-1,2,3,6-tetrahydroazepino[4,5-b]indole-5-carboxylate;
1-methylethyl 1,1-dimethyl-3-{[4-(1H-pyrazol-1-yl)phenyl]carbonyl}-1,2,3,6-tetrahydroazepino[4,5-b]indole-5-carboxylate;
1-methylethyl 3-({3-[(4-acetylpiperazin-1-yl)methyl]phenyl}carbonyl)-1,1-dimethyl-1,2,3,6-tetrahydroazepino[4,5-b]indole-5-carboxylate;
1-methylethyl 3-[(3-{[3-(dimethylamino)propyl]oxy}phenyl)carbonyl]-1,1-dimethyl-1,2,3,6-tetrahydroazepino[4,5-b]indole-5-carboxylate;
1-methylethyl 1,1-dimethyl-3-[(3-{[4-(methylsulfonyl)piperazin-1-yl]methyl}phenyl)carbonyl]-1,2,3,6-tetrahydroazepino[4,5-b]indole-5-carboxylate;
1-methylethyl 3-{[3-(azepan-1-ylmethyl)phenyl]carbonyl}-1,1-dimethyl-1,2,3,6-tetrahydroazepino[4,5-b]indole-5-carboxylate;
1-methylethyl 1,1-dimethyl-3-({3-[(4-methyl-1,4-diazepan-1-yl)methyl]phenyl}carbonyl)-1,2,3,6-tetrahydroazepino[4,5-b]indole-5-carboxylate;
1-methylethyl 3-{[2-fluoro-5-(morpholin-4-ylmethyl)phenyl]carbonyl}-1,1-dimethyl-1,2,3,6-tetrahydroazepino[4,5-b]indole-5-carboxylate;
1-methylethyl 3-{[4-fluoro-3-(morpholin-4-ylmethyl)phenyl]carbonyl}-1,1-dimethyl-1,2,3,6-tetrahydroazepino[4,5-b]indole-5-carboxylate;
1-methylethyl 1,1-dimethyl-3-[(1-methyl-1H-1,2,3-benzotriazol-5-yl)carbonyl]-1,2,3,6-tetrahydroazepino[4,5-b]indole-5-carboxylate;
1-methylethyl 1,1-dimethyl-3-({4-[4-(trifluoromethyl)-1H-pyrazol-1-yl]phenyl}carbonyl)-1,2,3,6-tetrahydroazepino[4,5-b]indole-5-carboxylate;
1-methylethyl 1,1-dimethyl-3-({3-[(2-piperidin-1-ylethyl)oxy]phenyl}carbonyl)-1,2,3,6-tetrahydroazepino[4,5-b]indole-5-carboxylate;
1-methylethyl 1,1-dimethyl-3-({3-[(2-morpholin-4-ylethyl)oxy]phenyl}carbonyl)-1,2,3,6-tetrahydroazepino[4,5-b]indole-5-carboxylate;
1-methylethyl 3-{[2-fluoro-5-(piperidin-1-ylmethyl)phenyl]carbonyl}-1,1-dimethyl-1,2,3,6-tetrahydroazepino[4,5-b]indole-5-carboxylate;
1-methylethyl 3-{[4-fluoro-3-(piperidin-1-ylmethyl)phenyl]carbonyl}-1,1-dimethyl-1,2,3,6-tetrahydroazepino[4,5-b]indole-5-carboxylate;
1-methylethyl 3-({3-[(4-{[(1,1-dimethylethyl)oxy]carbonyl}piperazin-1-yl)methyl]phenyl}carbonyl)-1,1-dimethyl-1,2,3,6-tetrahydroazepino[4,5-b]indole-5-carboxylate;
1-methylethyl 1,1-dimethyl-3-[(3-{[4-(phenylsulfonyl)piperazin-1-yl]methyl}phenyl)carbonyl]-1,2,3,6-tetrahydroazepino[4,5-b]indole-5-carboxylate;
1-methylethyl 3-{[3-({4-[(4-fluorophenyl)sulfonyl]piperazin-1-yl}methyl)phenyl]carbonyl}-1,1-dimethyl-1,2,3,6-tetrahydroazepino[4,5-b]indole-5-carboxylate;
1-methylethyl 3-[(3-{[4-(ethylsulfonyl)piperazin-1-yl]methyl}phenyl)carbonyl]-1,1-dimethyl-1,2,3,6-tetrahydroazepino[4,5-b]indole-5-carboxylate;
1-methylethyl 3-[(3-{[4-(cyclopropylcarbonyl)piperazin-1-yl]methyl}phenyl)carbonyl]-1,1-dimethyl-1,2,3,6-tetrahydroazepino[4,5-b]indole-5-carboxylate;
1-methylethyl 1,1-dimethyl-3-[(3-{[4-(2-methylpropanoyl)piperazin-1-yl]methyl}phenyl)carbonyl]-1,2,3,6-tetrahydroazepino[4,5-b]indole-5-carboxylate;
1-methylethyl 1,1-dimethyl-3-[(3-{[4-(phenylcarbonyl)piperazin-1-yl]methyl}phenyl)carbonyl]-1,2,3,6-tetrahydroazepino[4,5-b]indole-5-carboxylate;
1-methylethyl 3-{[3-(azocan-1-ylmethyl)phenyl]carbonyl}-1,1-dimethyl-1,2,3,6-tetrahydroazepino[4,5-b]indole-5-carboxylate;
1-methylethyl 3-({3-[(4-acetyl-1,4-diazepan-1-yl)methyl]phenyl}carbonyl)-1,1-dimethyl-1,2,3,6-tetrahydroazepino[4,5-b]indole-5-carboxylate;
1-methylethyl 1,1-dimethyl-3-{[3-(piperazin-1-ylmethyl)phenyl]carbonyl}-1,2,3,6-tetrahydroazepino[4,5-b]indole-5-carboxylate;
1-methylethyl 3-({3,4-difluoro-5-[(2-morpholin-4-ylethyl)oxy]phenyl}carbonyl)-1,1-dimethyl-1,2,3,6-tetrahydroazepino[4,5-b]indole-5-carboxylate;
1-methylethyl 3-({3,4-difluoro-5-[(2-piperidin-1-ylethyl)oxy]phenyl}carbonyl)-1,1-dimethyl-1,2,3,6-tetrahydroazepino[4,5-b]indole-5-carboxylate;
1-methylethyl 1,1-dimethyl-3-({4-[(2-morpholin-4-ylethyl)oxy]phenyl}carbonyl)-1,2,3,6-tetrahydroazepino[4,5-b]indole-5-carboxylate;
1-methylethyl 1,1-dimethyl-3-({4-[(2-piperidin-1-ylethyl)oxy]phenyl}carbonyl)-1,2,3,6-tetrahydroazepino[4,5-b]indole-5-carboxylate;
1-methylethyl 1,1-dimethyl-3-({3-[(3-morpholin-4-ylpropyl)oxy]phenyl}carbonyl)-1,2,3,6-tetrahydroazepino[4,5-b]indole-5-carboxylate;
1-methylethyl 1,1-dimethyl-3-{[3-({4-[(phenylamino)carbonyl]piperazin-1-yl}methyl)phenyl]carbonyl}-1,2,3,6-tetrahydroazepino[4,5-b]indole-5-carboxylate;
1-methylethyl 3-{[3-({4-[(ethylamino)carbonyl]piperazin-1-yl}methyl)phenyl]carbonyl}-1,1-dimethyl-1,2,3,6-tetrahydroazepino[4,5-b]indole-5-carboxylate;
1-methylethyl 1,1-dimethyl-3-({3-[(3-piperidin-1-ylpropyl)oxy]phenyl}carbonyl)-1,2,3,6-tetrahydroazepino[4,5-b]indole-5-carboxylate;
1-methylethyl 3-[(4-{[2-(dimethylamino)ethyl]oxy}phenyl)carbonyl]-1,1-dimethyl-1,2,3,6-tetrahydroazepino[4,5-b]indole-5-carboxylate;
1-methylethyl 3-[(3-{[3-(diethylamino)propyl]oxy}phenyl)carbonyl]-1,1-dimethyl-1,2,3,6-tetrahydroazepino[4,5-b]indole-5-carboxylate;
1-methylethyl 3-[(4-{[3-(dimethylamino)propyl]oxy}phenyl)carbonyl]-1,1-dimethyl-1,2,3,6-tetrahydroazepino[4,5-b]indole-5-carboxylate;
1-methylethyl 1,1-dimethyl-3-({4-[(2-pyrrolidin-1-ylethyl)oxy]phenyl}carbonyl)-1,2,3,6-tetrahydroazepino[4,5-b]indole-5-carboxylate;
1-methylethyl 1,1-dimethyl-3-({4-[(3-piperidin-1-ylpropyl)oxy]phenyl}carbonyl)-1,2,3,6-tetrahydroazepino[4,5-b]indole-5-carboxylate; and
1-methylethyl 1,1-dimethyl-3-({4-[(3-morpholin-4-ylpropyl)oxy]phenyl}carbonyl)-1,2,3,6-tetrahydroazepino[4,5-b]indole-5-carboxylate.

56. A compound having the formula Id

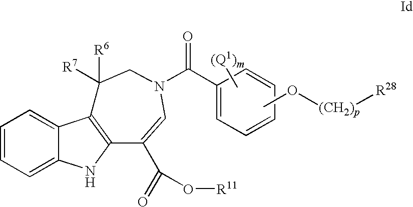

57. The compound of claim 56 wherein each R⁶ and R⁷ is independently optionally substituted alkyl; R¹¹ is independently optionally substituted alkyl, p is 1-3; m is 0-3; Q¹ is optionally substituted alkyl or halo; and R²⁸ is optionally substituted alkyl, optionally substituted aryl, optionally substituted aralkyl, optionally substituted heterocyclyl, or optionally substituted heterocyclylalkyl,

58. The compound of claim 56 wherein R²⁸ is phenyl, dimethylamino, diethylamino, N-ethyl, N-methyl amino, morpholinyl, piperidinyl, piperazinyl, pyrrolidinyl, morpholinyl, or 4-methyloxyphenyl.

59. The compound of claim 56 selected from the group consisting of:
1-methylethyl 1,1-dimethyl-3-({3-[(phenylmethyl)oxy]phenyl}carbonyl)-1,2,3,6-tetrahydroazepino[4,5-b]indole-5-carboxylate
1-methylethyl 3-[(3-{[2-(dimethylamino)ethyl]oxy}phenyl)carbonyl]-1,1-dimethyl-1,2,3,6-tetrahydroazepino[4,5-b]indole-5-carboxylate;
1-methylethyl 3-[(3-{[3-(dimethylamino)propyl]oxy}phenyl)carbonyl]-1,1-dimethyl-1,2,3,6-tetrahydroazepino[4,5-b]indole-5-carboxylate;
1-methylethyl 1,1-dimethyl-3-({3-[(2-piperidin-1-ylethyl)oxy]phenyl}carbonyl)-1,2,3,6-tetrahydroazepino[4,5-b]indole-5-carboxylate;
1-methylethyl 1,1-dimethyl-3-({3-[(2-morpholin-4-ylethyl)oxy]phenyl}carbonyl)-1,2,3,6-tetrahydroazepino[4,5-b]indole-5-carboxylate;
1-methylethyl 3-{[3,4-difluoro-5-({[4-(methyloxy)phenyl]methyl}oxy)phenyl]carbonyl}-1,1-dimethyl-1,2,3,6-tetrahydroazepino[4,5-b]indole-5-carboxylate;
1-methylethyl 3-({3,4-difluoro-5-[(2-morpholin-4-ylethyl)oxy]phenyl}carbonyl)-1,1-dimethyl-1,2,3,6-tetrahydroazepino[4,5-b]indole-5-carboxylate;
1-methylethyl 3-({3,4-difluoro-5-[(2-piperidin-1-ylethyl)oxy]phenyl}carbonyl)-1,1-dimethyl-1,2,3,6-tetrahydroazepino[4,5-b]indole-5-carboxylate;
1-methylethyl 1,1-dimethyl-3-({4-[(2-morpholin-4-ylethyl)oxy]phenyl}carbonyl)-1,2,3,6-tetrahydroazepino[4,5-b]indole-5-carboxylate;
1-methylethyl 1,1-dimethyl-3-({4-[(2-piperidin-1-ylethyl)oxy]phenyl}carbonyl)-1,2,3,6-tetrahydroazepino[4,5-b]indole-5-carboxylate;
1-methylethyl 1,1-dimethyl-3-({3-[(3-morpholin-4-ylpropyl)oxy]phenyl}carbonyl)-1,2,3,6-tetrahydroazepino[4,5-b]indole-5-carboxylate;
1-methylethyl 1,1-dimethyl-3-({3-[(3-piperidin-1-ylpropyl)oxy]phenyl}carbonyl)-1,2,3,6-tetrahydroazepino[4,5-b]indole-5-carboxylate;
1-methylethyl 3-[(4-{[2-(dimethylamino)ethyl]oxy}phenyl)carbonyl]-1,1-dimethyl-1,2,3,6-tetrahydroazepino[4,5-b]indole-5-carboxylate;
1-methylethyl 3-[(3-{[3-(diethylamino)propyl]oxy}phenyl)carbonyl]-1,1-dimethyl-1,2,3,6-tetrahydroazepino[4,5-b]indole-5-carboxylate;
1-methylethyl 3-[(4-{[3-(dimethylamino)propyl]oxy}phenyl)carbonyl]-1,1-dimethyl-1,2,3,6-tetrahydroazepino[4,5-b]indole-5-carboxylate;
1-methylethyl 1,1-dimethyl-3-({4-[(2-pyrrolidin-1-ylethyl)oxy]phenyl}carbonyl)-1,2,3,6-tetrahydroazepino[4,5-b]indole-5-carboxylate;
1-methylethyl 1,1-dimethyl-3-({4-[(3-piperidin-1-ylpropyl)oxy]phenyl}carbonyl)-1,2,3,6-tetrahydroazepino[4,5-b]indole-5-carboxylate; and
1-methylethyl 1,1-dimethyl-3-({4-[(3-morpholin-4-ylpropyl)oxy]phenyl}carbonyl)-1,2,3,6-tetrahydroazepino[4,5-b]indole-5-carboxylate.

60. A compound having formula Ie 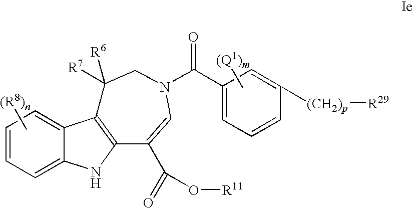
wherein each R⁶ and R⁷ is independently optionally substituted alkyl, n is 0; Each R¹¹ is independently optionally substituted alkyl, p is 1-3; R²⁹ is halogen, optionally substituted alkyl, optionally substituted aryl, optionally substituted aralkyl, optionally substituted heterocyclyl, or optionally substituted heterocyclylalkyl.

61. The compound of claim 60 wherein R²⁹ is dimethylamino, diethylamino, N-ethyl, N-methyl amino, chloro, morpholinyl, piperidinyl, piperazinyl, piperazin-1-ylmethyl, piperazin-1-ylethyl, pyrrolidinyl, morpholinyl, methyloxyphenyl; 4-acetylpiperazin-1-yl; 4-methylsulfonylpiperazin-1-yl; azepanyl; azocan-1-yl; 4-methyl-1,4-diazepan-1-yl; 4-acetyl-1,4-diazepan-1-yl; dimethylethyloxy carbonylpiperazin-1-yl; 4-phenylsulfonyl piperazin-1-yl; 4-fluorophenylsulfonylpiperazin-1-yl; ethylsulfonyl piperazin-1-yl; cyclopropylcarbonyl piperazin-1-yl; 2-methylpropanoyl piperazin-1-yl; phenylcarbonyl piperazin-1-yl; 4-phenylaminocarbonylpiperazin-1-yl; or 4-ethylaminocarbonylpiperazin-1-yl; Q¹ is halogen or optionally substituted alkyl, m is 0-3.

62. The compound of claim 60 selected from the group consisting of:
1-methylethyl 3-({3-[(dimethylamino)methyl]phenyl}carbonyl)-1,1-dimethyl-1,2,3,6-tetrahydroazepino[4,5-b]indole-5-carboxylate;
1-methylethyl 3-{[3-(chloromethyl)phenyl]carbonyl}-1,1-dimethyl-1,2,3,6-tetrahydroazepino[4,5-b]indole-5-carboxylate;
1-methylethyl 3-({3-[(diethylamino)methyl]phenyl}carbonyl)-1,1-dimethyl-1,2,3,6-tetrahydroazepino[4,5-b]indole-5-carboxylate;
1-methylethyl 1,1-dimethyl-3-{[3-(pyrrolidin-1-ylmethyl)phenyl]carbonyl}-1,2,3,6-tetrahydroazepino[4,5-b]indole-5-carboxylate;
1-methylethyl 1,1-dimethyl-3-{[3-(piperidin-1-ylmethyl)phenyl]carbonyl}-1,2,3,6-tetrahydroazepino[4,5-b]indole-5-carboxylate;
1-methylethyl 1,1-dimethyl-3-({3-[(4-methylpiperazin-1-yl)methyl]phenyl}carbonyl)-1,2,3,6-tetrahydroazepino[4,5-b]indole-5-carboxylate;
1-methylethyl 3-({3-[(4-ethylpiperazin-1-yl)methyl]phenyl}carbonyl)-1,1-dimethyl-1,2,3,6-tetrahydroazepino[4,5-b]indole-5-carboxylate;
1-methylethyl 1,1-dimethyl-3-{[3-(morpholin-4-ylmethyl)phenyl]carbonyl}-1,2,3,6-tetrahydroazepino[4,5-b]indole-5-carboxylate;
1-methylethyl 3-({3-[(4-acetylpiperazin-1-yl)methyl]phenyl}carbonyl)-1,1-dimethyl-1,2,3,6-tetrahydroazepino[4,5-b]indole-5-carboxylate;
1-methylethyl 1,1-dimethyl-3-[(3-{[4-(methylsulfonyl)piperazin-1-yl]methyl}phenyl)carbonyl]-1,2,3,6-tetrahydroazepino[4,5-b]indole-5-carboxylate;
1-methylethyl 3-{[3-(azepan-1-ylmethyl)phenyl]carbonyl}-1,1-dimethyl-1,2,3,6-tetrahydroazepino[4,5-b]indole-5-carboxylate;
1-methylethyl 1,1-dimethyl-3-({3-[(4-methyl-1,4-diazepan-1-yl)methyl]phenyl}carbonyl)-1,2,3,6-tetrahydroazepino[4,5-b]indole-5-carboxylate;
1-methylethyl 3-{[2-fluoro-5-(morpholin-4-ylmethyl)phenyl]carbonyl}-1,1-dimethyl-1,2,3,6-tetrahydroazepino[4,5-b]indole-5-carboxylate;
1-methylethyl 3-{[4-fluoro-3-(morpholin-4-ylmethyl)phenyl]carbonyl}-1,1-dimethyl-1,2,3,6-tetrahydroazepino[4,5-b]indole-5-carboxylate;
1-methylethyl 3-{[2-fluoro-5-(piperidin-1-ylmethyl)phenyl]carbonyl}-1,1-dimethyl-1,2,3,6-tetrahydroazepino[4,5-b]indole-5-carboxylate;
1-methylethyl 3-{[4-fluoro-3-(piperidin-1-ylmethyl)phenyl]carbonyl}-1,1-dimethyl-1,2,3,6-tetrahydroazepino[4,5-b]indole-5-carboxylate;
1-methylethyl 3-({3-[(4-{[(1,1-dimethylethyl)oxy]carbonyl}piperazin-1-yl)methyl]phenyl}carbonyl)-1,1-dimethyl-1,2,3,6-tetrahydroazepino[4,5-b]indole-5-carboxylate;
1-methylethyl 1,1-dimethyl-3-[(3-{[4-(phenylsulfonyl)piperazin-1-yl]methyl}phenyl)carbonyl]-1,2,3,6-tetrahydroazepino[4,5-b]indole-5-carboxylate;
1-methylethyl 3-{[3-({4-[(4-fluorophenyl)sulfonyl]piperazin-1-yl}methyl)phenyl]carbonyl}-1,1-dimethyl-1,2,3,6-tetrahydroazepino[4,5-b]indole-5-carboxylate;
1-methylethyl 3-[(3-{[4-(ethylsulfonyl)piperazin-1-yl]methyl}phenyl)carbonyl]-1,1-dimethyl-1,2,3,6-tetrahydroazepino[4,5-b]indole-5-carboxylate;
1-methylethyl 3-[(3-{[4-(cyclopropylcarbonyl)piperazin-1-yl]methyl}phenyl)carbonyl]-1,1-dimethyl-1,2,3,6-tetrahydroazepino[4,5-b]indole-5-carboxylate;
1-methylethyl 1,1-dimethyl-3-[(3-{[4-(2-methylpropanoyl)piperazin-1-yl]methyl}phenyl)carbonyl]-1,2,3,6-tetrahydroazepino[4,5-b]indole-5-carboxylate;
1-methylethyl 1,1-dimethyl-3-[(3-{[4-(phenylcarbonyl)piperazin-1-yl]methyl}phenyl)carbonyl]-1,2,3,6-tetrahydroazepino[4,5-b]indole-5-carboxylate;
1-methylethyl 3-{[3-(azocan-1-ylmethyl)phenyl]carbonyl}-1,1-dimethyl-1,2,3,6-tetrahydroazepino[4,5-b]indole-5-carboxylate;
1-methylethyl 3-({3-[(4-acetyl-1,4-diazepan-1-yl)methyl]phenyl}carbonyl)-1,1-dimethyl-1,2,3,6-tetrahydroazepino[4,5-b]indole-5-carboxylate;
1-methylethyl 1,1-dimethyl-3-{[3-(piperazin-1-ylmethyl)phenyl]carbonyl}-1,2,3,6-tetrahydroazepino[4,5-b]indole-5-carboxylate;
1-methylethyl 1,1-dimethyl-3-{[3-({4-[(phenylamino)carbonyl]piperazin-1-yl}methyl)phenyl]carbonyl}-1,2,3,6-tetrahydroazepino[4,5-b]indole-5-carboxylate; and
1-methylethyl 3-{[3-({4-[(ethylamino)carbonyl]piperazin-1-yl}methyl)phenyl]carbonyl}-1,1-dimethyl-1,2,3,6-tetrahydroazepino[4,5-b]indole-5-carboxylate.

63. A compound having the formula IIa 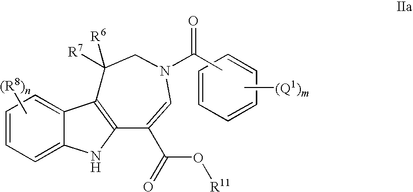
wherein each R⁶ and R⁷ is independently optionally substituted alkyl, n is 0. R¹¹ is independently optionally substituted alkyl, optionally substituted aryl, optionally substituted aralkyl, optionally substituted heterocyclyl, or optionally substituted heterocyclylalkyl.

64.-65. (canceled)

66. The compound of claim 63 selected from the group consisting of:
(2,2-dimethyl-1,3-dioxolan-4-yl)methyl 3-[(3,4-difluorophenyl)carbonyl]-1,1-dimethyl-1,2,3,6-tetrahydroazepino[4,5-b]indole-5-carboxylate;
2,3-dihydroxypropyl 3-[(3,4-difluorophenyl)carbonyl]-1,1-dimethyl-1,2,3,6-tetrahydroazepino[4,5-b]indole-5-carboxylate;
(2R)-2,3-dihydroxypropyl 3-[(3,4-difluorophenyl)carbonyl]-1,1-dimethyl-1,2,3,6-tetrahydroazepino[4,5-b]indole-5-carboxylate;
2-fluoro-1-(fluoromethyl)ethyl 3-[(3,4-difluorophenyl)carbonyl]-1,1-dimethyl-1,2,3,6-tetrahydroazepino[4,5-b]indole-5-carboxylate;
1-methylethyl 3-[(3,4-difluorophenyl)carbonyl]-1,1-dimethyl-8-({[(2-piperidin-1-ylethyl)amino]carbonyl}oxy)-1,2,3,6-tetrahydroazepino[4,5-b]indole-5-carboxylate;
(2S)-2,3-dihydroxypropyl 3-[(3,4-difluorophenyl)carbonyl]-1,1-dimethyl-1,2,3,6-tetrahydroazepino[4,5-b]indole-5-carboxylate;
2-hydroxy-1-methylethyl 3-[(3,4-difluorophenyl)carbonyl]-1,1-dimethyl-1,2,3,6-tetrahydroazepino[4,5-b]indole-5-carboxylate;
2-{[(3,4-difluorophenyl)carbonyl]oxy}-1-methylethyl 3-[(3,4-difluorophenyl)carbonyl]-1,1-dimethyl-1,2,3,6-tetrahydroazepino[4,5-b]indole-5-carboxylate;
2-[(phenylmethyl)oxy]ethyl 3-[(3,4-difluorophenyl)carbonyl]-1,1-dimethyl-1,2,3,6-tetrahydroazepino[4,5-b]indole-5-carboxylate; and
2-hydroxyethyl 3-[(3,4-difluorophenyl)carbonyl]-1,1-dimethyl-1,2,3,6-tetrahydroazepino[4,5-b]indole-5-carboxylate.

67. A compound having formula IIb 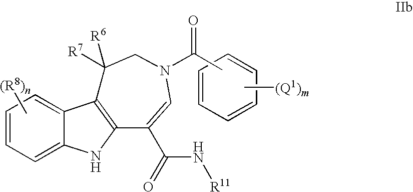
wherein each R⁶ and R⁷ is independently optionally substituted alkyl, n is 0-3; R¹¹ is optionally substituted alkyl or halo; Q¹ is halogen or optionally substituted alkyl; m is 0-3.

68.-69. (canceled)

70. The compound of claim 67 selected from the group consisting of:
N-({3-[(3,4-difluorophenyl)carbonyl]-1,1-dimethyl-1,2,3,4,5,6-hexahydroazepino[4,5-b]indol-5-yl}carbonyl)-beta-alanine;
N-({3-[(3,4-difluorophenyl)carbonyl]-1,1-dimethyl-1,2,3,6-tetrahydroazepino[4,5-b]indol-5-yl}carbonyl)-beta-alanine;
3-[(3,4-difluorophenyl)carbonyl]-N-[(2,3-dihydroxypropyl)oxy]-1,1-dimethyl-1,2,3,6-tetrahydroazepino[4,5-b]indole-5-carboxamide;
3-[(3,4-difluorophenyl)carbonyl]-N-(2,3-dihydroxypropyl)-1,1-dimethyl-1,2,3,6-tetrahydroazepino[4,5-b]indole-5-carboxamide;
3-[(3,4-difluorophenyl)carbonyl]-N-[2-hydroxy-1-(hydroxymethyl)ethyl]-1,1-dimethyl-1,2,3,6-tetrahydroazepino[4,5-b]indole-5-carboxamide; and
3-[(3,4-difluorophenyl)carbonyl]-1,1-dimethyl-N-(1-methylethyl)-1,2,3,4,5,6-hexahydroazepino[4,5-b]indole-5-carboxamide.

71. A compound having the formula III 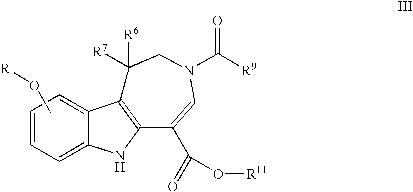
wherein each R⁶ and R⁷ is independently optionally substituted alkyl, R⁹ is optionally substituted aryl; R is independently hydrogen, optionally substituted alkyl, optionally substituted aryl, optionally substituted aralkyl, optionally substituted heterocyclyl or optionally substituted heterocyclylalkyl; R¹¹ is independently optionally substituted alkyl.

72. (canceled)

73. The compound of claim 71 selected from the group consisting of:
1-methylethyl 3-[(3,4-difluorophenyl)carbonyl]-8-[({[2-(dimethylamino)ethyl]amino}carbonyl)oxy]-1,1-dimethyl-1,2,3,6-tetrahydroazepino[4,5-b]indole-5-carboxylate;
1-methylethyl 3-[(3,4-difluorophenyl)carbonyl]-8-({[(1,1-dimethylethyl)oxy]carbonyl}oxy)-1,1-dimethyl-1,2,3,6-tetrahydroazepino[4,5-b]indole-5-carboxylate;
1-methylethyl 8-[({[2-(diethylamino)ethyl]amino}carbonyl)oxy]-3-[(3,4-difluorophenyl)carbonyl]-1,1-dimethyl-1,2,3,6-tetrahydroazepino[4,5-b]indole-5-carboxylate;
1-methylethyl 3-[(3,4-difluorophenyl)carbonyl]-8-{[2-(dimethylamino)ethyl]oxy}-1,1-dimethyl-1,2,3,6-tetrahydroazepino[4,5-b]indole-5-carboxylate;
1-methylethyl 3-[(3,4-difluorophenyl)carbonyl]-8-{[3-(dimethylamino)propyl]oxy}-1,1-dimethyl-1,2,3,6-tetrahydroazepino[4,5-b]indole-5-carboxylate;
1-methylethyl 3-[(3,4-difluorophenyl)carbonyl]-1,1-dimethyl-8-{[(methylamino)carbonyl]oxy}-1,2,3,6-tetrahydroazepino[4,5-b]indole-5-carboxylate;
1-methylethyl 8-{[2-(diethylamino)ethyl]oxy}-3-[(3,4-difluorophenyl)carbonyl]-1,1-dimethyl-1,2,3,6-tetrahydroazepino[4,5-b]indole-5-carboxylate;
1-methylethyl 8-{[3-(diethylamino)propyl]oxy}-3-[(3,4-difluorophenyl)carbonyl]-1,1-dimethyl-1,2,3,6-tetrahydroazepino[4,5-b]indole-5-carboxylate;
1-methylethyl 3-[(3,4-difluorophenyl)carbonyl]-1,1-dimethyl-8-{[2-(methyloxy)ethyl]oxy}-1,2,3,6-tetrahydroazepino[4,5-b]indole-5-carboxylate;
1-methylethyl 8-[({[3-(diethylamino)propyl]amino}carbonyl)oxy]-3-[(3,4-difluorophenyl)carbonyl]-1,1-dimethyl-1,2,3,6-tetrahydroazepino[4,5-b]indole-5-carboxylate;
1-methylethyl 3-[(3,4-difluorophenyl)carbonyl]-1,1-dimethyl-9-[(phenylmethyl)oxy]-1,2,3,6-tetrahydroazepino[4,5-b]indole-5-carboxylate;
1-methylethyl 3-[(3,4-difluorophenyl)carbonyl]-9-hydroxy-1,1-dimethyl-1,2,3,6-tetrahydroazepino[4,5-b]indole-5-carboxylate; and
1-methylethyl 3-[(3,4-difluorophenyl)carbonyl]-1,1-dimethyl-8-({[(2-pyrrolidin-1-ylethyl)amino]carbonyl}oxy)-1,2,3,6-tetrahydroazepino[4,5-b]indole-5-carboxylate.

74. A compound having the formula IV:

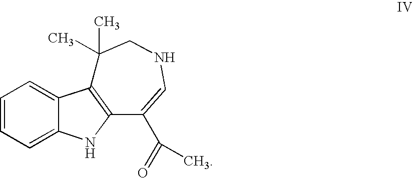

75. A pharmaceutical composition comprising a pharmaceutically acceptable carrier or excipient and a compound of any one of claims 1, 30 or 37 or a pharmaceutically acceptable derivative thereof.

76. (canceled)

77. A method of treating, preventing, inhibiting or ameliorating one or more symptoms of a disease or disorder in which nuclear receptor activity is implicated, comprising administering to a subject in need thereof an effective amount of a compound of any one of claims 1, 30, or 37 or a pharmaceutically acceptable derivative thereof.

78. The method of claim 77 wherein said nuclear receptor is farnesoid X receptor.

79.-80. (canceled)

81. The method of claim 77, wherein the disease or disorder is selected from hyperlipidemia, hypercholesterolemia, hypertriglyceridemia, dyslipidemia, lipodystrophy, atherosclerosis, atherosclerotic disease, atherosclerotic disease events, atherosclerotic cardiovascular disease, Syndrome X, diabetes mellitus, type II diabetes, insulin insensitivity, hyperglycemia, cholestasis and obesity.

82.-86. (canceled)

87. The method of claim 81 wherein the method further comprises administering at least one additional active agent selected from phenylpropanolamine, phentermine, diethylpropion, mazindol, fenfluramine, dexfenfluramine, phentiramine, β₃ adrenoceptor agonist agents, sibutramine, gastrointestinal lipase inhibitors, LXR α agonists, partial agonists or antagonists, LXR β agonists, partial agonists or antagonists, neuropeptide Y, enterostatin, cholecytokinin, bombesin, amylin, histamine H₃ receptor agonists or antagonists, dopamine D₂ receptor agonists or antagonists, melanocyte stimulating hormone, corticotrophin releasing factor, leptins, galanin or gamma amino butyric acid (GABA) simultaneously with, prior to, or after administration of the compound.

88. The method of claim 77 wherein the disease or disorder is selected from the group consisting of hyperlipidemia, hypertriglyceridemia, hypercholesterolemia and dyslipidemia.

89. The method of claim 88 wherein the method further comprises administering at least one additional active agent selected from antihyperlipidemic agents, plasma HDL-raising agents, antihypercholesterolemic agents, cholesterol biosynthesis inhibitors, HMG CoA reductase inhibitors, acyl-coenzyme A:cholesterol acytransferase (ACAT) inhibitors, probucol, raloxifene, nicotinic acid, niacinamide, cholesterol absorption inhibitors, bile acid sequestrants, low density lipoprotein receptor inducers, clofibrate, fenofibrate, benzofibrate, cipofibrate, gemfibrizol, vitamin B₆, vitamin B₁₂, anti-oxidant vitamins, β-blockers, anti-diabetes agents, angiotensin II antagonists, angiotensin converting enzyme inhibitors, platelet aggregation inhibitors, fibrinogen receptor antagonists, aspirin, LXR α agonists, partial agonists or antagonists, LXR β agonists, partial agonists or antagonists, or fibric acid derivatives, simultaneously with, prior to, or after administration of the compound of claim 1.

90. The method of claim 77, wherein the disease or disorder is selected from the group consisting of atherosclerosis, atherosclerotic disease, atherosclerotic disease events and atherosclerotic cardiovascular disease.

91. The method of claim 77, wherein the disease or disorder is selected from the group consisting of Syndrome X, diabetes mellitus, type II diabetes, insulin insensitivity and hyperglycemia.

92. The method of claim 91, wherein the method further comprises administering at least one additional active agent selected from sulfonylureas, biguanides, thiazolidinediones; activators of PPARα PPARβ and PPARγ; agonists, LXR α agonists, partial agonists or antagonists, LXR β agonists, partial agonists or antagonists, dehydroepiandrosterone; antiglucocorticoids; TNF α inhibitors; α-glucosidase inhibitors, pramlintide, amylin, insulin or insulin, simultaneously with, prior to, or after administration of the compound of claim 1 or a pharmaceutically acceptable derivative thereof.

93.-97. (canceled)

98. The compound of claim 1 selected from the compounds of Table 1.
